(19) United States
(12) Patent Application Publication
(10) Pub. No.: US 2024/0366623 A1
(43) Pub. Date: Nov. 7, 2024

(54) COMPOSITIONS FOR PREVENTING OR TREATING CHARCOT-MARIE-TOOTH DISEASE (CMT)

(71) Applicant: Chong Kun Dang Pharmaceutical Corp., Seoul (KR)

(72) Inventors: Nina Ha, Gyeonggi-do (KR); Daekwon Bae, Gyeonggi-do (KR); Min Cheol Kim, Gyeonggi-do (KR); Ju Young Song, Gyeonggi-do (KR); Dong-hyeon Suh, Gyeonggi-do (KR); Suk Jin Lee, Gyeonggi-do (KR); Jiyeon Baek, Gyeonggi-do (KR); Jin Sol Park, Gyeonggi-do (KR)

(21) Appl. No.: 18/686,193

(22) PCT Filed: Aug. 30, 2022
(86) PCT No.: PCT/IB2022/058096

§ 371 Date: Feb. 23, 2024

(30) Foreign Application Priority Data
Aug. 31, 2021 (KR) 10-2021-0115983

(51) Int. Cl. A61K 31/541; A61K 31/5355

(52) U.S. Cl. CPC A61K 31/541; A61K 31/5355

(57) ABSTRACT

The present invention relates to a pharmaceutical composition for preventing or treating Charcot-Marie-Tooth disease associated with a peripheral nervous system, comprising a compound represented by formula I, optical isomers thereof or pharmaceutically acceptable salts thereof as an active ingredient, a method for preventing or treating Charcot-Marie-Tooth disease associated with the peripheral nervous system using the compound, a use of the compound for preventing or treating Charcot-Marie-Tooth disease associated with the peripheral nervous system, and a use of the compound in preparing a medicament for preventing or treating Charcot-Marie-Tooth disease associated with the peripheral nervous system.

### TECHNICAL FIELD

The present disclosure relates to a pharmaceutical composition for preventing or treating Charcot-Marie-Tooth disease associated with a peripheral nervous system, comprising a compound represented by formula I, optical isomers thereof or pharmaceutically acceptable salts thereof as an active ingredient, a method for preventing or treating Charcot-Marie-Tooth disease associated with the peripheral nervous system using the compound, a use of the compound for preventing or treating Charcot-Marie-Tooth disease associated with the peripheral nervous system, and a use of the compound in preparing a medicament for preventing or treating Charcot-Marie-Tooth disease associated with the peripheral nervous system.

### BACKGROUND

Charcot-Marie-Tooth (CMT, hereditary motor and sensory neuropathy: HMSN) disease is the most common type of hereditary peripheral nerve disorder caused by a mutation of proteins that constitute nerves. More than 1,000 mutations have been identified from about 90 genes so far (Timmerman et al., (2014) Genes 5:13-32). Upon the development of Charcot-Marie-Tooth disease, progressive degeneration of peripheral nerves leads to atrophy of muscles affected by neural distribution, and thus patients show gradual atrophy in their muscles of hands and feet as well a symptom of deformed hands and feet. The CMT is genetically and clinically very diverse and complicated, and it is known that symptoms thereof vary ranging from a close-to-normal state to a wheelchair-bound state depending on mutation types. The CMT emerges mainly in teen years and occurs to one for every 2,500 people (Krajewski et al., (2000) Brain 123:1516).

The CMT belongs to rare diseases such as hereditary peripheral nerve disorder. However, a prevalence rate thereof amounts to one for every 2,500 people. There are about 20,000 patients in South Korea and 2,800,000 ones worldwide. Until now, a therapy for the CMT is limited only to rehabilitation, aids, pain control, surgical therapy, etc., but a successful therapeutic agent has not been developed yet. Thus, there is a great need for developing a therapeutic agent for the CMT.

For example, concerning the CMT, which is the most common type of hereditary motor and sensory neuropathy, a large-scale clinical trial was conducted on ascorbic acid, which had been proven as an essential material for myelination in the peripheral nervous system through an experiment on culturing lemmocytes and dorsal root ganglion cells together, but such trial was failed in proving efficacy (Pareyson et al., (2011) 10(4):3205).

### RELATED ART REFERENCE

### Patent Documents

(Patent Document 1) Korean Unexamined Patent Application Publication No. 2017-0017792

### Non-Patent Documents

(non-Patent Document 1) Krajewski et al., (2000) Brain 123:1516
(non-Patent Document 2) Pareyson et al., (2011) 10(4):3205

### DETAILED DESCRIPTION OF THE INVENTION

### Technical Problem

The present disclosure provides a pharmaceutical composition for preventing or treating Charcot-Marie-Tooth disease associated with a peripheral nervous system, including a compound represented by formula I, optical isomers thereof or pharmaceutically acceptable salts thereof as an active ingredient.

The present disclosure provides a method for preventing or treating Charcot-Marie-Tooth disease associated with the peripheral nervous system, including administering a compound represented by above formula I, optical isomers thereof or pharmaceutically acceptable salts thereof into an individual.

The present disclosure provides a use of a compound represented by the above formula I, optical isomers thereof or pharmaceutically acceptable salts thereof for preventing or treating Charcot-Marie-Tooth disease associated with the peripheral nervous system.

The present disclosure provides a use of a compound represented by the above formula I, optical isomers thereof or pharmaceutically acceptable salts thereof in preparing a medicament for preventing or treating Charcot-Marie-Tooth disease associated with the peripheral nervous system.

### Technical Solution

This is described in detail as follows. Meanwhile, each description and embodiment disclosed in the present invention may be also applied to other descriptions and embodiments thereof, respectively. In other words, all the combinations of various elements disclosed in the present invention fall within the scope of the present invention. Also, it cannot be seen that the scope of the present invention is limited to the specific description described below.

The present disclosure provides a pharmaceutical composition for preventing or treating Charcot-Marie-Tooth (CMT) disease associated with the peripheral nervous systems (PNS), including a compound represented by formula I below, optical isomers thereof or pharmaceutically acceptable salts thereof as an active ingredient.

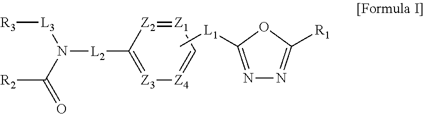

In Formula I,
wherein L₁, L₂ or L₃ are each independently a bond or —(C₁-C₂ alkylene)-;
R₁ is —CX₂H or —CX₃;

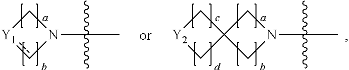

R₂ is —N^{A}R^{B}, —OR^{C},
{wherein at least one H of

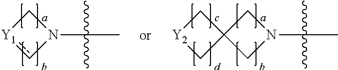

may be substituted with —X, —OH, —O(C₁-C₄ alkyl), —NR^{D}R^{E}, —(C₁-C₄ alkyl), —CF₃, —CF₂H, —CN, -aryl, -heteroaryl, —(C₁-C₄ alkyl)-aryl or —(C₁-C₄ alkyl)-heteroaryl, [wherein at least one H of the -aryl, -heteroaryl, —(C₁-C₄ alkyl)-aryl or —(C₁-C₄ alkyl)-heteroaryl may be substituted with —X, —OH, —CF₃ or —CF₂H]};
R₃ is —H, —(C₁-C₄ alkyl), —(C₁-C₄ alkyl)-O(C₁-C₄ alkyl), —(C₁-C₄ alkyl)-C(═O)—O(C₁-C₄ alkyl), —(C₃-C₇ cycloalkyl), —(C₂-C₆ cycloheteroalkyl), -aryl, -heteroaryl, -adamantyl,

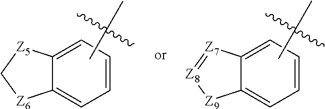

{wherein, at least one H of —(C₁-C₄ alkyl) may be substituted with —X or —OH,
at least one H of -aryl or -heteroaryl each independently may be substituted with —X, —OH, —O(C₁-C₄ alkyl), —OCF₃, —O-aryl, —NR^{D}R^{E}, —(C₁-C₄ alkyl), —CF₃, —CF₂H, —C(═O)—(C₁-C₄ alkyl), —C(═O)—O(C₁-C₄ alkyl), —C(═O)—NR^{D}R^{E}, —S(═O)₂—(C₁-C₄ alkyl), aryl, heteroaryl,

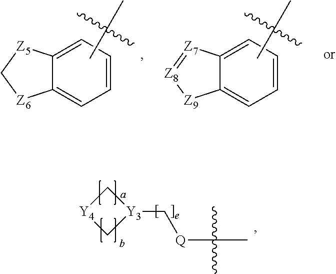

[wherein, at least one H of

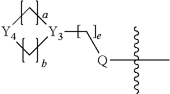

may be substituted with —X, —(C₁-C₄ alkyl), —NR^{D}R^{E}, —CF₃ or —CF₂H], at least one H of —(C₃-C₇ cycloalkyl), —(C₂-C₆ cycloheteroalkyl), adamantyl,

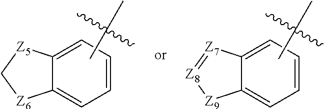

may be each independently substituted with —X, —OH or —(C₁-C₄ alkyl)};
Y₁, Y₂ and Y₄ are each independently —CH₂—, —NR^{F}—, —O—, —C(═O)— or —S(═O)₂—;
Y₃ is —CH— or —N—;
Z₁ to Z₄ are each independently N or CR^{Z}, {wherein at least three of Z₁ to Z₄ may not be simultaneously N, and R^{Z} is —H, —X or —O(C₁-C₄ alkyl)};
Z₅ and Z₆ are each independently —CH₂— or —O—;
Z₇ and Z₈ are each independently ═CH— or ═N—;
Z₉ is —NR^{G}— or —S—;
R^{A} and R^{B} are each independently —H, —(C₁-C₄ alkyl), —(C₁-C₄ alkyl)-OH, —(C₁-C₄ alkyl)-NR^{D}R^{E}, -aryl, —(C₁-C₄ alkyl)-aryl, -heteroaryl, —(C₁-C₄ aryl)-heteroaryl, —(C₃-C₇ cycloalkyl), —(C₂-C₆ heterocycloalkyl) or

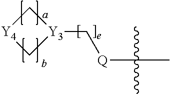

{wherein, at least one H of the —(C₁-C₄ alkyl), —(C₁-C₄ alkyl)-OH or —(C₁-C₄ alkyl)-NR^{D}R^{E} may be substituted with —X,
at least one H of the -aryl, —(C₁-C₄ alkyl)-aryl, -heteroaryl, —(C₁-C₄ alkyl)-heteroaryl, —(C₃-C₇ cycloalkyl) or —(C₂-C₆ heterocycloalkyl) may be substituted with —X, —OH, —O(C₁-C₄ alkyl), —(C₁-C₄ alkyl), —CF₃, —CF₂H or —CN,
   at least one H of

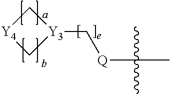

may be substituted with —X, —OH, —O(C₁-C₄ alkyl), —(C₁-C₄ alkyl), —CF₃, —CF₂H, —CN, —(C₂-C₆ heterocycloalkyl), -aryl, —(C₁-C₄ alkyl)-aryl, -heteroaryl or -heteroaryl-(C₁-C₄ alkyl)};
R^{C} is —(C₁-C₄ alkyl), -aryl, —(C₁-C₄ alkyl)-aryl, -heteroaryl or —(C₁-C₄ alkyl)-heteroaryl {wherein, at least one H of —(C₁-C₄ alkyl) may be substituted with —X or —OH, at least one H of -aryl, —(C₁-C₄ alkyl)-aryl, -heteroaryl or —(C₁-C₄ alkyl)-heteroaryl may be substituted with —X, —OH, —CF₃ or —CF₂H};
R^{D} and R^{E} are each independently —H, —(C₁-C₄ alkyl), -aryl or —(C₁-C₄ alkyl)-aryl {wherein, at least one H of —(C₁-C₄ alkyl) may be substituted with —X or —OH, at least one H of -aryl or —(C₁-C₄ alkyl)-aryl may be substituted with —X, —OH, —CF₃ or —CF₂H};
R^{F} is —H, —(C₁-C₆ alkyl), —(C₁-C₄ alkyl)-OH, —(C₁-C₄ alkyl)-O—(C₁-C₄ alkyl), —C(═O)—(C₁-C₄ alkyl), —C(═O)—O(C₁-C₄ alkyl), —(C₁-C₄ alkyl)-C(═O)—O(C₁-C₄ alkyl), —(C₁-C₄ alkyl)-NR^{D}R^{E}, —S(═O)₂—(C₁-C₄ alkyl), -aryl, —(C₁-C₄ alkyl)-aryl, —(C₂-C₄ alkenyl)-aryl, -heteroaryl, —(C₁-C₄ alkyl)-heteroaryl, —C(═O)—(C₃-C₇ cycloalkyl), —(C₂-C₆ heterocycloalkyl) or —(C₁-C₄ alkyl)-C(═O)—(C₂-C₆ heterocycloalkyl)
{wherein, at least one H of —(C₁-C₄ alkyl), —(C₁-C₄ alkyl)-OH, —(C₁-C₄ alkyl)-O—(C₁-C₄ alkyl), —C(═O)—(C₁-C₄ alkyl), —C(═O)—O(C₁-C₄ alkyl), —(C₁-C₄ alkyl)-C(═O)—O(C₁-C₄ alkyl), —(C₁-C₄ alkyl)-NR^{D}R^{E} or —S(═O)₂—(C₁-C₄ alkyl) may be substituted with —X, at least one H of -aryl, —(C₁-C₄alkyl)-aryl, —(C₂-C₄ alkenyl)-aryl, -heteroaryl, —(C₁-C₄ alkyl)-heteroaryl, —C(═O)—(C₃-C₇ cycloalkyl), —C₂-C₆ heterocycloalkyl or —(C₁-C₄alkyl)-C(═O)—(C₂-C₆heterocycloalkyl) may be substituted with —X, —OH, —CF₃ or —CF₂H};
R^{G} is —H or —(C₁-C₄ alkyl);
Q is —O— or a bond;
[symbol] is a single bond or double bond {provided that, [symbol] is a double bond, Y₁ is ═CH—};
a to e are each independently an integer of 0, 1, 2, 3 or 4 {provided that, a and b may not be simultaneously 0, and c and d may not be simultaneously 0};
X is each independently F, Cl, Br or I.

In the pharmaceutical composition according to the present disclosure,
in the compound represented by formula I, wherein
L₁, L₂ or L₃ are each independently a bond or —(C₁-C₂alkylene)-;
R₁ is —CX₂H or —CX₃;
R₂ is —N^{A}R^{B}, —OR^{C},

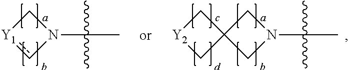

{wherein at least one of H of

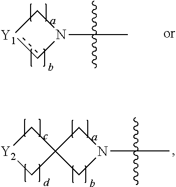

may be substituted with —X, —OH, —NR^{D}R^{E}, —(C₁-C₄ alkyl)};
R₃ is —(C₁-C₄ alkyl), —(C₃-C₇ cycloalkyl), -aryl, -heteroaryl, -adamantyl,

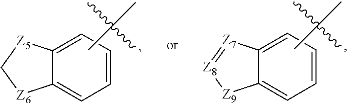

{wherein at least one H of -aryl or -heteroaryl may be each independently substituted with —X, —O(C₁-C₄alkyl), —OCF₃, —O-aryl, —NR^{D}R^{E}, —(C₁-C₄ alkyl), —CF₃, —S(═O)₂—(C₁-C₄alkyl), -aryl, -heteroaryl,

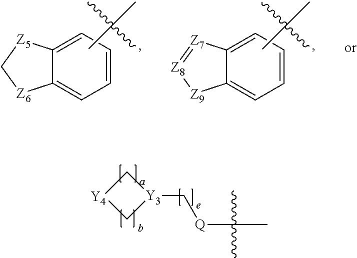

[wherein at least one H of

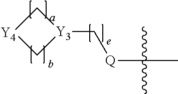

may be substituted with —NR^{D}R^{E} or —(C₁-C₄ alkyl)],
at least one H of

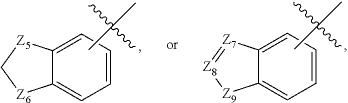

maybe each independently substituted with —(C₁-C₄ alkyl)}; Y₁, Y₂ and Y₄ are each independently —CH₂—, —NR^{F}—, —O—, —C(═O)— or —S(═O)₂—;
Y₃ is —CH— or —N—;
Z₁ to Z₄ is each independently N or CR^{Z} {wherein at least three of Z₁ to Z₄ may not be simultaneously N, and R^{Z} is —H, —X or —O(C₁-C₄ alkyl)};
Z₅ and Z₆ are each independently —CH₂— or —O—;
Z₇ and Z₈ are each independently ═CH— or ═N—;
Z₉ is —NR^{G}— or —S—;
R^{A} and R^{B} are each independently —H, —(C₁-C₄ alkyl), —(C₁-C₄ alkyl)-OH, —(C₁-C₄ alkyl)-NR^{D}R^{E}, -aryl, —(C₁-C₄ alkyl)-aryl, —(C₃-C₇ cycloalkyl) or

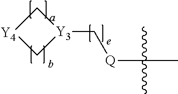

{wherein at least one H of

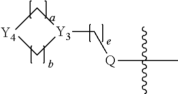

may be substituted with —X, —(C₁-C₄alkyl), —CF₃, —(C₂-C₆ heterocycloalkyl), —(C₁-C₄ alkyl)-aryl, -heteroaryl or heteroaryl-(C₁-C₄ alkyl)};
R^{C} is —(C₁-C₄ alkyl) or -aryl;
R^{D} and R^{E} are each independently —H, —(C₁-C₄alkyl) or —(C₁-C₄ alkyl)-aryl;
R^{F} is —H, —(C₁-C₆alkyl), —(C₁-C₄alkyl)-OH, —(C₁-C₄ alkyl)-O—(C₁-C₄ alkyl), —C(═O)—(C₁-C₄alkyl), —C(═O)—O(C₁-C₄ alkyl), —(C₁-C₄ alkyl)-C(═O)—O(C₁-C₄alkyl), —(C₁-C₄alkyl)-NR^{D}R^{E}, —S(═O)₂—(C₁-C₄ alkyl), -aryl, —(C₁-C₄ alkyl)-aryl, —(C₂-C₄ alkenyl)-aryl, -heteroaryl, —(C₁-C₄ alkyl)-heteroaryl, —C(═O)—(C₃-C₇ cycloalkyl), —(C₂-C₆ heterocycloalkyl) or —(C₁-C₄ alkyl)-C(═O)—(C₂-C₆ heterocycloalkyl)
{wherein at least one H of —(C₁-C₄alkyl) or —C(═O)—O(C₁-C₄alkyl) may be substituted with —X,
at least one H of -aryl may be substituted with —X};
R^{G} is —(C₁-C₄ alkyl);
Q is —O— or a bond;
[symbol] is a single bond or a double bond {provided that [symbol] is a double bond, Y₁ is —CH—};
a to e are each independently an integer of 0, 1, 2, 3 or 4 {provided that a and b may not be simultaneously 0, and c and d may not be simultaneously 0};
X is each independently F, Cl, Br or I.

In the pharmaceutical composition according to the present disclosure, the formula I is the compound represented by formula Ia:

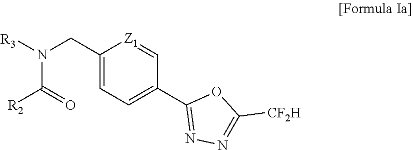

in Formula Ia,

R₂ is

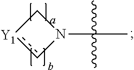

R₃ is -aryl {wherein at least one H of -aryl may be each independently substituted with —X};
Y₁ is —O— or —S(═O)₂—;
Z₁ is N or CR^{Z} {wherein R^{Z} is —X};
a and b are each independently an integer of 0, 1, 2, 3 or 4 {wherein a and b may not be simultaneously 0};
X is each independently F, Cl, Br or I.

In the pharmaceutical composition according to the present disclosure, in the compound represented by formula I,

R₂ is

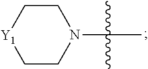

R₃ is -phenyl {wherein at least one H of -phenyl each independently may be substituted with —F or —C₁};
Y₁ is —O— or —S(═O)₂—;
Z₁ is N or CF.

In the pharmaceutical composition according to the present disclosure, the compounds represented by formula I may be shown in Table A below:

TABLE A

| Com- | |
|---|---|
| pound | Structure |
| 1 | 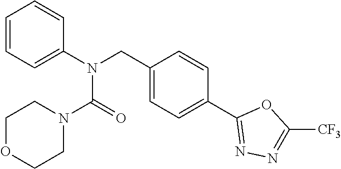 [SMILES: O=C(N1CCOCC1)N(Cc1ccc(-c2nnc(C(F)(F)F)o2)cc1)c1ccccc1] |
| 2 | 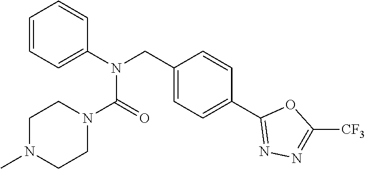 [SMILES: CN1CCN(C(=O)N(Cc2ccc(-c3nnc(C(F)(F)F)o3)cc2)c2ccccc2)CC1] |
| 3 | 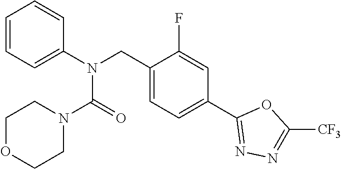 [SMILES: O=C(N1CCOCC1)N(Cc1ccc(-c2nnc(C(F)(F)F)o2)cc1F)c1ccccc1] |
| 4 | 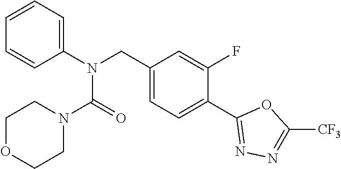 [SMILES: O=C(N1CCOCC1)N(Cc1ccc(-c2nnc(C(F)(F)F)o2)c(F)c1)c1ccccc1] |
| 5 | 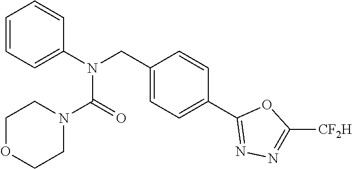 [SMILES: O=C(N1CCOCC1)N(Cc1ccc(-c2nnc(C(F)F)o2)cc1)c1ccccc1] |
| 6 | 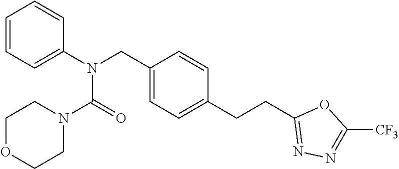 [SMILES: O=C(N1CCOCC1)N(Cc1ccc(CCc2nnc(C(F)(F)F)o2)cc1)c1ccccc1] |
| 7 | 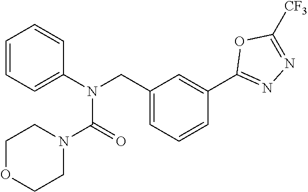 [SMILES: O=C(N1CCOCC1)N(Cc1cccc(-c2nnc(C(F)(F)F)o2)c1)c1ccccc1] |
| 8 | 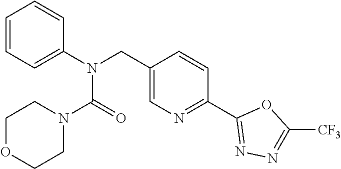 [SMILES: O=C(N1CCOCC1)N(Cc1ccc(-c2nnc(C(F)(F)F)o2)nc1)c1ccccc1] |
| 9 | 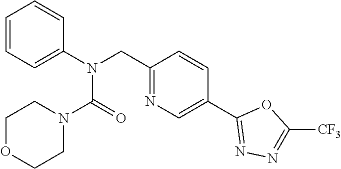 [SMILES: O=C(N1CCOCC1)N(Cc1ccc(-c2nnc(C(F)(F)F)o2)cn1)c1ccccc1] |
| 10 | 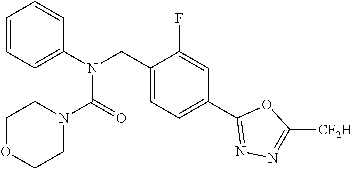 [SMILES: O=C(N1CCOCC1)N(Cc1ccc(-c2nnc(C(F)F)o2)cc1F)c1ccccc1] |
| 11 | 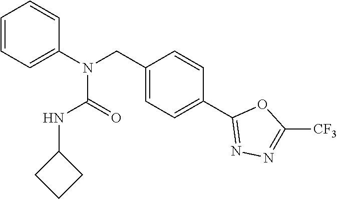 [SMILES: O=C(NC1CCC1)N(Cc1ccc(-c2nnc(C(F)(F)F)o2)cc1)c1ccccc1] |
| 12 | 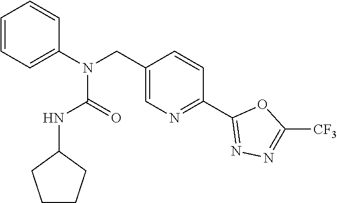 [SMILES: O=C(NC1CCCC1)N(Cc1ccc(-c2nnc(C(F)(F)F)o2)nc1)c1ccccc1] |
| 13 | 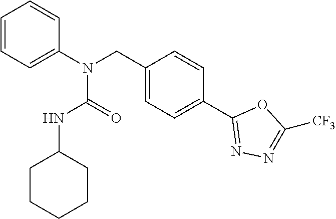 [SMILES: O=C(NC1CCCCC1)N(Cc1ccc(-c2nnc(C(F)(F)F)o2)cc1)c1ccccc1] |
| 14 | 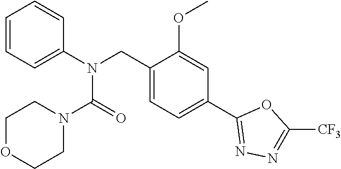 [SMILES: COc1cc(-c2nnc(C(F)(F)F)o2)ccc1CN(C(=O)N1CCOCC1)c1ccccc1] |
| 15 | 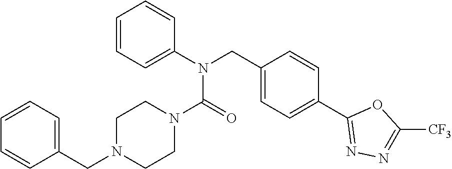 [SMILES: O=C(N1CCN(Cc2ccccc2)CC1)N(Cc1ccc(-c2nnc(C(F)(F)F)o2)cc1)c1ccccc1] |
| 16 | 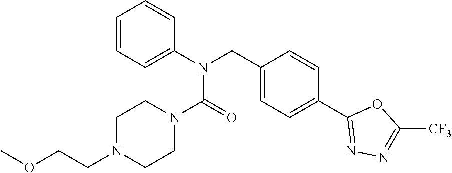 [SMILES: COCCN1CCN(C(=O)N(Cc2ccc(-c3nnc(C(F)(F)F)o3)cc2)c2ccccc2)CC1] |
| 17 | 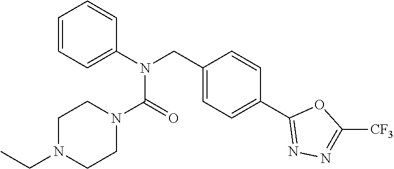 [SMILES: CCN1CCN(C(=O)N(Cc2ccc(-c3nnc(C(F)(F)F)o3)cc2)c2ccccc2)CC1] |
| 18 | 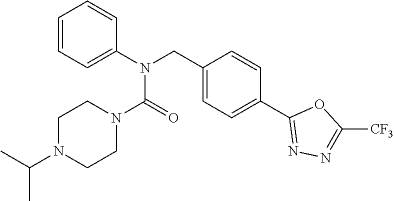 [SMILES: CC(C)N1CCN(C(=O)N(Cc2ccc(-c3nnc(C(F)(F)F)o3)cc2)c2ccccc2)CC1] |
| 19 | 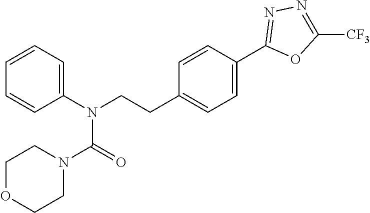 [SMILES: O=C(N1CCOCC1)N(CCc1ccc(-c2nnc(C(F)(F)F)o2)cc1)c1ccccc1] |
| 20 | 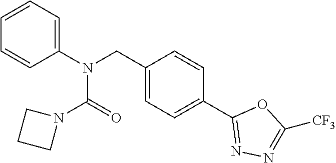 [SMILES: O=C(N1CCC1)N(Cc1ccc(-c2nnc(C(F)(F)F)o2)cc1)c1ccccc1] |
| 21 | 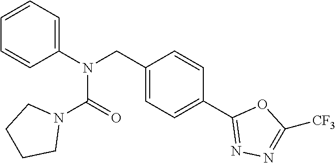 [SMILES: O=C(N1CCCC1)N(Cc1ccc(-c2nnc(C(F)(F)F)o2)cc1)c1ccccc1] |
| 22 | 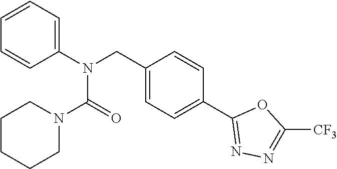 [SMILES: O=C(N1CCCCC1)N(Cc1ccc(-c2nnc(C(F)(F)F)o2)cc1)c1ccccc1] |
| 23 | 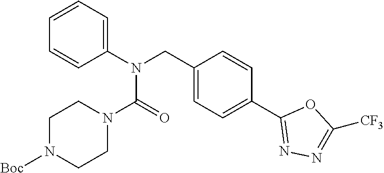 [SMILES: CC(C)(C)OC(=O)N1CCN(C(=O)N(Cc2ccc(-c3nnc(C(F)(F)F)o3)cc2)c2ccccc2)CC1] |
| 24 | 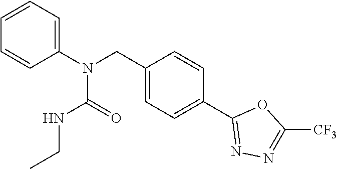 [SMILES: CCNC(=O)N(Cc1ccc(-c2nnc(C(F)(F)F)o2)cc1)c1ccccc1] |
| 25 | 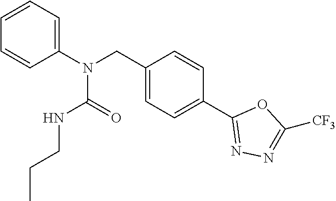 [SMILES: CCCNC(=O)N(Cc1ccc(-c2nnc(C(F)(F)F)o2)cc1)c1ccccc1] |
| 26 | 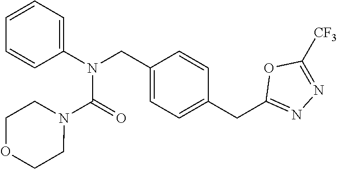 [SMILES: O=C(N1CCOCC1)N(Cc1ccc(Cc2nnc(C(F)(F)F)o2)cc1)c1ccccc1] |
| 27 | 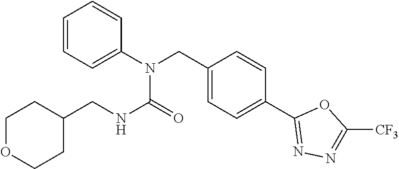 [SMILES: O=C(NCC1CCOCC1)N(Cc1ccc(-c2nnc(C(F)(F)F)o2)cc1)c1ccccc1] |
| 28 | 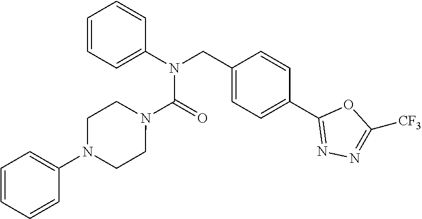 [SMILES: O=C(N1CCN(c2ccccc2)CC1)N(Cc1ccc(-c2nnc(C(F)(F)F)o2)cc1)c1ccccc1] |
| 29 | 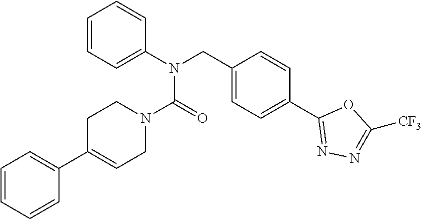 [SMILES: O=C(N1CC=C(c2ccccc2)CC1)N(Cc1ccc(-c2nnc(C(F)(F)F)o2)cc1)c1ccccc1] |
| 30 | 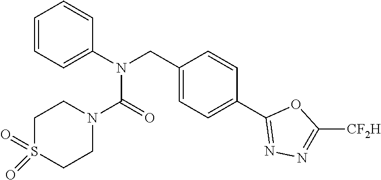 [SMILES: O=C(N1CCS(=O)(=O)CC1)N(Cc1ccc(-c2nnc(C(F)F)o2)cc1)c1ccccc1] |
| 31 | 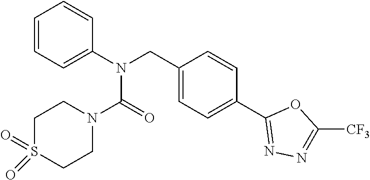 [SMILES: O=C(N1CCS(=O)(=O)CC1)N(Cc1ccc(-c2nnc(C(F)(F)F)o2)cc1)c1ccccc1] |
| 32 | 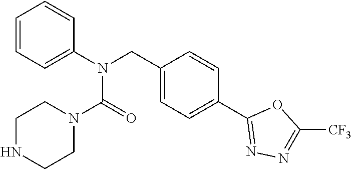 [SMILES: O=C(N1CCNCC1)N(Cc1ccc(-c2nnc(C(F)(F)F)o2)cc1)c1ccccc1] |
| 33 | 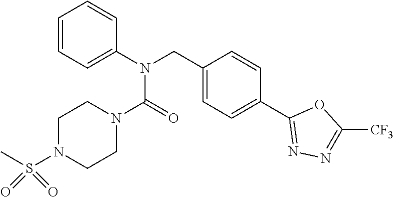 [SMILES: CS(=O)(=O)N1CCN(C(=O)N(Cc2ccc(-c3nnc(C(F)(F)F)o3)cc2)c2ccccc2)CC1] |
| 34 | 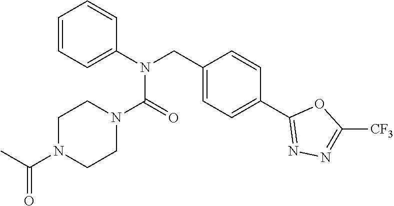 [SMILES: CC(=O)N1CCN(C(=O)N(Cc2ccc(-c3nnc(C(F)(F)F)o3)cc2)c2ccccc2)CC1] |
| 35 | 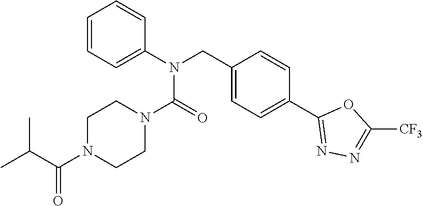 [SMILES: CC(C)C(=O)N1CCN(C(=O)N(Cc2ccc(-c3nnc(C(F)(F)F)o3)cc2)c2ccccc2)CC1] |
| 36 | 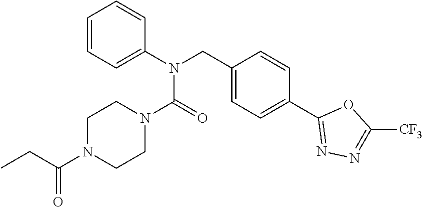 [SMILES: CCC(=O)N1CCN(C(=O)N(Cc2ccc(-c3nnc(C(F)(F)F)o3)cc2)c2ccccc2)CC1] |
| 37 | 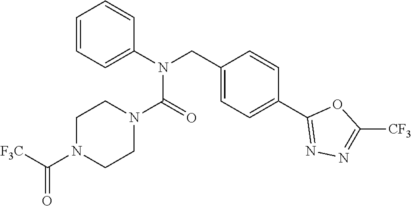 [SMILES: O=C(N1CCN(C(=O)C(F)(F)F)CC1)N(Cc1ccc(-c2nnc(C(F)(F)F)o2)cc1)c1ccccc1] |
| 38 | 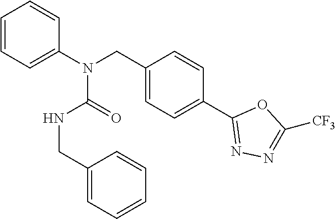 [SMILES: O=C(NCc1ccccc1)N(Cc1ccc(-c2nnc(C(F)(F)F)o2)cc1)c1ccccc1] |
| 39 | 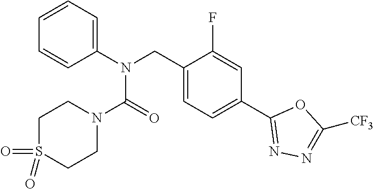 [SMILES: O=C(N1CCS(=O)(=O)CC1)N(Cc1ccc(-c2nnc(C(F)(F)F)o2)cc1F)c1ccccc1] |
| 40 | 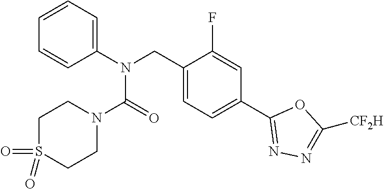 [SMILES: O=C(N1CCS(=O)(=O)CC1)N(Cc1ccc(-c2nnc(C(F)F)o2)cc1F)c1ccccc1] |
| 41 | 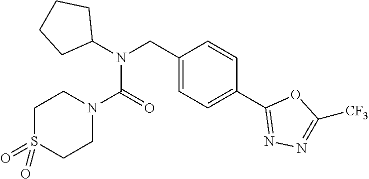 [SMILES: O=C(N1CCS(=O)(=O)CC1)N(Cc1ccc(-c2nnc(C(F)(F)F)o2)cc1)C1CCCC1] |
| 42 | 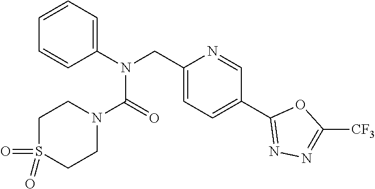 [SMILES: O=C(N1CCS(=O)(=O)CC1)N(Cc1ccc(-c2nnc(C(F)(F)F)o2)cn1)c1ccccc1] |
| 43 | 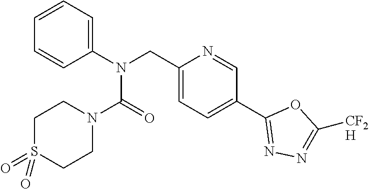 [SMILES: O=C(N1CCS(=O)(=O)CC1)N(Cc1ccc(-c2nnc(C(F)F)o2)cn1)c1ccccc1] |
| 44 | 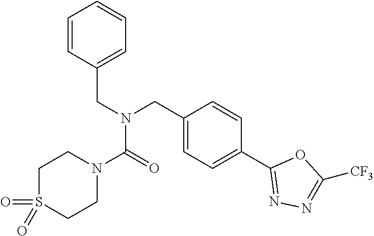 [SMILES: O=C(N1CCS(=O)(=O)CC1)N(Cc1ccccc1)Cc1ccc(-c2nnc(C(F)(F)F)o2)cc1] |
| 45 | 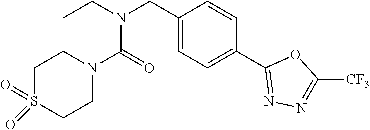 [SMILES: CCN(Cc1ccc(-c2nnc(C(F)(F)F)o2)cc1)C(=O)N1CCS(=O)(=O)CC1] |
| 46 | 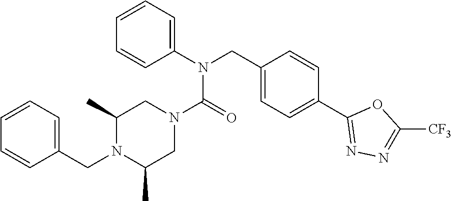 [SMILES: C[C@@H]1CN(C(=O)N(Cc2ccc(-c3nnc(C(F)(F)F)o3)cc2)c2ccccc2)C[C@H](C)N1Cc1ccccc1] |
| 47 | 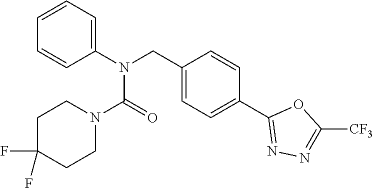 [SMILES: O=C(N1CCC(F)(F)CC1)N(Cc1ccc(-c2nnc(C(F)(F)F)o2)cc1)c1ccccc1] |
| 48 | 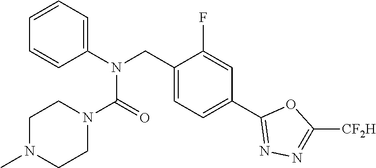 [SMILES: CN1CCN(C(=O)N(Cc2ccc(-c3nnc(C(F)F)o3)cc2F)c2ccccc2)CC1] |
| 49 | 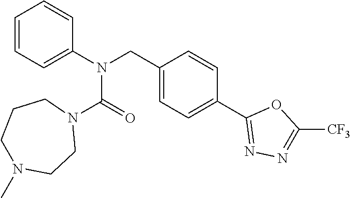 [SMILES: CN1CCCN(C(=O)N(Cc2ccc(-c3nnc(C(F)(F)F)o3)cc2)c2ccccc2)CC1] |
| 50 | 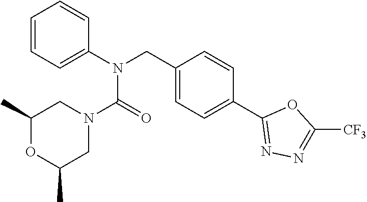 [SMILES: C[C@@H]1CN(C(=O)N(Cc2ccc(-c3nnc(C(F)(F)F)o3)cc2)c2ccccc2)C[C@H](C)O1] |
| 51 | 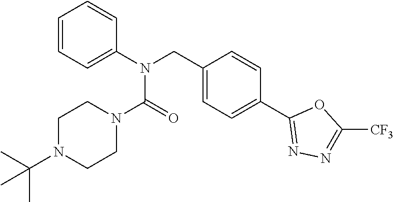 [SMILES: CC(C)(C)N1CCN(C(=O)N(Cc2ccc(-c3nnc(C(F)(F)F)o3)cc2)c2ccccc2)CC1] |
| 52 | 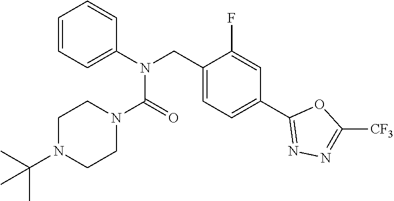 [SMILES: CC(C)(C)N1CCN(C(=O)N(Cc2ccc(-c3nnc(C(F)(F)F)o3)cc2F)c2ccccc2)CC1] |
| 53 | 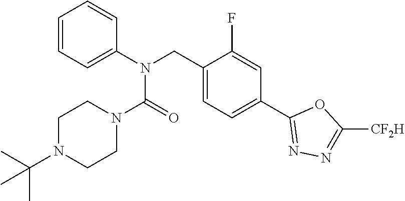 [SMILES: CC(C)(C)N1CCN(C(=O)N(Cc2ccc(-c3nnc(C(F)F)o3)cc2F)c2ccccc2)CC1] |
| 54 | 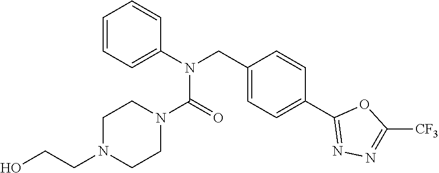 [SMILES: O=C(N1CCN(CCO)CC1)N(Cc1ccc(-c2nnc(C(F)(F)F)o2)cc1)c1ccccc1] |
| 55 | 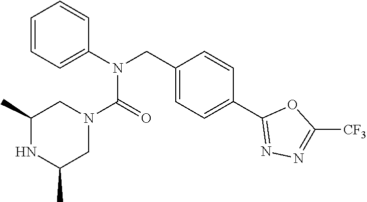 [SMILES: C[C@@H]1CN(C(=O)N(Cc2ccc(-c3nnc(C(F)(F)F)o3)cc2)c2ccccc2)C[C@H](C)N1] |
| 56 | 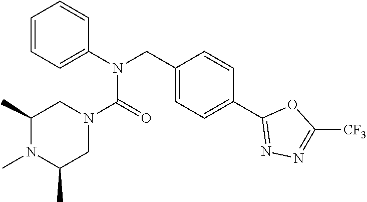 [SMILES: C[C@@H]1CN(C(=O)N(Cc2ccc(-c3nnc(C(F)(F)F)o3)cc2)c2ccccc2)C[C@H](C)N1C] |
| 57 | 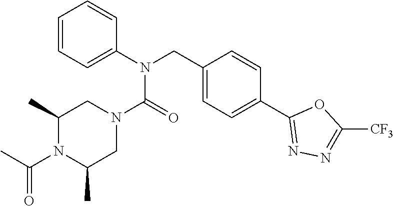 [SMILES: CC(=O)N1[C@H](C)CN(C(=O)N(Cc2ccc(-c3nnc(C(F)(F)F)o3)cc2)c2ccccc2)C[C@@H]1C] |
| 58 | 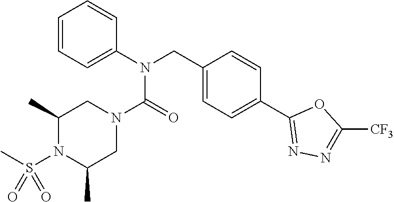 [SMILES: C[C@@H]1CN(C(=O)N(Cc2ccc(-c3nnc(C(F)(F)F)o3)cc2)c2ccccc2)C[C@H](C)N1S(C)(=O)=O] |
| 59 | 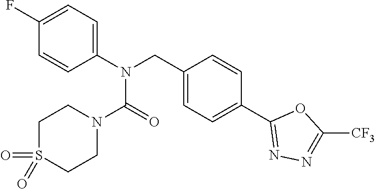 [SMILES: O=C(N1CCS(=O)(=O)CC1)N(Cc1ccc(-c2nnc(C(F)(F)F)o2)cc1)c1ccc(F)cc1] |
| 60 | 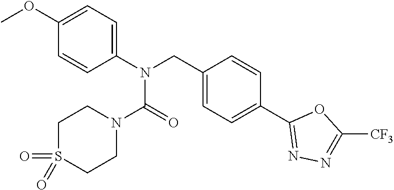 [SMILES: COc1ccc(N(Cc2ccc(-c3nnc(C(F)(F)F)o3)cc2)C(=O)N2CCS(=O)(=O)CC2)cc1] |
| 61 | 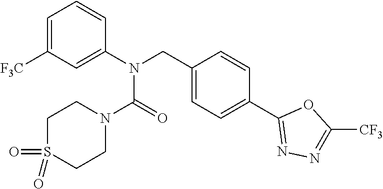 [SMILES: O=C(N1CCS(=O)(=O)CC1)N(Cc1ccc(-c2nnc(C(F)(F)F)o2)cc1)c1cccc(C(F)(F)F)c1] |
| 62 | 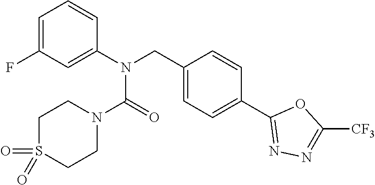 [SMILES: O=C(N1CCS(=O)(=O)CC1)N(Cc1ccc(-c2nnc(C(F)(F)F)o2)cc1)c1cccc(F)c1] |
| 63 | 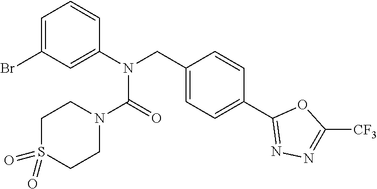 [SMILES: O=C(N1CCS(=O)(=O)CC1)N(Cc1ccc(-c2nnc(C(F)(F)F)o2)cc1)c1cccc(Br)c1] |
| 64 | 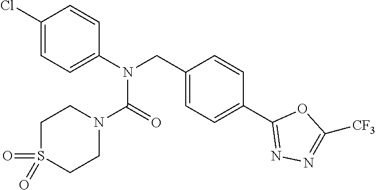 [SMILES: O=C(N1CCS(=O)(=O)CC1)N(Cc1ccc(-c2nnc(C(F)(F)F)o2)cc1)c1ccc(Cl)cc1] |
| 65 | 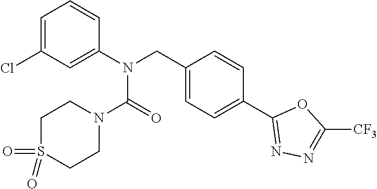 [SMILES: O=C(N1CCS(=O)(=O)CC1)N(Cc1ccc(-c2nnc(C(F)(F)F)o2)cc1)c1cccc(Cl)c1] |
| 66 | 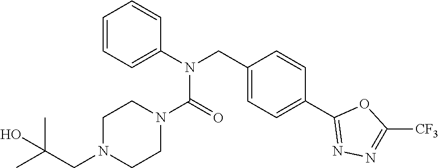 [SMILES: CC(C)(O)CN1CCN(C(=O)N(Cc2ccc(-c3nnc(C(F)(F)F)o3)cc2)c2ccccc2)CC1] |
| 67 | 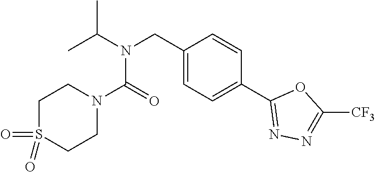 [SMILES: CC(C)N(Cc1ccc(-c2nnc(C(F)(F)F)o2)cc1)C(=O)N1CCS(=O)(=O)CC1] |
| 68 | 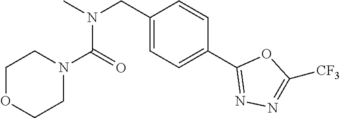 [SMILES: CN(Cc1ccc(-c2nnc(C(F)(F)F)o2)cc1)C(=O)N1CCOCC1] |
| 69 | 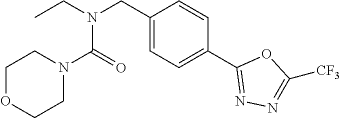 [SMILES: CCN(Cc1ccc(-c2nnc(C(F)(F)F)o2)cc1)C(=O)N1CCOCC1] |
| 70 | 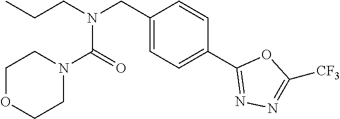 [SMILES: CCCN(Cc1ccc(-c2nnc(C(F)(F)F)o2)cc1)C(=O)N1CCOCC1] |
| 71 | 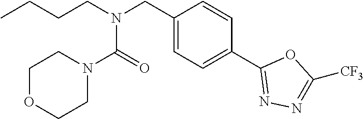 [SMILES: CCCCN(Cc1ccc(-c2nnc(C(F)(F)F)o2)cc1)C(=O)N1CCOCC1] |
| 72 | 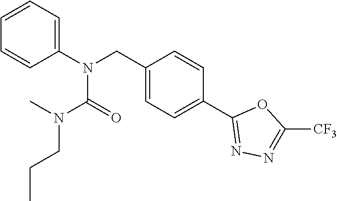 [SMILES: CCCN(C)C(=O)N(Cc1ccc(-c2nnc(C(F)(F)F)o2)cc1)c1ccccc1] |
| 73 | 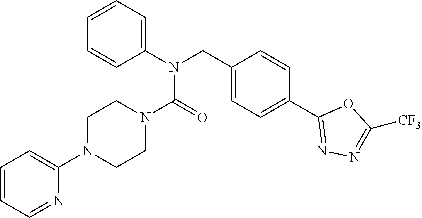 [SMILES: O=C(N1CCN(c2ccccn2)CC1)N(Cc1ccc(-c2nnc(C(F)(F)F)o2)cc1)c1ccccc1] |
| 74 | 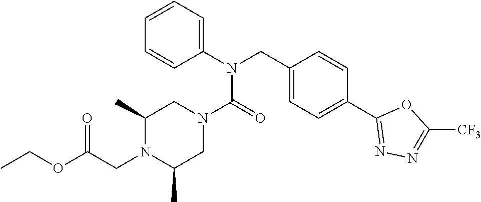 [SMILES: CCOC(=O)CN1[C@H](C)CN(C(=O)N(Cc2ccc(-c3nnc(C(F)(F)F)o3)cc2)c2ccccc2)C[C@@H]1C] |
| 75 | 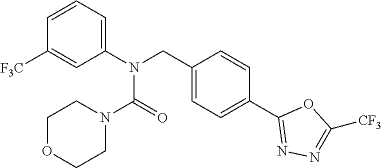 [SMILES: O=C(N1CCOCC1)N(Cc1ccc(-c2nnc(C(F)(F)F)o2)cc1)c1cccc(C(F)(F)F)c1] |
| 76 | 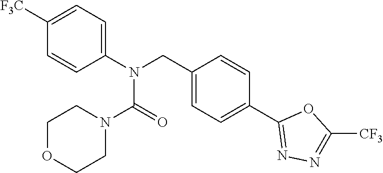 [SMILES: O=C(N1CCOCC1)N(Cc1ccc(-c2nnc(C(F)(F)F)o2)cc1)c1ccc(C(F)(F)F)cc1] |
| 77 | 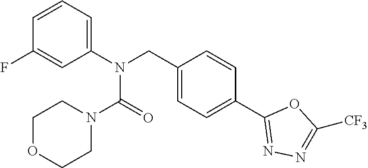 [SMILES: O=C(N1CCOCC1)N(Cc1ccc(-c2nnc(C(F)(F)F)o2)cc1)c1cccc(F)c1] |
| 78 | 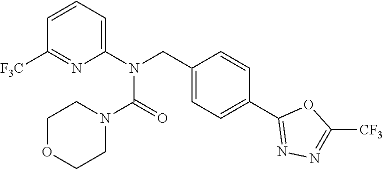 [SMILES: O=C(N1CCOCC1)N(Cc1ccc(-c2nnc(C(F)(F)F)o2)cc1)c1cccc(C(F)(F)F)n1] |
| 79 | 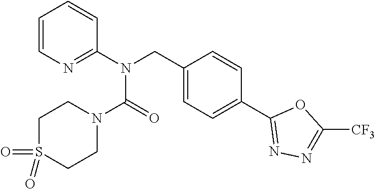 [SMILES: O=C(N1CCS(=O)(=O)CC1)N(Cc1ccc(-c2nnc(C(F)(F)F)o2)cc1)c1ccccn1] |
| 80 | 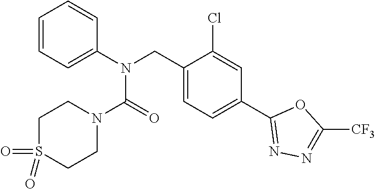 [SMILES: O=C(N1CCS(=O)(=O)CC1)N(Cc1ccc(-c2nnc(C(F)(F)F)o2)cc1Cl)c1ccccc1] |
| 81 | 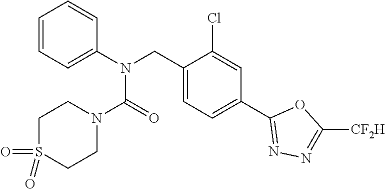 [SMILES: O=C(N1CCS(=O)(=O)CC1)N(Cc1ccc(-c2nnc(C(F)F)o2)cc1Cl)c1ccccc1] |
| 82 | 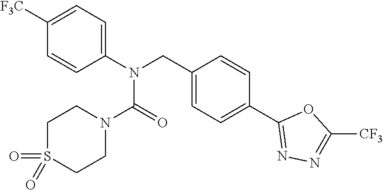 [SMILES: O=C(N1CCS(=O)(=O)CC1)N(Cc1ccc(-c2nnc(C(F)(F)F)o2)cc1)c1ccc(C(F)(F)F)cc1] |
| 83 | 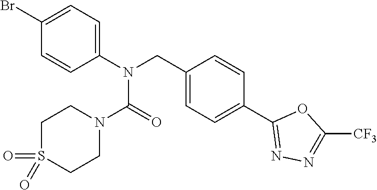 [SMILES: O=C(N1CCS(=O)(=O)CC1)N(Cc1ccc(-c2nnc(C(F)(F)F)o2)cc1)c1ccc(Br)cc1] |
| 84 | 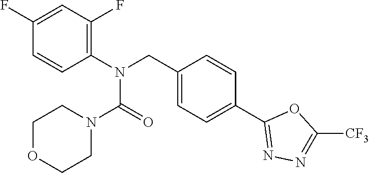 [SMILES: O=C(N1CCOCC1)N(Cc1ccc(-c2nnc(C(F)(F)F)o2)cc1)c1ccc(F)cc1F] |
| 85 | 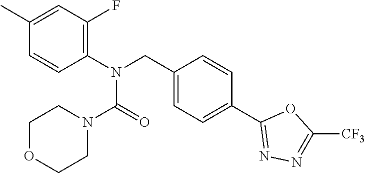 [SMILES: Cc1ccc(N(Cc2ccc(-c3nnc(C(F)(F)F)o3)cc2)C(=O)N2CCOCC2)c(F)c1] |
| 86 | 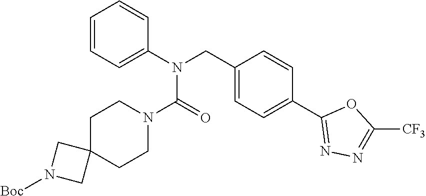 [SMILES: CC(C)(C)OC(=O)N1CC2(CCN(C(=O)N(Cc3ccc(-c4nnc(C(F)(F)F)o4)cc3)c3ccccc3)CC2)C1] |
| 87 | 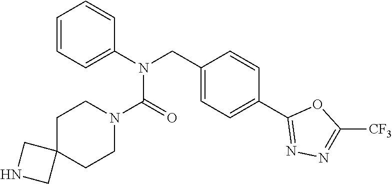 [SMILES: O=C(N1CCC2(CC1)CNC2)N(Cc1ccc(-c2nnc(C(F)(F)F)o2)cc1)c1ccccc1] |
| 88 | 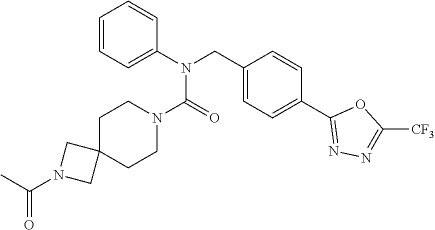 [SMILES: CC(=O)N1CC2(CCN(C(=O)N(Cc3ccc(-c4nnc(C(F)(F)F)o4)cc3)c3ccccc3)CC2)C1] |
| 89 | 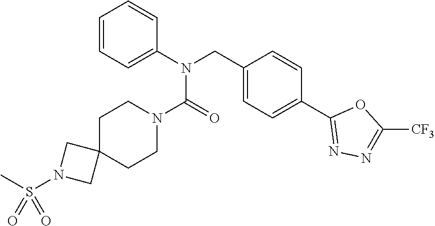 [SMILES: CS(=O)(=O)N1CC2(CCN(C(=O)N(Cc3ccc(-c4nnc(C(F)(F)F)o4)cc3)c3ccccc3)CC2)C1] |
| 90 | 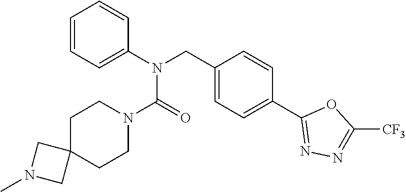 [SMILES: CN1CC2(CCN(C(=O)N(Cc3ccc(-c4nnc(C(F)(F)F)o4)cc3)c3ccccc3)CC2)C1] |
| 91 | 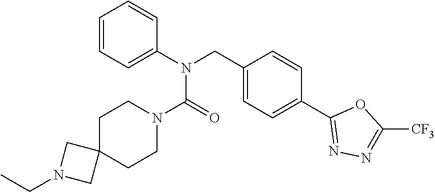 [SMILES: CCN1CC2(CCN(C(=O)N(Cc3ccc(-c4nnc(C(F)(F)F)o4)cc3)c3ccccc3)CC2)C1] |
| 92 | 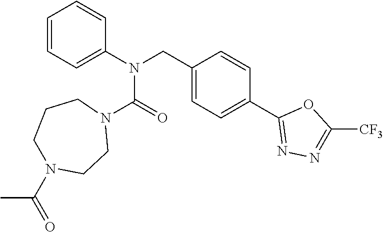 [SMILES: CC(=O)N1CCCN(C(=O)N(Cc2ccc(-c3nnc(C(F)(F)F)o3)cc2)c2ccccc2)CC1] |
| 93 | 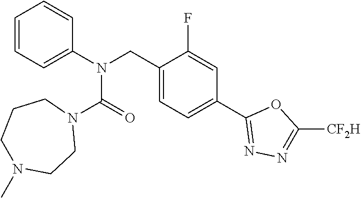 [SMILES: CN1CCCN(C(=O)N(Cc2ccc(-c3nnc(C(F)F)o3)cc2F)c2ccccc2)CC1] |
| 94 | 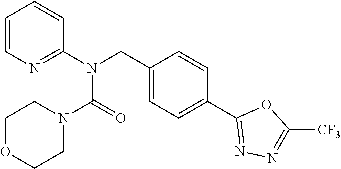 [SMILES: O=C(N1CCOCC1)N(Cc1ccc(-c2nnc(C(F)(F)F)o2)cc1)c1ccccn1] |
| 95 | 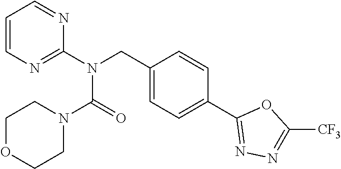 [SMILES: O=C(N1CCOCC1)N(Cc1ccc(-c2nnc(C(F)(F)F)o2)cc1)c1ncccn1] |
| 96 | 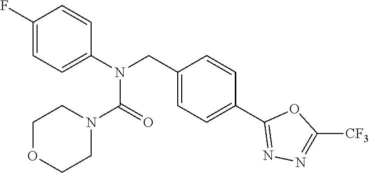 [SMILES: O=C(N1CCOCC1)N(Cc1ccc(-c2nnc(C(F)(F)F)o2)cc1)c1ccc(F)cc1] |
| 97 | 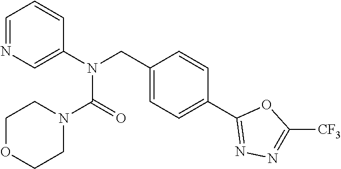 [SMILES: O=C(N1CCOCC1)N(Cc1ccc(-c2nnc(C(F)(F)F)o2)cc1)c1cccnc1] |
| 98 | 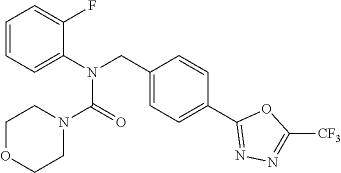 [SMILES: O=C(N1CCOCC1)N(Cc1ccc(-c2nnc(C(F)(F)F)o2)cc1)c1ccccc1F] |
| 99 | 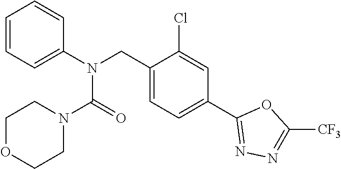 [SMILES: O=C(N1CCOCC1)N(Cc1ccc(-c2nnc(C(F)(F)F)o2)cc1Cl)c1ccccc1] |
| 100 | 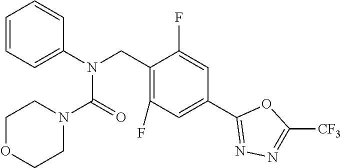 [SMILES: O=C(N1CCOCC1)N(Cc1c(F)cc(-c2nnc(C(F)(F)F)o2)cc1F)c1ccccc1] |
| 101 | 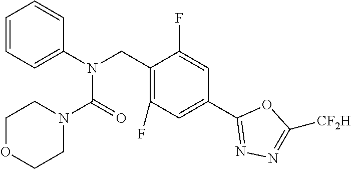 [SMILES: O=C(N1CCOCC1)N(Cc1c(F)cc(-c2nnc(C(F)F)o2)cc1F)c1ccccc1] |
| 102 | 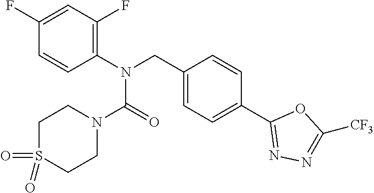 [SMILES: O=C(N1CCS(=O)(=O)CC1)N(Cc1ccc(-c2nnc(C(F)(F)F)o2)cc1)c1ccc(F)cc1F] |
| 103 | 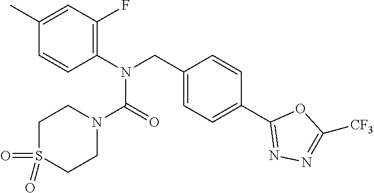 [SMILES: Cc1ccc(N(Cc2ccc(-c3nnc(C(F)(F)F)o3)cc2)C(=O)N2CCS(=O)(=O)CC2)c(F)c1] |
| 104 | 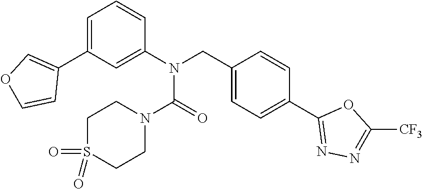 [SMILES: O=C(N1CCS(=O)(=O)CC1)N(Cc1ccc(-c2nnc(C(F)(F)F)o2)cc1)c1cccc(-c2ccoc2)c1] |
| 105 | 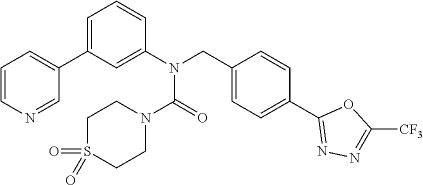 [SMILES: O=C(N1CCS(=O)(=O)CC1)N(Cc1ccc(-c2nnc(C(F)(F)F)o2)cc1)c1cccc(-c2cccnc2)c1] |
| 106 | 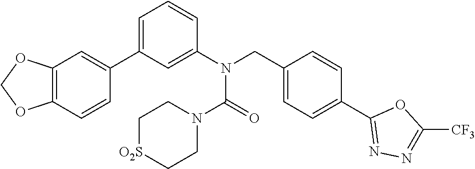 [SMILES: O=C(N1CCS(=O)(=O)CC1)N(Cc1ccc(-c2nnc(C(F)(F)F)o2)cc1)c1cccc(-c2ccc3c(c2)OCO3)c1] |
| 107 | 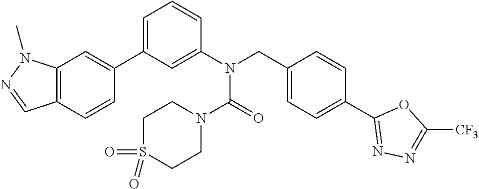 [SMILES: Cn1ncc2ccc(-c3cccc(N(Cc4ccc(-c5nnc(C(F)(F)F)o5)cc4)C(=O)N4CCS(=O)(=O)CC4)c3)cc21] |
| 108 | 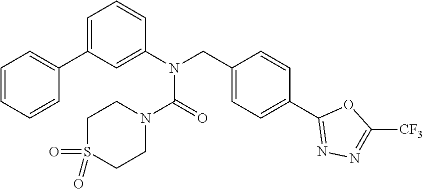 [SMILES: O=C(N1CCS(=O)(=O)CC1)N(Cc1ccc(-c2nnc(C(F)(F)F)o2)cc1)c1cccc(-c2ccccc2)c1] |
| 109 | 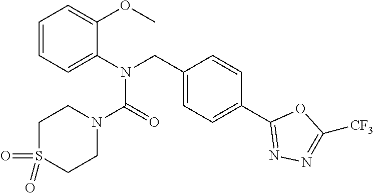 [SMILES: COc1ccccc1N(Cc1ccc(-c2nnc(C(F)(F)F)o2)cc1)C(=O)N1CCS(=O)(=O)CC1] |
| 110 | 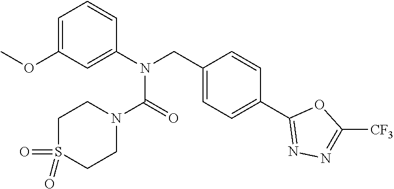 [SMILES: COc1cccc(N(Cc2ccc(-c3nnc(C(F)(F)F)o3)cc2)C(=O)N2CCS(=O)(=O)CC2)c1] |
| 111 | 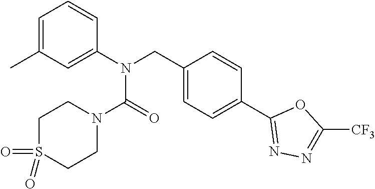 [SMILES: Cc1cccc(N(Cc2ccc(-c3nnc(C(F)(F)F)o3)cc2)C(=O)N2CCS(=O)(=O)CC2)c1] |
| 112 | 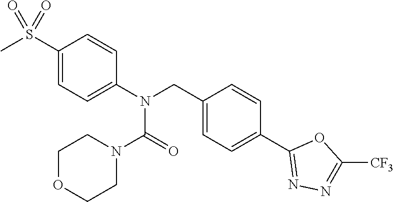 [SMILES: CS(=O)(=O)c1ccc(N(Cc2ccc(-c3nnc(C(F)(F)F)o3)cc2)C(=O)N2CCOCC2)cc1] |
| 113 | 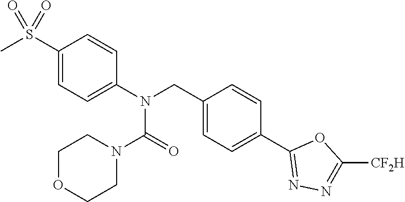 [SMILES: CS(=O)(=O)c1ccc(N(Cc2ccc(-c3nnc(C(F)F)o3)cc2)C(=O)N2CCOCC2)cc1] |
| 114 | 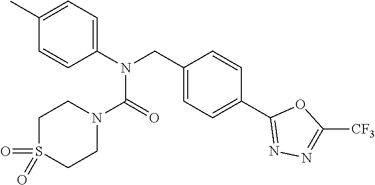 [SMILES: Cc1ccc(N(Cc2ccc(-c3nnc(C(F)(F)F)o3)cc2)C(=O)N2CCS(=O)(=O)CC2)cc1] |
| 115 | 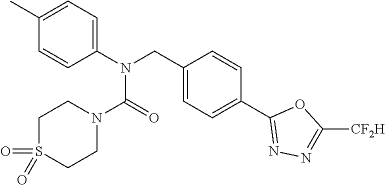 [SMILES: Cc1ccc(N(Cc2ccc(-c3nnc(C(F)F)o3)cc2)C(=O)N2CCS(=O)(=O)CC2)cc1] |
| 116 | 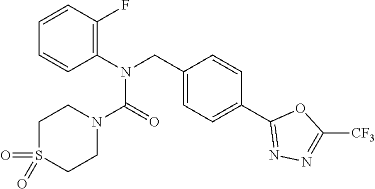 [SMILES: O=C(N1CCS(=O)(=O)CC1)N(Cc1ccc(-c2nnc(C(F)(F)F)o2)cc1)c1ccccc1F] |
| 117 | 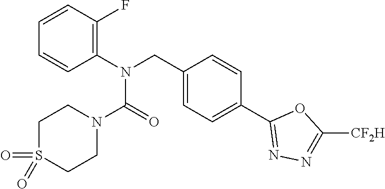 [SMILES: O=C(N1CCS(=O)(=O)CC1)N(Cc1ccc(-c2nnc(C(F)F)o2)cc1)c1ccccc1F] |
| 118 | 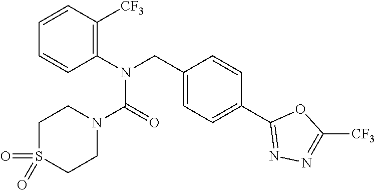 [SMILES: O=C(N1CCS(=O)(=O)CC1)N(Cc1ccc(-c2nnc(C(F)(F)F)o2)cc1)c1ccccc1C(F)(F)F] |
| 119 | 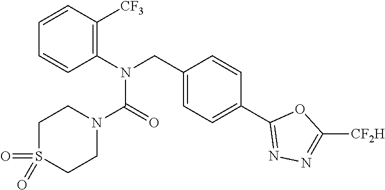 [SMILES: O=C(N1CCS(=O)(=O)CC1)N(Cc1ccc(-c2nnc(C(F)F)o2)cc1)c1ccccc1C(F)(F)F] |
| 120 | 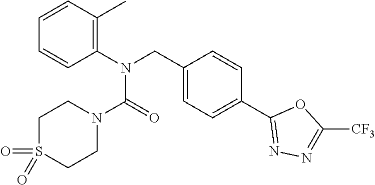 [SMILES: Cc1ccccc1N(Cc1ccc(-c2nnc(C(F)(F)F)o2)cc1)C(=O)N1CCS(=O)(=O)CC1] |
| 121 | 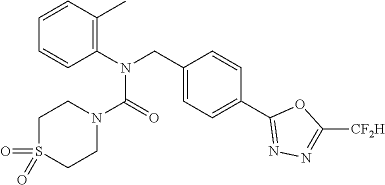 [SMILES: Cc1ccccc1N(Cc1ccc(-c2nnc(C(F)F)o2)cc1)C(=O)N1CCS(=O)(=O)CC1] |
| 122 | 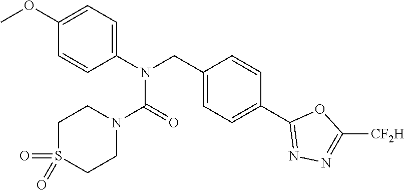 [SMILES: COc1ccc(N(Cc2ccc(-c3nnc(C(F)F)o3)cc2)C(=O)N2CCS(=O)(=O)CC2)cc1] |
| 123 | 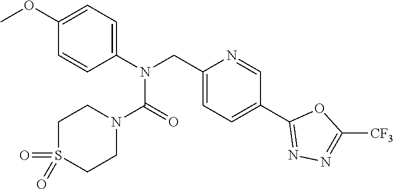 [SMILES: COc1ccc(N(Cc2ccc(-c3nnc(C(F)(F)F)o3)cn2)C(=O)N2CCS(=O)(=O)CC2)cc1] |
| 124 | 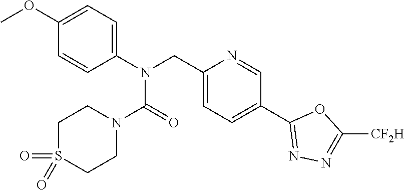 [SMILES: COc1ccc(N(Cc2ccc(-c3nnc(C(F)F)o3)cn2)C(=O)N2CCS(=O)(=O)CC2)cc1] |
| 125 | 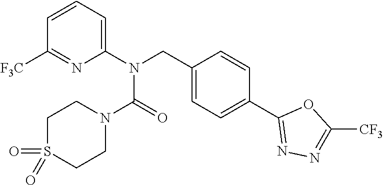 [SMILES: O=C(N1CCS(=O)(=O)CC1)N(Cc1ccc(-c2nnc(C(F)(F)F)o2)cc1)c1cccc(C(F)(F)F)n1] |
| 126 | 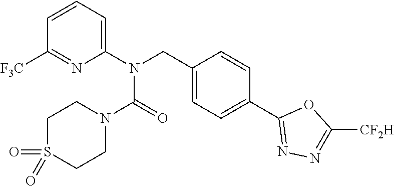 [SMILES: O=C(N1CCS(=O)(=O)CC1)N(Cc1ccc(-c2nnc(C(F)F)o2)cc1)c1cccc(C(F)(F)F)n1] |
| 127 | 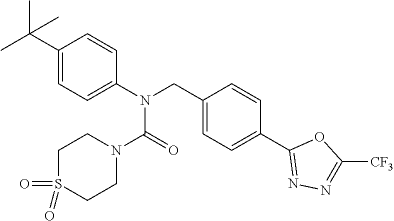 [SMILES: CC(C)(C)c1ccc(N(Cc2ccc(-c3nnc(C(F)(F)F)o3)cc2)C(=O)N2CCS(=O)(=O)CC2)cc1] |
| 128 | 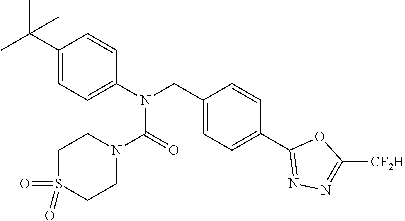 [SMILES: CC(C)(C)c1ccc(N(Cc2ccc(-c3nnc(C(F)F)o3)cc2)C(=O)N2CCS(=O)(=O)CC2)cc1] |
| 129 | 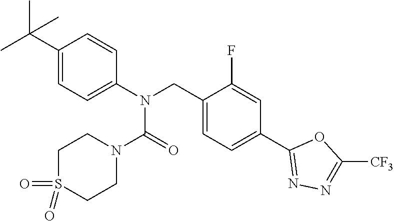 [SMILES: CC(C)(C)c1ccc(N(Cc2ccc(-c3nnc(C(F)(F)F)o3)cc2F)C(=O)N2CCS(=O)(=O)CC2)cc1] |
| 130 | 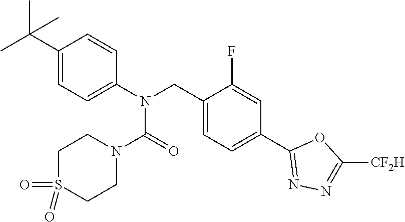 [SMILES: CC(C)(C)c1ccc(N(Cc2ccc(-c3nnc(C(F)F)o3)cc2F)C(=O)N2CCS(=O)(=O)CC2)cc1] |
| 131 | 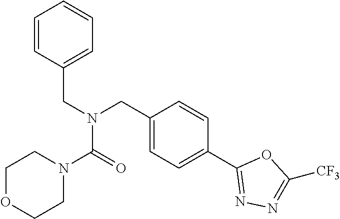 [SMILES: O=C(N1CCOCC1)N(Cc1ccccc1)Cc1ccc(-c2nnc(C(F)(F)F)o2)cc1] |
| 132 | 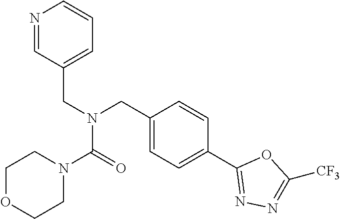 [SMILES: O=C(N1CCOCC1)N(Cc1ccc(-c2nnc(C(F)(F)F)o2)cc1)Cc1cccnc1] |
| 133 | 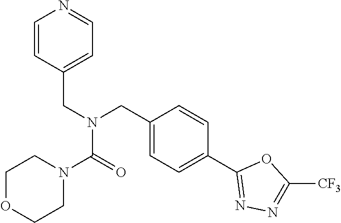 [SMILES: O=C(N1CCOCC1)N(Cc1ccncc1)Cc1ccc(-c2nnc(C(F)(F)F)o2)cc1] |
| 134 | 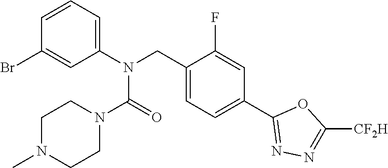 [SMILES: CN1CCN(C(=O)N(Cc2ccc(-c3nnc(C(F)F)o3)cc2F)c2cccc(Br)c2)CC1] |
| 135 | 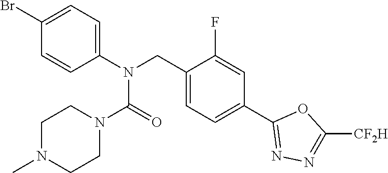 [SMILES: CN1CCN(C(=O)N(Cc2ccc(-c3nnc(C(F)F)o3)cc2F)c2ccc(Br)cc2)CC1] |
| 136 | 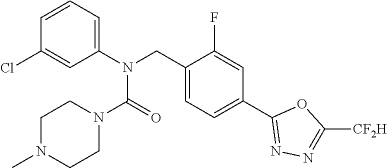 [SMILES: CN1CCN(C(=O)N(Cc2ccc(-c3nnc(C(F)F)o3)cc2F)c2cccc(Cl)c2)CC1] |
| 137 | 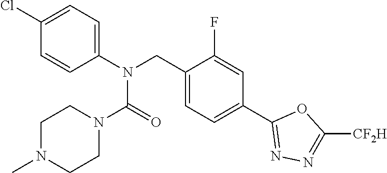 [SMILES: CN1CCN(C(=O)N(Cc2ccc(-c3nnc(C(F)F)o3)cc2F)c2ccc(Cl)cc2)CC1] |
| 138 | 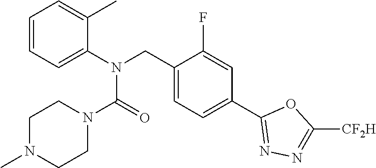 [SMILES: Cc1ccccc1N(Cc1ccc(-c2nnc(C(F)F)o2)cc1F)C(=O)N1CCN(C)CC1] |
| 139 | 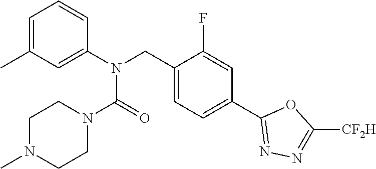 [SMILES: Cc1cccc(N(Cc2ccc(-c3nnc(C(F)F)o3)cc2F)C(=O)N2CCN(C)CC2)c1] |
| 140 | 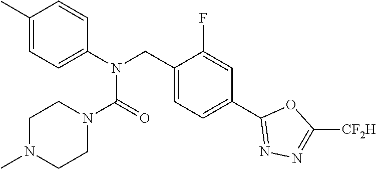 [SMILES: Cc1ccc(N(Cc2ccc(-c3nnc(C(F)F)o3)cc2F)C(=O)N2CCN(C)CC2)cc1] |
| 141 | 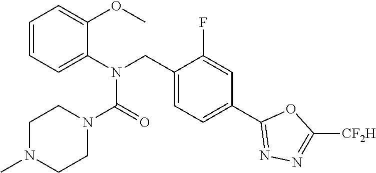 [SMILES: COc1ccccc1N(Cc1ccc(-c2nnc(C(F)F)o2)cc1F)C(=O)N1CCN(C)CC1] |
| 142 | 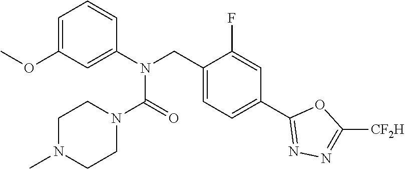 [SMILES: COc1cccc(N(Cc2ccc(-c3nnc(C(F)F)o3)cc2F)C(=O)N2CCN(C)CC2)c1] |
| 143 | 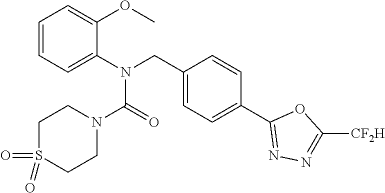 [SMILES: COc1ccccc1N(Cc1ccc(-c2nnc(C(F)F)o2)cc1)C(=O)N1CCS(=O)(=O)CC1] |
| 144 | 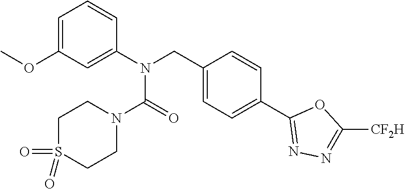 [SMILES: COc1cccc(N(Cc2ccc(-c3nnc(C(F)F)o3)cc2)C(=O)N2CCS(=O)(=O)CC2)c1] |
| 145 | 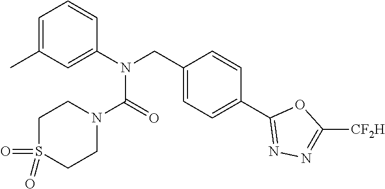 [SMILES: Cc1cccc(N(Cc2ccc(-c3nnc(C(F)F)o3)cc2)C(=O)N2CCS(=O)(=O)CC2)c1] |
| 146 | 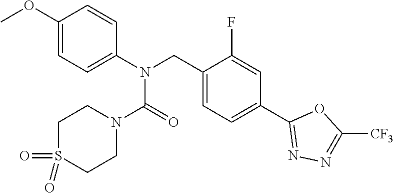 [SMILES: COc1ccc(N(Cc2ccc(-c3nnc(C(F)(F)F)o3)cc2F)C(=O)N2CCS(=O)(=O)CC2)cc1] |
| 147 | 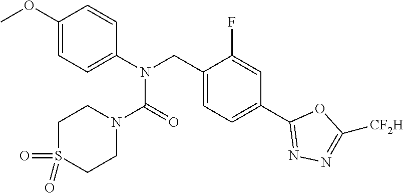 [SMILES: COc1ccc(N(Cc2ccc(-c3nnc(C(F)F)o3)cc2F)C(=O)N2CCS(=O)(=O)CC2)cc1] |
| 148 | 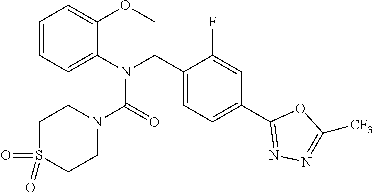 [SMILES: COc1ccccc1N(Cc1ccc(-c2nnc(C(F)(F)F)o2)cc1F)C(=O)N1CCS(=O)(=O)CC1] |
| 149 | 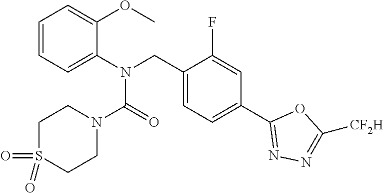 [SMILES: COc1ccccc1N(Cc1ccc(-c2nnc(C(F)F)o2)cc1F)C(=O)N1CCS(=O)(=O)CC1] |
| 150 | 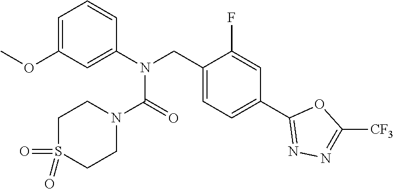 [SMILES: COc1cccc(N(Cc2ccc(-c3nnc(C(F)(F)F)o3)cc2F)C(=O)N2CCS(=O)(=O)CC2)c1] |
| 151 | 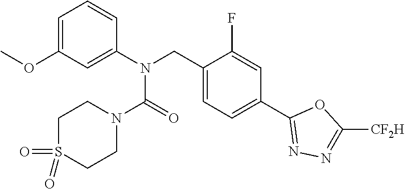 [SMILES: COc1cccc(N(Cc2ccc(-c3nnc(C(F)F)o3)cc2F)C(=O)N2CCS(=O)(=O)CC2)c1] |
| 152 | 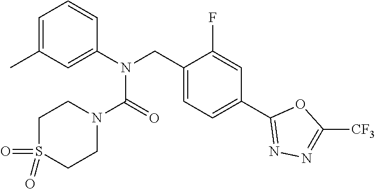 [SMILES: Cc1cccc(N(Cc2ccc(-c3nnc(C(F)(F)F)o3)cc2F)C(=O)N2CCS(=O)(=O)CC2)c1] |
| 153 | 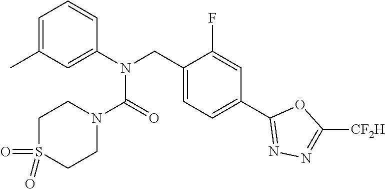 [SMILES: Cc1cccc(N(Cc2ccc(-c3nnc(C(F)F)o3)cc2F)C(=O)N2CCS(=O)(=O)CC2)c1] |
| 154 | 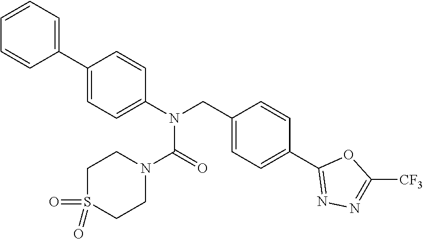 [SMILES: O=C(N1CCS(=O)(=O)CC1)N(Cc1ccc(-c2nnc(C(F)(F)F)o2)cc1)c1ccc(-c2ccccc2)cc1] |
| 155 | 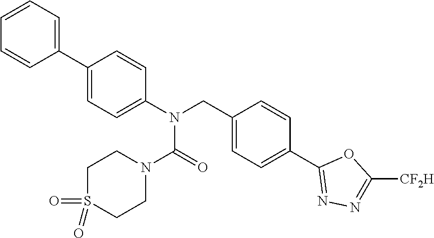 [SMILES: O=C(N1CCS(=O)(=O)CC1)N(Cc1ccc(-c2nnc(C(F)F)o2)cc1)c1ccc(-c2ccccc2)cc1] |
| 156 | 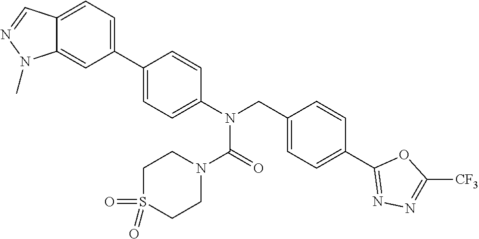 [SMILES: Cn1ncc2ccc(-c3ccc(N(Cc4ccc(-c5nnc(C(F)(F)F)o5)cc4)C(=O)N4CCS(=O)(=O)CC4)cc3)cc21] |
| 157 | 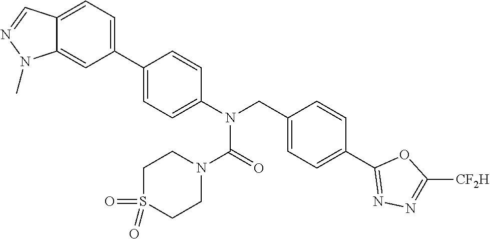 [SMILES: Cn1ncc2ccc(-c3ccc(N(Cc4ccc(-c5nnc(C(F)F)o5)cc4)C(=O)N4CCS(=O)(=O)CC4)cc3)cc21] |
| 158 | 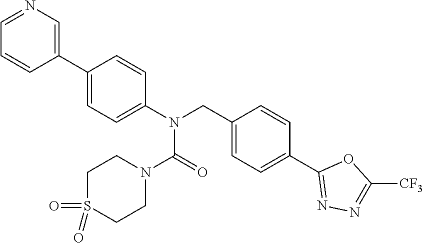 [SMILES: O=C(N1CCS(=O)(=O)CC1)N(Cc1ccc(-c2nnc(C(F)(F)F)o2)cc1)c1ccc(-c2cccnc2)cc1] |
| 159 | 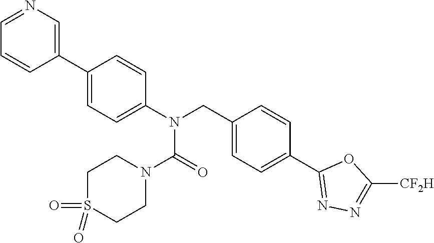 [SMILES: O=C(N1CCS(=O)(=O)CC1)N(Cc1ccc(-c2nnc(C(F)F)o2)cc1)c1ccc(-c2cccnc2)cc1] |
| 160 | 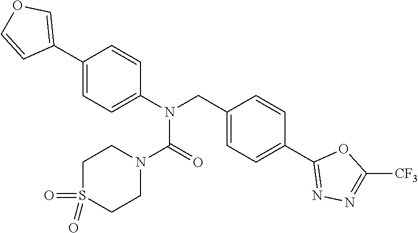 [SMILES: O=C(N1CCS(=O)(=O)CC1)N(Cc1ccc(-c2nnc(C(F)(F)F)o2)cc1)c1ccc(-c2ccoc2)cc1] |
| 161 | 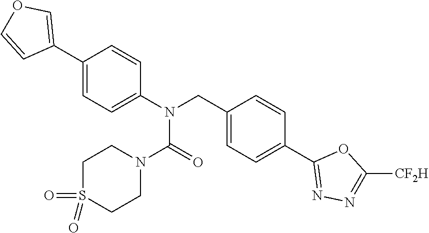 [SMILES: O=C(N1CCS(=O)(=O)CC1)N(Cc1ccc(-c2nnc(C(F)F)o2)cc1)c1ccc(-c2ccoc2)cc1] |
| 162 | 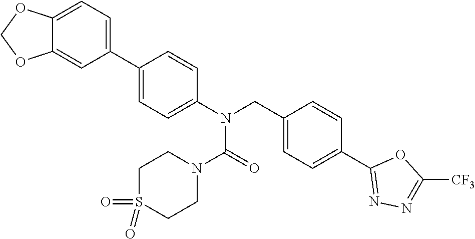 [SMILES: O=C(N1CCS(=O)(=O)CC1)N(Cc1ccc(-c2nnc(C(F)(F)F)o2)cc1)c1ccc(-c2ccc3c(c2)OCO3)cc1] |
| 163 | 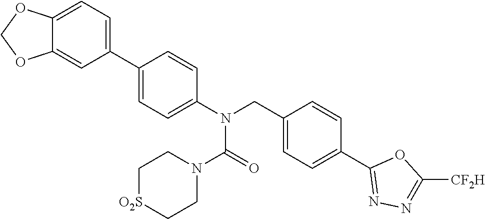 [SMILES: O=C(N1CCS(=O)(=O)CC1)N(Cc1ccc(-c2nnc(C(F)F)o2)cc1)c1ccc(-c2ccc3c(c2)OCO3)cc1] |
| 164 | 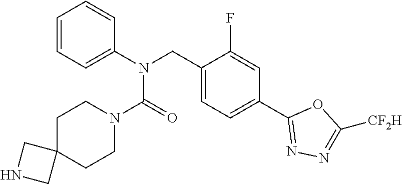 [SMILES: O=C(N1CCC2(CC1)CNC2)N(Cc1ccc(-c2nnc(C(F)F)o2)cc1F)c1ccccc1] |
| 165 | 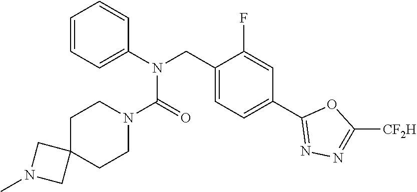 [SMILES: CN1CC2(CCN(C(=O)N(Cc3ccc(-c4nnc(C(F)F)o4)cc3F)c3ccccc3)CC2)C1] |
| 166 | 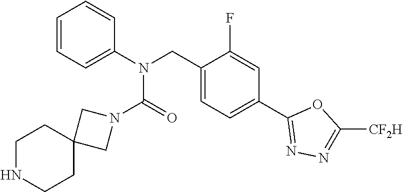 [SMILES: O=C(N1CC2(CCNCC2)C1)N(Cc1ccc(-c2nnc(C(F)F)o2)cc1F)c1ccccc1] |
| 167 | 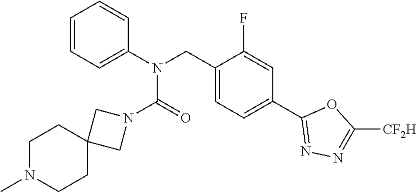 [SMILES: CN1CCC2(CC1)CN(C(=O)N(Cc1ccc(-c3nnc(C(F)F)o3)cc1F)c1ccccc1)C2] |
| 168 | 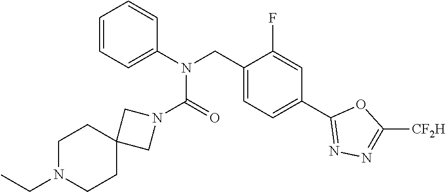 [SMILES: CCN1CCC2(CC1)CN(C(=O)N(Cc1ccc(-c3nnc(C(F)F)o3)cc1F)c1ccccc1)C2] |
| 169 | 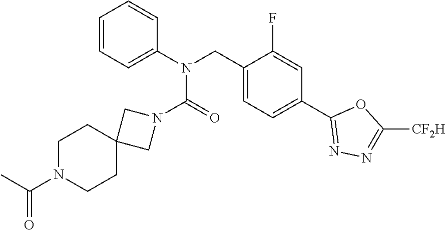 [SMILES: CC(=O)N1CCC2(CC1)CN(C(=O)N(Cc1ccc(-c3nnc(C(F)F)o3)cc1F)c1ccccc1)C2] |
| 170 | 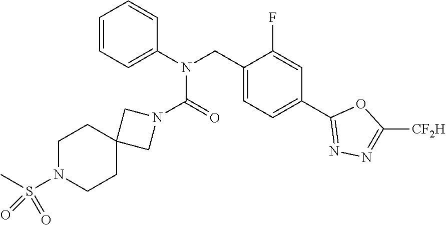 [SMILES: CS(=O)(=O)N1CCC2(CC1)CN(C(=O)N(Cc1ccc(-c3nnc(C(F)F)o3)cc1F)c1ccccc1)C2] |
| 171 | 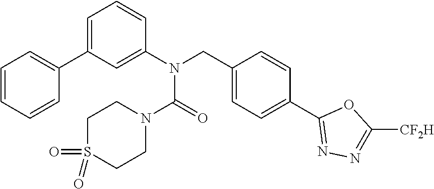 [SMILES: O=C(N1CCS(=O)(=O)CC1)N(Cc1ccc(-c2nnc(C(F)F)o2)cc1)c1cccc(-c2ccccc2)c1] |
| 172 | 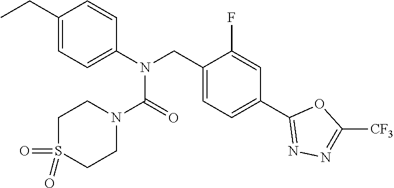 [SMILES: CCc1ccc(N(Cc2ccc(-c3nnc(C(F)(F)F)o3)cc2F)C(=O)N2CCS(=O)(=O)CC2)cc1] |
| 173 | 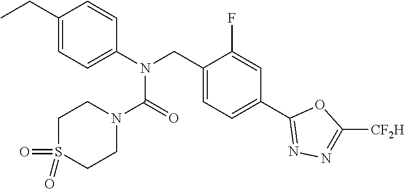 [SMILES: CCc1ccc(N(Cc2ccc(-c3nnc(C(F)F)o3)cc2F)C(=O)N2CCS(=O)(=O)CC2)cc1] |
| 174 | 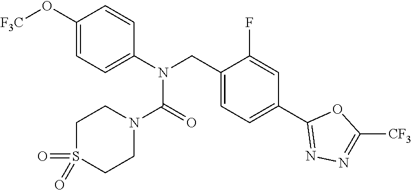 [SMILES: O=C(N1CCS(=O)(=O)CC1)N(Cc1ccc(-c2nnc(C(F)(F)F)o2)cc1F)c1ccc(OC(F)(F)F)cc1] |
| 175 | 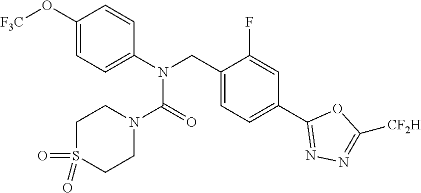 [SMILES: O=C(N1CCS(=O)(=O)CC1)N(Cc1ccc(-c2nnc(C(F)F)o2)cc1F)c1ccc(OC(F)(F)F)cc1] |
| 176 | 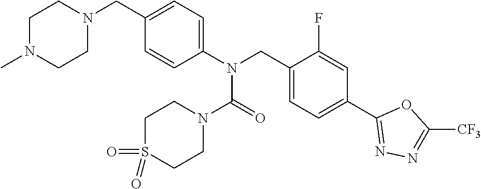 [SMILES: CN1CCN(Cc2ccc(N(Cc3ccc(-c4nnc(C(F)(F)F)o4)cc3F)C(=O)N3CCS(=O)(=O)CC3)cc2)CC1] |
| 177 | 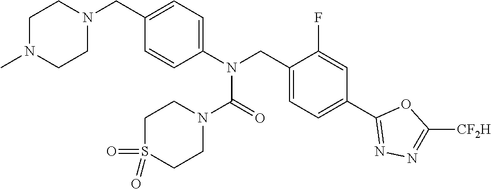 [SMILES: CN1CCN(Cc2ccc(N(Cc3ccc(-c4nnc(C(F)F)o4)cc3F)C(=O)N3CCS(=O)(=O)CC3)cc2)CC1] |
| 178 | 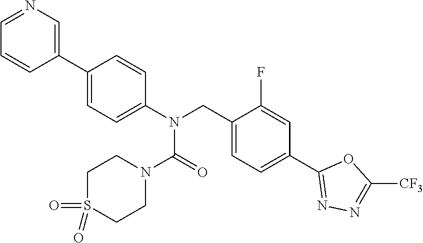 [SMILES: O=C(N1CCS(=O)(=O)CC1)N(Cc1ccc(-c2nnc(C(F)(F)F)o2)cc1F)c1ccc(-c2cccnc2)cc1] |
| 179 | 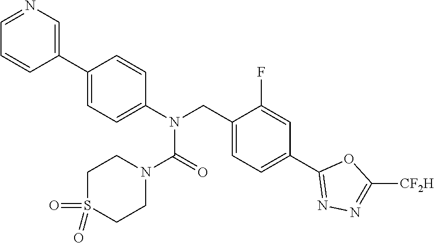 [SMILES: O=C(N1CCS(=O)(=O)CC1)N(Cc1ccc(-c2nnc(C(F)F)o2)cc1F)c1ccc(-c2cccnc2)cc1] |
| 180 | 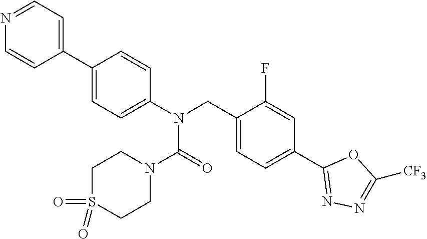 [SMILES: O=C(N1CCS(=O)(=O)CC1)N(Cc1ccc(-c2nnc(C(F)(F)F)o2)cc1F)c1ccc(-c2ccncc2)cc1] |
| 181 | 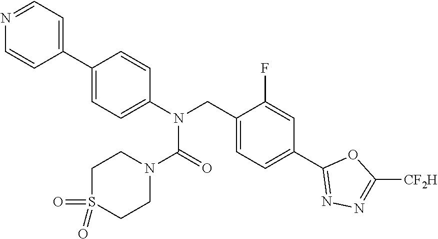 [SMILES: O=C(N1CCS(=O)(=O)CC1)N(Cc1ccc(-c2nnc(C(F)F)o2)cc1F)c1ccc(-c2ccncc2)cc1] |
| 182 | 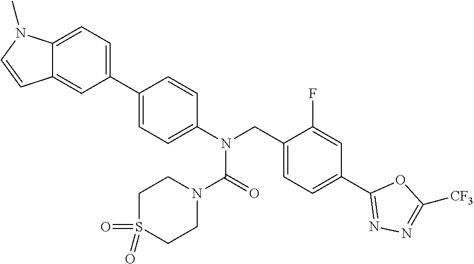 [SMILES: Cn1ccc2cc(-c3ccc(N(Cc4ccc(-c5nnc(C(F)(F)F)o5)cc4F)C(=O)N4CCS(=O)(=O)CC4)cc3)ccc21] |
| 183 | 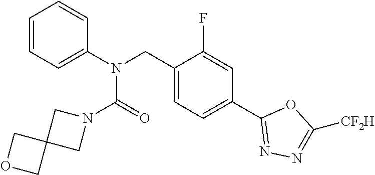 [SMILES: O=C(N1CC2(COC2)C1)N(Cc1ccc(-c2nnc(C(F)F)o2)cc1F)c1ccccc1] |
| 184 | 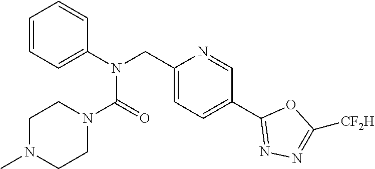 [SMILES: CN1CCN(C(=O)N(Cc2ccc(-c3nnc(C(F)F)o3)cn2)c2ccccc2)CC1] |
| 185 | 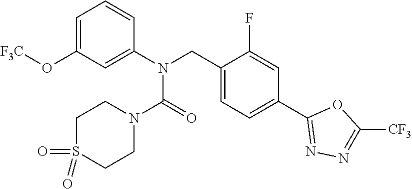 [SMILES: O=C(N1CCS(=O)(=O)CC1)N(Cc1ccc(-c2nnc(C(F)(F)F)o2)cc1F)c1cccc(OC(F)(F)F)c1] |
| 186 | 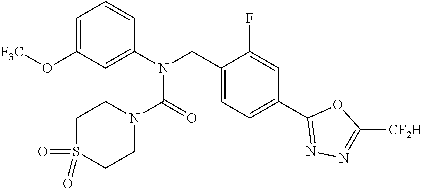 [SMILES: O=C(N1CCS(=O)(=O)CC1)N(Cc1ccc(-c2nnc(C(F)F)o2)cc1F)c1cccc(OC(F)(F)F)c1] |
| 187 | 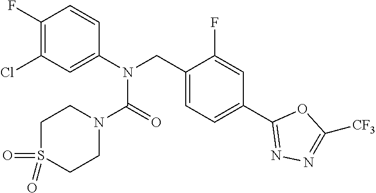 [SMILES: O=C(N1CCS(=O)(=O)CC1)N(Cc1ccc(-c2nnc(C(F)(F)F)o2)cc1F)c1ccc(F)c(Cl)c1] |
| 188 | 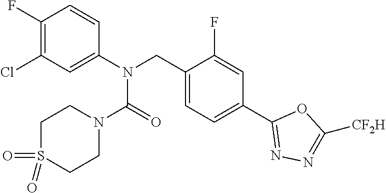 [SMILES: O=C(N1CCS(=O)(=O)CC1)N(Cc1ccc(-c2nnc(C(F)F)o2)cc1F)c1ccc(F)c(Cl)c1] |
| 189 | 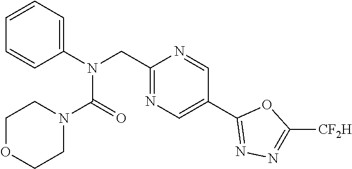 [SMILES: O=C(N1CCOCC1)N(Cc1ncc(-c2nnc(C(F)F)o2)cn1)c1ccccc1] |
| 190 | 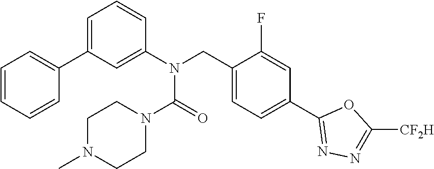 [SMILES: CN1CCN(C(=O)N(Cc2ccc(-c3nnc(C(F)F)o3)cc2F)c2cccc(-c3ccccc3)c2)CC1] |
| 191 | 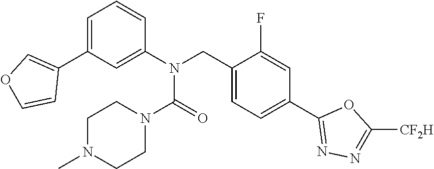 [SMILES: CN1CCN(C(=O)N(Cc2ccc(-c3nnc(C(F)F)o3)cc2F)c2cccc(-c3ccoc3)c2)CC1] |
| 192 | 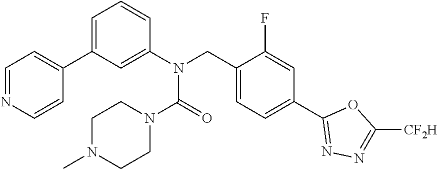 [SMILES: CN1CCN(C(=O)N(Cc2ccc(-c3nnc(C(F)F)o3)cc2F)c2cccc(-c3ccncc3)c2)CC1] |
| 193 | 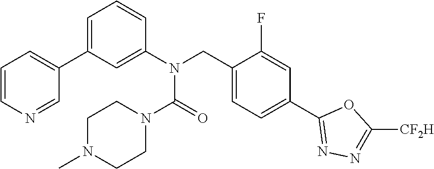 [SMILES: CN1CCN(C(=O)N(Cc2ccc(-c3nnc(C(F)F)o3)cc2F)c2cccc(-c3cccnc3)c2)CC1] |
| 194 | 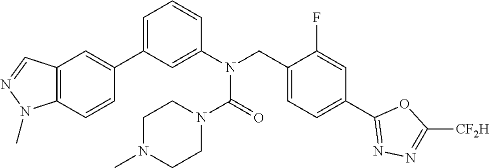 [SMILES: CN1CCN(C(=O)N(Cc2ccc(-c3nnc(C(F)F)o3)cc2F)c2cccc(-c3ccc4c(cnn4C)c3)c2)CC1] |
| 195 | 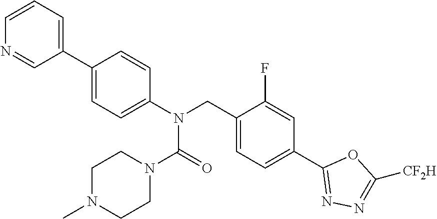 [SMILES: CN1CCN(C(=O)N(Cc2ccc(-c3nnc(C(F)F)o3)cc2F)c2ccc(-c3cccnc3)cc2)CC1] |
| 196 | 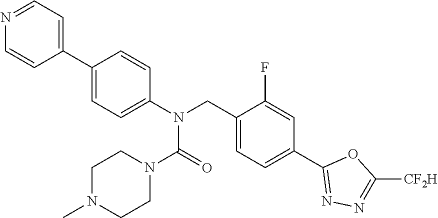 [SMILES: CN1CCN(C(=O)N(Cc2ccc(-c3nnc(C(F)F)o3)cc2F)c2ccc(-c3ccncc3)cc2)CC1] |
| 197 | 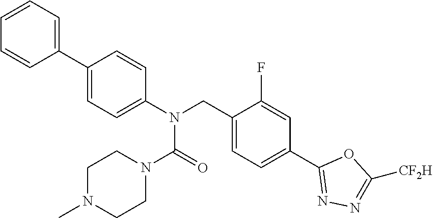 [SMILES: CN1CCN(C(=O)N(Cc2ccc(-c3nnc(C(F)F)o3)cc2F)c2ccc(-c3ccccc3)cc2)CC1] |
| 198 | 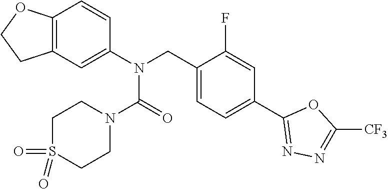 [SMILES: O=C(N1CCS(=O)(=O)CC1)N(Cc1ccc(-c2nnc(C(F)(F)F)o2)cc1F)c1ccc2c(c1)CCO2] |
| 199 | 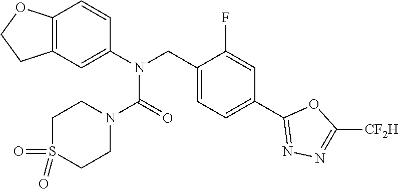 [SMILES: O=C(N1CCS(=O)(=O)CC1)N(Cc1ccc(-c2nnc(C(F)F)o2)cc1F)c1ccc2c(c1)CCO2] |
| 200 | 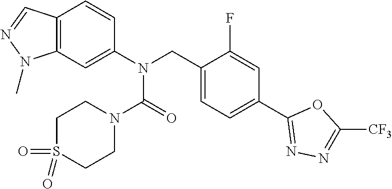 [SMILES: Cn1ncc2ccc(N(Cc3ccc(-c4nnc(C(F)(F)F)o4)cc3F)C(=O)N3CCS(=O)(=O)CC3)cc21] |
| 201 | 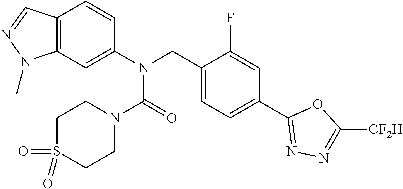 [SMILES: Cn1ncc2ccc(N(Cc3ccc(-c4nnc(C(F)F)o4)cc3F)C(=O)N3CCS(=O)(=O)CC3)cc21] |
| 202 | 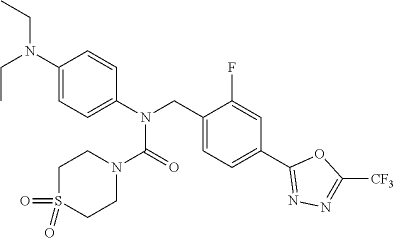 [SMILES: CCN(CC)c1ccc(N(Cc2ccc(-c3nnc(C(F)(F)F)o3)cc2F)C(=O)N2CCS(=O)(=O)CC2)cc1] |
| 203 | 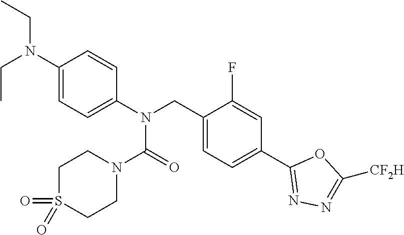 [SMILES: CCN(CC)c1ccc(N(Cc2ccc(-c3nnc(C(F)F)o3)cc2F)C(=O)N2CCS(=O)(=O)CC2)cc1] |
| 204 | 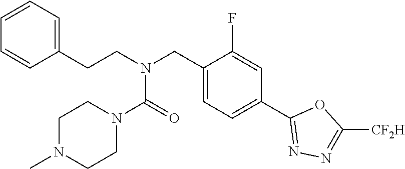 [SMILES: CN1CCN(C(=O)N(CCc2ccccc2)Cc2ccc(-c3nnc(C(F)F)o3)cc2F)CC1] |
| 205 | 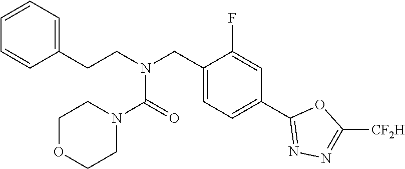 [SMILES: O=C(N1CCOCC1)N(CCc1ccccc1)Cc1ccc(-c2nnc(C(F)F)o2)cc1F] |
| 206 | 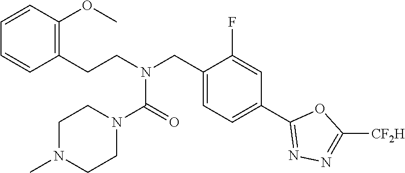 [SMILES: COc1ccccc1CCN(Cc1ccc(-c2nnc(C(F)F)o2)cc1F)C(=O)N1CCN(C)CC1] |
| 207 | 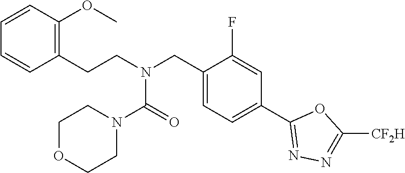 [SMILES: COc1ccccc1CCN(Cc1ccc(-c2nnc(C(F)F)o2)cc1F)C(=O)N1CCOCC1] |
| 208 | 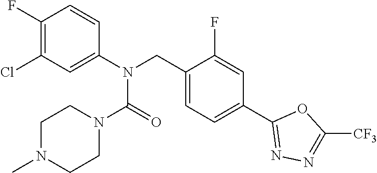 [SMILES: CN1CCN(C(=O)N(Cc2ccc(-c3nnc(C(F)(F)F)o3)cc2F)c2ccc(F)c(Cl)c2)CC1] |
| 209 | 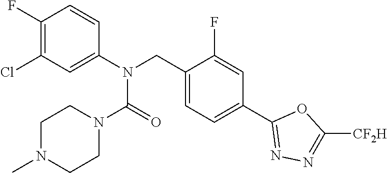 [SMILES: CN1CCN(C(=O)N(Cc2ccc(-c3nnc(C(F)F)o3)cc2F)c2ccc(F)c(Cl)c2)CC1] |
| 210 | 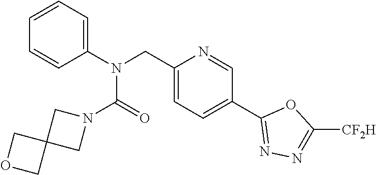 [SMILES: O=C(N1CC2(COC2)C1)N(Cc1ccc(-c2nnc(C(F)F)o2)cn1)c1ccccc1] |
| 211 | 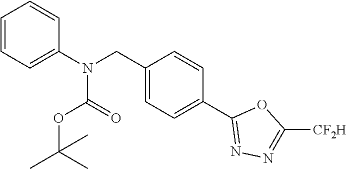 [SMILES: CC(C)(C)OC(=O)N(Cc1ccc(-c2nnc(C(F)F)o2)cc1)c1ccccc1] |
| 212 | 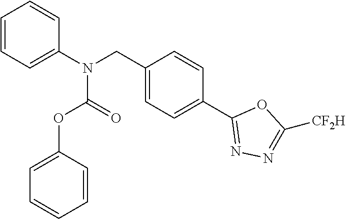 [SMILES: O=C(Oc1ccccc1)N(Cc1ccc(-c2nnc(C(F)F)o2)cc1)c1ccccc1] |
| 213 | 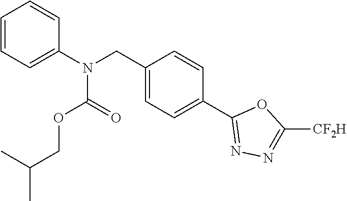 [SMILES: CC(C)COC(=O)N(Cc1ccc(-c2nnc(C(F)F)o2)cc1)c1ccccc1] |
| 214 | 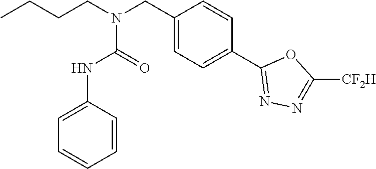 [SMILES: CCCCN(Cc1ccc(-c2nnc(C(F)F)o2)cc1)C(=O)Nc1ccccc1] |
| 215 | 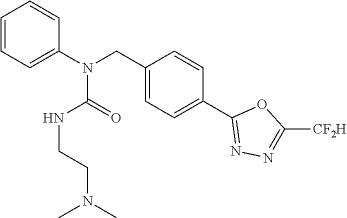 [SMILES: CN(C)CCNC(=O)N(Cc1ccc(-c2nnc(C(F)F)o2)cc1)c1ccccc1] |
| 216 | 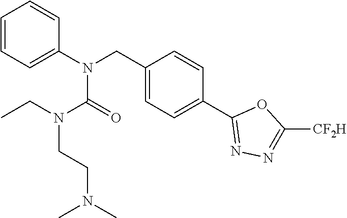 [SMILES: CCN(CCN(C)C)C(=O)N(Cc1ccc(-c2nnc(C(F)F)o2)cc1)c1ccccc1] |
| 217 | 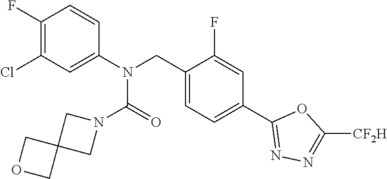 [SMILES: O=C(N1CC2(COC2)C1)N(Cc1ccc(-c2nnc(C(F)F)o2)cc1F)c1ccc(F)c(Cl)c1] |
| 218 | 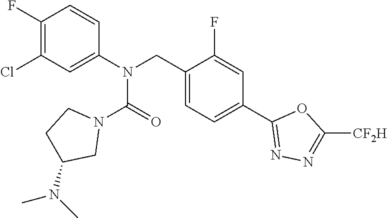 [SMILES: CN(C)[C@@H]1CCN(C(=O)N(Cc2ccc(-c3nnc(C(F)F)o3)cc2F)c2ccc(F)c(Cl)c2)C1] |
| 219 | 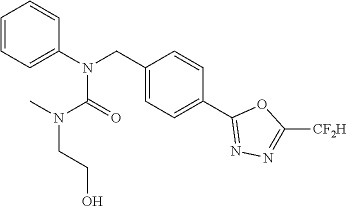 [SMILES: CN(CCO)C(=O)N(Cc1ccc(-c2nnc(C(F)F)o2)cc1)c1ccccc1] |
| 220 | 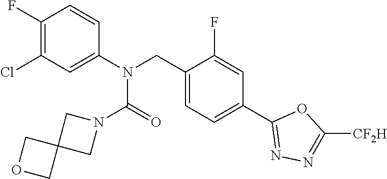 [SMILES: O=C(N1CC2(COC2)C1)N(Cc1ccc(-c2nnc(C(F)F)o2)cc1F)c1ccc(F)c(Cl)c1] |
| 221 | 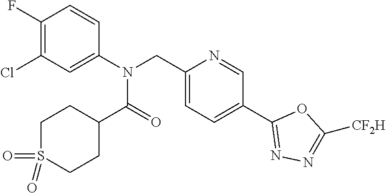 [SMILES: O=C(C1CCS(=O)(=O)CC1)N(Cc1ccc(-c2nnc(C(F)F)o2)cn1)c1ccc(F)c(Cl)c1] |
| 222 | 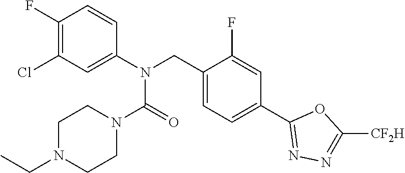 [SMILES: CCN1CCN(C(=O)N(Cc2ccc(-c3nnc(C(F)F)o3)cc2F)c2ccc(F)c(Cl)c2)CC1] |
| 223 | 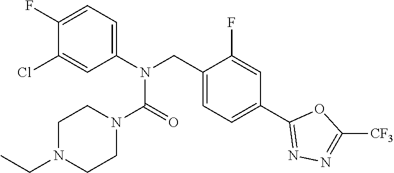 [SMILES: CCN1CCN(C(=O)N(Cc2ccc(-c3nnc(C(F)(F)F)o3)cc2F)c2ccc(F)c(Cl)c2)CC1] |
| 224 | 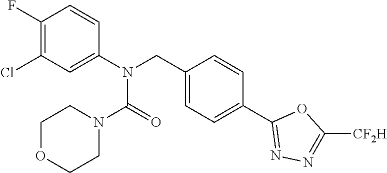 [SMILES: O=C(N1CCOCC1)N(Cc1ccc(-c2nnc(C(F)F)o2)cc1)c1ccc(F)c(Cl)c1] |
| 225 | 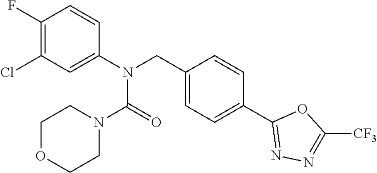 [SMILES: O=C(N1CCOCC1)N(Cc1ccc(-c2nnc(C(F)(F)F)o2)cc1)c1ccc(F)c(Cl)c1] |
| 226 | 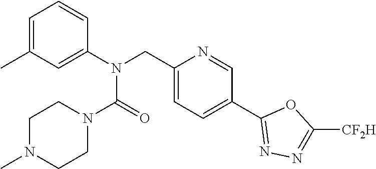 [SMILES: Cc1cccc(N(Cc2ccc(-c3nnc(C(F)F)o3)cn2)C(=O)N2CCN(C)CC2)c1] |
| 227 | 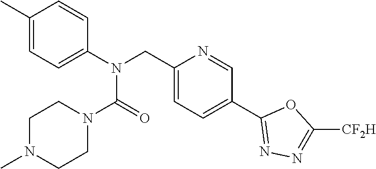 [SMILES: Cc1ccc(N(Cc2ccc(-c3nnc(C(F)F)o3)cn2)C(=O)N2CCN(C)CC2)cc1] |
| 228 | 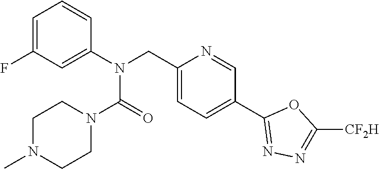 [SMILES: CN1CCN(C(=O)N(Cc2ccc(-c3nnc(C(F)F)o3)cn2)c2cccc(F)c2)CC1] |
| 229 | 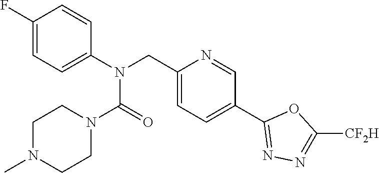 [SMILES: CN1CCN(C(=O)N(Cc2ccc(-c3nnc(C(F)F)o3)cn2)c2ccc(F)cc2)CC1] |
| 230 | 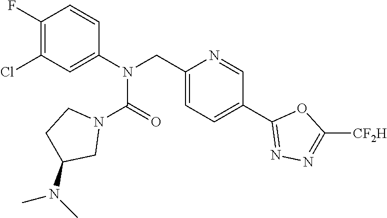 [SMILES: CN(C)[C@H]1CCN(C(=O)N(Cc2ccc(-c3nnc(C(F)F)o3)cn2)c2ccc(F)c(Cl)c2)C1] |
| 231 | 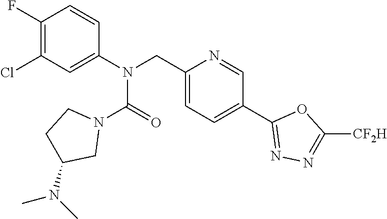 [SMILES: CN(C)[C@@H]1CCN(C(=O)N(Cc2ccc(-c3nnc(C(F)F)o3)cn2)c2ccc(F)c(Cl)c2)C1] |
| 232 | 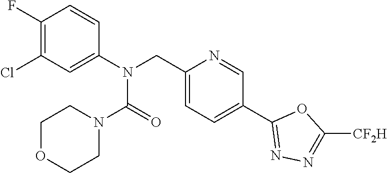 [SMILES: O=C(N1CCOCC1)N(Cc1ccc(-c2nnc(C(F)F)o2)cn1)c1ccc(F)c(Cl)c1] |
| 233 | 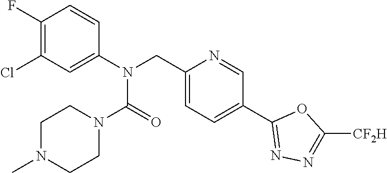 [SMILES: CN1CCN(C(=O)N(Cc2ccc(-c3nnc(C(F)F)o3)cn2)c2ccc(F)c(Cl)c2)CC1] |
| 234 | 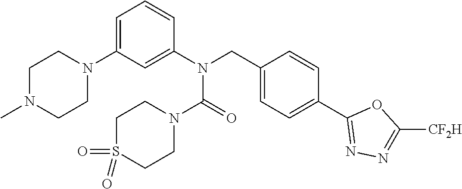 [SMILES: CN1CCN(c2cccc(N(Cc3ccc(-c4nnc(C(F)F)o4)cc3)C(=O)N3CCS(=O)(=O)CC3)c2)CC1] |
| 235 | 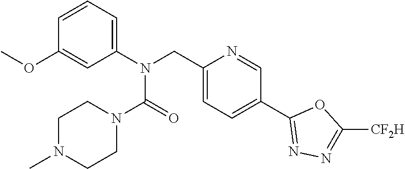 [SMILES: COc1cccc(N(Cc2ccc(-c3nnc(C(F)F)o3)cn2)C(=O)N2CCN(C)CC2)c1] |
| 236 | 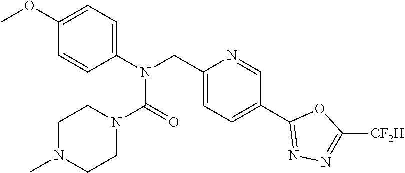 [SMILES: COc1ccc(N(Cc2ccc(-c3nnc(C(F)F)o3)cn2)C(=O)N2CCN(C)CC2)cc1] |
| 237 | 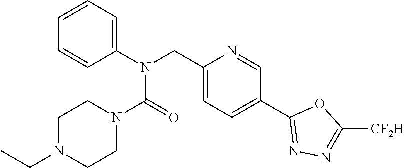 [SMILES: CCN1CCN(C(=O)N(Cc2ccc(-c3nnc(C(F)F)o3)cn2)c2ccccc2)CC1] |
| 238 | 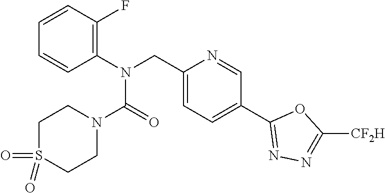 [SMILES: O=C(N1CCS(=O)(=O)CC1)N(Cc1ccc(-c2nnc(C(F)F)o2)cn1)c1ccccc1F] |
| 239 | 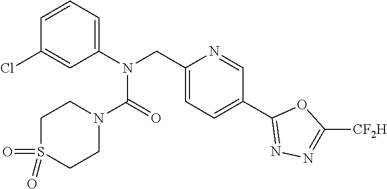 [SMILES: O=C(N1CCS(=O)(=O)CC1)N(Cc1ccc(-c2nnc(C(F)F)o2)cn1)c1cccc(Cl)c1] |
| 240 | 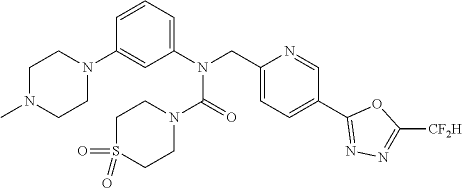 [SMILES: CN1CCN(c2cccc(N(Cc3ccc(-c4nnc(C(F)F)o4)cn3)C(=O)N3CCS(=O)(=O)CC3)c2)CC1] |
| 241 | 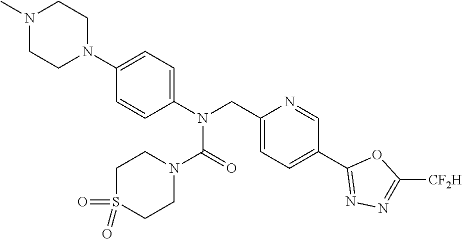 [SMILES: CN1CCN(c2ccc(N(Cc3ccc(-c4nnc(C(F)F)o4)cn3)C(=O)N3CCS(=O)(=O)CC3)cc2)CC1] |
| 242 | 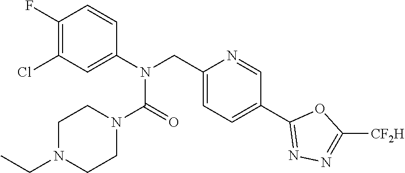 [SMILES: CCN1CCN(C(=O)N(Cc2ccc(-c3nnc(C(F)F)o3)cn2)c2ccc(F)c(Cl)c2)CC1] |
| 243 | 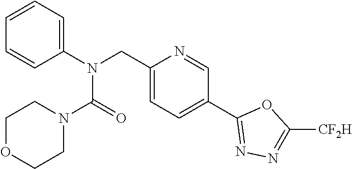 [SMILES: O=C(N1CCOCC1)N(Cc1ccc(-c2nnc(C(F)F)o2)cn1)c1ccccc1] |
| 244 | 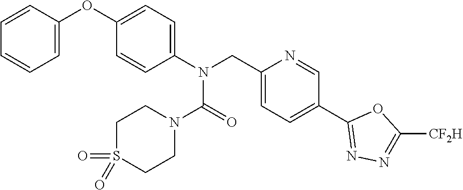 [SMILES: O=C(N1CCS(=O)(=O)CC1)N(Cc1ccc(-c2nnc(C(F)F)o2)cn1)c1ccc(Oc2ccccc2)cc1] |
| 245 | 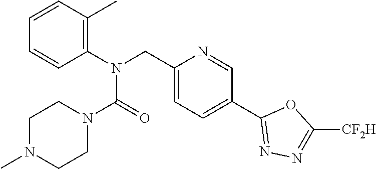 [SMILES: Cc1ccccc1N(Cc1ccc(-c2nnc(C(F)F)o2)cn1)C(=O)N1CCN(C)CC1] |
| 246 | 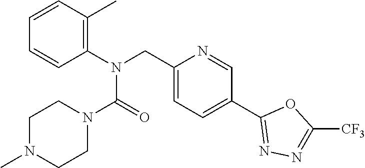 [SMILES: Cc1ccccc1N(Cc1ccc(-c2nnc(C(F)(F)F)o2)cn1)C(=O)N1CCN(C)CC1] |
| 247 | 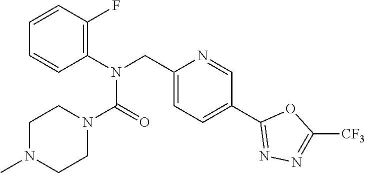 [SMILES: CN1CCN(C(=O)N(Cc2ccc(-c3nnc(C(F)(F)F)o3)cn2)c2ccccc2F)CC1] |
| 248 | 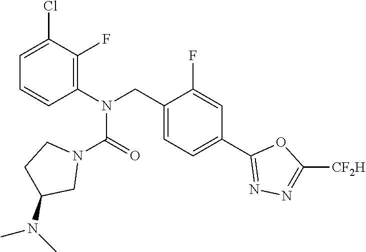 [SMILES: CN(C)[C@H]1CCN(C(=O)N(Cc2ccc(-c3nnc(C(F)F)o3)cc2F)c2cccc(Cl)c2F)C1] |
| 249 | 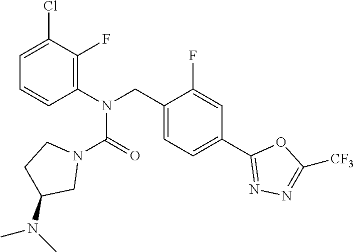 [SMILES: CN(C)[C@H]1CCN(C(=O)N(Cc2ccc(-c3nnc(C(F)(F)F)o3)cc2F)c2cccc(Cl)c2F)C1] |
| 250 | 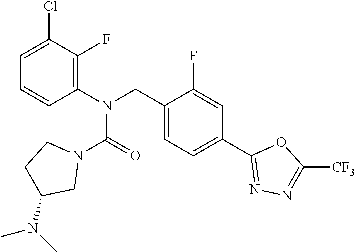 [SMILES: CN(C)[C@@H]1CCN(C(=O)N(Cc2ccc(-c3nnc(C(F)(F)F)o3)cc2F)c2cccc(Cl)c2F)C1] |
| 251 | 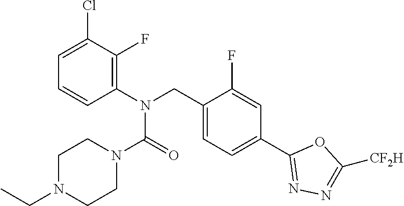 [SMILES: CCN1CCN(C(=O)N(Cc2ccc(-c3nnc(C(F)F)o3)cc2F)c2cccc(Cl)c2F)CC1] |
| 252 | 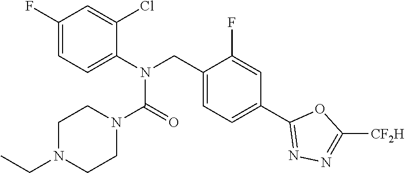 [SMILES: CCN1CCN(C(=O)N(Cc2ccc(-c3nnc(C(F)F)o3)cc2F)c2ccc(F)cc2Cl)CC1] |
| 253 | 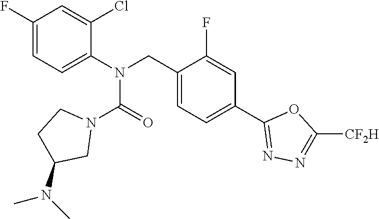 [SMILES: CN(C)[C@H]1CCN(C(=O)N(Cc2ccc(-c3nnc(C(F)F)o3)cc2F)c2ccc(F)cc2Cl)C1] |
| 254 | 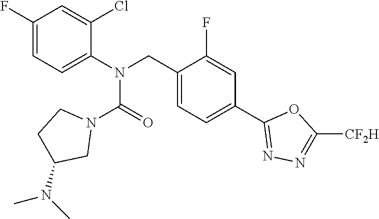 [SMILES: CN(C)[C@@H]1CCN(C(=O)N(Cc2ccc(-c3nnc(C(F)F)o3)cc2F)c2ccc(F)cc2Cl)C1] |
| 255 | 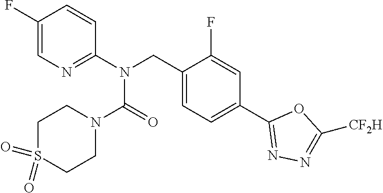 [SMILES: O=C(N1CCS(=O)(=O)CC1)N(Cc1ccc(-c2nnc(C(F)F)o2)cc1F)c1ccc(F)cn1] |
| 256 | 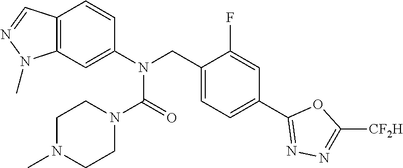 [SMILES: CN1CCN(C(=O)N(Cc2ccc(-c3nnc(C(F)F)o3)cc2F)c2ccc3cnn(C)c3c2)CC1] |
| 257 | 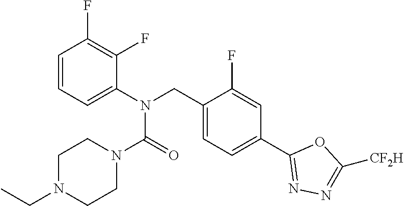 [SMILES: CCN1CCN(C(=O)N(Cc2ccc(-c3nnc(C(F)F)o3)cc2F)c2cccc(F)c2F)CC1] |
| 258 | 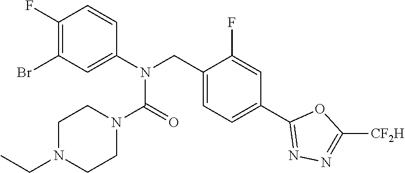 [SMILES: CCN1CCN(C(=O)N(Cc2ccc(-c3nnc(C(F)F)o3)cc2F)c2ccc(F)c(Br)c2)CC1] |
| 259 | 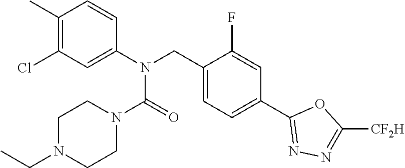 [SMILES: CCN1CCN(C(=O)N(Cc2ccc(-c3nnc(C(F)F)o3)cc2F)c2ccc(C)c(Cl)c2)CC1] |
| 260 | 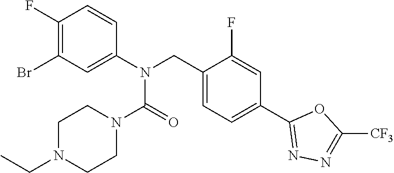 [SMILES: CCN1CCN(C(=O)N(Cc2ccc(-c3nnc(C(F)(F)F)o3)cc2F)c2ccc(F)c(Br)c2)CC1] |
| 261 | 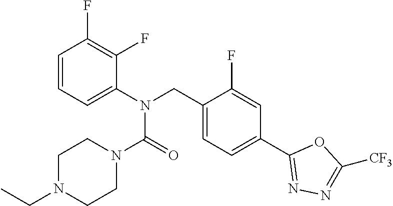 [SMILES: CCN1CCN(C(=O)N(Cc2ccc(-c3nnc(C(F)(F)F)o3)cc2F)c2cccc(F)c2F)CC1] |
| 262 | 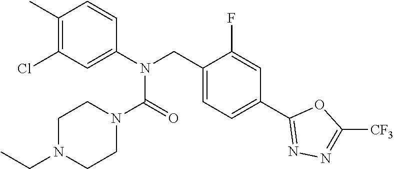 [SMILES: CCN1CCN(C(=O)N(Cc2ccc(-c3nnc(C(F)(F)F)o3)cc2F)c2ccc(C)c(Cl)c2)CC1] |
| 263 | 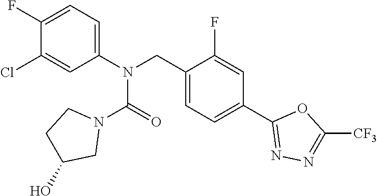 [SMILES: O=C(N1CC[C@@H](O)C1)N(Cc1ccc(-c2nnc(C(F)(F)F)o2)cc1F)c1ccc(F)c(Cl)c1] |
| 264 | 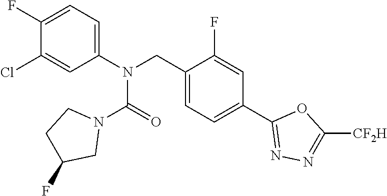 [SMILES: O=C(N1CC[C@H](F)C1)N(Cc1ccc(-c2nnc(C(F)F)o2)cc1F)c1ccc(F)c(Cl)c1] |
| 265 | 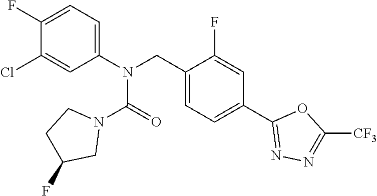 [SMILES: O=C(N1CC[C@H](F)C1)N(Cc1ccc(-c2nnc(C(F)(F)F)o2)cc1F)c1ccc(F)c(Cl)c1] |
| 266 | 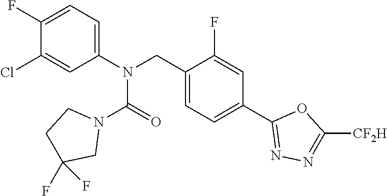 [SMILES: O=C(N1CCC(F)(F)C1)N(Cc1ccc(-c2nnc(C(F)F)o2)cc1F)c1ccc(F)c(Cl)c1] |
| 267 | 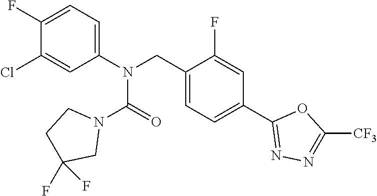 [SMILES: O=C(N1CCC(F)(F)C1)N(Cc1ccc(-c2nnc(C(F)(F)F)o2)cc1F)c1ccc(F)c(Cl)c1] |
| 268 | 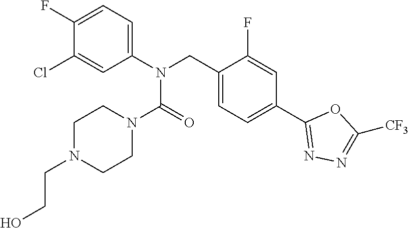 [SMILES: O=C(N1CCN(CCO)CC1)N(Cc1ccc(-c2nnc(C(F)(F)F)o2)cc1F)c1ccc(F)c(Cl)c1] |
| 269 | 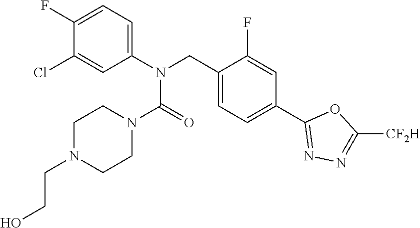 [SMILES: O=C(N1CCN(CCO)CC1)N(Cc1ccc(-c2nnc(C(F)F)o2)cc1F)c1ccc(F)c(Cl)c1] |
| 270 | 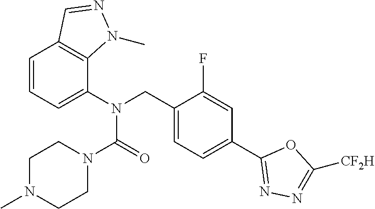 [SMILES: CN1CCN(C(=O)N(Cc2ccc(-c3nnc(C(F)F)o3)cc2F)c2cccc3cnn(C)c23)CC1] |
| 271 | 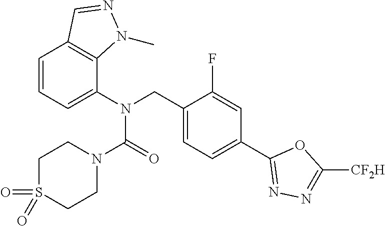 [SMILES: Cn1ncc2cccc(N(Cc3ccc(-c4nnc(C(F)F)o4)cc3F)C(=O)N3CCS(=O)(=O)CC3)c21] |
| 272 | 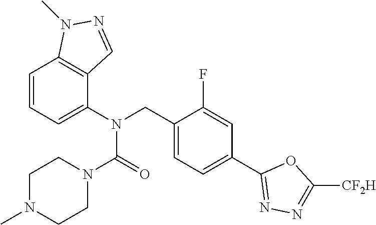 [SMILES: CN1CCN(C(=O)N(Cc2ccc(-c3nnc(C(F)F)o3)cc2F)c2cccc3c2cnn3C)CC1] |
| 273 | 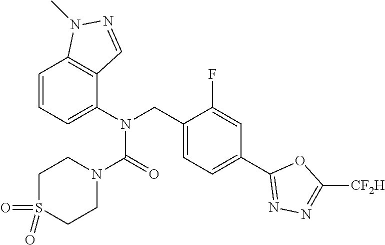 [SMILES: Cn1ncc2c(N(Cc3ccc(-c4nnc(C(F)F)o4)cc3F)C(=O)N3CCS(=O)(=O)CC3)cccc21] |
| 274 | 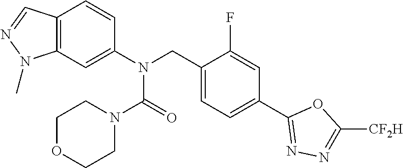 [SMILES: Cn1ncc2ccc(N(Cc3ccc(-c4nnc(C(F)F)o4)cc3F)C(=O)N3CCOCC3)cc21] |
| 275 | 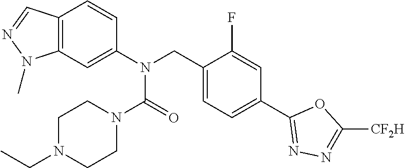 [SMILES: CCN1CCN(C(=O)N(Cc2ccc(-c3nnc(C(F)F)o3)cc2F)c2ccc3cnn(C)c3c2)CC1] |
| 276 | 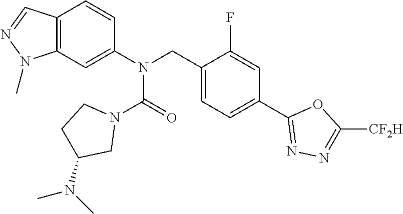 [SMILES: CN(C)[C@@H]1CCN(C(=O)N(Cc2ccc(-c3nnc(C(F)F)o3)cc2F)c2ccc3cnn(C)c3c2)C1] |
| 277 | 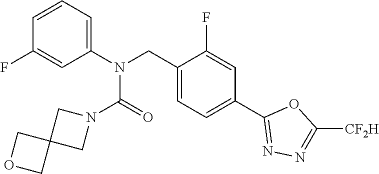 [SMILES: O=C(N1CC2(COC2)C1)N(Cc1ccc(-c2nnc(C(F)F)o2)cc1F)c1cccc(F)c1] |
| 278 | 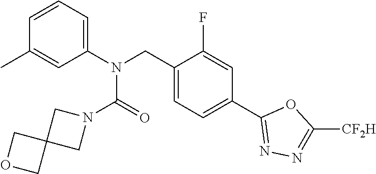 [SMILES: Cc1cccc(N(Cc2ccc(-c3nnc(C(F)F)o3)cc2F)C(=O)N2CC3(COC3)C2)c1] |
| 279 | 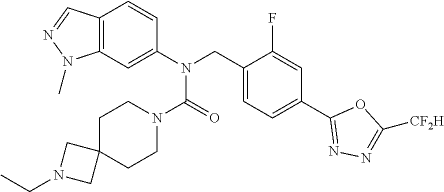 [SMILES: CCN1CC2(CCN(C(=O)N(Cc3ccc(-c4nnc(C(F)F)o4)cc3F)c3ccc4cnn(C)c4c3)CC2)C1] |
| 280 | 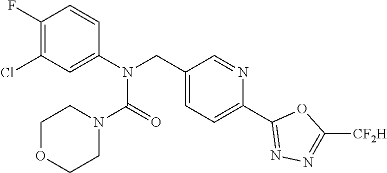 [SMILES: O=C(N1CCOCC1)N(Cc1ccc(-c2nnc(C(F)F)o2)nc1)c1ccc(F)c(Cl)c1] |
| 281 | 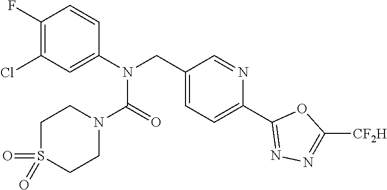 [SMILES: O=C(N1CCS(=O)(=O)CC1)N(Cc1ccc(-c2nnc(C(F)F)o2)nc1)c1ccc(F)c(Cl)c1] |
| 282 | 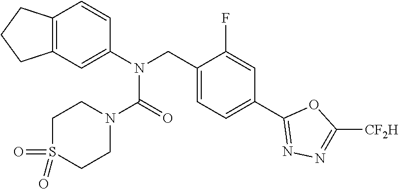 [SMILES: O=C(N1CCS(=O)(=O)CC1)N(Cc1ccc(-c2nnc(C(F)F)o2)cc1F)c1ccc2c(c1)CCC2] |
| 283 | 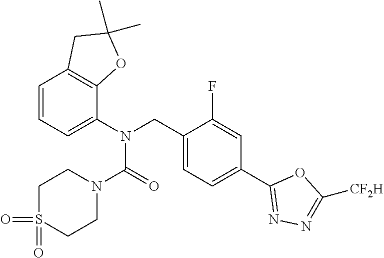 [SMILES: CC1(C)Cc2cccc(N(Cc3ccc(-c4nnc(C(F)F)o4)cc3F)C(=O)N3CCS(=O)(=O)CC3)c2O1] |
| 284 | 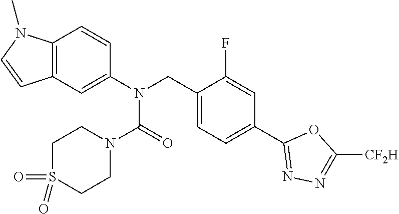 [SMILES: Cn1ccc2cc(N(Cc3ccc(-c4nnc(C(F)F)o4)cc3F)C(=O)N3CCS(=O)(=O)CC3)ccc21] |
| 285 | 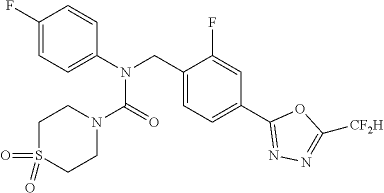 [SMILES: O=C(N1CCS(=O)(=O)CC1)N(Cc1ccc(-c2nnc(C(F)F)o2)cc1F)c1ccc(F)cc1] |
| 286 | 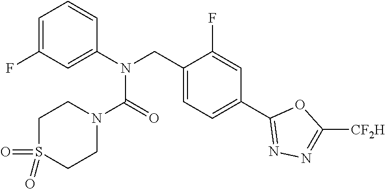 [SMILES: O=C(N1CCS(=O)(=O)CC1)N(Cc1ccc(-c2nnc(C(F)F)o2)cc1F)c1cccc(F)c1] |
| 287 | 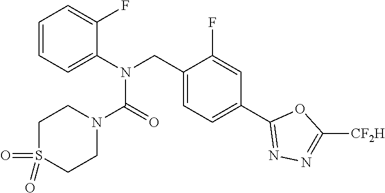 [SMILES: O=C(N1CCS(=O)(=O)CC1)N(Cc1ccc(-c2nnc(C(F)F)o2)cc1F)c1ccccc1F] |
| 288 | 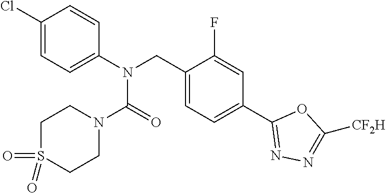 [SMILES: O=C(N1CCS(=O)(=O)CC1)N(Cc1ccc(-c2nnc(C(F)F)o2)cc1F)c1ccc(Cl)cc1] |
| 289 | 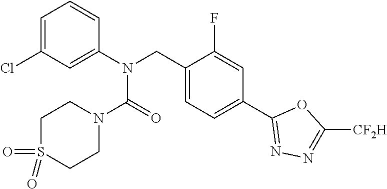 [SMILES: O=C(N1CCS(=O)(=O)CC1)N(Cc1ccc(-c2nnc(C(F)F)o2)cc1F)c1cccc(Cl)c1] |
| 290 | 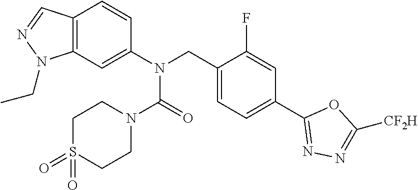 [SMILES: CCn1ncc2ccc(N(Cc3ccc(-c4nnc(C(F)F)o4)cc3F)C(=O)N3CCS(=O)(=O)CC3)cc21] |
| 291 | 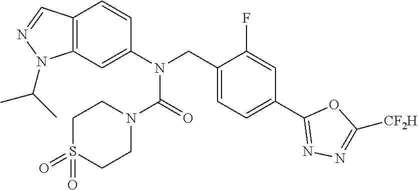 [SMILES: CC(C)n1ncc2ccc(N(Cc3ccc(-c4nnc(C(F)F)o4)cc3F)C(=O)N3CCS(=O)(=O)CC3)cc21] |
| 292 | 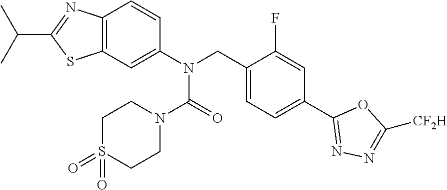 [SMILES: CC(C)c1nc2ccc(N(Cc3ccc(-c4nnc(C(F)F)o4)cc3F)C(=O)N3CCS(=O)(=O)CC3)cc2s1] |
| 293 | 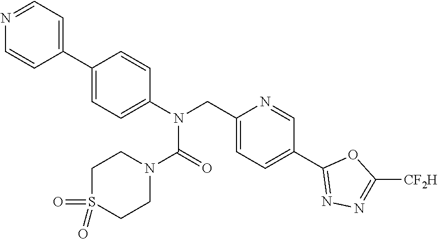 [SMILES: O=C(N1CCS(=O)(=O)CC1)N(Cc1ccc(-c2nnc(C(F)F)o2)cn1)c1ccc(-c2ccncc2)cc1] |
| 294 | 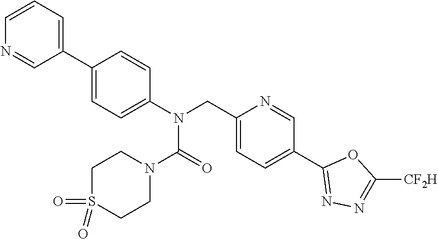 [SMILES: O=C(N1CCS(=O)(=O)CC1)N(Cc1ccc(-c2nnc(C(F)F)o2)cn1)c1ccc(-c2cccnc2)cc1] |
| 295 | 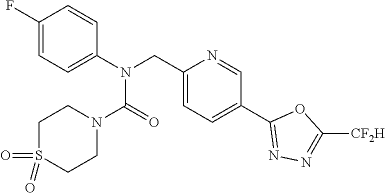 [SMILES: O=C(N1CCS(=O)(=O)CC1)N(Cc1ccc(-c2nnc(C(F)F)o2)cn1)c1ccc(F)cc1] |
| 296 | 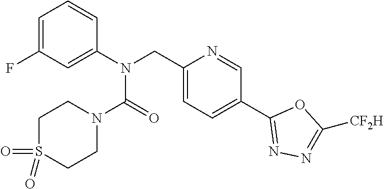 [SMILES: O=C(N1CCS(=O)(=O)CC1)N(Cc1ccc(-c2nnc(C(F)F)o2)cn1)c1cccc(F)c1] |
| 297 | 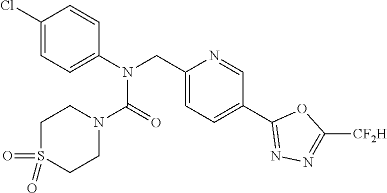 [SMILES: O=C(N1CCS(=O)(=O)CC1)N(Cc1ccc(-c2nnc(C(F)F)o2)cn1)c1ccc(Cl)cc1] |
| 298 | 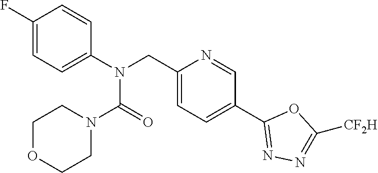 [SMILES: O=C(N1CCOCC1)N(Cc1ccc(-c2nnc(C(F)F)o2)cn1)c1ccc(F)cc1] |
| 299 | 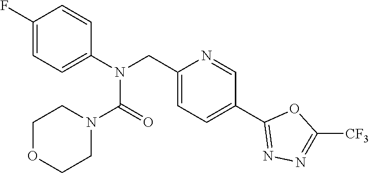 [SMILES: O=C(N1CCOCC1)N(Cc1ccc(-c2nnc(C(F)(F)F)o2)cn1)c1ccc(F)cc1] |
| 300 | 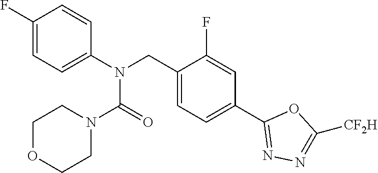 [SMILES: O=C(N1CCOCC1)N(Cc1ccc(-c2nnc(C(F)F)o2)cc1F)c1ccc(F)cc1] |
| 301 | 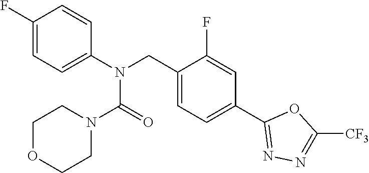 [SMILES: O=C(N1CCOCC1)N(Cc1ccc(-c2nnc(C(F)(F)F)o2)cc1F)c1ccc(F)cc1] |
| 302 | 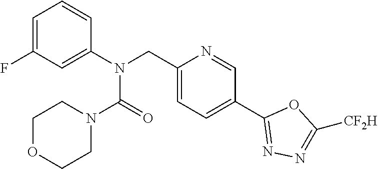 [SMILES: O=C(N1CCOCC1)N(Cc1ccc(-c2nnc(C(F)F)o2)cn1)c1cccc(F)c1] |
| 303 | 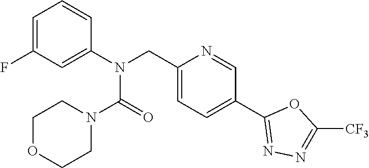 [SMILES: O=C(N1CCOCC1)N(Cc1ccc(-c2nnc(C(F)(F)F)o2)cn1)c1cccc(F)c1] |
| 304 | 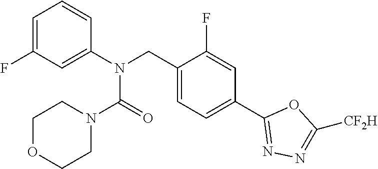 [SMILES: O=C(N1CCOCC1)N(Cc1ccc(-c2nnc(C(F)F)o2)cc1F)c1cccc(F)c1] |
| 305 | 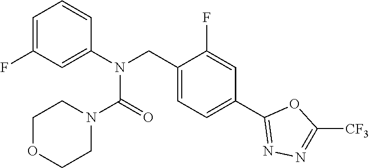 [SMILES: O=C(N1CCOCC1)N(Cc1ccc(-c2nnc(C(F)(F)F)o2)cc1F)c1cccc(F)c1] |
| 306 | 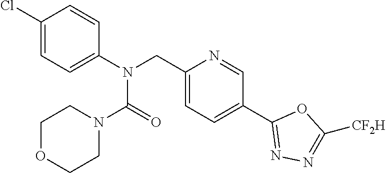 [SMILES: O=C(N1CCOCC1)N(Cc1ccc(-c2nnc(C(F)F)o2)cn1)c1ccc(Cl)cc1] |
| 307 | 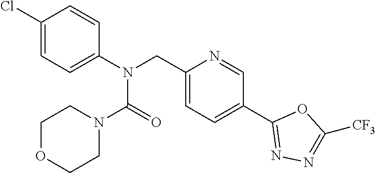 [SMILES: O=C(N1CCOCC1)N(Cc1ccc(-c2nnc(C(F)(F)F)o2)cn1)c1ccc(Cl)cc1] |
| 308 | 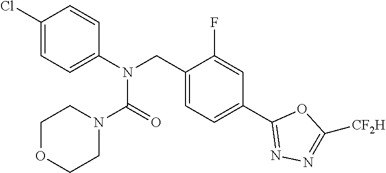 [SMILES: O=C(N1CCOCC1)N(Cc1ccc(-c2nnc(C(F)F)o2)cc1F)c1ccc(Cl)cc1] |
| 309 | 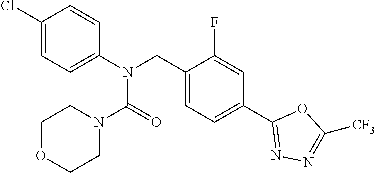 [SMILES: O=C(N1CCOCC1)N(Cc1ccc(-c2nnc(C(F)(F)F)o2)cc1F)c1ccc(Cl)cc1] |
| 310 | 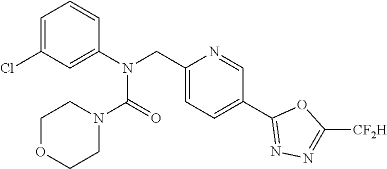 [SMILES: O=C(N1CCOCC1)N(Cc1ccc(-c2nnc(C(F)F)o2)cn1)c1cccc(Cl)c1] |
| 311 | 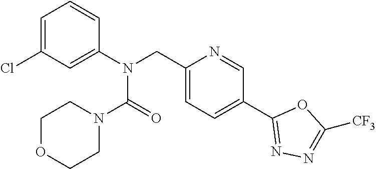 [SMILES: O=C(N1CCOCC1)N(Cc1ccc(-c2nnc(C(F)(F)F)o2)cn1)c1cccc(Cl)c1] |
| 312 | 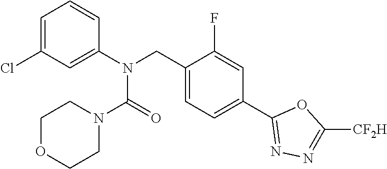 [SMILES: O=C(N1CCOCC1)N(Cc1ccc(-c2nnc(C(F)F)o2)cc1F)c1cccc(Cl)c1] |
| 313 | 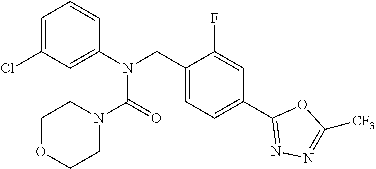 [SMILES: O=C(N1CCOCC1)N(Cc1ccc(-c2nnc(C(F)(F)F)o2)cc1F)c1cccc(Cl)c1] |
| 314 | 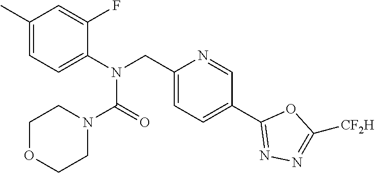 [SMILES: Cc1ccc(N(Cc2ccc(-c3nnc(C(F)F)o3)cn2)C(=O)N2CCOCC2)c(F)c1] |
| 315 | 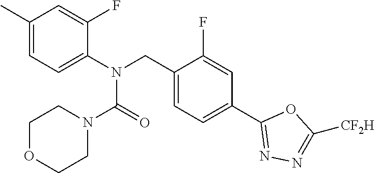 [SMILES: Cc1ccc(N(Cc2ccc(-c3nnc(C(F)F)o3)cc2F)C(=O)N2CCOCC2)c(F)c1] |
| 316 | 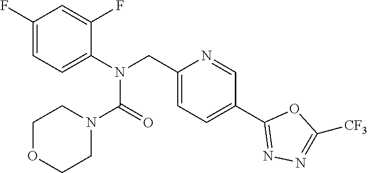 [SMILES: O=C(N1CCOCC1)N(Cc1ccc(-c2nnc(C(F)(F)F)o2)cn1)c1ccc(F)cc1F] |
| 317 | 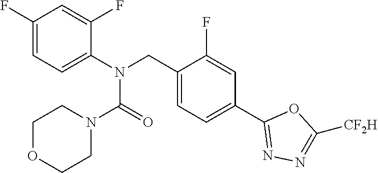 [SMILES: O=C(N1CCOCC1)N(Cc1ccc(-c2nnc(C(F)F)o2)cc1F)c1ccc(F)cc1F] |
| 318 | 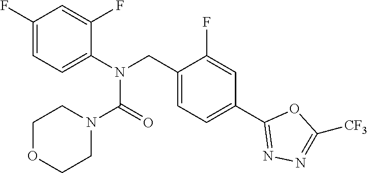 [SMILES: O=C(N1CCOCC1)N(Cc1ccc(-c2nnc(C(F)(F)F)o2)cc1F)c1ccc(F)cc1F] |
| 319 | 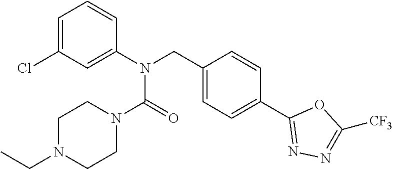 [SMILES: CCN1CCN(C(=O)N(Cc2ccc(-c3nnc(C(F)(F)F)o3)cc2)c2cccc(Cl)c2)CC1] |
| 320 | 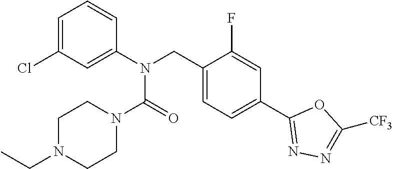 [SMILES: CCN1CCN(C(=O)N(Cc2ccc(-c3nnc(C(F)(F)F)o3)cc2F)c2cccc(Cl)c2)CC1] |
| 321 | 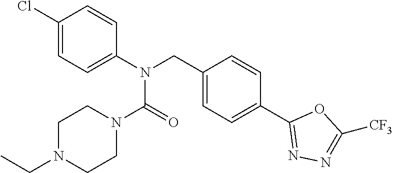 [SMILES: CCN1CCN(C(=O)N(Cc2ccc(-c3nnc(C(F)(F)F)o3)cc2)c2ccc(Cl)cc2)CC1] |
| 322 | 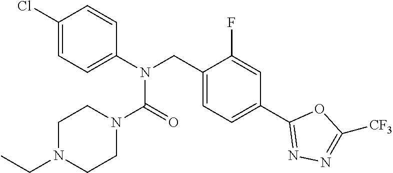 [SMILES: CCN1CCN(C(=O)N(Cc2ccc(-c3nnc(C(F)(F)F)o3)cc2F)c2ccc(Cl)cc2)CC1] |
| 323 | 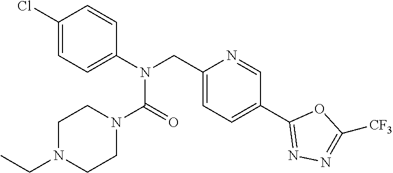 [SMILES: CCN1CCN(C(=O)N(Cc2ccc(-c3nnc(C(F)(F)F)o3)cn2)c2ccc(Cl)cc2)CC1] |
| 324 | 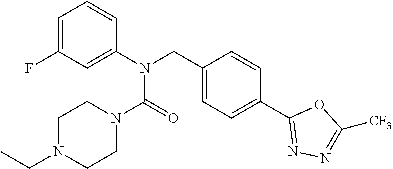 [SMILES: CCN1CCN(C(=O)N(Cc2ccc(-c3nnc(C(F)(F)F)o3)cc2)c2cccc(F)c2)CC1] |
| 325 | 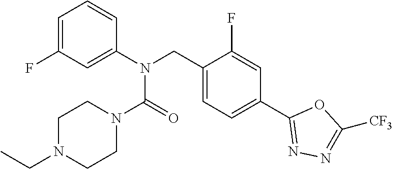 [SMILES: CCN1CCN(C(=O)N(Cc2ccc(-c3nnc(C(F)(F)F)o3)cc2F)c2cccc(F)c2)CC1] |
| 326 | 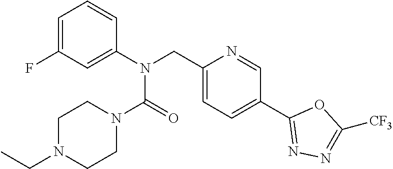 [SMILES: CCN1CCN(C(=O)N(Cc2ccc(-c3nnc(C(F)(F)F)o3)cn2)c2cccc(F)c2)CC1] |
| 327 | 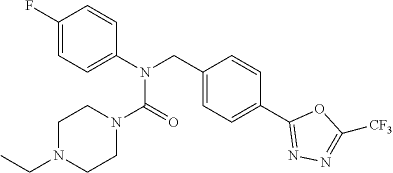 [SMILES: CCN1CCN(C(=O)N(Cc2ccc(-c3nnc(C(F)(F)F)o3)cc2)c2ccc(F)cc2)CC1] |
| 328 | 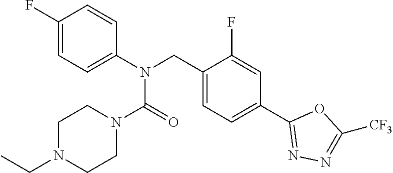 [SMILES: CCN1CCN(C(=O)N(Cc2ccc(-c3nnc(C(F)(F)F)o3)cc2F)c2ccc(F)cc2)CC1] |
| 329 | 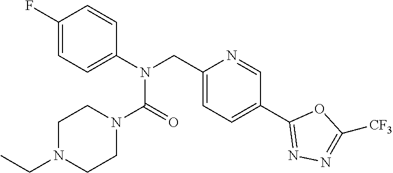 [SMILES: CCN1CCN(C(=O)N(Cc2ccc(-c3nnc(C(F)(F)F)o3)cn2)c2ccc(F)cc2)CC1] |
| 330 | 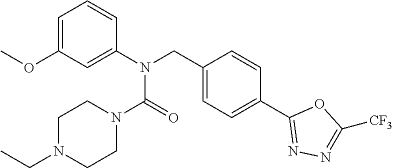 [SMILES: CCN1CCN(C(=O)N(Cc2ccc(-c3nnc(C(F)(F)F)o3)cc2)c2cccc(OC)c2)CC1] |
| 331 | 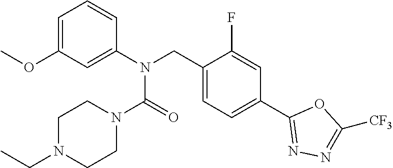 [SMILES: CCN1CCN(C(=O)N(Cc2ccc(-c3nnc(C(F)(F)F)o3)cc2F)c2cccc(OC)c2)CC1] |
| 332 | 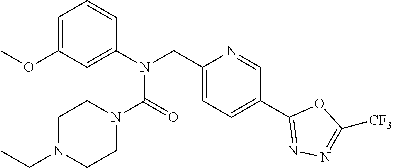 [SMILES: CCN1CCN(C(=O)N(Cc2ccc(-c3nnc(C(F)(F)F)o3)cn2)c2cccc(OC)c2)CC1] |
| 333 | 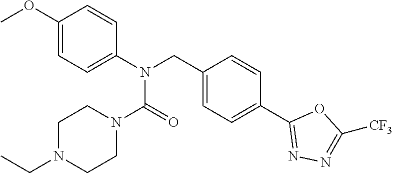 [SMILES: CCN1CCN(C(=O)N(Cc2ccc(-c3nnc(C(F)(F)F)o3)cc2)c2ccc(OC)cc2)CC1] |
| 334 | 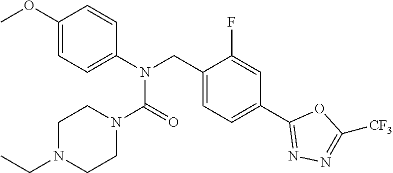 [SMILES: CCN1CCN(C(=O)N(Cc2ccc(-c3nnc(C(F)(F)F)o3)cc2F)c2ccc(OC)cc2)CC1] |
| 335 | 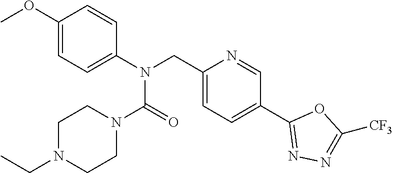 [SMILES: CCN1CCN(C(=O)N(Cc2ccc(-c3nnc(C(F)(F)F)o3)cn2)c2ccc(OC)cc2)CC1] |
| 336 | 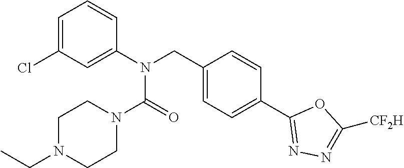 [SMILES: CCN1CCN(C(=O)N(Cc2ccc(-c3nnc(C(F)F)o3)cc2)c2cccc(Cl)c2)CC1] |
| 337 | 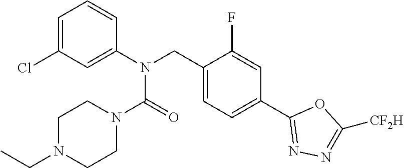 [SMILES: CCN1CCN(C(=O)N(Cc2ccc(-c3nnc(C(F)F)o3)cc2F)c2cccc(Cl)c2)CC1] |
| 338 | 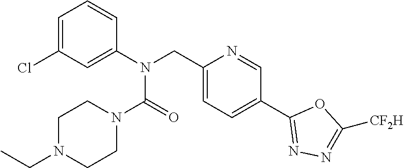 [SMILES: CCN1CCN(C(=O)N(Cc2ccc(-c3nnc(C(F)F)o3)cn2)c2cccc(Cl)c2)CC1] |
| 339 | 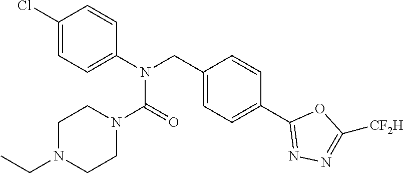 [SMILES: CCN1CCN(C(=O)N(Cc2ccc(-c3nnc(C(F)F)o3)cc2)c2ccc(Cl)cc2)CC1] |
| 340 | 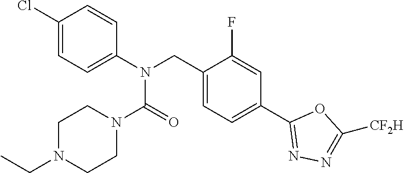 [SMILES: CCN1CCN(C(=O)N(Cc2ccc(-c3nnc(C(F)F)o3)cc2F)c2ccc(Cl)cc2)CC1] |
| 341 | 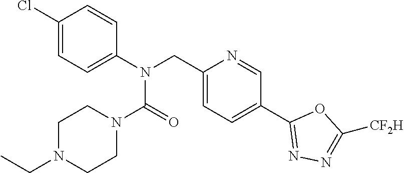 [SMILES: CCN1CCN(C(=O)N(Cc2ccc(-c3nnc(C(F)F)o3)cn2)c2ccc(Cl)cc2)CC1] |
| 342 | 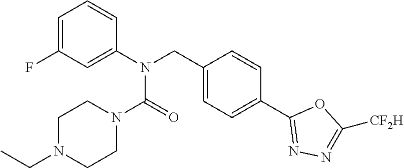 [SMILES: CCN1CCN(C(=O)N(Cc2ccc(-c3nnc(C(F)F)o3)cc2)c2cccc(F)c2)CC1] |
| 343 | 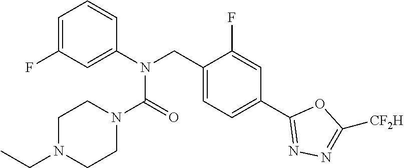 [SMILES: CCN1CCN(C(=O)N(Cc2ccc(-c3nnc(C(F)F)o3)cc2F)c2cccc(F)c2)CC1] |
| 344 | 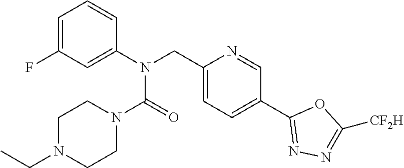 [SMILES: CCN1CCN(C(=O)N(Cc2ccc(-c3nnc(C(F)F)o3)cn2)c2cccc(F)c2)CC1] |
| 345 | 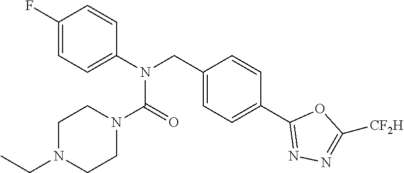 [SMILES: CCN1CCN(C(=O)N(Cc2ccc(-c3nnc(C(F)F)o3)cc2)c2ccc(F)cc2)CC1] |
| 346 | 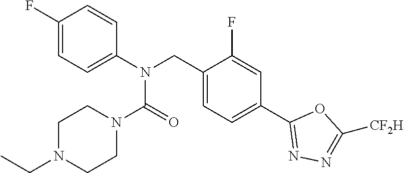 [SMILES: CCN1CCN(C(=O)N(Cc2ccc(-c3nnc(C(F)F)o3)cc2F)c2ccc(F)cc2)CC1] |
| 347 | 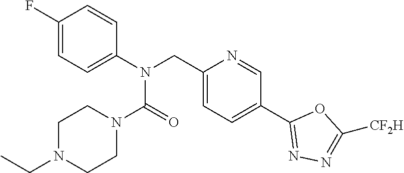 [SMILES: CCN1CCN(C(=O)N(Cc2ccc(-c3nnc(C(F)F)o3)cn2)c2ccc(F)cc2)CC1] |
| 348 | 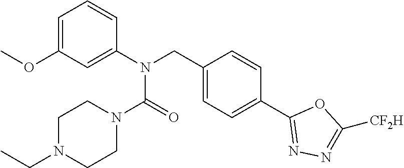 [SMILES: CCN1CCN(C(=O)N(Cc2ccc(-c3nnc(C(F)F)o3)cc2)c2cccc(OC)c2)CC1] |
| 349 | 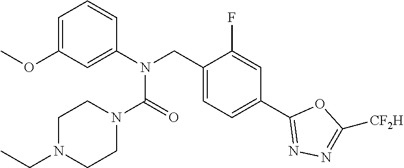 [SMILES: CCN1CCN(C(=O)N(Cc2ccc(-c3nnc(C(F)F)o3)cc2F)c2cccc(OC)c2)CC1] |
| 350 | 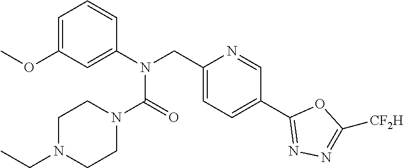 [SMILES: CCN1CCN(C(=O)N(Cc2ccc(-c3nnc(C(F)F)o3)cn2)c2cccc(OC)c2)CC1] |
| 351 | 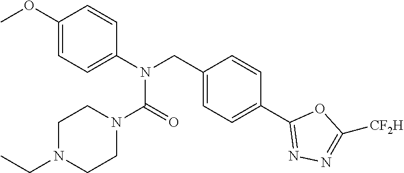 [SMILES: CCN1CCN(C(=O)N(Cc2ccc(-c3nnc(C(F)F)o3)cc2)c2ccc(OC)cc2)CC1] |
| 352 | 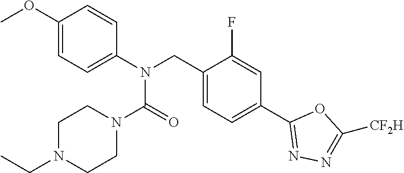 [SMILES: CCN1CCN(C(=O)N(Cc2ccc(-c3nnc(C(F)F)o3)cc2F)c2ccc(OC)cc2)CC1] |
| 353 | 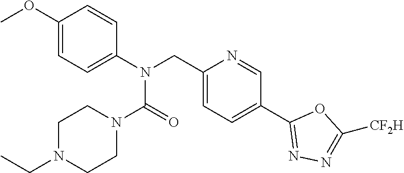 [SMILES: CCN1CCN(C(=O)N(Cc2ccc(-c3nnc(C(F)F)o3)cn2)c2ccc(OC)cc2)CC1] |
| 354 | 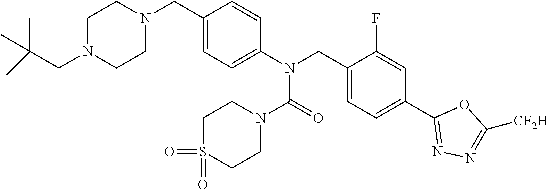 [SMILES: CC(C)(C)CN1CCN(Cc2ccc(N(Cc3ccc(-c4nnc(C(F)F)o4)cc3F)C(=O)N3CCS(=O)(=O)CC3)cc2)CC1] |
| 355 | 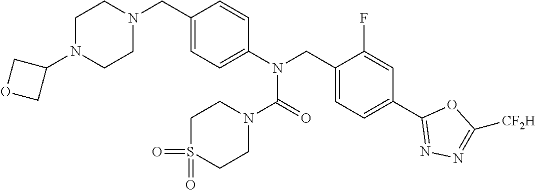 [SMILES: O=C(N1CCS(=O)(=O)CC1)N(Cc1ccc(-c2nnc(C(F)F)o2)cc1F)c1ccc(CN2CCN(C3COC3)CC2)cc1] |
| 356 | 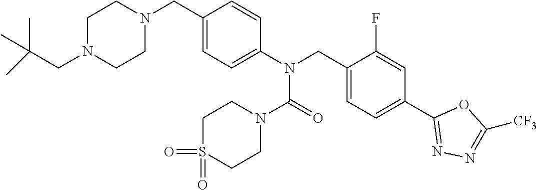 [SMILES: CC(C)(C)CN1CCN(Cc2ccc(N(Cc3ccc(-c4nnc(C(F)(F)F)o4)cc3F)C(=O)N3CCS(=O)(=O)CC3)cc2)CC1] |
| 357 | 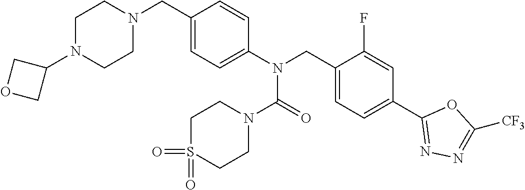 [SMILES: O=C(N1CCS(=O)(=O)CC1)N(Cc1ccc(-c2nnc(C(F)(F)F)o2)cc1F)c1ccc(CN2CCN(C3COC3)CC2)cc1] |
| 358 | 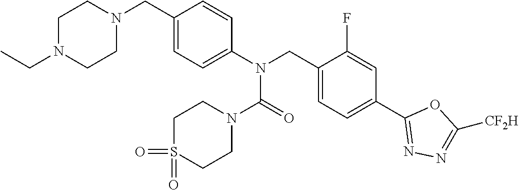 [SMILES: CCN1CCN(Cc2ccc(N(Cc3ccc(-c4nnc(C(F)F)o4)cc3F)C(=O)N3CCS(=O)(=O)CC3)cc2)CC1] |
| 359 | 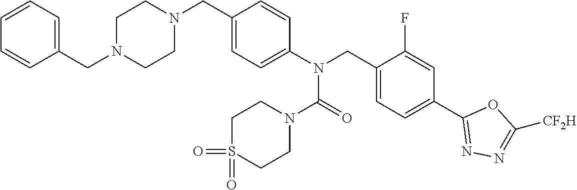 [SMILES: O=C(N1CCS(=O)(=O)CC1)N(Cc1ccc(-c2nnc(C(F)F)o2)cc1F)c1ccc(CN2CCN(Cc3ccccc3)CC2)cc1] |
| 360 | 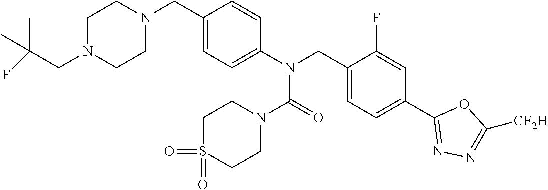 [SMILES: CC(C)(F)CN1CCN(Cc2ccc(N(Cc3ccc(-c4nnc(C(F)F)o4)cc3F)C(=O)N3CCS(=O)(=O)CC3)cc2)CC1] |
| 361 | 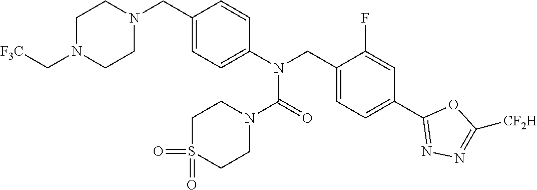 [SMILES: O=C(N1CCS(=O)(=O)CC1)N(Cc1ccc(-c2nnc(C(F)F)o2)cc1F)c1ccc(CN2CCN(CC(F)(F)F)CC2)cc1] |
| 362 | 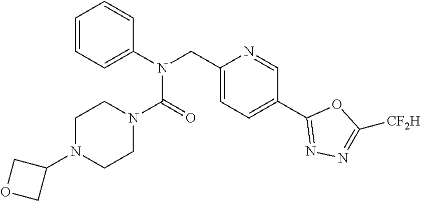 [SMILES: O=C(N1CCN(C2COC2)CC1)N(Cc1ccc(-c2nnc(C(F)F)o2)cn1)c1ccccc1] |
| 363 | 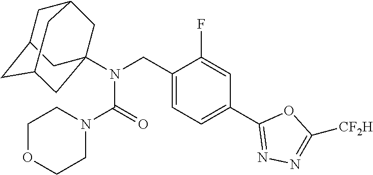 [SMILES: O=C(N1CCOCC1)N(Cc1ccc(-c2nnc(C(F)F)o2)cc1F)C12CC3CC(CC(C3)C1)C2] |
| 364 | 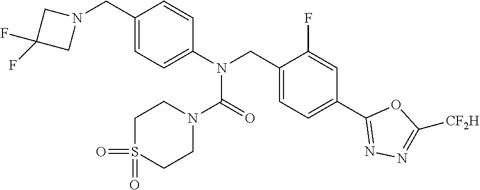 [SMILES: O=C(N1CCS(=O)(=O)CC1)N(Cc1ccc(-c2nnc(C(F)F)o2)cc1F)c1ccc(CN2CC(F)(F)C2)cc1] |
| 365 | 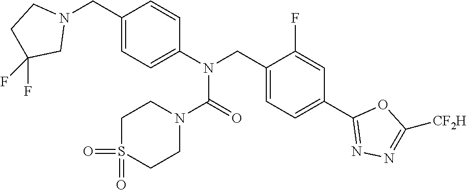 [SMILES: O=C(N1CCS(=O)(=O)CC1)N(Cc1ccc(-c2nnc(C(F)F)o2)cc1F)c1ccc(CN2CCC(F)(F)C2)cc1] |
| 366 | 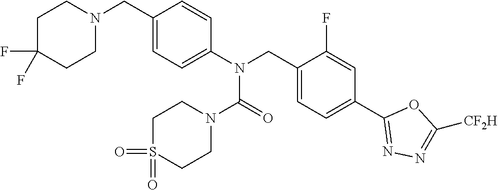 [SMILES: O=C(N1CCS(=O)(=O)CC1)N(Cc1ccc(-c2nnc(C(F)F)o2)cc1F)c1ccc(CN2CCC(F)(F)CC2)cc1] |
| 367 | 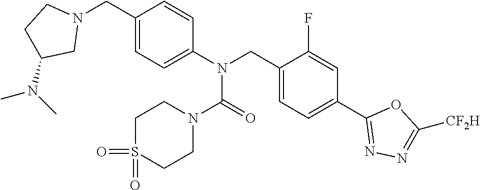 [SMILES: CN(C)[C@@H]1CCN(Cc2ccc(N(Cc3ccc(-c4nnc(C(F)F)o4)cc3F)C(=O)N3CCS(=O)(=O)CC3)cc2)C1] |
| 368 | 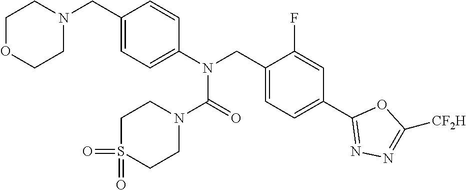 [SMILES: O=C(N1CCS(=O)(=O)CC1)N(Cc1ccc(-c2nnc(C(F)F)o2)cc1F)c1ccc(CN2CCOCC2)cc1] |
| 369 | 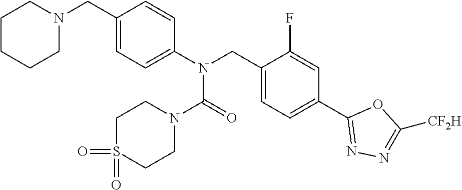 [SMILES: O=C(N1CCS(=O)(=O)CC1)N(Cc1ccc(-c2nnc(C(F)F)o2)cc1F)c1ccc(CN2CCCCC2)cc1] |
| 370 | 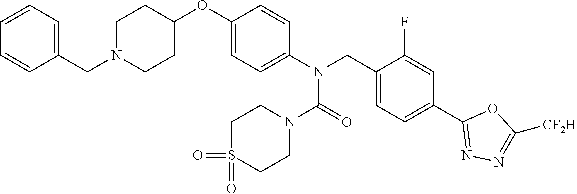 [SMILES: O=C(N1CCS(=O)(=O)CC1)N(Cc1ccc(-c2nnc(C(F)F)o2)cc1F)c1ccc(OC2CCN(Cc3ccccc3)CC2)cc1] |
| 371 | 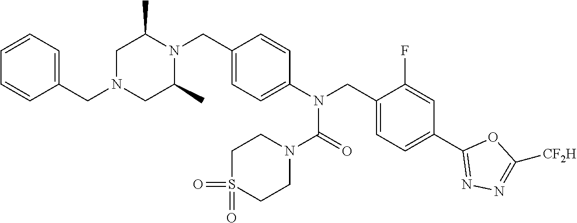 [SMILES: C[C@@H]1CN(Cc2ccccc2)C[C@H](C)N1Cc1ccc(N(Cc2ccc(-c3nnc(C(F)F)o3)cc2F)C(=O)N2CCS(=O)(=O)CC2)cc1] |
| 372 | 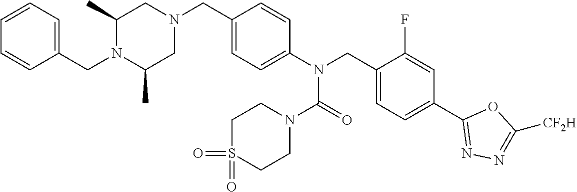 [SMILES: C[C@@H]1CN(Cc2ccc(N(Cc3ccc(-c4nnc(C(F)F)o4)cc3F)C(=O)N3CCS(=O)(=O)CC3)cc2)C[C@H](C)N1Cc1ccccc1] |
| 373 | 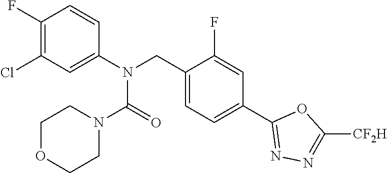 [SMILES: O=C(N1CCOCC1)N(Cc1ccc(-c2nnc(C(F)F)o2)cc1F)c1ccc(F)c(Cl)c1] |
| 374 | 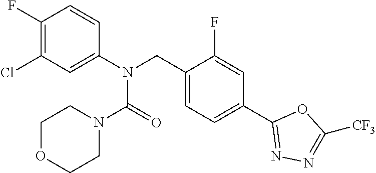 [SMILES: O=C(N1CCOCC1)N(Cc1ccc(-c2nnc(C(F)(F)F)o2)cc1F)c1ccc(F)c(Cl)c1] |
| 375 | 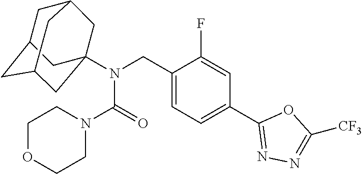 [SMILES: O=C(N1CCOCC1)N(Cc1ccc(-c2nnc(C(F)(F)F)o2)cc1F)C12CC3CC(CC(C3)C1)C2] |
| 376 | 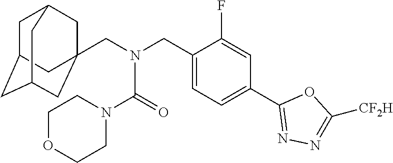 [SMILES: O=C(N1CCOCC1)N(Cc1ccc(-c2nnc(C(F)F)o2)cc1F)CC12CC3CC(CC(C3)C1)C2] |
| 377 | 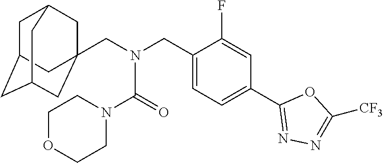 [SMILES: O=C(N1CCOCC1)N(Cc1ccc(-c2nnc(C(F)(F)F)o2)cc1F)CC12CC3CC(CC(C3)C1)C2] |
| 378 | 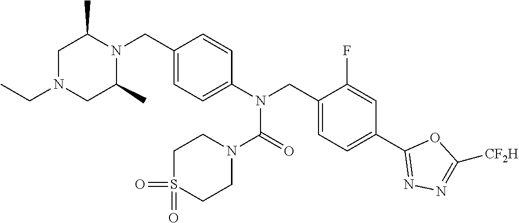 [SMILES: CCN1C[C@@H](C)N(Cc2ccc(N(Cc3ccc(-c4nnc(C(F)F)o4)cc3F)C(=O)N3CCS(=O)(=O)CC3)cc2)[C@@H](C)C1] |
| 379 | 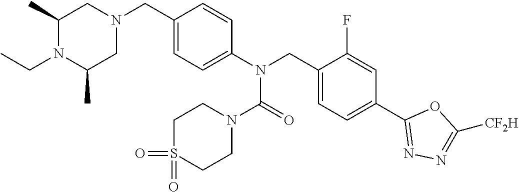 [SMILES: CCN1[C@H](C)CN(Cc2ccc(N(Cc3ccc(-c4nnc(C(F)F)o4)cc3F)C(=O)N3CCS(=O)(=O)CC3)cc2)C[C@@H]1C] |
| 380 | 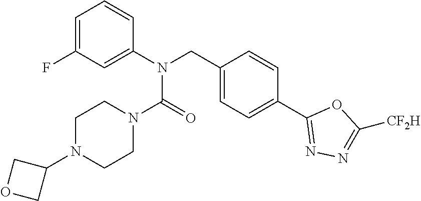 [SMILES: O=C(N1CCN(C2COC2)CC1)N(Cc1ccc(-c2nnc(C(F)F)o2)cc1)c1cccc(F)c1] |
| 381 | 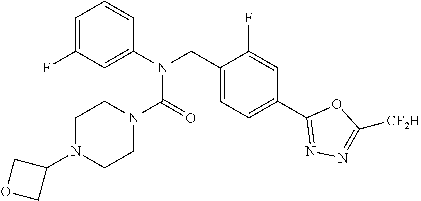 [SMILES: O=C(N1CCN(C2COC2)CC1)N(Cc1ccc(-c2nnc(C(F)F)o2)cc1F)c1cccc(F)c1] |
| 382 | 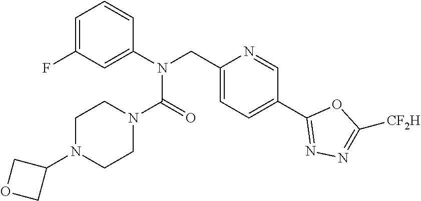 [SMILES: O=C(N1CCN(C2COC2)CC1)N(Cc1ccc(-c2nnc(C(F)F)o2)cn1)c1cccc(F)c1] |
| 383 | 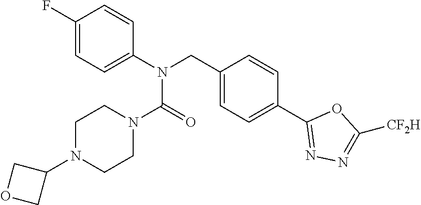 [SMILES: O=C(N1CCN(C2COC2)CC1)N(Cc1ccc(-c2nnc(C(F)F)o2)cc1)c1ccc(F)cc1] |
| 384 | 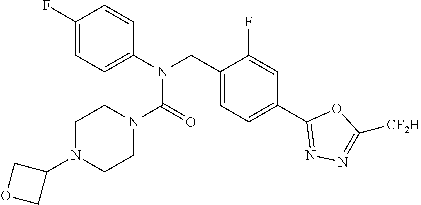 [SMILES: O=C(N1CCN(C2COC2)CC1)N(Cc1ccc(-c2nnc(C(F)F)o2)cc1F)c1ccc(F)cc1] |
| 385 | 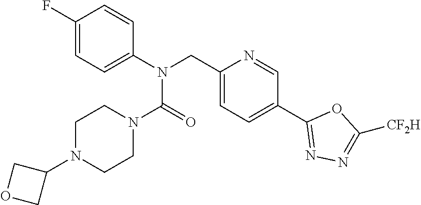 [SMILES: O=C(N1CCN(C2COC2)CC1)N(Cc1ccc(-c2nnc(C(F)F)o2)cn1)c1ccc(F)cc1] |
| 386 | 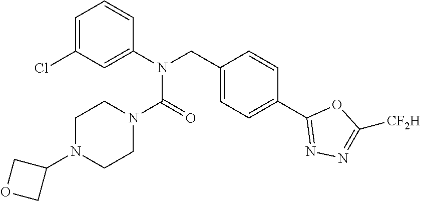 [SMILES: O=C(N1CCN(C2COC2)CC1)N(Cc1ccc(-c2nnc(C(F)F)o2)cc1)c1cccc(Cl)c1] |
| 387 | 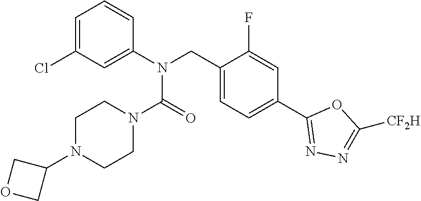 [SMILES: O=C(N1CCN(C2COC2)CC1)N(Cc1ccc(-c2nnc(C(F)F)o2)cc1F)c1cccc(Cl)c1] |
| 388 | 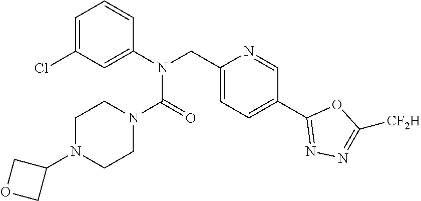 [SMILES: O=C(N1CCN(C2COC2)CC1)N(Cc1ccc(-c2nnc(C(F)F)o2)cn1)c1cccc(Cl)c1] |
| 389 | 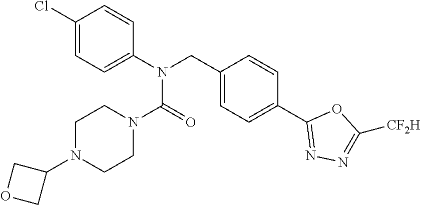 [SMILES: O=C(N1CCN(C2COC2)CC1)N(Cc1ccc(-c2nnc(C(F)F)o2)cc1)c1ccc(Cl)cc1] |
| 390 | 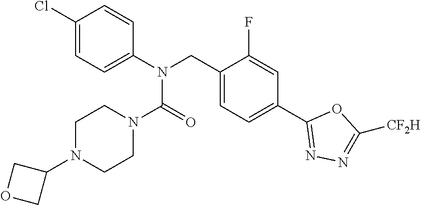 [SMILES: O=C(N1CCN(C2COC2)CC1)N(Cc1ccc(-c2nnc(C(F)F)o2)cc1F)c1ccc(Cl)cc1] |
| 391 | 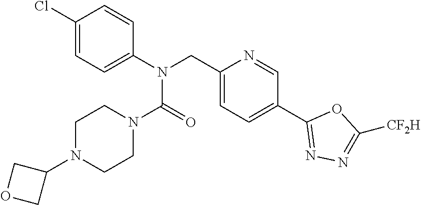 [SMILES: O=C(N1CCN(C2COC2)CC1)N(Cc1ccc(-c2nnc(C(F)F)o2)cn1)c1ccc(Cl)cc1] |
| 392 | 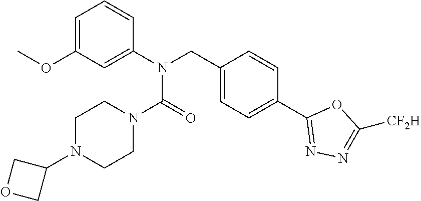 [SMILES: COc1cccc(N(Cc2ccc(-c3nnc(C(F)F)o3)cc2)C(=O)N2CCN(C3COC3)CC2)c1] |
| 393 | 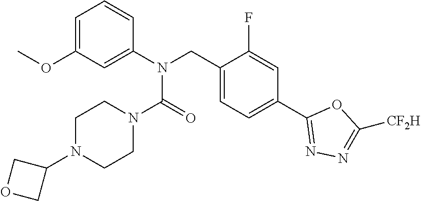 [SMILES: COc1cccc(N(Cc2ccc(-c3nnc(C(F)F)o3)cc2F)C(=O)N2CCN(C3COC3)CC2)c1] |
| 394 | 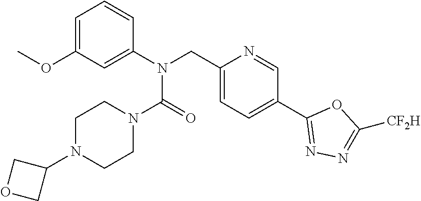 [SMILES: COc1cccc(N(Cc2ccc(-c3nnc(C(F)F)o3)cn2)C(=O)N2CCN(C3COC3)CC2)c1] |
| 395 | 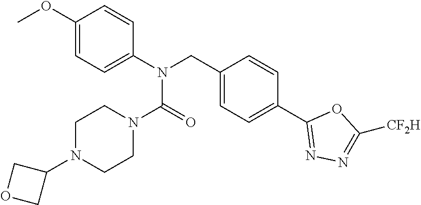 [SMILES: COc1ccc(N(Cc2ccc(-c3nnc(C(F)F)o3)cc2)C(=O)N2CCN(C3COC3)CC2)cc1] |
| 396 | 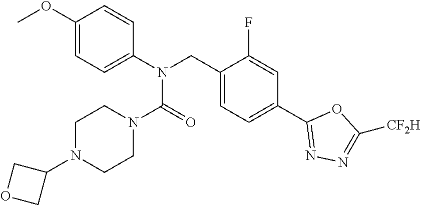 [SMILES: COc1ccc(N(Cc2ccc(-c3nnc(C(F)F)o3)cc2F)C(=O)N2CCN(C3COC3)CC2)cc1] |
| 397 | 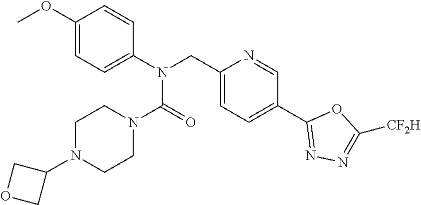 [SMILES: COc1ccc(N(Cc2ccc(-c3nnc(C(F)F)o3)cn2)C(=O)N2CCN(C3COC3)CC2)cc1] |
| 398 | 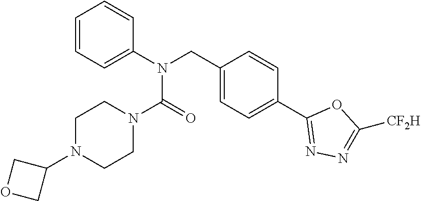 [SMILES: O=C(N1CCN(C2COC2)CC1)N(Cc1ccc(-c2nnc(C(F)F)o2)cc1)c1ccccc1] |
| 399 | 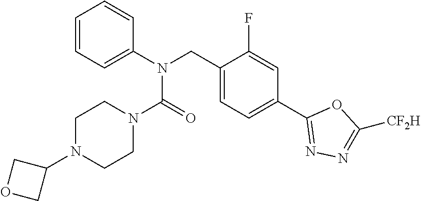 [SMILES: O=C(N1CCN(C2COC2)CC1)N(Cc1ccc(-c2nnc(C(F)F)o2)cc1F)c1ccccc1] |
| 400 | 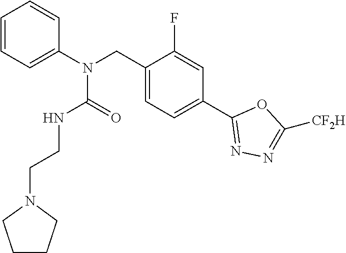 [SMILES: O=C(NCCN1CCCC1)N(Cc1ccc(-c2nnc(C(F)F)o2)cc1F)c1ccccc1] |
| 401 | 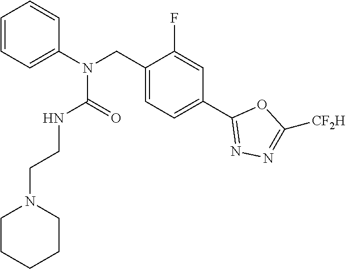 [SMILES: O=C(NCCN1CCCCC1)N(Cc1ccc(-c2nnc(C(F)F)o2)cc1F)c1ccccc1] |
| 402 | 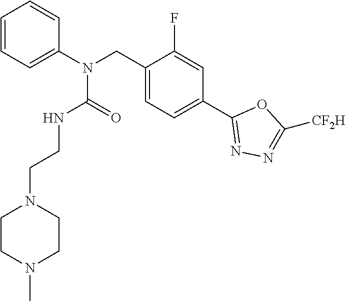 [SMILES: CN1CCN(CCNC(=O)N(Cc2ccc(-c3nnc(C(F)F)o3)cc2F)c2ccccc2)CC1] |
| 403 | 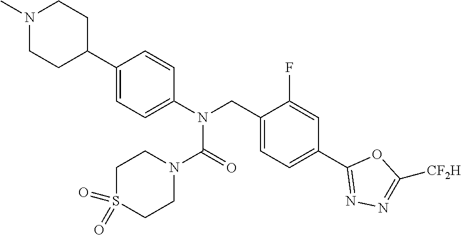 [SMILES: CN1CCC(c2ccc(N(Cc3ccc(-c4nnc(C(F)F)o4)cc3F)C(=O)N3CCS(=O)(=O)CC3)cc2)CC1] |
| 404 | 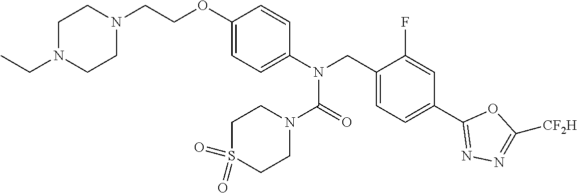 [SMILES: CCN1CCN(CCOc2ccc(N(Cc3ccc(-c4nnc(C(F)F)o4)cc3F)C(=O)N3CCS(=O)(=O)CC3)cc2)CC1] |
| 405 | 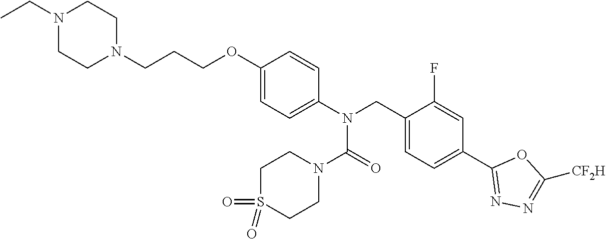 [SMILES: CCN1CCN(CCCOc2ccc(N(Cc3ccc(-c4nnc(C(F)F)o4)cc3F)C(=O)N3CCS(=O)(=O)CC3)cc2)CC1] |
| 406 | 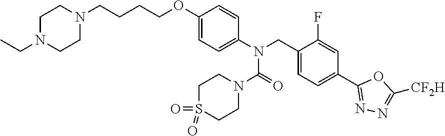 [SMILES: CCN1CCN(CCCCOc2ccc(N(Cc3ccc(-c4nnc(C(F)F)o4)cc3F)C(=O)N3CCS(=O)(=O)CC3)cc2)CC1] |
| 407 | 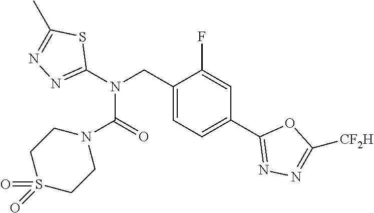 [SMILES: Cc1nnc(N(Cc2ccc(-c3nnc(C(F)F)o3)cc2F)C(=O)N2CCS(=O)(=O)CC2)s1] |
| 408 | 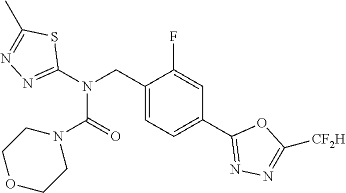 [SMILES: Cc1nnc(N(Cc2ccc(-c3nnc(C(F)F)o3)cc2F)C(=O)N2CCOCC2)s1] |
| 409 | 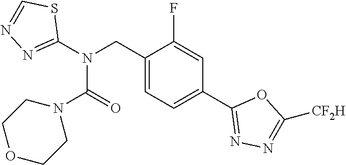 [SMILES: O=C(N1CCOCC1)N(Cc1ccc(-c2nnc(C(F)F)o2)cc1F)c1nncs1] |
| 410 | 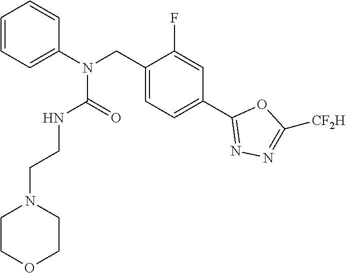 [SMILES: O=C(NCCN1CCOCC1)N(Cc1ccc(-c2nnc(C(F)F)o2)cc1F)c1ccccc1] |
| 411 | 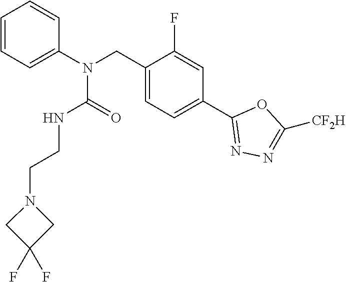 [SMILES: O=C(NCCN1CC(F)(F)C1)N(Cc1ccc(-c2nnc(C(F)F)o2)cc1F)c1ccccc1] |
| 412 | 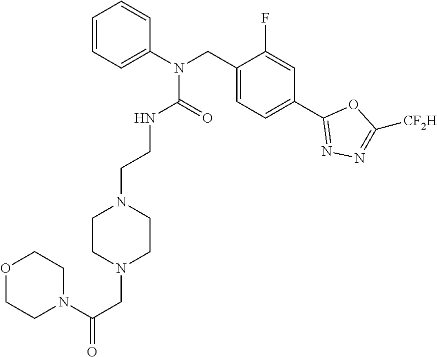 [SMILES: O=C(CN1CCN(CCNC(=O)N(Cc2ccc(-c3nnc(C(F)F)o3)cc2F)c2ccccc2)CC1)N1CCOCC1] |
| 413 | 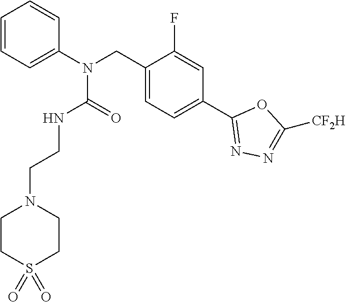 [SMILES: O=C(NCCN1CCS(=O)(=O)CC1)N(Cc1ccc(-c2nnc(C(F)F)o2)cc1F)c1ccccc1] |
| 414 | 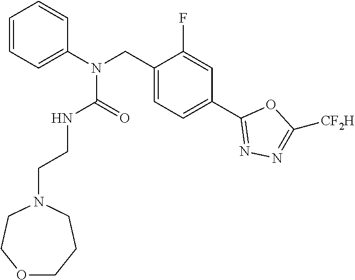 [SMILES: O=C(NCCN1CCCOCC1)N(Cc1ccc(-c2nnc(C(F)F)o2)cc1F)c1ccccc1] |
| 415 | 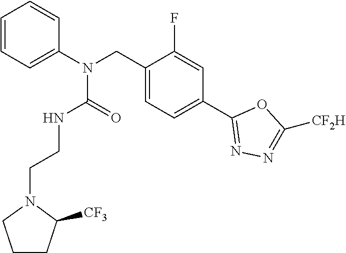 [SMILES: O=C(NCCN1CCC[C@@H]1C(F)(F)F)N(Cc1ccc(-c2nnc(C(F)F)o2)cc1F)c1ccccc1] |
| 416 | 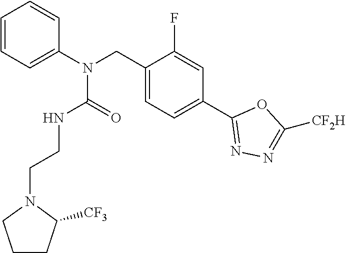 [SMILES: O=C(NCCN1CCC[C@H]1C(F)(F)F)N(Cc1ccc(-c2nnc(C(F)F)o2)cc1F)c1ccccc1] |
| 417 | 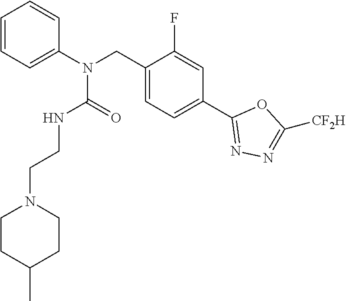 [SMILES: CC1CCN(CCNC(=O)N(Cc2ccc(-c3nnc(C(F)F)o3)cc2F)c2ccccc2)CC1] |
| 418 | 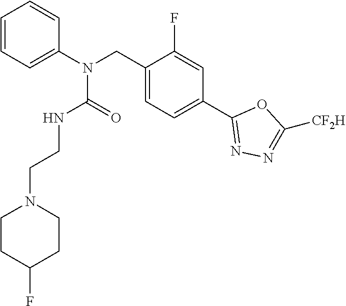 [SMILES: O=C(NCCN1CCC(F)CC1)N(Cc1ccc(-c2nnc(C(F)F)o2)cc1F)c1ccccc1] |
| 419 | 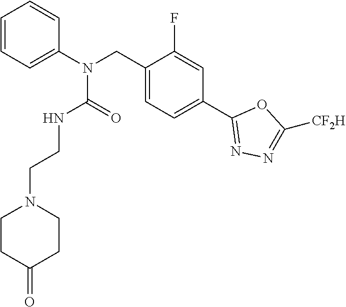 [SMILES: O=C1CCN(CCNC(=O)N(Cc2ccc(-c3nnc(C(F)F)o3)cc2F)c2ccccc2)CC1] |
| 420 | 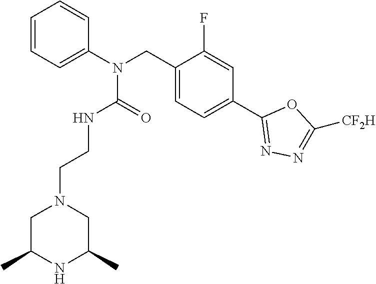 [SMILES: C[C@@H]1CN(CCNC(=O)N(Cc2ccc(-c3nnc(C(F)F)o3)cc2F)c2ccccc2)C[C@H](C)N1] |
| 421 | 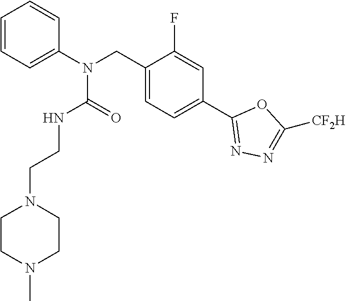 [SMILES: CN1CCN(CCNC(=O)N(Cc2ccc(-c3nnc(C(F)F)o3)cc2F)c2ccccc2)CC1] |
| 422 | 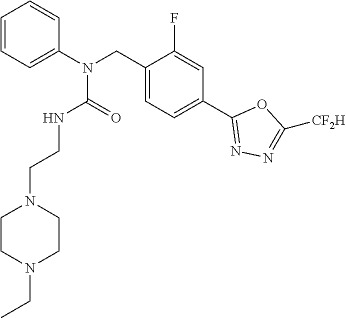 [SMILES: CCN1CCN(CCNC(=O)N(Cc2ccc(-c3nnc(C(F)F)o3)cc2F)c2ccccc2)CC1] |
| 423 | 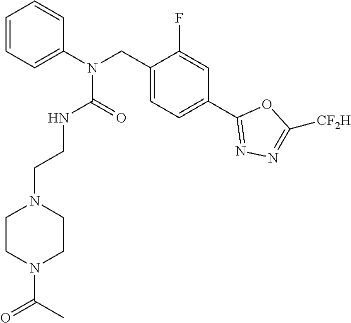 [SMILES: CC(=O)N1CCN(CCNC(=O)N(Cc2ccc(-c3nnc(C(F)F)o3)cc2F)c2ccccc2)CC1] |
| 424 | 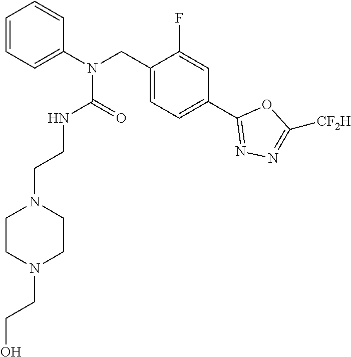 [SMILES: O=C(NCCN1CCN(CCO)CC1)N(Cc1ccc(-c2nnc(C(F)F)o2)cc1F)c1ccccc1] |
| 425 | 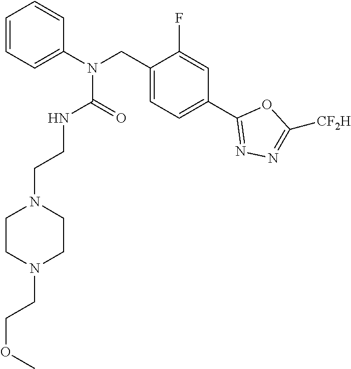 [SMILES: COCCN1CCN(CCNC(=O)N(Cc2ccc(-c3nnc(C(F)F)o3)cc2F)c2ccccc2)CC1] |
| 426 | 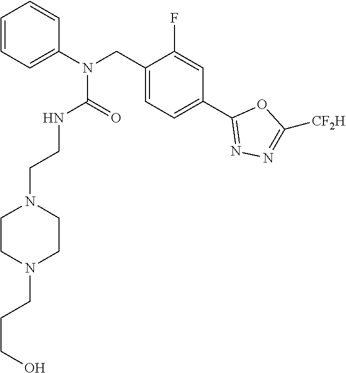 [SMILES: O=C(NCCN1CCN(CCCO)CC1)N(Cc1ccc(-c2nnc(C(F)F)o2)cc1F)c1ccccc1] |
| 427 | 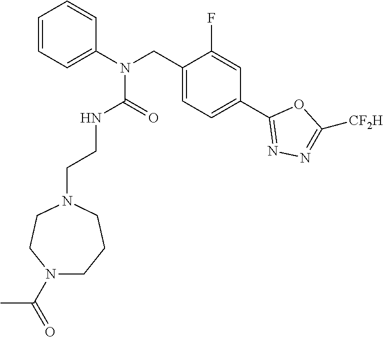 [SMILES: CC(=O)N1CCCN(CCNC(=O)N(Cc2ccc(-c3nnc(C(F)F)o3)cc2F)c2ccccc2)CC1] |
| 428 | 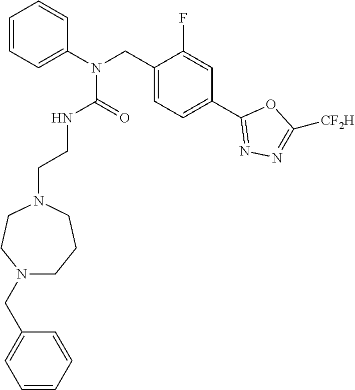 [SMILES: O=C(NCCN1CCCN(Cc2ccccc2)CC1)N(Cc1ccc(-c2nnc(C(F)F)o2)cc1F)c1ccccc1] |
| 429 | 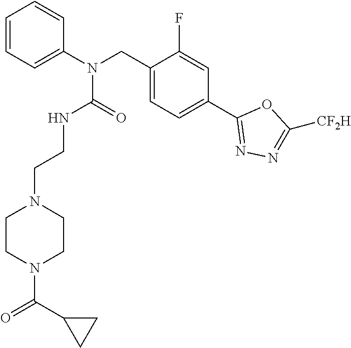 [SMILES: O=C(C1CC1)N1CCN(CCNC(=O)N(Cc2ccc(-c3nnc(C(F)F)o3)cc2F)c2ccccc2)CC1] |
| 430 | 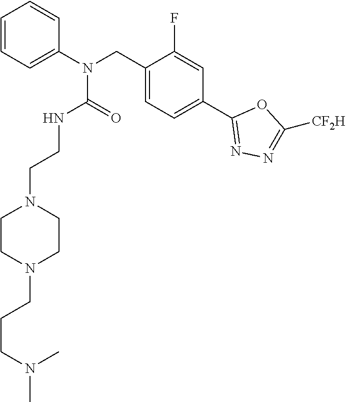 [SMILES: CN(C)CCCN1CCN(CCNC(=O)N(Cc2ccc(-c3nnc(C(F)F)o3)cc2F)c2ccccc2)CC1] |
| 431 | 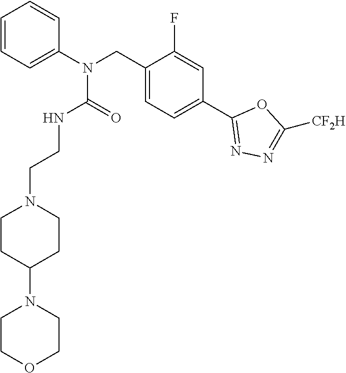 [SMILES: O=C(NCCN1CCC(N2CCOCC2)CC1)N(Cc1ccc(-c2nnc(C(F)F)o2)cc1F)c1ccccc1] |
| 432 | 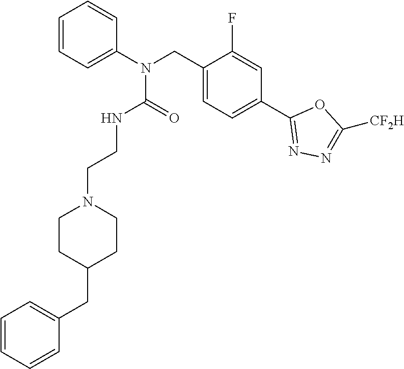 [SMILES: O=C(NCCN1CCC(Cc2ccccc2)CC1)N(Cc1ccc(-c2nnc(C(F)F)o2)cc1F)c1ccccc1] |
| 433 | 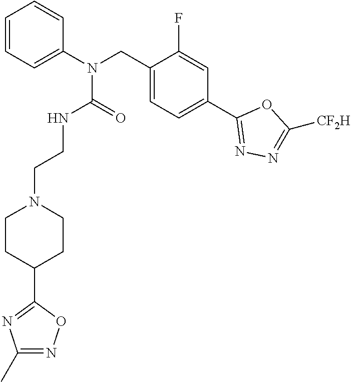 [SMILES: Cc1noc(C2CCN(CCNC(=O)N(Cc3ccc(-c4nnc(C(F)F)o4)cc3F)c3ccccc3)CC2)n1] |
| 434 | 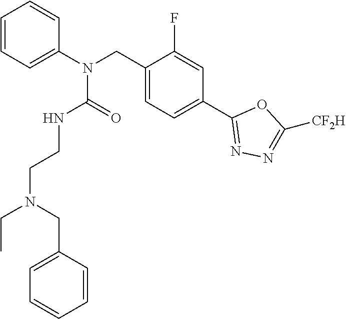 [SMILES: CCN(CCNC(=O)N(Cc1ccc(-c2nnc(C(F)F)o2)cc1F)c1ccccc1)Cc1ccccc1] |
| 435 | 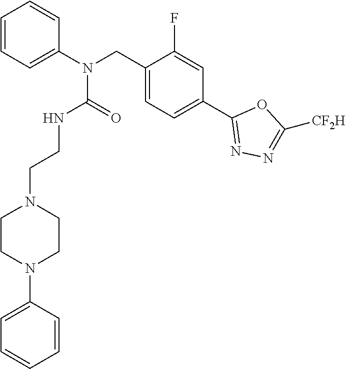 [SMILES: O=C(NCCN1CCN(c2ccccc2)CC1)N(Cc1ccc(-c2nnc(C(F)F)o2)cc1F)c1ccccc1] |
| 436 | 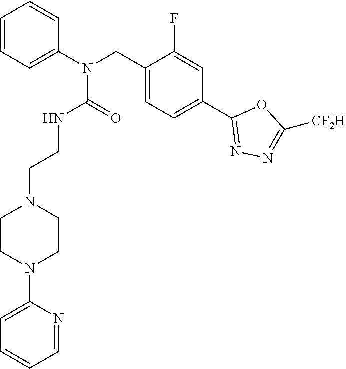 [SMILES: O=C(NCCN1CCN(c2ccccn2)CC1)N(Cc1ccc(-c2nnc(C(F)F)o2)cc1F)c1ccccc1] |
| 437 | 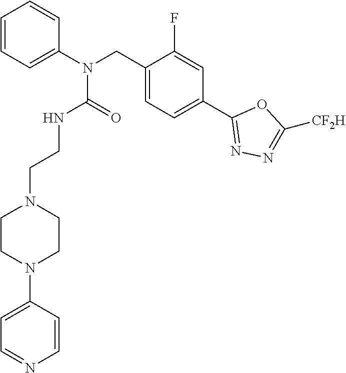 [SMILES: O=C(NCCN1CCN(c2ccncc2)CC1)N(Cc1ccc(-c2nnc(C(F)F)o2)cc1F)c1ccccc1] |
| 438 | 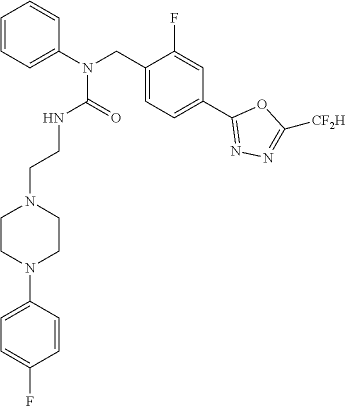 [SMILES: O=C(NCCN1CCN(c2ccc(F)cc2)CC1)N(Cc1ccc(-c2nnc(C(F)F)o2)cc1F)c1ccccc1] |
| 439 | 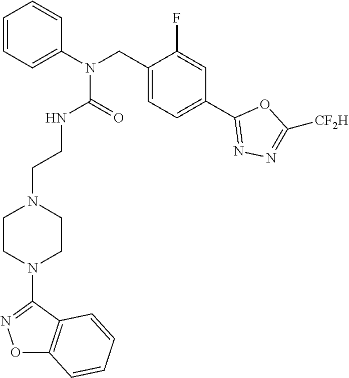 [SMILES: O=C(NCCN1CCN(c2noc3ccccc23)CC1)N(Cc1ccc(-c2nnc(C(F)F)o2)cc1F)c1ccccc1] |
| 440 | 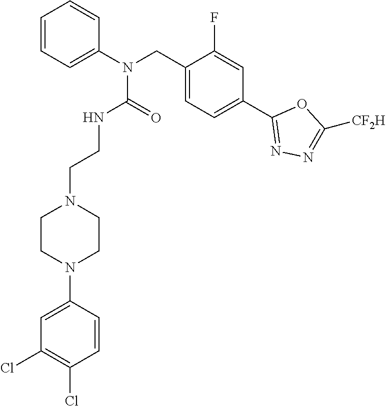 [SMILES: O=C(NCCN1CCN(c2ccc(Cl)c(Cl)c2)CC1)N(Cc1ccc(-c2nnc(C(F)F)o2)cc1F)c1ccccc1] |
| 441 | 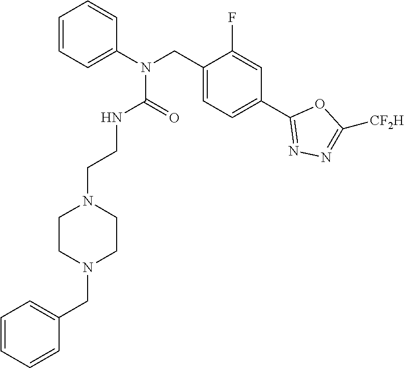 [SMILES: O=C(NCCN1CCN(Cc2ccccc2)CC1)N(Cc1ccc(-c2nnc(C(F)F)o2)cc1F)c1ccccc1] |
| 442 | 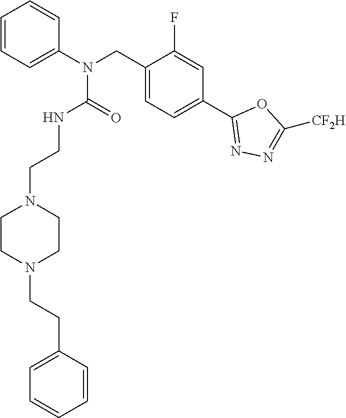 [SMILES: O=C(NCCN1CCN(CCc2ccccc2)CC1)N(Cc1ccc(-c2nnc(C(F)F)o2)cc1F)c1ccccc1] |
| 443 | 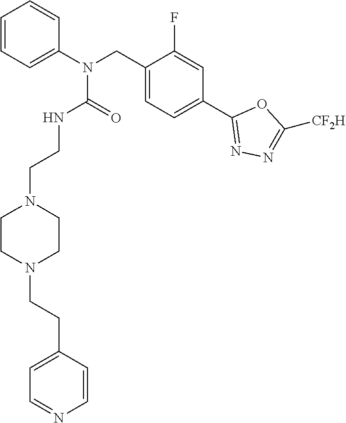 [SMILES: O=C(NCCN1CCN(CCc2ccncc2)CC1)N(Cc1ccc(-c2nnc(C(F)F)o2)cc1F)c1ccccc1] |
| 444 | 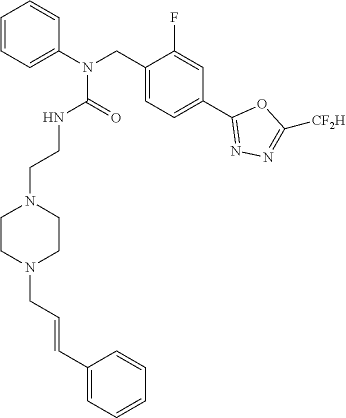 [SMILES: O=C(NCCN1CCN(C/C=C/c2ccccc2)CC1)N(Cc1ccc(-c2nnc(C(F)F)o2)cc1F)c1ccccc1] |
| 445 | 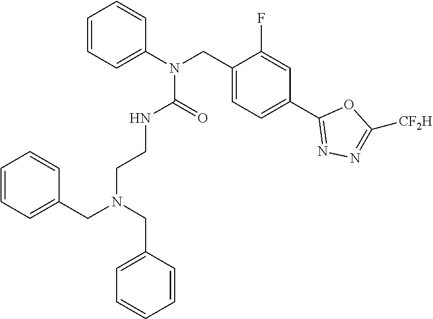 [SMILES: O=C(NCCN(Cc1ccccc1)Cc1ccccc1)N(Cc1ccc(-c2nnc(C(F)F)o2)cc1F)c1ccccc1] |
| 446 | 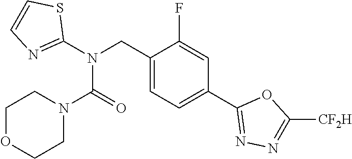 [SMILES: O=C(N1CCOCC1)N(Cc1ccc(-c2nnc(C(F)F)o2)cc1F)c1nccs1] |
| 447 | 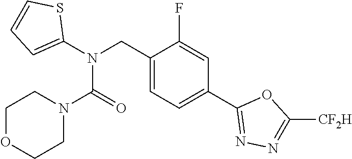 [SMILES: O=C(N1CCOCC1)N(Cc1ccc(-c2nnc(C(F)F)o2)cc1F)c1cccs1] |
| 448 | 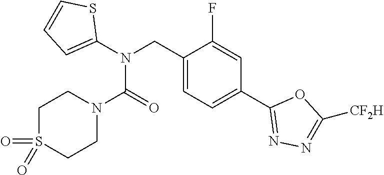 [SMILES: O=C(N1CCS(=O)(=O)CC1)N(Cc1ccc(-c2nnc(C(F)F)o2)cc1F)c1cccs1] |
| 449 | 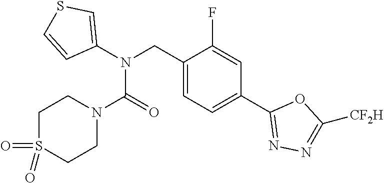 [SMILES: O=C(N1CCS(=O)(=O)CC1)N(Cc1ccc(-c2nnc(C(F)F)o2)cc1F)c1ccsc1] |
| 450 | 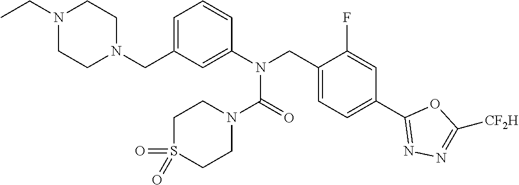 [SMILES: CCN1CCN(Cc2cccc(N(Cc3ccc(-c4nnc(C(F)F)o4)cc3F)C(=O)N3CCS(=O)(=O)CC3)c2)CC1] |

In example embodiment of the present invention, the pharmaceutical composition including a compound of Table A, optical isomers thereof or pharmaceutically acceptable salts thereof as an active ingredient may prevent or treat Charcot-Marie-Tooth (CMT) disease associated with the peripheral nervous systems (PNS).

In the pharmaceutical composition according to the disclosure, the compounds represented by formula I may be shown in Table B below:

TABLE B

| Com- | |
|---|---|
| pound | Structure |
| 43 | 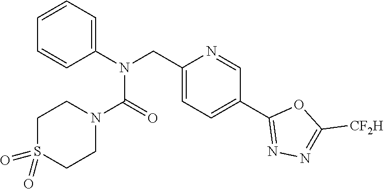 [SMILES: O=C(N1CCS(=O)(=O)CC1)N(Cc1ccc(-c2nnc(C(F)F)o2)cn1)c1ccccc1] |
| 232 | 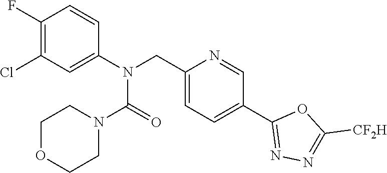 [SMILES: O=C(N1CCOCC1)N(Cc1ccc(-c2nnc(C(F)F)o2)cn1)c1ccc(F)c(Cl)c1] |
| 239 | 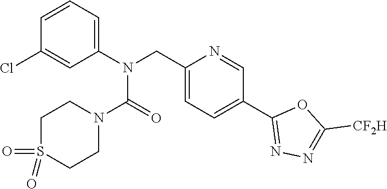 [SMILES: O=C(N1CCS(=O)(=O)CC1)N(Cc1ccc(-c2nnc(C(F)F)o2)cn1)c1cccc(Cl)c1] |
| 243 | 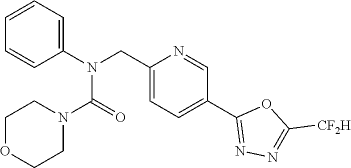 [SMILES: O=C(N1CCOCC1)N(Cc1ccc(-c2nnc(C(F)F)o2)cn1)c1ccccc1] |
| 286 | 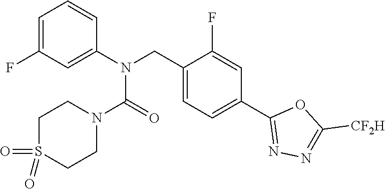 [SMILES: O=C(N1CCS(=O)(=O)CC1)N(Cc1ccc(-c2nnc(C(F)F)o2)cc1F)c1cccc(F)c1] |

In example embodiment of the present invention, the pharmaceutical composition including a compound of Table B, optical isomers thereof or pharmaceutically acceptable salts thereof as an active ingredient may prevent or treat Charcot-Marie-Tooth (CMT) disease associated with the peripheral nervous systems (PNS).

In the present disclosure, the compound represented by above formula I may be prepared by a method disclosed in Korean Unexamined Patent Application Publication No. 10-2017-0017792, but is not limited thereto.

In the present disclosure, the compound represented by the above formula I may contain at least one asymmetric carbon, and thus may be present as a racemic mixture, a single enantiomer (optical isomer), a mixture of diastereomers, and a single diastereomer. Such isomer may be separated by being split according to the prior art, for example, column chromatography, HPLC or the like. Alternatively, the isomer may be stereospecifically synthesized with a known array of optically pure starting materials and/or reagents. Particularly, said isomer may be an optical isomer(enantiomer).

In the present disclosure, the term “pharmaceutically acceptable” may refer to the one which is physiologically acceptable and does not conventionally cause gastrointestinal disturbance, an allergic response such as dizziness or other responses similar thereto, when being administered to an individual.

The pharmaceutically acceptable salts according to the embodiments of the present invention may be prepared by a conventional method known to those skilled in the art.

The pharmaceutically acceptable salts according to the embodiment of the present invention may include, for example, inorganic ion salts prepared from calcium, potassium, sodium, magnesium, etc.; inorganic acid salts prepared from hydrochloric acid, nitric acid, phosphoric acid, bromic acid, iodic acid, perchloric acid, sulfuric acid, hydroiodic acid, etc.; organic acid salts prepared from acetic acid, trifluoroacetic acid, citric acid, maleic acid, succinic acid, oxalic acid, benzoic acid, tartaric acid, fumaric acid, mandelic acid, propionic acid, lactic acid, glycolic acid, gluconic acid, galacturonic acid, glutamic acid, glutaric acid, glucuronic acid, aspartic acid, ascorbic acid, carbonic acid, vanillic acid, etc.; sulfonic acid salts prepared from methanesulfonic acid, ethanesulfonic acid, benzenesulfonic acid, p-toluenesulfonic acid, naphthalene sulfonic acid, etc.; amino acid salts prepared from glycine, arginine, lysine, etc.; amine salts prepared from trimethylamine, triethylamine, ammonia, pyridine, picoline, etc.; and the like, but are not limited thereto. In the embodiments of the present invention, salts may include hydrochloric acid, trifluoroacetic acid, citric acid, bromic acid, maleic acid, phosphoric acid, sulfuric acid, tartaric acid or a mixture thereof.

In the present disclosure, the term “Charcot-Marie-Tooth (CMT) disease” may refer to a degenerative peripheral neuropathy that causes dysfunction and death of peripheral nerve cells due to various genetic factors, indicating a disease, of which the main etiological cause is an axonal transport defect.

In the present disclosure, Charcot-Marie-Tooth disease associated with the peripheral nervous system may be at least one selected from the group consisting of CMT1 type, CMT2 type, CMT4 type, degerine-sottas syndrome (DSN), congenital hypomyelination (CH), hereditary neuropathy with liability to pressure palsy (HNPP) and giant axonal neuropathy (GAN), but is not limited thereto.

The CMT1 type may be at least one selected from the group consisting of CMT1A, CMT1B, CMT1C, CMT1D and CMTX, and the CMT2 type may be at least one selected from the group consisting of CMT2A, CMT2B, CMT2C, CMT2D, CMT2E and CMT2F, and the CMT4 type may be at least one selected from the group consisting of CMT4A, CMT4B1, CMT4B2, CMT4C, CMT4D, CMT4E and CMT4F.

In the present disclosure, said Charcot-Marie-Tooth disease associated with the peripheral nervous system may be at least one selected from the group consisting of CMT1A, CMT2D and CMT2F, but is not limited thereto.

In example embodiments of the present invention, the compound according to the present disclosure may prevent or treat the symptom associated with degeneration of the peripheral nervous systems in a subject with Charcot-Marie-Tooth disease.

In example embodiments of the present invention, the compound according to the present disclosure may prevent or treat the symptom associated with dysfunction and/or death of peripheral nerve cells in a subject with Charcot-Marie-Tooth disease.

In example embodiments of the present invention, the compound according to the present disclosure may prevent or treat a degenerative peripheral neuropathy that is caused by dysfunction and/or death of peripheral nerve cells in a subject with Charcot-Marie-Tooth disease.

In the present disclosure, the term “prevention” may refer to all the acts, which inhibit or delay the occurrence of a disease by administering the compound of formula I of the present disclosure, optical isomers thereof or pharmaceutically acceptable salts thereof.

In the present disclosure, the term “treatment” may refer to all the acts, by which a symptom of an individual likely to develop or suffering from a disease gets better or takes a favorable turn by administering the compound of formula I of the present disclosure, optical isomers thereof or pharmaceutically acceptable salts thereof.

The compound represented by formula I of the present disclosure, optical isomers thereof or pharmaceutically acceptable salts thereof may be advantageously used in preventing or treating Charcot-Marie-Tooth disease associated with the peripheral nervous system.

A pharmaceutical composition including the compound represented by formula I of the present disclosure, optical isomers thereof, or pharmaceutically acceptable salts thereof as an active ingredient may be advantageously used in preventing or treating Charcot-Marie-Tooth disease associated with the peripheral nervous system.

In example embodiments of the present invention, the pharmaceutical composition including the compound according to the present disclosure may prevent or treat the symptom associated with degeneration of the peripheral nervous systems in a subject with Charcot-Marie-Tooth disease.

In example embodiments of the present invention, the pharmaceutical composition including the compound according to the present disclosure may prevent or treat the symptom associated with dysfunction and/or death of peripheral nerve cells in a subject with Charcot-Marie-Tooth disease.

In example embodiments of the present invention, the pharmaceutical composition including the compound according to the present disclosure may prevent or treat a degenerative peripheral neuropathy that is caused by dysfunction and/or death of peripheral nerve cells in a subject with Charcot-Marie-Tooth disease.

In this regard, in one particular embodiment of the present invention, it was confirmed that the compound represented by formula I of the present disclosure, optical isomers thereof, or pharmaceutically acceptable salts thereof improve and restore an axonal mitochondrial movement velocity (tables 1 and 2, FIGS. 1 and 2) and suppress the induced PMP22 protein expression (FIG. 3).

In addition, it was confirmed that the compound represented by formula I of the present disclosure, optical isomers thereof, or pharmaceutically acceptable salts thereof enhance a motor function (CRT, GST, BBT) of a mouse (FIGS. 4 to 8), and a nerve conduction velocity of the mouse (FIGS. 9 and 10). Furthermore, it was confirmed that the compound represented by formula I of the present disclosure, optical isomers thereof, or pharmaceutically acceptable salts thereof improved and restored the size of the axon is (FIG. 11).

In other words, the compound represented by formula I, optical isomers thereof or pharmaceutically acceptable salts thereof according to the present disclosure may effectively treat or alleviate symptoms shown in Charcot-Marie-Tooth disease associated with the peripheral nervous system, such as a decrease in motor nerve conduction velocity, a decrease in compound muscle action potential, progressive degeneration of nerve cells, muscle weakness, abnormal sense, axonal atrophy, etc., and may inhibit or delay an expression of such symptoms.

In one particular embodiment of the present invention, the compound represented by formula I of the present disclosure, optical isomers thereof, or pharmaceutically acceptable salts thereof may adjust the genetic properties of patients with Charcot Marie Tooth disease to a normal level or to a level similar thereto, suggesting that the compound represented by formula I, optical isomers thereof or pharmaceutically acceptable salts thereof may ameliorate or treat the symptoms of Charcot Marie Tooth disease (FIGS. 13 to 15).

In one particular embodiment of the present invention, the compound represented by formula I of the present disclosure, optical isomers thereof, or pharmaceutically acceptable salts thereof may raise the proportion of atrophic muscle fibers and increase the cross-sectional area of muscle fibers in patients with Charcot Marie Tooth disease (FIGS. 16 and 17).

In one particular embodiment of the present invention, the compound represented by formula I of the present disclosure, optical isomers thereof, or pharmaceutically acceptable salts thereof may increase fully innervated neuromuscular junctions in patients with Charcot Marie Tooth disease (FIGS. 19 and 20).

In one particular embodiment of the present invention, the compound represented by formula I of the present disclosure, optical isomers thereof, or pharmaceutically acceptable salts thereof may increase a diameter of axons and/or a thickness of myelin sheaths in sensory nerves, decrease abnormal myelination, and increase the proportion of axons with a large diameter in patients with Charcot Marie Tooth disease (FIGS. 21 to 23).

In one particular embodiment of the present invention, the compound represented by formula I of the present disclosure, optical isomers thereof, or pharmaceutically acceptable salts thereof may increase a sensory nerve conduction velocity (SNCV) and an amplitude of a sensory nerve action potential (SNAP) in patients with Charcot Marie Tooth disease (FIGS. 24 and 25).

The compound represented by formula I of the present disclosure, optical isomers thereof or pharmaceutically acceptable salts thereof according to the present disclosure may show an effect of preventing or treating Charcot-Marie-Tooth disease associated with the peripheral nervous system at a level that is similar to or substantially the same as or superior to a conventionally known drug for preventing or treating Charcot-Marie-Tooth disease associated with the peripheral nervous system.

The pharmaceutical composition of the present disclosure may further include at least one pharmaceutically acceptable carrier, in addition to the compound represented by above formula I, optical isomers thereof or pharmaceutically acceptable salts thereof. The pharmaceutically acceptable carrier may be the one which is conventionally used in the art, specifically including, but not limited thereto, lactose, dextrose, sucrose, sorbitol, mannitol, starch, acacia rubber, calcium phosphate, alginate, gelatin, calcium silicate, microcrystalline cellulose, polyvinyl pyrrolidine, cellulose, water, syrup, methylcellulose, methyl hydroxybenzoate, propyl hydroxybenzoate, talc, magnesium stearate, mineral, or oil. The pharmaceutical composition of the present invention may further include lubricants, humectants, sweetening agents, flavoring agents, emulsifiers, suspending agents, preservatives, dispersing agents, stabilizing agents, etc., in addition to the above ingredients. In addition, the pharmaceutical composition of the present invention may be formulated into an oral dosage form such as a tablet, powder, granule, pill, capsule, suspension, emulsion, liquid for internal use, oiling agent, syrup, etc., as well as a form of external application, suppository or sterile solution for injection, by using pharmaceutically acceptable carriers and excipients and thus may be prepared in a unit dose form or prepared by being inserted into a multi-dose container. Such preparations may be prepared according to a conventional method used for formulation in the art or a method disclosed in Remington's Pharmaceutical Science (19^{th} ed., 1995), and may be formulated into various preparations depending on each disease or ingredient.

A non-limiting example of preparations for oral administration using the pharmaceutical composition of the present invention may include tablets, troches, lozenges, water-soluble suspensions, oil suspensions, prepared powders, granules, emulsions, hard capsules, soft capsules, syrups, elixirs or the like. To formulate the pharmaceutical composition according to the embodiments of the present invention into preparation for oral administration, the followings may be used: binders such as lactose, saccharose, sorbitol, mannitol, starch, amylopectin, cellulose, gelatin or the like; excipients such as dicalcium phosphate, etc.; disintegrants such as maize starch, sweet potato starch or the like; lubricants such as magnesium stearate, calcium stearate, sodium stearyl fumarate, polyethylene glycol wax, or the like; etc., in which sweetening agents, flavoring agents, syrups, etc. may also be used. Furthermore, in the case of the capsules, liquid carriers such as fatty oil, etc. may be further used in addition to the above-mentioned materials.

A non-limiting example of parenteral preparations using the pharmaceutical composition according to the embodiments of the present invention may include injectable solutions, suppositories, powders for respiratory inhalation, aerosols for spray, ointments, powders for application, oils, creams, etc. To formulate the pharmaceutical composition according to the embodiments of the present invention into preparation for parenteral administration, the following may be used: sterilized aqueous solutions, non-aqueous solvents, suspensions, emulsions, freeze-dried preparations, external preparations, etc. As said non-aqueous solvents and suspensions, the following may be used, but without limitation thereto: propylene glycol, polyethylene glycol, vegetable oils such as olive oil, injectable esters such as ethyl oleate, etc.

The pharmaceutical composition according to the embodiments of the present invention may be subjected to oral administration or parenteral administration according to a targeted method, for example, intravenous, subcutaneous, intraperitoneal or local administration, particularly oral administration, but is not limited thereto.

A daily dosage of the compound represented by formula I, optical isomers thereof or pharmaceutically acceptable salts thereof according to the present disclosure may be particularly about 0.1 to about 10,000 mg/kg, about 1 to about 8,000 mg/kg, about 5 to about 6,000 mg/kg, or about 10 to about 4,000 mg/kg, and more particularly about 50 to about 2,000 mg/kg, but is not limited thereto and may be also administered once a day or several times a day by dividing the daily dosage of the compound.

A pharmaceutically effective dose and an effective dosage of the pharmaceutical composition according to the embodiments of the present invention may vary depending on a method for formulating the pharmaceutical composition, an administration mode, an administration time, an administration route, and/or the like, and may be diversified according to various factors including a type and degree of reaction to be achieved by administration of the pharmaceutical composition, a type of an individual for administration, the individual's age, weight, general health condition, disease symptom or severity, gender, diet and excretion, ingredients of other drug compositions to be used for the corresponding individual at the same time or different times, etc., as well as other similar factors well known in a pharmaceutical field, and those skilled in the art may easily determine and prescribe an effective dosage for the intended treatment.

The pharmaceutical composition according to the embodiments of the present invention may be administered once a day or several times a day by dividing the daily dosage of the composition. The pharmaceutical composition of the present invention may be administered as an individual therapeutic agent or in combination with other therapeutic agents, and may be administered sequentially or simultaneously with a conventional therapeutic agent. Considering all the above factors, the pharmaceutical composition of the present invention may be administered in such an amount that a maximum effect may be achieved by a minimum amount without a side effect, and such amount may be easily determined by those skilled in the art to which the present invention pertains.

The pharmaceutical composition according to the embodiments of the present invention may show an excellent effect even when solely used, but may be further used in combination with various methods such as hormone therapy, drug treatment, etc. to increase therapeutic efficiency.

The present disclosure may provide a method for preventing or treating Charcot-Marie-Tooth disease associated with the peripheral nervous system, including administering a compound represented by above formula I, optical isomers thereof or pharmaceutically acceptable salts thereof into an individual.

The present disclosure may provide a method for preventing or treating Charcot-Marie-Tooth disease associated with the peripheral nervous system, including administering a compound of the above Table A, optical isomers thereof or pharmaceutically acceptable salts thereof into an individual.

The present disclosure may provide a method for preventing or treating Charcot-Marie-Tooth disease associated with the peripheral nervous system, including administering a compound of the above Table B, optical isomers thereof or pharmaceutically acceptable salts thereof into an individual.

Said terms “Charcot-Marie-Tooth disease,” “prevention” and “treatment” may be the same as described above.

In the present disclosure, the term “administration” may refer to introducing a predetermined substance into an individual by an appropriate method.

In the present disclosure, the term “individual” may refer to all the animals such as rats, mice, livestock, etc., including humans, who have developed or are likely to develop Charcot-Marie-Tooth disease associated with the peripheral nervous system, and may be particularly mammals including humans, but is not limited thereto.

The method for preventing or treating Charcot-Marie-Tooth disease associated with the peripheral nervous system according to the embodiments of the present invention may include administering a therapeutically effective amount of the compound represented by above formula I, optical isomers thereof or pharmaceutically acceptable salts thereof.

In the present disclosure, the term “therapeutically effective amount” may refer to an amount enough to treat a disease at a reasonable risk/benefit ratio applicable to medical treatment and not to cause a side effect, and may be determined by those skilled in the art according to factors including a patient's gender, age, weight and health condition, a type of disease, severity, the activity of a drug, sensitivity to a drug, an administration method, an administration time, an administration route, an excretion rate, a treatment period, a drug combined or concurrently used, as well as other factors well known in a pharmaceutical field. It is preferable to differently apply a particular therapeutically effective amount for a certain patient depending on various factors including a type and degree of reaction to be achieved therefrom, a particular composition including a presence of other preparations used in some cases, a patient's age, weight, general health condition, gender and diet, an administration time, an administration route, a secretion rate of the composition, a treatment period and a drug used together with the particular composition or simultaneously therewith, as well as other similar factors well known in a pharmaceutical field.

The method for preventing or treating Charcot-Marie-Tooth disease associated with the peripheral nervous system of the present invention may include not only dealing with the disease per se before expression of its symptoms, but also inhibiting or avoiding such symptoms by administering the compound represented by above formula I, isomers thereof or pharmaceutically acceptable salts thereof. In managing the disease, a preventive or therapeutic dose of a certain active ingredient may vary depending on the characteristics and severity of the disease or conditions, and a route in which the active ingredient is administered. A dose and a frequency thereof may vary depending on an individual patient's age, weight and reactions. A suitable dose and usage may be easily selected by those skilled in the art, naturally considering such factors.

In addition, the method for preventing or treating Charcot-Marie-Tooth disease associated with the peripheral nervous system of the present invention may further include administering a therapeutically effective amount of an additional active agent, which helps prevent or treat the disease, along with the compound represented by above formula I, optical isomers thereof or pharmaceutically acceptable salts thereof, and the additional active agent may show a synergy effect or an additive effect together with the compound represented by above formula I, optical isomers thereof or pharmaceutically acceptable salts thereof.

The present disclosure may provide a use of the compound represented by the above formula I, optical isomers thereof or pharmaceutically acceptable salts thereof for preventing or treating Charcot-Marie-Tooth disease associated with the peripheral nervous system.

The present disclosure may provide a use of the compound of the above Table A, optical isomers thereof or pharmaceutically acceptable salts thereof for preventing or treating Charcot-Marie-Tooth disease associated with the peripheral nervous system.

The present disclosure may provide a use of the compound of the above Table B, optical isomers thereof or pharmaceutically acceptable salts thereof for preventing or treating Charcot-Marie-Tooth disease associated with the peripheral nervous system.

The present disclosure may provide a use of the compound represented by above formula I, optical isomers thereof or pharmaceutically acceptable salts thereof in preparing a medicament for preventing or treating Charcot-Marie-Tooth disease associated with the peripheral nervous system.

The present disclosure may provide a use of the compound of the above Table A, optical isomers thereof or pharmaceutically acceptable salts thereof in preparing a medicament for preventing or treating Charcot-Marie-Tooth disease associated with the peripheral nervous system.

The present disclosure may provide a use of the compound of the above Table B, optical isomers thereof or pharmaceutically acceptable salts thereof in preparing a medicament for preventing or treating Charcot-Marie-Tooth disease associated with the peripheral nervous system.

Said terms “Charcot-Marie-Tooth disease,” “prevention” and “treatment” may be the same as described above.

For the preparation of the medicament, the compound represented by above formula I, optical isomers thereof or pharmaceutically acceptable salts thereof may be mixed with pharmaceutically acceptable adjuvants, diluents, carriers, etc., and may be prepared into a complex preparation together with other active agents, thus providing a synergy action.

Matters mentioned in the pharmaceutical composition, treatment method and use of the present disclosure are applied the same, if not contradictory to each other.

### Advantageous Effects

The compound represented by formula I, optical isomers thereof or pharmaceutically acceptable salts thereof according to the present disclosure and the pharmaceutical composition including the same as an active ingredient may be advantageously used in preventing or treating Charcot-Marie-Tooth disease associated with the peripheral nervous system.

### BRIEF DESCRIPTION OF THE DRAWINGS

FIG. 1 is a view of showing a velocity distribution of each group as a result of evaluating the effect of the compound of the present disclosure on mitochondrial axon transport in HSPB1^{S135F} CMT2F motor neurons. (*p<0.05, **p<0.01, ***p<0.001)

FIG. 2 is a view of showing a velocity distribution of each group as a result of evaluating the effect of the compound of the present disclosure on mitochondrial axon transport in Gars^{P234KY} CMT2D motor neurons. (##p<0.01, *p<0.05, **p<0.01)

FIG. 3 is a view of showing a level of protein expression in each group as a result of evaluating the effect of the compound of the present disclosure on an expression of myelin sheath-associated protein (PMP22). (###p<0.001)

FIG. 4 is a view of showing a latency to fall in each group as a result of a Rotarod test, which evaluates the effect of the compound of the present disclosure in C22 CMT1A mice. (###p<0.001)

FIG. 5 is a view of showing a grip force in each group as a result of a grip strength test, which evaluates the effect of the compound of the present disclosure in C22 CMT1A mice. (###p<0.001, *p<0.05)

FIG. 6 is a view of showing the number of slips in each group as a result of a balance beam test, which evaluates the effect of the compound of the present disclosure in C22 CMT1A mice. (###p<0.001, *p<0.05)

FIG. 7 is a view of showing a latency to fall in each group as a result of a Rotarod test, which evaluates the effect of the compound of the present disclosure in HSPB1^{S135F} CMT2F mice. (*p<0.05, **p<0.01)

FIG. 8 is a view of showing a grip force in each group as a result of a grip strength test, which evaluates the effect of the compound of the present disclosure in HSPB1^{S135F} CMT2F mice. (***p<0.001)

FIG. 9 is a view of showing a level of CMAP and MNCV in each group as a result of a nerve conduction study, which evaluates the effect of the compound of the present disclosure in 2.5-week-old C22 CMT1A mice. (###p<0.001, **p<0.01)

FIG. 10 is a view of showing a level of CMAP and MNCV in each group as a result of a nerve conduction study, which evaluates the effect of the compound of the present disclosure in eight-month-old C22 CMT1A mice. (###p<0.001, **p<0.01)

FIG. 11 is a view of showing a ratio (%) of axons included in each axon diameter range as a result of evaluating the effect of the compound of the present disclosure on an axon size of sciatic nerve fibers. (##p<0.01, *p<0.05, **p<0.01)

FIGS. 12 to 15 are views showing the genetic properties of sciatic nerves in C3 CMT1A mice as a result of evaluating the effect of the compound of the present disclosure in C3 CMT1A mice.

FIG. 16 is a view showing the effect of the compound of the present disclosure on myofibrillar atrophic neuromuscular junctions in C3 CMT1A mice, which shows a proportion of atrophic muscle fibers in each group ([###p<0.001, Veh vs WT], [***p<0.001 Veh vs compound 43]).

FIG. 17 is a view showing the effect of the compound of the present disclosure on the atrophy of muscle fibers in C3 CMT1A mice, which shows an area of muscle fibers and a distribution of areas in each group ([#p<0.05, ##p<0.01, ###p<0.001, Veh vs WT], [**p<0.01, ***p<0.001 Veh vs compound 43]).

FIG. 18 is a view showing criteria for evaluating a degree of innervation at neuromuscular junctions.

FIGS. 19 and 20 are views of showing the results of evaluating the effect of the compound of the present disclosure on neuromuscular junctions in C3 CMT1A mice, FIG. 19 is a view showing a degree of innervation, and FIG. 20 is a view showing a proportion of fully innervated neuromuscular junctions ([###p<0.001, Veh vs WT], [**p<0.01, ***p<0.001 Veh vs compound 43]).

FIG. 21 is a view showing the results of evaluating the effect of the compound of the present disclosure on sural nerves in C3 CMT1A mice through optical microscopic pictures.

FIGS. 22 and 23 are views showing the results of evaluating the effect of the compound of the present disclosure on sural nerves in C3 CMT1A mice. FIGS. 22A (WT), 22B (TG), 22C (Compound 43) are views showing a slope of g-ratio, and FIG. 23 is a view showing a diameter of axons ([##p<0.01, ###p<0.001, Veh vs WT], [***p<0.001 Veh vs compound 43]).

FIGS. 24 and 25 are views showing the results of evaluating the effect of the compound of the present disclosure on sural nerves in C3 CMT1A mice, FIG. 24 is a view showing a sensory nerve conduction velocity (SNCV), and FIG. 25 is a view showing an amplitude value of a sensory nerve action potential (SNAP) ([###p<0.001, Veh vs WT], [**p<0.01, Veh vs compound 43]).

### MODE FOR INVENTION

The present disclosure will be described in detail with reference to Examples hereinafter. However, the Examples are only for the purpose of illustrating the present invention and it is obvious to those skilled in the art that the scope of the present invention is not limited to the Examples disclosed hereinafter.

### Synthesis Example 1. Synthesis of Compound 43, N-((5-(5-(difluoromethyl)-1,3,4-oxadiazol-2-yl)pyridin-2-yl)methyl)-N-phenylthiomorpholine-4-carboxamide 1,1-dioxide

### [Step 1] N-phenylthiomorpholine-4-carboxamide 1,1-dioxide

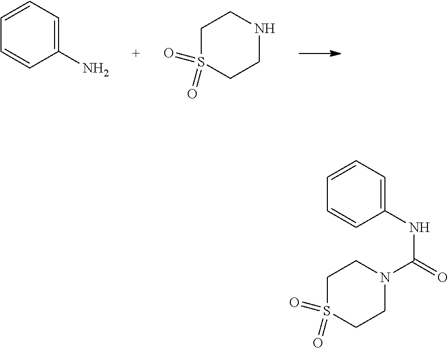 [SMILES: Nc1ccccc1 ; O=S1(=O)CCNCC1 ; O=C(Nc1ccccc1)N1CCS(=O)(=O)CC1]

To a solution of aniline (3.000 g, 32,213 mmol) and N,N-diisopropylethylamine (33.439 mL, 193.278 mmol) in dichloromethane (100 mL) was added at 0° C. triphosgene (4.780 g, 16.107 mmol) and was stirred at the same temperature. Thiomorpholine 1,1-dioxide (4.790 g, 35.434 mmol) was added to the reaction mixture and stirred for an additional 16 hr at room temperature. Then, water was added to the reaction mixture, followed by extraction with ethyl acetate. The organic layer was washed with brine, dried (anhydrous MgSO₄), filtered, and concentrated under the reduced pressure. The concentrate was purified and concentrated by column chromatography (SiO₂, 40 g cartridge; methanol/dichloromethane=2%) to give the title compound as yellow solid (1.325 g, 16.2%).

### [Step 2] Synthesis of Methyl 6-((1,1-dioxido-N-phenylthiomorpholine-4-carboxamido)methyl)nicotinate

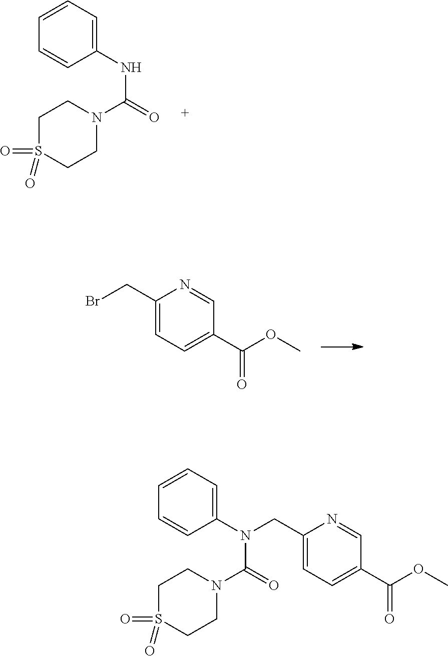 [SMILES: O=C(Nc1ccccc1)N1CCS(=O)(=O)CC1 ; COC(=O)c1ccc(CBr)nc1 ; COC(=O)c1ccc(CN(C(=O)N2CCS(=O)(=O)CC2)c2ccccc2)nc1]

A solution of N-phenylthiomorpholine-4-carboxamide 1,1-dioxide (1.000 g, 3.932 mmol) prepared in Step 1 and sodium hydride (60.00%, 0.157 g, 3.932 mmol) in N,N-dimethylformamide (10 mL) was stirred at 0° C. for 1 hr, and mixed with methyl 4-(bromomethyl)-3-fluorobenzoate (0.905 g, 3.932 mmol). The reaction mixture was stirred at room temperature for an additional 2 hr. The reaction mixture was concentrated under the reduced pressure to remove the solvent, and water was added to the concentrate, followed by extraction with ethyl acetate. The organic layer was washed with brine, dried (anhydrous MgSO₄), filtered, and concentrated under the reduced pressure. The crude product was crystallized at room temperature using methanol (20 mL). The resulting precipitates obtained by filtration were washed by methanol, and dried to give the title compound as brown solid (0.816 g. 51.4%).

### [Step 3] Synthesis of N-((5-(hydrazinecarbonyl)pyridin-2-yl)methyl)-N-phenylthiomorpholine-4-carboxamide 1,1-dioxide

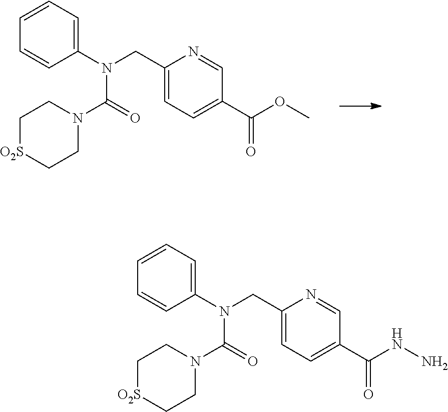 [SMILES: COC(=O)c1ccc(CN(C(=O)N2CCS(=O)(=O)CC2)c2ccccc2)nc1 ; NNC(=O)c1ccc(CN(C(=O)N2CCS(=O)(=O)CC2)c2ccccc2)nc1]

Methyl 6-((1,1-dioxido-N-phenylthiomorpholine-4-carboxamido)methyl)nicotinate (0.816 g, 2.023 mmol) prepared in Step 2 and hydrazine monohydrate (1.910 mL, 40.451 mmol) was mixed in ethanol (10 mL) at the room temperature and then heated at 100° C. under the microwaves for 1 hr, and cooled down to the room temperature to terminate the reaction. The reaction mixture was concentrated under the reduced pressure to remove the solvent. The crude product was crystallized at room temperature using dichloromethane (20 mL). The resulting precipitates obtained by filtration were washed by dichloromethane, and dried to give the title compound as light brown solid (0.560 g, 68.6%).

### [Step 4] Synthesis of N-((5-(2-(2,2-difluoroacetyl)hydrazine-1-carbonyl)pyridin-2-yl)methyl)-N-phenylthiomorpholine-4-carboxamide 1,1-dioxide

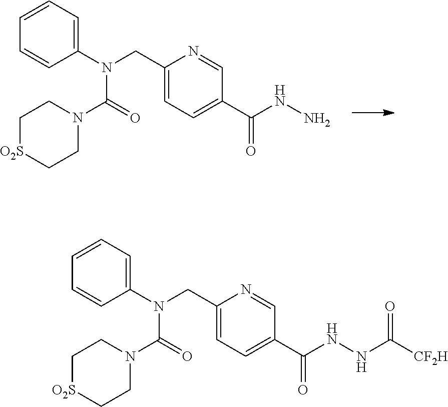 [SMILES: NNC(=O)c1ccc(CN(C(=O)N2CCS(=O)(=O)CC2)c2ccccc2)nc1 ; O=C(NNC(=O)C(F)F)c1ccc(CN(C(=O)N2CCS(=O)(=O)CC2)c2ccccc2)nc1]

A solution of N-((5-(hydrazinecarbonyl)pyridin-2-yl)methyl)-N-phenylthiomorpholine-4-carboxamide 1,1-dioxide (0.260 g, 0.644 mmol) prepared in Step 3 and triethylamine (0.178 mL, 1.289 mmol) in dichloromethane (2 mL) was mixed with Difluoroacetic Anhydride (0.087 mL, 0.580 mmol) at the room temperature. The reaction mixture was stirred at the same temperature for 16 hr. Then, water was added to the reaction mixture, followed by extraction with dichloromethane. The mixture was passed through a plastic frit to remove solid residues and an aqueous layer, and the organic layer collected was concentrated under the reduced pressure. The concentrate was purified and concentrated by column chromatography (SiO₂, 4 g cartridge; methanol/dichloromethane=0% to 5%) to give the title compound as white foam (0.156 g, 50.3%).

### [Step 5] Synthesis of Compound 43

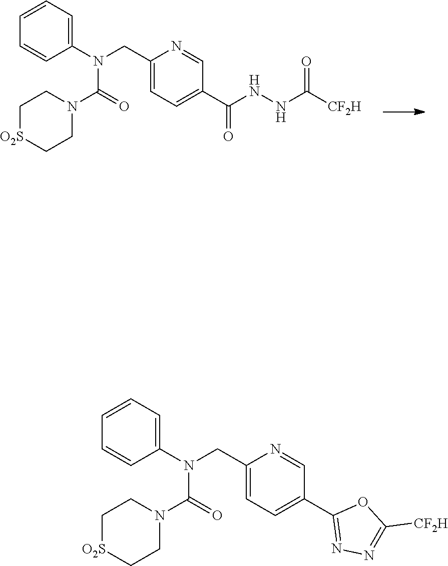 [SMILES: O=C(NNC(=O)C(F)F)c1ccc(CN(C(=O)N2CCS(=O)(=O)CC2)c2ccccc2)nc1 ; O=C(N1CCS(=O)(=O)CC1)N(Cc1ccc(-c2nnc(C(F)F)o2)cn1)c1ccccc1]

A mixture of N-((5-(2-(2,2-difluoroacetyl)hydrazine-1-carbonyl)pyridin-2-yl)methyl)-N-phenylthiomorpholine-4-carboxamide 1,1-dioxide (0.156 g, 0.324 mmol) prepared in Step 4 and 1-methoxy-N-triethylammoniosulfonyl-methanimidate (Burgess reagent, 0.116 g, 0.486 mmol) in tetrahydrofuran (2 mL) was heated at 150° C. for 30 min under the microwaves, and cooled down to the room temperature to terminate the reaction. Then, water was added to the reaction mixture, followed by extraction with dichloromethane. The biphasic mixture was passed through a plastic frit to remove solid residues and an aqueous layer, and the organic layer collected was concentrated under the reduced pressure. The concentrate was purified and concentrated by column chromatography (SiO₂, 4 g cartridge; methanol/dichloromethane=3%) to give the title compound as colorless oil (0.078 g, 51.9%).

¹H NMR (400 MHz, CDCl₃) δ 9.23 (d, 1H, J=2.2 Hz), 8.38 (dd, 1H, J=8.2, 2.2 Hz), 7.54 (d, 1H, J=8.2 Hz), 7.41-7.31 (m, 2H), 7.19 (ddd, 3H, J=6.4, 3.0, 1.6 Hz), 6.94 (m, 1H), 5.10 (s, 2H), 3.72 (dd, 4H, J=6.9, 3.7 Hz), 2.97-2.90 (m, 4H); LRMS (ES) m/z 464.2 (M⁺+1)

### Synthesis Example 2. Synthesis of Compound 232, N-(3-chloro-4-fluorophenyl)-N-((5-(5-(difluoromethyl)-1,3,4-oxadiazol-2-yl)pyridin-2-yl)methyl)morpholine-4-carboxamide

### [Step 1] Synthesis of N-(3-chloro-4-fluorophenyl)morpholine-4-carboxamide

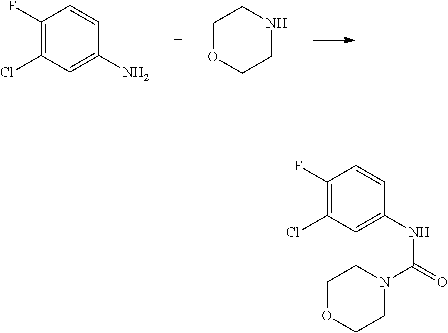 [SMILES: Nc1ccc(F)c(Cl)c1 ; C1COCCN1 ; O=C(Nc1ccc(F)c(Cl)c1)N1CCOCC1]

A solution of 3-chloro-4-fluoroaniline (0.500 μg, 3.435 mmol), 1,1′-carbonyldiimidazole (0.613 g, 3.779 mmol) and triethylamine (0.575 mL, 4.122 mmol) in acetonitrile (10 mL) was mixed at the room temperature with morpholine (0.311 mL, 3.607 mmol). The reaction mixture was stirred at the same temperature for 18 hr. Then, water was added to the reaction mixture, followed by extraction with ethyl acetate. The organic layer was washed with aqueous saturated sodium chloride solution, dried (anhydrous MgSO₄), filtered, and concentrated under the reduced pressure. The residue was purified and concentrated by chromatography (SiO₂, 12 g cartridge; methanol/dichloromethane=0% to 5%) to give the title compound as purple solid (0.200 g, 22.5%).

### [Step 2] Synthesis of Methyl 6-((N-(3-chloro-4-fluorophenyl)morpholine-4-carboxamido)methyl)nicotinate

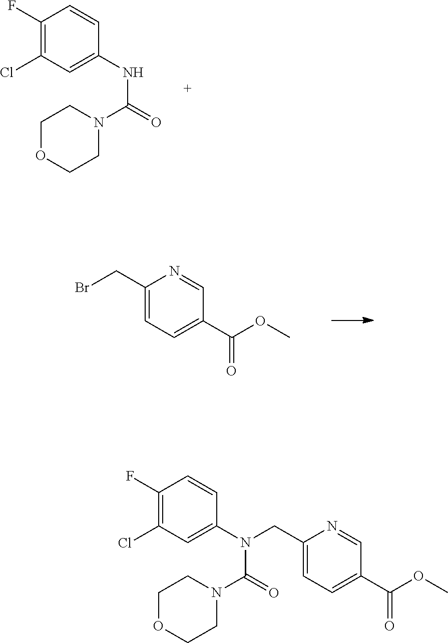 [SMILES: O=C(Nc1ccc(F)c(Cl)c1)N1CCOCC1 ; COC(=O)c1ccc(CBr)nc1 ; COC(=O)c1ccc(CN(C(=O)N2CCOCC2)c2ccc(F)c(Cl)c2)nc1]

To a solution of N-(3-chloro-4-fluorophenyl)morpholine-4-carboxamide (0.200 g, 0.773 mmol) prepared in Step 1 in N,N-dimethylformide (5 mL) was added at 0° C. sodium hydride (60.00%, 0.037 g, 0.928 mmol) and was stirred at the same temperature. Methyl 6-(bromomethyl)nicotinate (0.196 g, 0.850 mmol) was added in the reaction mixture, and stirred for an additional 3 hr. Then, water was added to the reaction mixture, followed by extraction with ethyl acetate. The organic layer was washed with aqueous saturated sodium chloride solution, dried (anhydrous MgSO₄), filtered, and concentrated under the reduced pressure. The residue was purified and concentrated by chromatography (SiO₂, 4 g cartridge; ethyl acetate/hexane=0% to 70%) to give the title compound as brown oil (0.110 g, 34.9%).

### [Step 3] Synthesis of N-(3-chloro-4-fluorophenyl)-N-((5-(hydrazinecarbonyl)pyridin-2-yl)methyl)morpholine-4-carboxamide

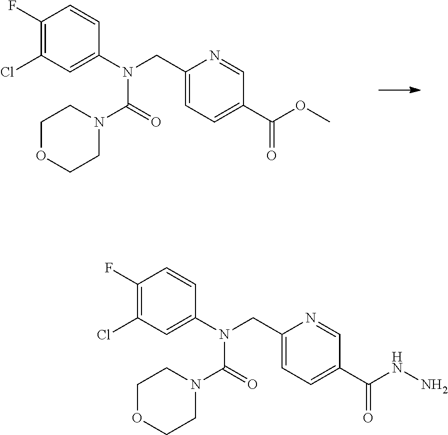 [SMILES: COC(=O)c1ccc(CN(C(=O)N2CCOCC2)c2ccc(F)c(Cl)c2)nc1 ; NNC(=O)c1ccc(CN(C(=O)N2CCOCC2)c2ccc(F)c(Cl)c2)nc1]

A mixture of methyl 6-((N-(3-chloro-4-fluorophenyl)morpholine-4-carboxamido)methyl)nicotinate (0.110 g, 0.270 mmol) prepared in Step 2 and hydrazine monohydrate (0.262 mL, 5.394 mmol) in ethanol (5 mL) was heated at reflux for 18 hr, and cooled down to the ambient temperature. Then, water was added to the reaction mixture, followed by extraction with ethyl acetate. The organic layer was washed with aqueous saturated sodium chloride solution, dried (anhydrous MgSO₄), filtered, and concentrated under the reduced pressure. The obtained title compound was used without further purification (0.110 g, 100.0%, light yellow solid).

### [Step 4] Synthesis of the Compound 232

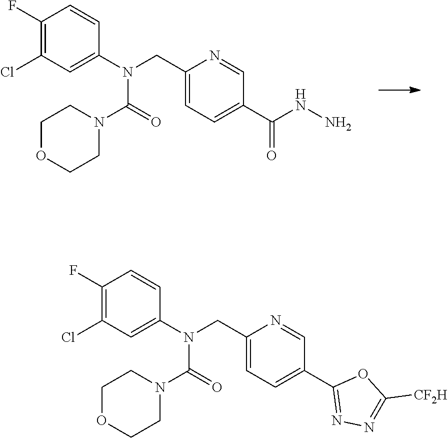 [SMILES: NNC(=O)c1ccc(CN(C(=O)N2CCOCC2)c2ccc(F)c(Cl)c2)nc1 ; O=C(N1CCOCC1)N(Cc1ccc(-c2nnc(C(F)F)o2)cn1)c1ccc(F)c(Cl)c1]

A solution of N-(3-chloro-4-fluorophenyl)-N-((5-(hydrazinecarbonyl)pyridin-2-yl)methyl)morpholine-4-carboxamide (0.110 g, 0.270 mmol) prepared in Step 3 and N.N-diisopropylethylamine (0.070 mL, 0.405 mmol) in dichloromethane (3 mL) was mixed at 0° C. with 2,2-difluoroacetic anhydride (0.059 mL, 0.539 mmol), and stirred at the room temperature for 16 hr. Then, saturated aqueous sodium bicarbonate solution was added to the reaction mixture, followed by extraction with dichloromethane. The biphasic mixture was passed through a plastic frit to remove the solid residues and aqueous layer, and the organic layer collected was concentrated under the reduced pressure. The residue was purified and concentrated by chromatography (SiO₂, 4 g cartridge; methanol/dichloromethane=0% to 5%) to give the title compound as yellow solid (0.057 g, 45.2%).

¹H NMR (400 MHz, CDCl₃) δ 9.24-9.24 (m, 1H), 8.36 (dd, 1H, J=8.2, 2.2 Hz), 7.59 (dd, 1H, J=8.2, 0.8 Hz), 7.30-7.28 (m, 1H), 7.10-7.08 (m, 2H), 6.93 (t, 1H, J=51.6 Hz), 5.05 (s, 2H), 3.54-3.52 (m, 4H), 3.27-3.26 (m, 4H); LRMS (ES) m/z 468.2 (M⁺+1).

### Synthesis Example 3. Synthesis of the Compound 239, N-(3-chlorophenyl)-N-((5-(5-(difluoromethyl)-1,3,4-oxadiazol-2-yl)pyridin-2-yl)methyl)thiomorpholine-4-carboxamide 1,1-dioxide

### [Step 1] Synthesis of N-(3-chlorophenyl)thiomorpholine-4-carboxamide 1,1-dioxide

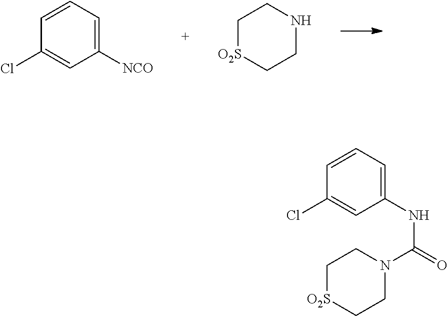

A solution of 1-chloro-3-isocyanatobenzene (1.000 g, 6.512 mmol) and thiomorpholine 1,1-dioxide (0.871 g, 6.447 mmol) in diethyl ether (20 mL) was stirred at the room temperature for 18 hr. The precipitates were filtered, washed by diethyl ether, and dried to give the title compound as white solid (1.811 g, 96.3%).

### [Step 2] Synthesis of Methyl 6-((N-(3-chlorophenyl)-1,1-dioxidothiomorpholine-4-carboxamido)methyl)nicotinate

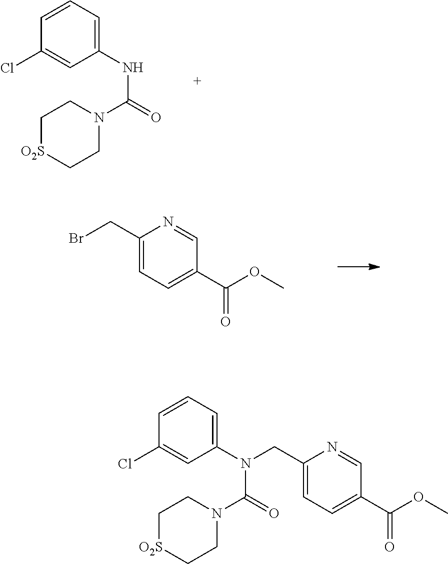 [SMILES: O=C(Nc1cccc(Cl)c1)N1CCS(=O)(=O)CC1 ; COC(=O)c1ccc(CBr)nc1 ; COC(=O)c1ccc(CN(C(=O)N2CCS(=O)(=O)CC2)c2cccc(Cl)c2)nc1]

To a solution of N-(3-chlorophenyl)thiomorpholine-4-carboxamide 1,1-dioxide (0.200 g, 0.693 mmol) prepared in Step 1 in N,N-dimethylformamide (5 mL) was added at 0° C. sodium hydride (60.00%, 0.028 g, 0.693 mmol). The reaction mixture was stirred at the same temperature for 1 hr, added at the same temperature with methyl 6-(bromomethyl)nicotinate (0.159 g, 0.693 mmol), and stirred for additional 2 hr. Then, water was added to the reaction mixture, followed by extraction with ethyl acetate. The organic layer was washed with aqueous saturated sodium chloride solution, dried (anhydrous MgSO₄), filtered, and concentrated under the reduced pressure. The residue was purified and concentrated by chromatography (SiO₂, 12 g cartridge; methanol/dichloromethane=0% to 5%) to give the title compound as brown oil (0.261 g, 86.0%).

### [Step 3] Synthesis of N-(3-chlorophenyl)-N-((5-(hydrazinecarbonyl)pyridin-2-yl)methyl)thiomorpholine-4-carboxamide 1,1-dioxide

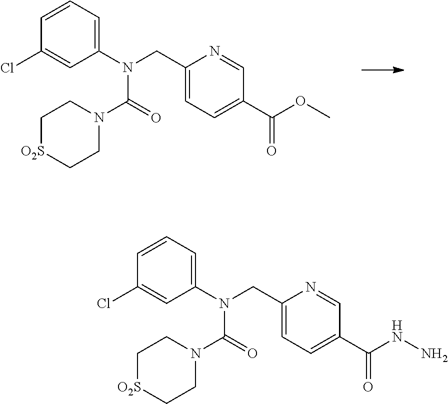 [SMILES: COC(=O)c1ccc(CN(C(=O)N2CCS(=O)(=O)CC2)c2cccc(Cl)c2)nc1 ; NNC(=O)c1ccc(CN(C(=O)N2CCS(=O)(=O)CC2)c2cccc(Cl)c2)nc1]

Methyl 6-((N-(3-chlorophenyl)-1,1-dioxidothiomorpholine-4-carboxamido)methyl)nicotinate (0.261 g, 0.596 mmol) prepared in Step 2 and hydrazine monohydrate (0.290 mL, 5.958 mmol) were mixed at the room temperature in ethanol (2 mL) and then stirred at 110° C. for 18 hr and cooled down to the room temperature to terminate the reaction. The reaction mixture was concentrated under the reduced pressure to remove the solvent. Then, water was added to the obtained concentrate, followed by extraction with dichloromethane. The biphasic mixture was passed through a plastic frit to remove the solid residues and aqueous layer, and the organic layer collected was concentrated under the reduced pressure. The residue was purified and concentrated by chromatography (SiO₂, 4 g cartridge; methanol/dichloromethane=5% to 15%) to give the title compound as brown oil (0.261 g, 100.0%).

### [Step 4] Synthesis of Compound 239

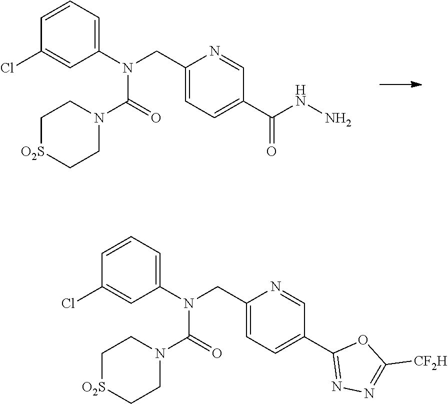 [SMILES: NNC(=O)c1ccc(CN(C(=O)N2CCS(=O)(=O)CC2)c2cccc(Cl)c2)nc1 ; O=C(N1CCS(=O)(=O)CC1)N(Cc1ccc(-c2nnc(C(F)F)o2)cn1)c1cccc(Cl)c1]

N-(3-chlorophenyl)-N-((5-(hydrazinecarbonyl)pyridin-2-yl)methyl)thiomorpholine-4-carboxamide 1,1-dioxide (0.261 g, 0.596 mmol) prepared in Step 3, triethylamine (0.415 mL, 2.980 mmol) and 2,2-difluoroacetic anhydride (0.195 mL, 1.788 mmol) were mixed at the room temperature in tetrahydrofuran (2 mL) and then the obtained solution was stirred at 80° C. for 18 hr and cooled down to the room temperature to terminate the reaction. The reaction mixture was concentrated under the reduced pressure to remove the solvent. Then, water was added to the obtained concentrate, followed by extraction with dichloromethane. The biphasic mixture was passed through a plastic frit to remove the solid residues and aqueous layer, and the organic layer collected was concentrated under the reduced pressure. The residue was purified and concentrated by chromatography (SiO₂, 4 g cartridge; methanol/dichloromethane=0% to 3%) to give the title compound as yellow foam (0.087 g, 29.3%).

¹H NMR (400 MHz, CDCl₃) δ 9.27 (dd, 1H, J=2.2, 0.8 Hz), 8.43 (dd, 1H, J=8.2, 2.2 Hz), 7.55 (dd, 1H, J=8.2, 0.9 Hz), 7.31 (t, 1H, J=8.0 Hz), 7.23 (t, 1H, J=2.1 Hz), 7.21-7.10 (m, 2H), 7.10 (t, 1H), 5.12 (s, 2H), 3.75 (t, 4H, J=5.3 Hz), 3.06-2.99 (m, 4H); LRMS (ES) m/z 498.3 (M⁺+1).

### Example 4. Synthesis of the Compound 243, N-((5-(5-(difluoromethyl)-1,3,4-oxadiazol-2-yl)pyridin-2-yl)methyl)-N-phenylmorpholine-4-carboxamide

### [Step 1] Synthesis of N-phenylmorpholine-4-carboxamide

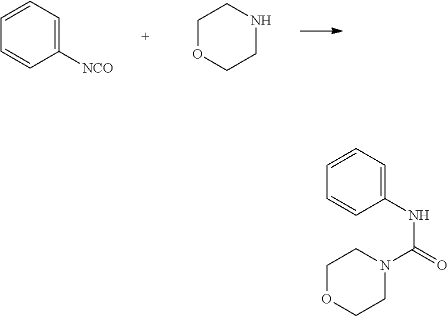

A solution of isocyanatobenzene (1.000 g, 8.395 mmol) and morpholine (0.726 mL, 8.395 mmol) in diethyl ether (20 mL) was stirred at room temperature for 18 hr. The precipitates were collected by filtration, washed by diethyl ether, and dried to give the title compound as a white solid (1.717 g, 99.2%).

### [Step 2] Synthesis of Methyl 6-((N-phenylmorpholine-4-carboxamido)methyl)nicotinate

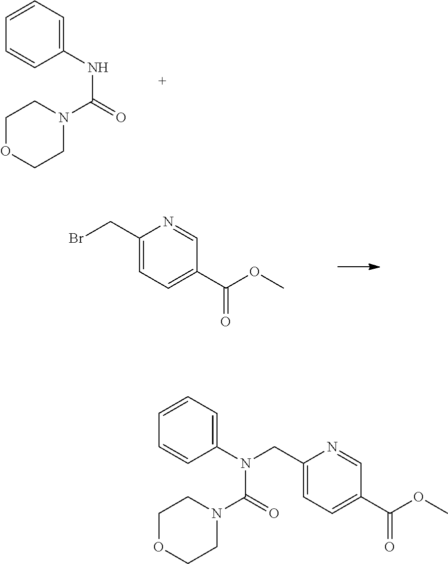 [SMILES: O=C(Nc1ccccc1)N1CCOCC1 ; COC(=O)c1ccc(CBr)nc1 ; COC(=O)c1ccc(CN(C(=O)N2CCOCC2)c2ccccc2)nc1]

To a solution of N-phenylmorpholine-4-carboxamide (0.200 g, 0.970 mmol) prepared in Step 1 in N,N-dimethylformamide (5 mL) was added at 0° C. sodium hydride (60.00%, 0.039 g, 0.970 mmol). The reaction mixture was stirred at the same temperature for 1 hr, added at the same temperature with methyl 6-(bromomethyl)nicotinate (0.223 g, 0.970 mmol), and stirred for an additional 2 hr. Then, water was added to the reaction mixture, followed by extraction with ethyl acetate. The organic layer was washed with aqueous saturated sodium chloride solution, dried (anhydrous MgSO₄), filtered, and concentrated under the reduced pressure. The residue was purified and concentrated chromatography (SiO₂, 4 g cartridge; methanol/dichloromethane=0% to 5%) to give the title compound as brown oil (0.294 g, 85.4%).

### [Step 3] Synthesis of N-((5-(hydrazinecarbonyl)pyridin-2-yl)methyl)-N-phenylmorpholine-4-carboxamide

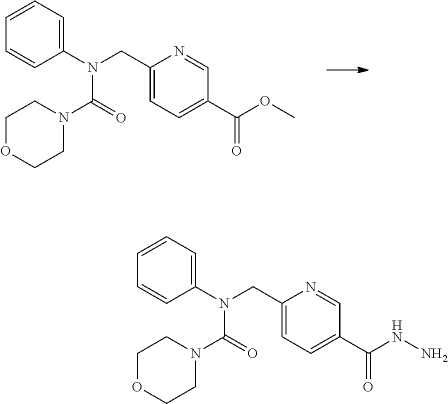 [SMILES: COC(=O)c1ccc(CN(C(=O)N2CCOCC2)c2ccccc2)nc1 ; NNC(=O)c1ccc(CN(C(=O)N2CCOCC2)c2ccccc2)nc1]

Methyl 6-((N-phenylmorpholine-4-carboxamido)methyl)nicotinate (0.294 g, 0.828 mmol) prepared in Step 2 and hydrazine monohydrate (0.403 mL, 8.284 mmol) were mixed at the room temperature in ethanol (2 mL) and then the obtained solution was stirred at 110° C. for 18 hr and cooled down to the room temperature to terminate the reaction. The reaction mixture was concentrated under reduced pressure to remove the solvent. Then, water was added to the obtained concentrate, followed by extraction with dichloromethane. The biphasic mixture was passed through a plastic frit to remove the solid residues and aqueous layer, and the organic layer collected was concentrated under the reduced pressure. The residue was purified and concentrated chromatography (SiO₂, 4 g cartridge; methanol/dichloromethane=5% to 15%) to give the title compound as brown oil (0.294 g, 100.0%).

### [Step 4] Synthesis of N-((5-(2-(2,2-difluoroacetyl)hydrazine-1-carbonyl)pyridin-2-yl)methyl)-N-phenylmorpholine-4-carboxamide

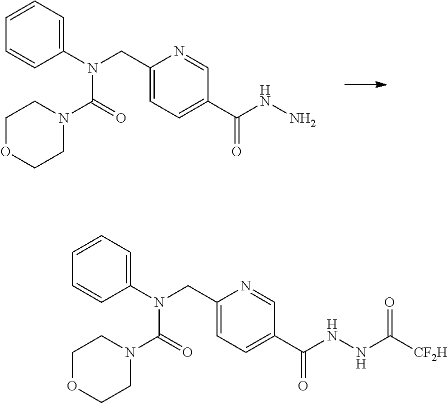 [SMILES: NNC(=O)c1ccc(CN(C(=O)N2CCOCC2)c2ccccc2)nc1 ; O=C(NNC(=O)C(F)F)c1ccc(CN(C(=O)N2CCOCC2)c2ccccc2)nc1]

N-((5-(hydrazinecarbonyl)pyridin-2-yl)methyl)-N-phenylmorpholine-4-carboxamide (0.294 g, 0.828 mmol) prepared in Step 3, triethylamine (0.577 mL, 4.142 mmol) and 2,2-difluoroacetic anhydride (0.270 mL, 2.485 mmol) were mixed at room temperature in tetrahydrofuran (2 mL) and then the obtained solution was stirred at 80° C. for 18 hr and cooled down to the room temperature to terminate the reaction. The reaction mixture was concentrated under the reduced pressure to remove the solvent. Then, water was added to the obtained concentrate, followed by extraction with dichloromethane. The biphasic mixture was passed through a plastic frit to remove the solid residues and aqueous layer, and the organic layer collected was concentrated under the reduced pressure. The residue was purified and concentrated by chromatography (SiO₂, 4 g cartridge; methanol/dichloromethane=0% to 3%) to give the title compound as yellow solid (0.130 g, 44.2%).

### [Step 5] Synthesis of the Compound 243

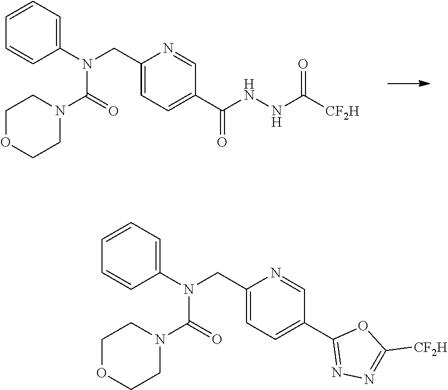 [SMILES: O=C(NNC(=O)C(F)F)c1ccc(CN(C(=O)N2CCOCC2)c2ccccc2)nc1 ; O=C(N1CCOCC1)N(Cc1ccc(-c2nnc(C(F)F)o2)cn1)c1ccccc1]

N-((5-(2-(2,2-difluoroacetyl)hydrazine-1-carbonyl)pyridin-2-yl)methyl)-N-phenylmorpholine-4-carboxamide (0.130 g, 0.300 mmol) prepared in Step 4 and 1-methoxy-N-triethylammoniosulfonyl-methanimidate (Burgess reagent, 0.107 g, 0.450 mmol) were mixed at room temperature in tetrahydrofuran (2 mL) and then the obtained solution was stirred at 80° C. for 18 hr and cooled down to the room temperature to terminate the reaction. The reaction mixture was concentrated under the reduced pressure to remove the solvent. Then, water was added to the obtained concentrate, followed by extraction with dichloromethane. The biphasic mixture was passed through a plastic frit to remove the solid residues and aqueous layer, and the organic layer collected was concentrated under the reduced pressure. The residue was purified and concentrated by chromatography (SiO₂, 4 g cartridge: methanol/dichloromethane=0% to 3%) to give the title compound as yellow oil (0.089 g, 71.0%).

¹H NMR (400 MHz, CDCl₃) δ 9.24 (dd, 1H, J=2.2, 0.8 Hz), 8.37 (dd, 1H, J=8.2, 2.2 Hz), 7.66 (dd, 1H, J=8.2, 0.8 Hz), 7.39-7.30 (m, 2H), 7.24-7.17 (m, 2H), 7.15-7.09 (m, 1H), 7.01 (t, 1H, J=51.7 Hz), 5.15 (s, 2H), 3.53 (dd, 4H, J=5.6, 4.0 Hz), 3.28 (dd, 4H, J=5.6, 4.0 Hz); LRMS (ES) m/z 416.1 (M⁺+1).

### Example 5. Synthesis of the Compound 286, N-(4-(5-(difluoromethyl)-1,3,4-oxadiazol-2-yl)-2-fluorobenzyl)-N-(3-fluorophenyl)thiomorpholine-4-carboxamide 1,1-dioxide

### [Step 1] Synthesis of N-(3-fluorophenyl)thiomorpholine-4-carboxamide 1,1-dioxide

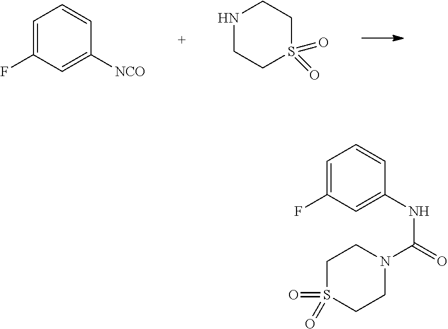

A solution of 1-fluoro-3-isocyanatobenzene (0.500 g. 3.647 mmol) in diethyl ether (10 mL) was mixed at 0° C. with thiomorpholine 1,1-dioxide (0.493 g, 3.647 mmol). and stirred at the same temperature for 1 hr. The reaction mixture was stirred at room temperature for an additional 4 hr. The precipitates were collected by filtration, washed by diethyl ether, and dried to give the title compound as white solid (0.870 g, 87.6%).

### [Step 2] Synthesis of Methyl 3-fluoro-4-((N-(3-fluorophenyl)-1,1-dioxidothiomorpholine-4-carboxamido)methyl)benzoate

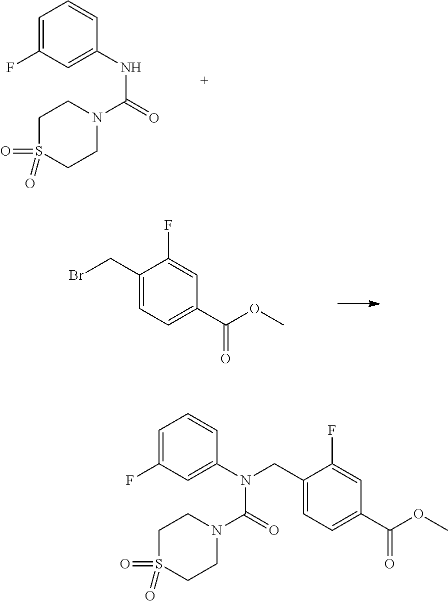 [SMILES: O=C(Nc1cccc(F)c1)N1CCS(=O)(=O)CC1 ; COC(=O)c1ccc(CBr)c(F)c1 ; COC(=O)c1ccc(CN(C(=O)N2CCS(=O)(=O)CC2)c2cccc(F)c2)c(F)c1]

A solution of N-(3-fluorophenyl)thiomorpholine-4-carboxamide 1,1-dioxide (0.300 g, 1.102 mmol) prepared in Step 1 and sodium hydride (60.00%. 0.048 g, 1.212 mmol) in N, N-dimethylformamide (5 mL) was stirred at 0° C. for 2 hr, and mixed with methyl 4-(bromomethyl)-3-fluorobenzoate (0.299 g, 1.212 mmol). The reaction mixture was stirred at room temperature for an additional 17 hr, the reaction was quenched by the addition of water (2 mL, 10 min stirring) at the room temperature. Then, water was added to the reaction mixture, followed by extraction with dichloromethane. The biphasic mixture was passed through a plastic frit to remove the solid residues and aqueous layer, and the organic layer collected was concentrated under the reduced pressure. The residue was purified and concentrated by chromatography (SiO₂, 4 g cartridge; ethyl acetate/hexane=0% to 40%) to the title compound as white solid (0.300 g, 62.1%).

### [Step 3] Synthesis of N-(2-fluoro-4-(hydrazinecarbonyl)benzyl)-N-(3-fluorophenyl)thiomorpholine-4-carboxamide 1,1-dioxide

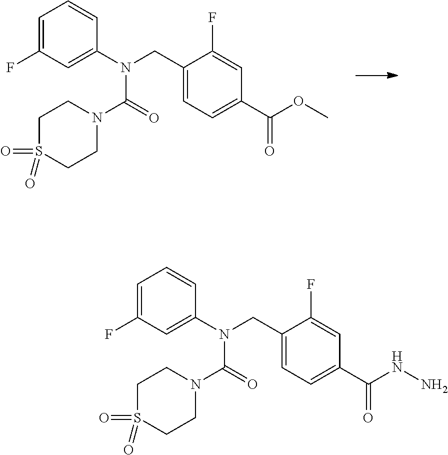 [SMILES: COC(=O)c1ccc(CN(C(=O)N2CCS(=O)(=O)CC2)c2cccc(F)c2)c(F)c1 ; NNC(=O)c1ccc(CN(C(=O)N2CCS(=O)(=O)CC2)c2cccc(F)c2)c(F)c1]

Methyl 3-fluoro-4-((N-(3-fluorophenyl)-1,1-dioxidothiomorpholine-4-carboxamido)methyl)benzoate (0.300 s, 0.684 mmol) prepared in Step 2 and hydrazine monohydrate (0.665 mL, 13.685 mmol) in ethanol (4 mL) was mixed at the room temperature and then heated at 120° C. under the microwaves for 1 hr and cooled down to the room temperature to terminate the reaction. The reaction mixture was concentrated under the reduced pressure to remove the solvent. Then, water was added to the obtained concentrate, followed by extraction with dichloromethane. The biphasic mixture was passed through a plastic frit to remove the solid residues and aqueous layer, and the organic layer collected was concentrated under the reduced pressure. Diethyl ether (5 mL) and ethyl acetate (1 mL) was added into the residue and stirred at ambient temperature. The resulting precipitates were collected by filtration, washed by hexane, and dried to give the title compound as white solid (0.270 g, 90.0%).

### [Step 4] Synthesis of the Compound 286

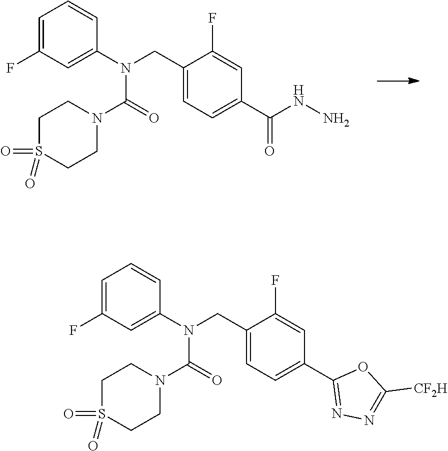 [SMILES: NNC(=O)c1ccc(CN(C(=O)N2CCS(=O)(=O)CC2)c2cccc(F)c2)c(F)c1 ; O=C(N1CCS(=O)(=O)CC1)N(Cc1ccc(-c2nnc(C(F)F)o2)cc1F)c1cccc(F)c1]

A solution of N-(2-fluoro-4-(hydrazinecarbonyl)benzyl)-N-(3-fluorophenyl)thiomorpholine-4-carboxamide 1,1-dioxide (0.100 g, 0.228 mmol) prepared in Step 3 and triethylamine (0.095 mL, 0.684 mmol) in dichloromethane (4 mL) was mixed at the room temperature with 2,2-difluoroacetic anhydride (0.028 mL, 0.228 mmol), and stirred at the same temperature for 17 hr. Then, saturated aqueous sodium bicarbonate solution was added to the reaction mixture, followed by extraction with dichloromethane. The biphasic mixture was passed through a plastic frit to remove the solid residues and aqueous layer, and the organic layer collected was concentrated under the reduced pressure. The residue was purified and concentrated by chromatography (SiO₂, 4 g cartridge; ethyl acetate/hexane=20% to 50%) to give the title compound as white solid (0.034 g, 29.9%).

¹H NMR (400 MHz, CDCl₃) δ 7.90 (dd, 1H, J=8.0, 1.6 Hz), 7.79 (dd, 1H, J=10.1, 1.6 Hz), 7.68 (t, 1H, J=7.6 Hz), 7.38-7.33 (m, 1H), 7.07-6.81 (m, 4H), 4.95 (s, 2H), 3.75 (t, 4H, J=5.2 Hz), 2.87 (t, 4H, J=5.2 Hz); LRMS (ES) m/z 499.0 (M⁺+1).

### <Experimental Example 1> Mitochondrial Axon Transport Assay (MATA)

This experiment was made to confirm the effect of the compound of the present disclosure on mitochondrial axon transport.

### <Experimental Example 1-1> Mitochondrial Axon Transport in HSPB1^{S135F} CMT2F Motor Neurons

The effect of the compound of the present disclosure on axonal mitochondrial movement in motor neurons derived from a human iPSC-based model of HSPB1^{S135F} CMT2F (hereinafter referred to as “CMT2F MNs”) was evaluated as follows.

Wild type (WT) and HSPB1^{S135F} induced pluripotent stem cell (iPSC) were provided from Samsung Medical Center (ECT 11-58-37, Ewha Womans University, IRB No. 2013-10-124), transferred to a CKD pharmacology research laboratory before an experiment, and differentiated into motor neurons according to the following protocol. To produce embryoid bodies, a colony of iPSC was dissociated into small clumps by using enzymes and cultured in suspension in a Petri dish for two days. Culture medium was supplemented with 10 μM Y27632 (Rho-associated kinase inhibitor; Y₀₅₀₃, Tocris Bioscience, Bristol, UK), 20 ng/mL bFGF (PHG0024, Gibco), 10 μM SB435142 (SMAD inhibitor; S4317, Sigma), 0.2 μM LDN193189 (SMAD inhibitor; SML0559, Sigma) and penicillin/streptomycin (15140122, Gibco). On day 5, for casualization, 1 μM retinoic acid (R2625, Sigma), 0.4 μg/mL ascorbic acid (A4544, Sigma), 10 ng/mL brain-derived neurotrophic factor (BDNF; SRP3014, Sigma) and 1% N2 supplement (17502048, Gibco) were added. On day 7, for ventralization, SB435142 and LDN193189 (SMAD inhibitor) were washed, and 1 μM purmorphamine (Sonic hedgehog agonist; SML0868, Sigma) was added. On day 17, cells were cultured in Neurobasal medium [prepared on day 7 and supplemented with 10 ng/mL IGF-1 (I3769, Sigma), 10 ng/mL GDNF (G1777, Gibco) and 10 ng/mL 2% B-27™ (A1486701, Gibco)]. The cells were cultured in suspension in a Petri dish. After day 21, neurospheres were isolated with Accutase™ (A1110501, Gibco), plated on a poly-L-lysine/laminin-coated culture dish or a confocal dish (211350, SPL), and supplemented with all previous factors and Neurobasal medium containing 25 μM β-mercaptoethanol (21985023, Gibco) and 25 μM glutamic acid (G1626, Sigma).

Differentiated nerve cells were treated with compounds 43,232,239, 243, and 286 (300 nM) of the present disclosure for three hours, respectively. For the control group, the cells were incubated in a medium containing DMSO (D2650-5X 10 ML, Sigma) at a final concentration of 0.05%. For mitochondrial staining, 0.01 nM Mitotracker Red CMXRos (M7512, Life technologies, NY, USA) was treated for 15 minutes immediately before imaging.

The axonal movement of mitochondria from the neuron's axon was imaged with a confocal microscope (Leica SP8; Leica microsystems, UK) equipped with a live cell imaging chamber (Live-cell instrument, Seoul, Korea), which maintains the cells at 37° C. in an atmosphere of 5% CO₂/95% air. Time-lapse image recordings were taken at an exposure time of 500 m/s at an interval of one second for one minute, and the images were analyzed with IMARIS software (BITPLANE, Zurich, Switzerland) to calculate an instantaneous velocity. By using the IMARIS, each mitochondria in an axonal portion of a neuron were captured as an individual particle for each frame. To select an optimal velocity range, the instantaneous velocity of each mitochondrion at each time point was plotted as a histogram at an interval of 0.5 μm/sec. A velocity range in which an instantaneous velocity of the CMT2F MNs group was reduced by about 30-40% compared to an instantaneous velocity of the WT MNs group was selected, and then the data were normalized to set the velocity of the WT MNs group to 1. In this way, the recovery effect of the compound of the present disclosure on mitochondrial velocity was calculated as a single relative value, and GraphPad Prism 5 (GraphPad Software, Inc., USA) was used to count and plot the frequency of the instantaneous velocity.

One-way ANOVA was performed with GraphPad Prism 5 software and all data were expressed as mean±SEM.

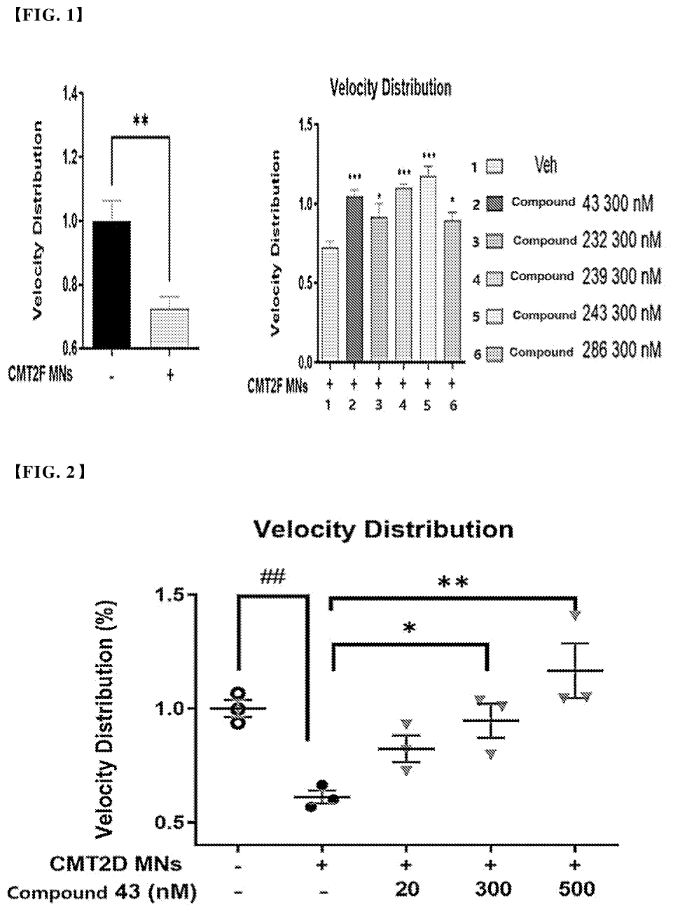

The results of evaluating the effect of the compound of the present disclosure on mitochondrial axon transport are shown in table 1 as follows (FIG. 1).

TABLE 1

| | Relative velocity | Increased |
|---|---|---|
| Group | (Mean ± SEM) | velocity²⁾ |
| WT MNs + Vehicle¹⁾ | 1.000 ± 0.181 | 100 |
| CMT2F MNs + Vehicle | 0.724 ± 0.110 | 0 |
| CMT2F MNs + Compound 43 | 1.046 ± 0.072 | 116.7 |
| CMT2F MNs + Compound 232 | 0.918 ± 0.145 | 70.3 |
| CMT2F MNs + Compound 239 | 1.100 ± 0.042 | 136.2 |
| CMT2F MNs + Compound 243 | 1.135 ± 0.110 | 148.9 |
| CMT2F MNs + Compound 286 | 0.896 ± 0.086 | 62.3 |

¹⁾0.05% DMSO
²⁾Increased velocity(%) = (Group − [CMT2F MNs + Vehicle])/([WT MNs + Vehicle] − [CMT2F MNs + Vehicle])*100

From above table 1, it was confirmed that the compound of the present disclosure exhibits an effect of improving and restoring the axonal mitochondrial movement velocity.

### <Experimental Example 1-2> Mitochondrial Axon Transport in Gars^{P234KY} CMT2D Motor Neurons

The effect of the compound of the present disclosure on axonal mitochondrial movement in motor neurons derived from a human iPSC-based model of Gars^{P234KY} CMT2D (hereinafter referred to as “CMT2D MNs”) was evaluated as follows.

Wild type (WT) and Gars^{P234KY} induced pluripotent stem cell (iPSC) were provided from Samsung Medical Center (IRB No. 2016-12-001), transferred to a CKD pharmacology research laboratory before an experiment, and differentiated into motor neurons according to the following protocol. To produce embryoid bodies, a colony of iPSC was dissociated into small clumps by using enzymes and cultured in suspension in a Petri dish for two days. Culture medium was supplemented with 10 μM Y27632 (Rho-associated kinase inhibitor; Y0503, Tocris Bioscience, Bristol, UK), 20 ng/mL bFGF (PHG0024, Gibco), 10 μM SB435142 (SMAD inhibitor; S4317, Sigma), 0.2 μM LDN193189 (SMAD inhibitor; SML0559, Sigma) and penicillin/streptomycin (15140122, Gibco). On day 5, for casualization, 1 μM retinoic acid (R2625, Sigma), 0.4 μg/mL ascorbic acid (A4544, Sigma), 10 ng/mL brain-derived neurotrophic factor (BDNF; SRP3014, Sigma) and 1% N2 supplement (17502048, Gibco) were added. On day 7, for ventralization, SB435142 and LDN193189 (SMAD inhibitor) were washed, and 1 μM purmorphamine (Sonic hedgehog agonist; SML0868, Sigma) were added. On day 17, cells were cultured in Neurobasal medium [prepared on day 7 and supplemented with 10 ng/mL IGF-1 (I3769, Sigma), 10 ng/mL GDNF (G1777, Gibco) and 10 ng/mL 2% B-27™ (A1486701, Gibco)]. The cells were cultured in suspension in a Petri dish. After day 21, neurospheres were isolated with Accutase™ (A1110501, Gibco), plated on a poly-L-lysine/laminin-coated culture dish or a confocal dish (211350, SPL), and supplemented with all previous factors and Neurobasal medium containing 25 μM β-mercaptoethanol (21985023, Gibco) and 25 μM glutamic acid (G1626, Sigma).

Differentiated nerve cells were treated with compound 43 (20, 300, and 500 nM) of the present disclosure for two hours, respectively. For the control group, the cells were incubated in a medium containing DMSO (D2650-5X 10 ML, Sigma) at a final concentration of 0.05%. For mitochondrial staining, 0.01 nM Mitotracker Red CMXRos (M7512, Life technologies, NY, USA) was treated for 15 minutes immediately before imaging.

The axonal movement of mitochondria from the neuron's axon was imaged with a confocal microscope (Leica SP8; Leica microsystems, UK) equipped with a live cell imaging chamber (Live-cell instrument, Seoul, Korea), which maintains the cells at 37° C. in an atmosphere of 5% CO₂/95% air. Time-lapse image recordings were taken at an exposure time of 500 m/s at an interval of one second for one minute, and the images were analyzed with IMARIS software (BITPLANE, Zurich, Switzerland) to calculate an instantaneous velocity. By using the IMARIS, each mitochondria in an axonal portion of a neuron were captured as an individual particle for each frame. To select an optimal velocity range, the instantaneous velocity of each mitochondrion at each time point was plotted as a histogram at an interval of 0.5 μm/sec. A velocity range in which an instantaneous velocity of the CMT2D MNs group was reduced by about 40% compared to an instantaneous velocity of the WT MNs group was selected, and then the data were normalized to set the velocity of the WT MNs group to 1. In this way, the recovery effect of the compound of the present disclosure on mitochondrial velocity was calculated as a single relative value, and GraphPad Prism 5 (GraphPad Software, Inc., USA) was used to count and plot the frequency of the instantaneous velocity.

One-way ANOVA was performed with GraphPad Prism 5.0 software (post hoc: Dunnett's multiple comparison test) and all data were expressed as mean±SEM.

The results of evaluating the effect of the compound of the present invention on mitochondrial axon transport are shown in table 2 as follows (FIG. 2).

TABLE 2

| | Relative velocity | Increased |
|---|---|---|
| Group | (Mean ± SEM) | velocity²⁾ |
| WT MNs + Vehicle¹⁾ | 1.000 ± 0.065 | 100 |
| CMT2D MNs + Vehicle | 0.611 ± 0.049 | 0 |
| CMT2D MNs + compound 43 (20 nM) | 0.823 ± 0.102 | 54.5 |
| CMT2D MNs + compound 43 (300 nM) | 0.946 ± 0.130 | 86.1 |
| CMT2D MNs + compound 43 (500 nM) | 1.166 ± 0.207 | 142.7 |

¹⁾0.05% DMSO
²⁾Increased velocity(%) = (Group − [CMT2D MNs + Vehicle])/([WT MNs + Vehicle] − [CMT2D MNs + Vehicle])*100

From above table 2, it was confirmed that the compound of the present disclosure exhibits the effect of improving and restoring the axonal mitochondrial movement velocity in a dose-dependent manner.

### <Experimental Example 2> Evaluation of Protein Expression

This experiment was made to confirm the effect of the compound of the present disclosure on the expression of the myelin sheath-associated protein (PMP22) in C22 mice.

2.5-week-old WT and C22 mice (TG(PMP22)C22Clh) were transferred to an animal facility of the CKD research laboratory and kept for one week for acclimatization. Animals were provided with a standard diet (Central Lab Animal, Inc.) and water ad libitum and were housed in a controlled environment with a temperature (22±2° C.), humidity (44-56%), and a 12-hour light-dark cycle. All experimental procedures were approved and performed according to the Institutional Animal Care and Use Committee (IACUC) of the Korea CKD Laboratory Animal Center (with the IACUC animal study protocol approval number: S-17-033).

Each group was classified as shown in table 3 below.

TABLE 3

| | | Dosage | The route of |
|---|---|---|---|
| Animal | Group | (mg/kg) | administration |
| WT | Vehicle¹⁾ | — | PO²⁾, |
| C22 | Vehicle | — | BID³⁾ |
| CMT1A | Compound 43 | 3 | |

¹⁾Vehicle(Veh): 0.5% MC
²⁾PO: Oral administration
³⁾BID: Twice per day

Compound 43 (3 mg/kg) of the present disclosure was administered repeatedly twice a day for two weeks. The sciatic nerve was collected within 0.5 hours after the last dosing and the collected sciatic nerve was dissolved in ice-cold RIPA buffer (Cell signaling, 9803) containing a cocktail of protease inhibitor (Roche, 04693132001) and a phosphatase inhibitor (Roche, 04906837001).

A protein lysate was loaded on SDS-PAGE and transferred to the NC membrane. The membrane was blocked with 30% BSA-TBST for one hour and then incubated with tubulin antibodies at 4° C. overnight. After washing three times, the membrane was incubated with secondary antibodies for one hour and washed three times. The membrane was visualized with ECL detection reagent (GE healthcare, RPN2235) and band intensity was measured with ChemiDoc™ MP (BIO-RAD, 12003154).

All results were expressed as mean±SEM and statistical significance was analyzed with unpaired t-test, one-tailed, and GraphPad Prism 5 (GraphPad Software, Inc., USA).

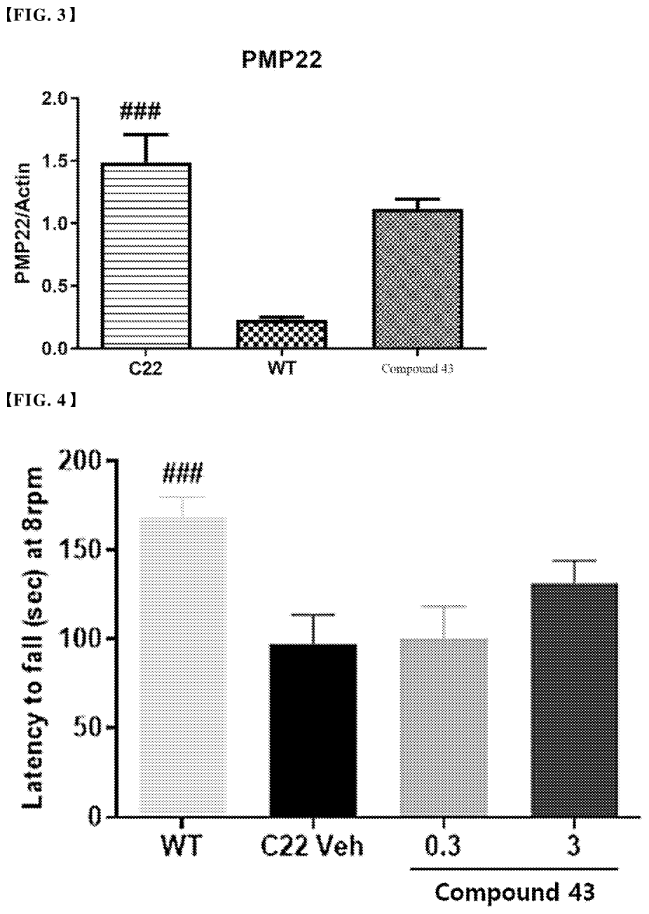

As a result, as shown in FIG. 3, it was confirmed that the compound of the present disclosure exhibits an effect of inhibiting PMP22 protein expression induced in C22 mice.

### <Experimental Example 3> Evaluation of Behaviors

This experiment was made to evaluate the efficacy of the compound of the present disclosure by confirming the effect of the compound of the present disclosure on the motor functions of animals.

### <Experimental Example 3-1> Evaluation of Behaviors in CMT1A Mouse Model

2.5-week-old male C22 CMT1A mice were provided with a standard diet (Central Lab Animal, Inc.) and water ad libitum and were housed in a controlled environment with a temperature (22±2° C.), humidity (44-56%), and a 12-hour light-dark cycle. All experimental procedures were approved and performed according to the Institutional Animal Care and Use Committee (IACUC) of the Korea CKD Laboratory Animal Center (with an approved number: S-17_033).

A rotarod test, a balance beam test, and a grip strength test were performed once a day for two weeks, and then the animals were classified into each group as shown in table 4 below according to a Z-array method based on the rotarod test, the balance beam test, the grip strength test and weight values.

TABLE 4

| | | Dosage | |
|---|---|---|---|
| Animal | Group | (mg/kg) | Route |
| WT | Vehicle¹⁾ | — | PO²⁾, |
| C22 | Vehicle | — | BID³⁾ |
| CMT1A | Compound 43 | 0.3 | |
| | | 3 | |

¹⁾Vehicle(Veh): 0.5% MC
²⁾PO: Oral administration
³⁾BID: Twice a day

Compound 43 (0.3 and 3 mg/kg) of the present disclosure was orally administered repeatedly twice a day for two weeks and a behavioral test was performed 30 minutes after dosing. The behavioral test was performed once a week in a behavioral assessment room in the CKD research laboratory.

Data were expressed as mean±SEM, and statistical significance between the group treated with the compound of the present disclosure and the vehicle group was analyzed with unpaired t-test for comparison of two groups, or one-way ANOVA (post-hoc analysis using Dunnett's test) for comparison of three or more groups. When an independent variable (e.g., duration of administration) was added, statistical significance was analyzed with two-way ANOVA (post-hoc analysis using Bonferroni's test). All statistical analyses were performed with GraphPad Prism (ver 5.0).

### Constant Rotarod Test (CRT)

The rotarod test (LE 8205, Panlab) was performed to evaluate the forced motor activity and coordination function. For acclimatization, all test animals were subjected to an orientation training at 8 rpm five times a day over a three-day course, and the animals satisfying a category of latency to fall of 150-180 seconds were used for further experiments (about 80% of the animals satisfied said category). The latency to fall was measured three times at a fixed speed of 8 rpm for three minutes. The rotarod test was repeated three times for each experiment and the maximum value of the three measured values was used as a test result (latency to fall).

As a result, as shown in FIG. 4, it was confirmed that the compound of the present disclosure exhibits an effect of remarkably enhancing the latency to fall.

### Grip Strength Test (GST)

One of the main symptoms of CMT is muscle degeneration due to neurodegeneration for motor control. In clinical practice, a degree of muscle degeneration is evaluated by grip strength and ankle dorsiflexion. The GST (BIO-GS 3, BIOSEB) was performed to evaluate the functions of the quadrupeds by using a grid (wire mesh). All experiments were performed by one person and the maximum value of five consecutive measurements was used as the test result. After allowing a mouse to hold the wire mesh, a weak tension was applied to the mouse, which was then gently pulled out at an inclination of about 15°, to measure a maximum tension.

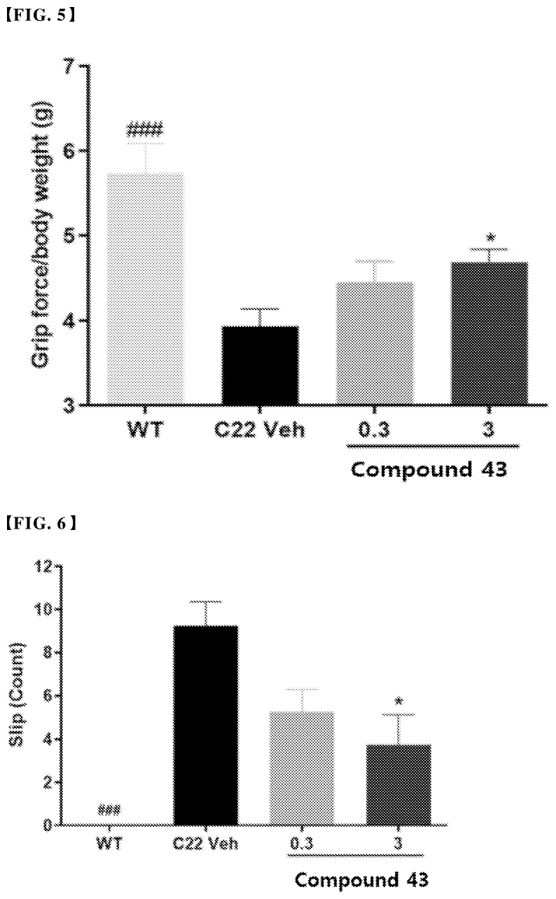

As a result, as shown in FIG. 5, it was confirmed that the compound of the present disclosure exhibits an effect of remarkably enhancing the grip strength in a dose-dependent manner.

### Balance Beam Test (BBT)

The balance beam test was performed to measure a motor coordination function.

From the test, the function of hind limbs was measured and the sense of balance was observed. A rod (1.2 cm wide, 0.6 cm high, and 1.0 m long) was fixed at an inclination of 9° (45 cm high from a starting point and 60 cm high from an ending point). At the starting point, a mouse was stimulated with a light of 60 W and an ending point was equipped with a dark box without light so that the mice could feel a sense of relief. All experimental animals were acclimatized 30 minutes before an evaluation under the same conditions as the experimental conditions. The mouse was placed at the starting point to walk toward the ending point, and the number of slips after departure was measured. Before the test, the mouse was trained three times a day for two days and the results on day 3 were used for grouping. Each experiment was independently evaluated by two individuals.

As a result, as shown in FIG. 6, it was confirmed that the compound of the present invention exhibits an effect of remarkably reducing the number of slips.

In other words, it was confirmed that the compound of the present disclosure may be advantageously used for the prevention and treatment of the CMT by remarkably enhancing the motor functions (CRT, GST, and BBT) of CMT1A mice.

### <Experimental Example 3-2> Evaluation of Behaviors in CMT2F Mouse Model

Seven-month-old male HSPB1^{S135F} CMT2F mice were provided with a standard diet (Central Lab Animal, Inc.) and water ad libitum and were housed in a controlled environment with a temperature (22±2° C.), humidity (44-56%) and a 12-hour light-dark cycle. All experimental procedures were approved and performed according to the Institutional Animal Care and Use Committee (IACUC) of the Korea CKD Laboratory Animal Center (with an approved number: S-17_033).

A rotarod test and a grip strength test were performed once a day for two weeks, and then the animals were classified into each group as shown in table 5 below according to a Z-array method based on the rotarod test, the grip strength test, and weight values.

TABLE 5

| | | Administered | |
|---|---|---|---|
| Animal | Group | dose (mg/kg) | Route |
| WT | Vehicle¹⁾ | — | PO²⁾, |
| HSPB1^{S135F} | Vehicle | — | BID³⁾ |
| CMT2F | Compound 43 | 1 | |
| | | 10 | |

¹⁾Vehicle(Veh): 0.5% MC
²⁾PO: Oral administration
³⁾BID: Twice a day

Compound 43 (1 and 10 mg/kg) of the present disclosure was orally administered repeatedly twice a day for eight weeks and a behavioral test was performed in one hour after dosing. Behavioral tests were conducted at weeks 0, 4, and 8.

Data were expressed as mean±SEM and statistical significance was analyzed with two-way ANOVA (posttest, Bonfeeroni). All statistical analyses were performed with GraphPad Prism 5.0.

### Constant Rotarod Test (CRT)

The rotarod test (LE 8205, Panlab) was performed to evaluate the forced motor activity and coordination function. For acclimatization, all test animals were subjected to orientation training at 8 rpm five times a day over a three-day course, and the animals satisfying a category of latency to fall of 150-180 seconds were used for further experiments (about 80% of the animals satisfied said category). The latency to fall was measured three times at a fixed speed of 8 rpm for three minutes. The rotarod test was repeated three times for each experiment and the maximum value of the three measured values was used as a test result (latency to fall).

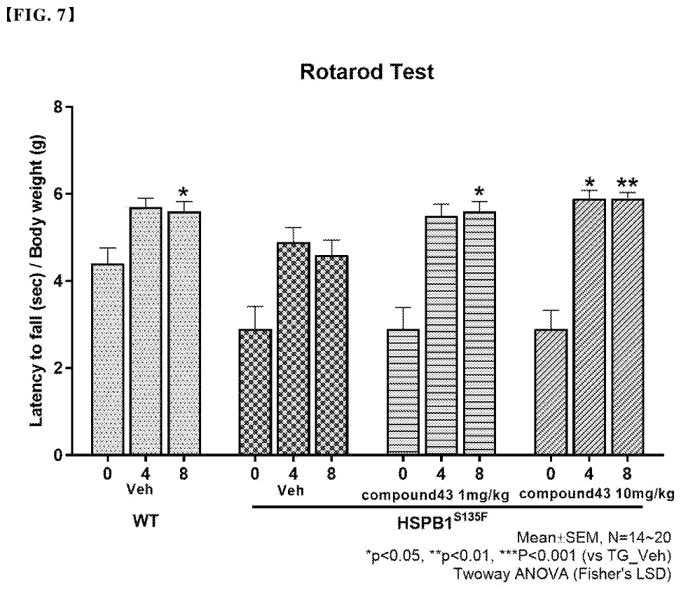

As a result, as shown in FIG. 7, it was confirmed that the compound of the present disclosure exhibits an effect of remarkably enhancing the latency to fall in a dose-dependent manner.

### Grip Strength Test (GST)

One of the main symptoms of CMT is muscle degeneration due to neurodegeneration for motor control. In clinical practice, a degree of muscle degeneration is evaluated by grip strength and ankle dorsiflexion. The GST (BIO-GS 3, BIOSEB) was performed to evaluate the functions of the quadrupeds by using a grid (wire mesh). All experiments were performed by one person and the maximum value of five consecutive measurements was used as the test result. After allowing a mouse to hold the wire mesh, a weak tension was applied to the mouse, which was then gently pulled out at an inclination of about 15°, to measure a maximum tension.

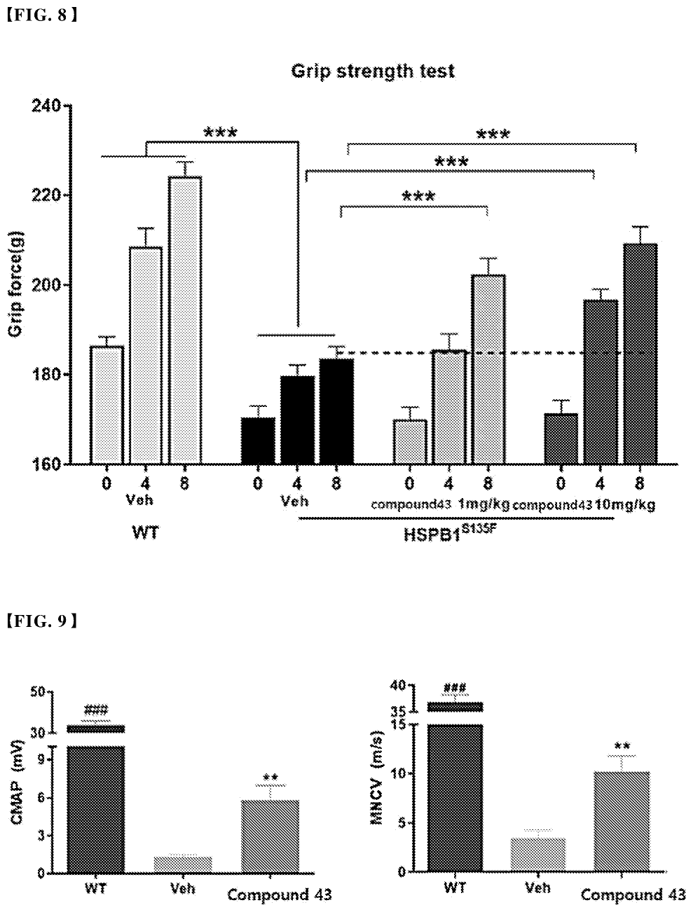

As a result, as shown in FIG. 8, it was confirmed that the compound of the present disclosure exhibits an effect of remarkably enhancing the grip strength in a dose-dependent manner.

In other words, it was confirmed that the compound of the present disclosure may be advantageously used for the prevention and treatment of the CMT by remarkably enhancing the motor functions (CRT and GST) of CMT2F mice.

### <Experimental Example 4> Nerve Conduction Study (NCS)

This experiment was made to evaluate the efficacy of the compound of the present disclosure by confirming the effect of the compound of the present disclosure on the nerve conduction velocity of animals.

### <Experimental Example 4-1> 2.5-Week-Old CMT1A Mouse Model

2.5-week-old male C22 CMT1A mice were provided with a standard diet (Central Lab Animal, Inc.) and water ad libitum and were housed in a controlled environment with a temperature (22±2° C.), humidity (44-56%), and a 12-hour light-dark cycle. All experimental procedures were approved and performed according to the Institutional Animal Care and Use Committee (IACUC) of the Korea CKD Laboratory Animal Center (with an approved number: S-17_033).

Each group was classified as shown in table 6 below.

TABLE 6

| | | Administered | |
|---|---|---|---|
| Animal | Group | dose (mg/kg) | Route |
| WT | Vehicle¹⁾ | — | PO²⁾, |
| C22 | Vehicle | — | BID³⁾ |
| CMT1A | Compound 43 | 1 | |

¹⁾Vehicle(Veh): 0.5% MC
²⁾PO: Oral administration
³⁾BID: Twice a day

Compound 43 (1 mg/kg) of the present disclosure was orally administered repeatedly twice a day for two weeks.

Animals were anesthetized with isoflurane (USP Terrel, Piramal Critical Care, Inc., NDC 66794-017-25) in 30% oxygen (Daehan gas) and 70% nitrogen (Daehan gas), and the hairs of a distal back and hind legs were completely removed. The skin was maintained at >32 É with an external heating device. Electrophysiological recordings were performed on the sciatic nerve, the largest nerve in the peripheral nervous system (PNS), and the nerve conduction study (NCS) was performed with a Nicolet Viking Quest. A compound motor action potential (CMAP) amplitude and a motor neuron conduction velocity (MNCV) were measured.

Data were expressed as mean±SEM, and statistical significance between the group treated with the compound of the present invention and the vehicle group was analyzed with unpaired t-test for comparison of two groups, or one-way ANOVA (post-hoc analysis using Dunnett's test) for comparison of three or more groups. When an independent variable (e.g., duration of administration) was added, statistical significance was analyzed with two-way ANOVA (post-hoc analysis using Bonferroni's test). All statistical analyses were performed with GraphPad Prism (ver 5.0).

As a result, as shown in FIG. 9, it was confirmed that the compound of the present disclosure exhibits an effect of remarkably enhancing MNCV and CMAP.

In other words, it was confirmed that the compound of the present disclosure may be advantageously used for the prevention and treatment of the CMT by enhancing the nerve conduction velocity.

### <Experimental Example 4-2>8-Month-Old CMT1A Mouse Model

Eight-month-old male/female C22 CMT1A mice were provided with a standard diet (Central Lab Animal, Inc.) and water ad libitum and were housed in a controlled environment with a temperature (22±2° C.), humidity (44-56%), and a 12-hour light-dark cycle. All experimental procedures were approved and performed according to the Institutional Animal Care and Use Committee (IACUC) of the Korea CKD (IACUC No: S-17-033).

Each group was classified as shown in table 7 below.

TABLE 7

| | | | Gender | |
|---|---|---|---|---|
| | | Administered | (number of | |
| Animal | Group | dose(mg/kg) | individuals) | |
| WT | Vehicle¹⁾ | — | Male(4)/Female(3) | PO²⁾, |
| C22 | Vehicle | — | Male(6)/Female(6) | BID³⁾ |
| CMT1A | Compound 43 | 1 | Male(6)/Female(6) | |

¹⁾Vehicle(Veh): 0.5% MC
²⁾PO: Oral administration
³⁾BID: Twice a day

Compound 43 (1 mg/kg) of the present disclosure was orally administered repeatedly twice a day for 12 weeks.

Animals were anesthetized with isoflurane (USP Terrel, Piramal Critical Care, Inc., NDC 66794-017-25) in 30% oxygen (Daehan gas) and 70% nitrogen (Daehan gas), and the hairs of a distal back and hind legs were completely removed. The skin was maintained at >32 É by using an external heating device. Electrophysiological recordings were performed on the sciatic nerve, the largest nerve in the peripheral nervous system (PNS), and the nerve conduction study (NCS) was performed with a Nicolet Viking Quest. A compound motor action potential (CMAP) amplitude and a motor neuron conduction velocity (MNCV) were measured.

Data were expressed as mean±SEM, and statistical significance was analyzed with unpaired student's t-test for comparison of two groups, or one-way ANOVA (post-hoc analysis using Dunnett's test) for comparison of three or more groups. All statistical analyses were performed with GraphPad Prism (ver 5.0).

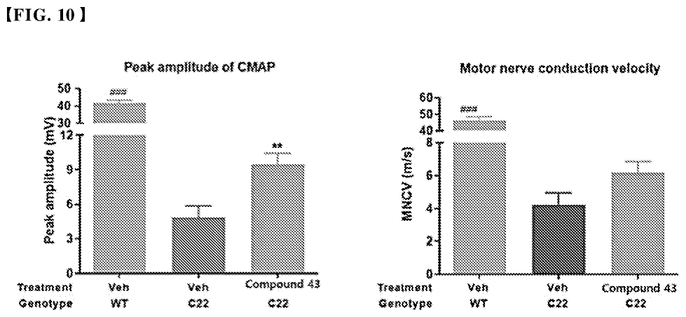

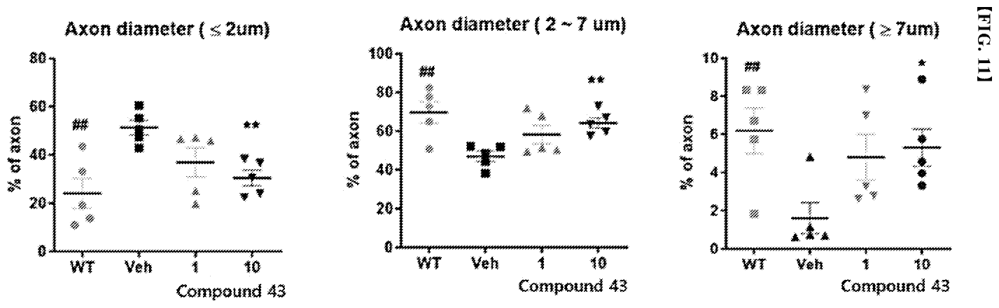

As a result, as shown in FIG. 10, it was confirmed that the compound of the present disclosure exhibits an effect of remarkably enhancing MNCV and CMAP.

In other words, it was confirmed that the compound of the present disclosure may be advantageously used for the prevention and treatment of the CMT by enhancing the nerve conduction velocity.

### <Experimental Example 5> Histopathological Analysis

This experiment was made to confirm the effect of the compound of the present disclosure on the axon size of the sciatic nerve fiber.

6.5 to 8.5-month-old male HSPB1^{S135F} CMT2F mice were provided with a standard diet (Central Lab Animal, Inc.) and water ad libitum and were housed in a controlled environment with a temperature (22±2° C.), humidity (44-56%), and a 12-hour light-dark cycle.

Each group was classified as shown in table 8 below.

TABLE 8

| | | Administered | |
|---|---|---|---|
| Animal | Group | dose(mg/kg) | Route |
| WT | Vehicle¹⁾ | — | PO²⁾, |
| HSPB1^{S135F} | Vehicle | — | BID³⁾ |
| CMT2F | Compound 43 | 1 | |
| | | 10 | |

¹⁾Vehicle(Veh): 0.5% MC, 10 mL/kg
²⁾PO: Oral administration
³⁾BID: Twice a day

Compound 43 (1 and 10 mg/kg) of the present disclosure was orally administered repeatedly twice a day for 12 weeks. After the final treatment, the sciatic nerve was collected at 0.5 hours and fixed overnight in a 2.5% glutaraldehyde solution (340855, Sigma). A sample was transferred to the Department of Pathology in Asan Hospital for semithin sections and toluidine blue (T3260, Sigma) staining.

The fixed sample was processed conventionally for image analysis. A 0.5 m section was prepared and stained with toluidine blue.

A histological evaluation was performed under optical microscopy. Pathological changes including demyelination, remyelination, abnormally thin myelin, and axonal morphological change were investigated from the section. At last, an axon diameter was analyzed with Image J software.

Data were expressed as mean±SEM. One-way ANOVA and post-hoc analysis using Dunnett's test were used for statistical significance between the HSPB1^{S135F} CMT2F (vehicle) group and the HSPB1^{S135F} CMT2F (Compound 43) group, and the paired T-test was used for statistical significance between the WT group and the HSPB1^{S135F} CMT2F (vehicle) group. All statistical analyses were performed with GraphPad Prism 5.0.

The results of evaluating the effect of the compound of the present disclosure on the axon size of the sciatic nerve fiber are shown in table 9 as follows (FIG. 11).

TABLE 9

| | | Diameter of Axon (μm) | | | | | | | | | | |
|---|---|---|---|---|---|---|---|---|---|---|---|---|
| Group | | <1 | 1-2 | 2-3 | 3-4 | 4-5 | 5-6 | 6-7 | 7-8 | 8-9 | 9-10 | >10 |
| WT | | 73 ± 32 | 439 ± 128 | 489 ± 25 | 389 ± 50 | 295 ± 56 | 182 ± 30 | 120 ± 21 | 76 ± 16 | 35 ± 7 | 17 ± 6 | 6 ± 2 |
| HSPB1^{S135F} | Vehicle | 378 ± 58 | 758 ± 26 | 493 ± 38 | 284 ± 17 | 140 ± 13 | 85 ± 11 | 45 ± 7 | 24 ± 11 | 9 ± 6 | 3 ± 2 | 1 ± 1 |
| CMT2F | Compound | 273 ± 64 | 691 ± 74 | 574 ± 54 | 429 ± 70 | 269 ± 57 | 196 ± 40 | 128 ± 26 | 83 ± 21 | 36 ± 13 | 12 ± 5 | 5 ± 3 |
| | 43 | | | | | | | | | | | |
| | (1 mg/kg) | | | | | | | | | | | |
| | Compound | 124 ± 42 | 565 ± 26 | 561 ± 49 | 588 ± 29 | 265 ± 31 | 161 ± 21 | 112 ± 20 | 67 ± 12 | 39 ± 12 | 16 ± 7 | 6 ± 5 |
| | 43 | | | | | | | | | | | |
| | (10 mg/kg) | | | | | | | | | | | |

From above table 9, it was confirmed that the compound of the present disclosure exhibits an effect of increasing and restoring the axon size.

### <Experimental Example 6> Genetic Analysis

This experiment was made to evaluate the efficacy of the compound of the present disclosure through a genetic analysis of animals.

Ten-week-old C3 CMT1A mice were provided with a standard diet (Central Lab Animal, Inc.) and water ad libitum and were housed in a controlled environment with a temperature of 22±2° C., a humidity of 44-56% and a 12-hour light-dark cycle. All experimental procedures were approved and performed according to the Institutional Animal Care and Use Committee (IACUC) of the Korea CKD Laboratory Animal Center (with an approved number: S-17_033).

Each group was classified as shown in table 10 below.

TABLE 10

| | | Administered | Administration |
|---|---|---|---|
| Animals | Group | dose (mg/kg) | route |
| WT | Vehicle¹⁾ | — | PO²⁾, |
| C3 CMT1A | Vehicle(C3) | — | BID³⁾ |
| | Compound 43 | 10 mg/Kg | |

¹⁾Vehicle (Veh): 0.5% MC
²⁾PO: Oral administration
³⁾BID: Twice a day

Vehicle was 0.5% methylene chloride (MC), and each group was administered vehicle or compound 43 for eight weeks. TG or C3 refers to C3 CMT1Amice; a wild type (WT) is a group in which only vehicle is administered to normal mice; Vehicle(C3) or C3 group is a group in which only vehicle is administered to C3 CMT1A mice; and compound 43 is a group in which compound 43 is administered to C3 CMT1A mice with five animals in each group.

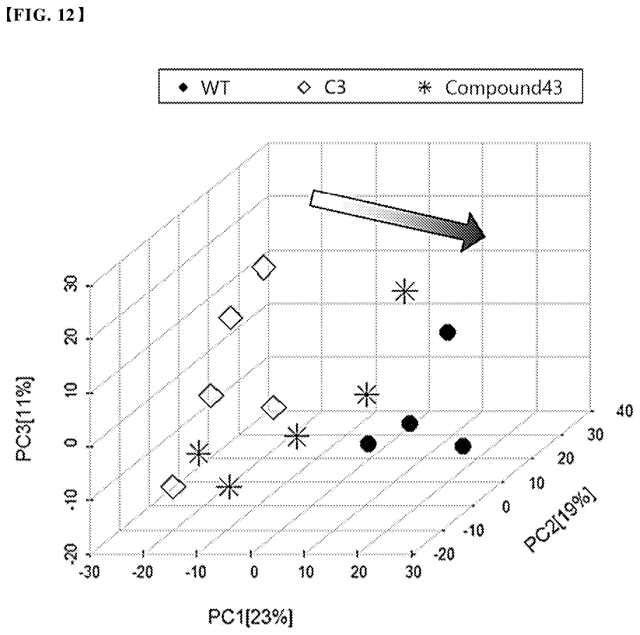

After the last administration of vehicle or compound 43, the sciatic nerves were collected at 0.5 hours, from which RNA was extracted with a kit (RNeasy Kit, Qiagen, Venlo, Netherlands), and then RNA quantification and integrity evaluation was carried out evaluate through Quant-IT RiboGreen (Invitrogen) and apeStation RNA screentape (Agilent, Santa Clara, CA, USA). Then, the corresponding RNA was quantified at a gene level through RNA sequencing using more than 24 million leads. A gene set enrichment analysis was performed from a quantified gene level to confirm various biological effects (gene sets) among WT, TG(C3), and drug administration groups (compound 43), and then the significance of the results was analyzed and confirmed with a threshold value set to nominal p<0.05, false detection rate (FDR) q<0.25. The gene set, of which significance was finally confirmed, was graphed through Prism 9, and the results are shown in FIGS. 12 to 15.

As confirmed above in FIG. 12, it was shown that the group dosed with compound 43 shows genetic properties close to those of normal mice (WT) compared with the C3 group, when divided into the groups of WT, C3 group, and compound 43 group on the basis of specific criteria (three PC values) of the gene module.

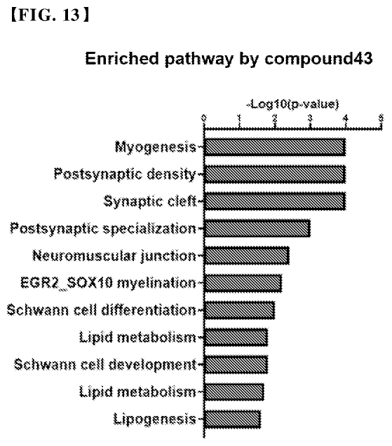

In addition, as confirmed in FIGS. 13 to 15, it was understood that compound 43 raises gene levels associated with myogenesis, postsynaptic density, postsynaptic specialization, neuromuscular junction, synaptic cleft, EGR2_SOX10 myelination, Schwann cell myelination/development, Schwann cell differentiation, lipid metabolism, lipogenesis, etc., which were down-regulated in mice with CMT disease (TG, C3 CMT1A mice).

It can be confirmed from FIG. 13 that the genes associated with myogenesis, postsynaptic density, postsynaptic specialization, neuromuscular junction, synaptic cleft, EGR2_SOX10 myelination, Schwann cell myelination/development, Schwann cell differentiation, lipid metabolism, lipogenesis, etc. are remarkably up-regulated in the group dosed with compound 43 compared with in mice with CMT disease (TG, C3 CMT1A mice).

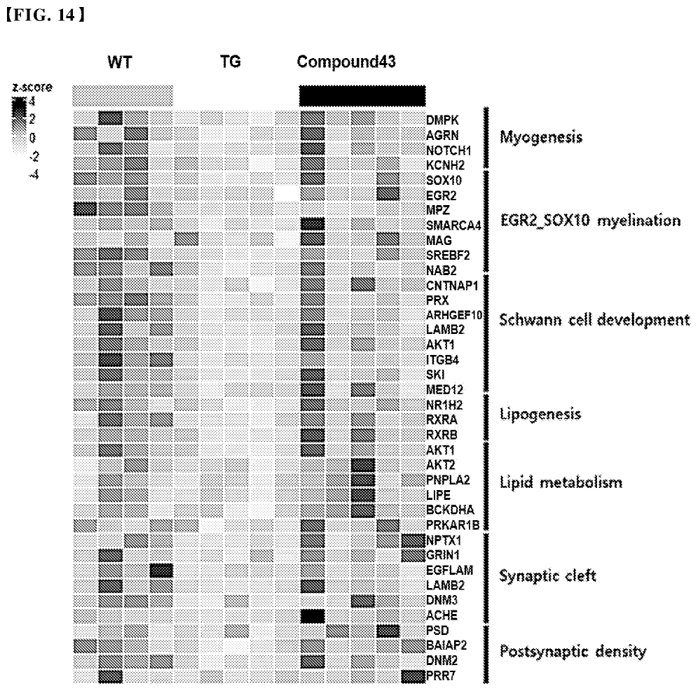

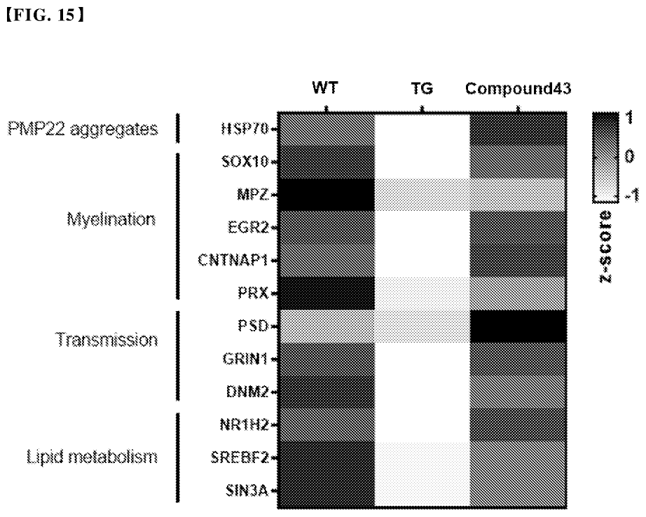

In particular, it can be understood from FIG. 14 that the expression of individual genes included in the gene set varies according to the administration of compound 43; it was confirmed from FIG. 15 that genes associated with PM22 aggregates, myelination, nerotransmission, and lipid metabolism, which appear to be related to CMT disease, vary according to the administration of compound 43; and it could be seen that the transcription of genes involved in increasing neurotransmission and myelination and controlling PM22 aggregations and lipid metabolism is up-regulated according to the administration of compound 43.

### <Experimental Example 7> Analysis of Muscle Fiber Atrophy Amelioration

This experiment was made to evaluate the efficacy of the compound of the present disclosure by confirming the effect of the compound of the present disclosure on the myofibrillar atrophic neuromuscular junctions in CMT mice.

### <Experimental Example 7-1> 10-Week-Old CMT1A Mouse Model

Ten-week-old C3 CMT1A mice were provided with a standard diet (Central Lab Animal, Inc.) and water ad libitum and were housed in a controlled environment with a temperature of 22±2° C., a humidity of 44-56% and a 12-hour light-dark cycle. All experimental procedures were approved and performed according to the Institutional Animal Care and Use Committee (IACUC) of the Korea CKD Laboratory Animal Center (with an approved number: S-17_033).

Each group was classified as shown in table 11 below.

TABLE 11

| | | Administered | Administration |
|---|---|---|---|
| Animals | Group | dose (mg/kg) | route |
| WT | Vehicle¹⁾ | — | PO²⁾, |
| C3 CMT1A | Vehicle(C3) | — | BID³⁾ |
| | Compound 43 | 10 mg/Kg | |

¹⁾Vehicle (Veh): 0.5% MC
²⁾PO: Oral administration
³⁾BID: Twice a day

Vehicle is 0.5% methylene chloride (MC), and each group was administered vehicle or compound 43 for seven weeks. TG or C3 refers to C3 CMT1A mice; a wild type (WT) is a group in which only vehicle is administered to normal mice; Vehicle(C3) or C3 group is a group in which only vehicle is administered to C3 CMT1A mice; and compound 43 is a group in which compound 43 is administered to C3 CMT1A mice with five animals in each group.

### <Experimental Example 7-2> Analysis of Muscle Fiber Atrophy and Neuromuscular Junction

### 1) Autopsy

At seven weeks after the administration of vehicle or drug (compound 43), gastrocnemius muscles were collected for analysis of innervation and muscle tissues at the neuromuscular junctions. The corresponding procedure was performed 30 minutes after the last administration of vehicle or drug.

### 2) Analysis of Muscle Fiber Atrophy

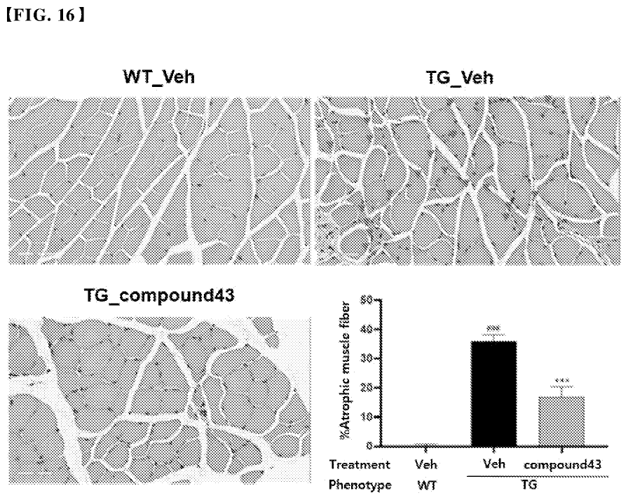

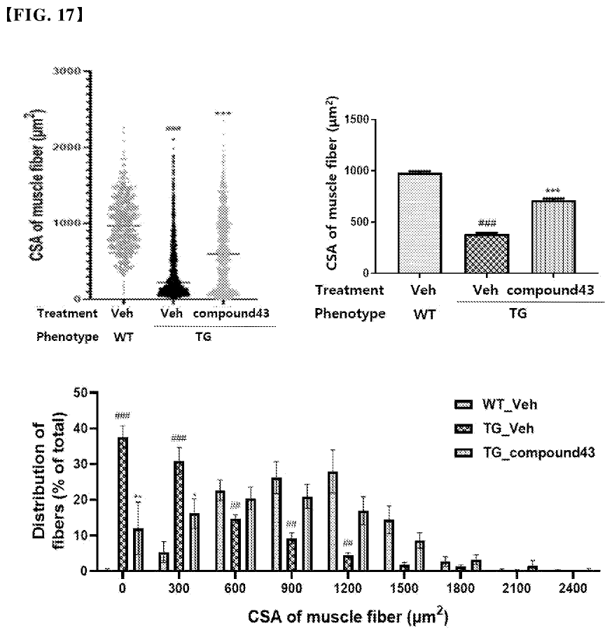

Mice were anesthetized with isoflurane and systemic perfusion through the heart was performed with saline solution (3 mL). The gastrocnemius muscles were excised and fixed with 10% neutral buffered formalin at room temperature. The samples were cut to include a site for evaluation, after which tissues were treated and embedded in paraffin and cut into 4 m thick sections using a sliding microtome. Sectioned slides were stained with H&E and observed under an optical microscope. The proportion of muscle fibers with atrophy was measured by dividing the number of muscle fibers with atrophy by the number of normal muscle fibers, and the cross-sectional area of the muscle fibers was analyzed using NIS element software, and the results are shown in FIGS. 16 and 17.

As can be confirmed from FIGS. 16 and 17, it was shown that C3 CMT1A (TG) have a higher proportion of atrophic muscle fibers and a significant decrease in the cross-sectional area of muscle fibers compared to normal mice (WT). However, in the group of C3 CMT1A mice dosed with compound 43, the proportion of atrophic muscle fibers in C3 CMT1A mice was lowered and the cross-sectional area of muscle fibers was increased.

### 3) Innervation of Neuromuscular Junctions

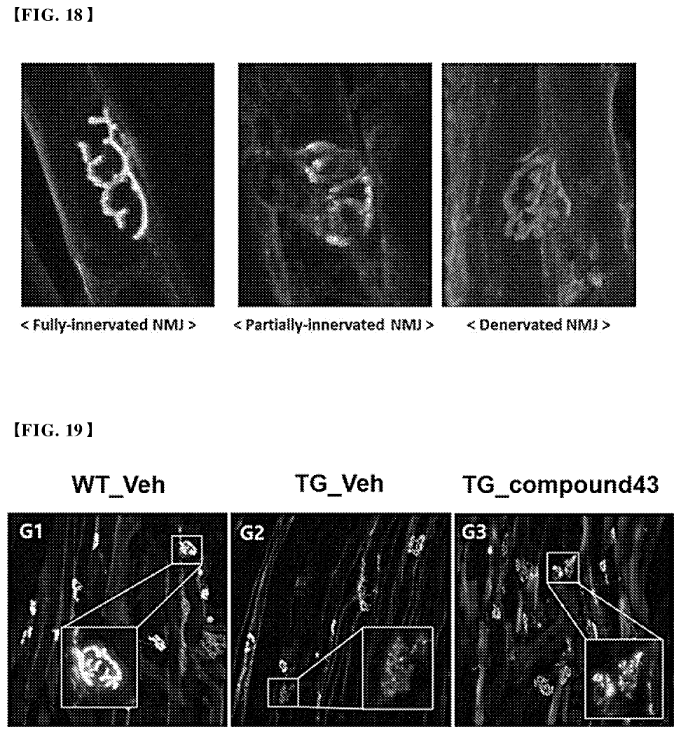

The gastrocnemius muscles collected in 1) were frozen and sectioned with a thickness of 30 m so as to make a slide. The prepared section slides were double immuno-stained with anti-synaptotagmin-2 and α-bungarotoxin, and then a structure of having two antibodies positively stained for each neuromuscular junction was analyzed through a fluorescence microscope, so as to qualitatively analyze a level of innervation per junction of each neuromuscular junction (NMJ) as shown in FIG. 18, and the results thereof are shown in FIG. 19.

As can be confirmed from FIG. 19, it was found that a fully innervated NMJ is decreased in gastrocnemius muscles of C3 CMT1A mice compared to normal mice (WT). However, it could be confirmed that a fully innervated NMJ is increased in the group of C3 CMT1A mice dosed with compound 43.

In addition, the section slide samples with at least 80 neuromuscular junctions per sample were divided into: fully innervation for a case in which patterns of having two antibodies positively stained are fully overlapped on the double staining with anti-synaptotagmin-2 and α-bungarotoxin; partially innervation for a case in which the patterns are partially overlapped; and denervation for a case in which no pattern is overlapped, and the each percentage value was calculated and the significance and graph of the results were analyzed and expressed through Prism 9, and are shown in FIG. 20.

As can be seen from FIG. 20, it was found that the C3 CMT1A mice show at least 10% decrease in the fully innervated NMJ and a high proportion of partially innervated and denervated NMJs compared with normal mice (WT). In contrast, it was confirmed with the group of C3 CMT1A mice dosed with compound 43 that the fully innervated NMJ is increased and the proportion of partially innervated NMJ and denervated NMJ is remarkably decreased.

From the above, it could be seen that the compound of the present disclosure may effectively treat peripheral neuropathic CMT disease by significantly increasing innervation at the nerve junctions in CMT disease.

### <Experimental Example 8> Analysis of Effect on Sensory Nerves 1

This experiment was made to evaluate the efficacy of the compound of the present disclosure by confirming the effect of the compound of the present disclosure on the animal sensory nerves in CMT mice.

### <Experimental Example 8-1> 10-Week-Old CMT1A Mouse Model

Ten-week-old C3 CMT1A mice were provided with a standard diet (Central Lab Animal, Inc.) and water ad libitum and were housed in a controlled environment with a temperature of 22±2° C., a humidity of 44-56% and a 12-hour light-dark cycle. All experimental procedures were approved and performed according to the Institutional Animal Care and Use Committee (IACUC) of the Korea CKD Laboratory Animal Center (with an approved number: S-17_033).

Each group was classified as shown in table 12 below.

TABLE 12

| | | Administered | Administration |
|---|---|---|---|
| Animals | Group | dose (mg/kg) | route |
| WT | Vehicle¹⁾ | — | PO²⁾, |
| C3 CMT1A | Vehicle(C3) | — | BID³⁾ |
| | Compound 43 | 10 mg/Kg | |

¹⁾Vehicle (Veh): 0.5% MC
²⁾PO: Oral administration
³⁾BID: Twice a day

Vehicle is 0.5% methylene chloride (MC), and each group was administered vehicle or compound 43 for eight weeks. TG or C3 refers to C3 CMT1A mice; a wild type (WT) is a group in which only vehicle is administered to normal mice; Vehicle(C3) or C3 group is a group in which only vehicle is administered to C3 CMT1A mice; and compound 43 is a group in which compound 43 is administered to C3 CMT1A mice with five animals in each group.

### <Experimental Example 8-2> Histopathologic Evaluation

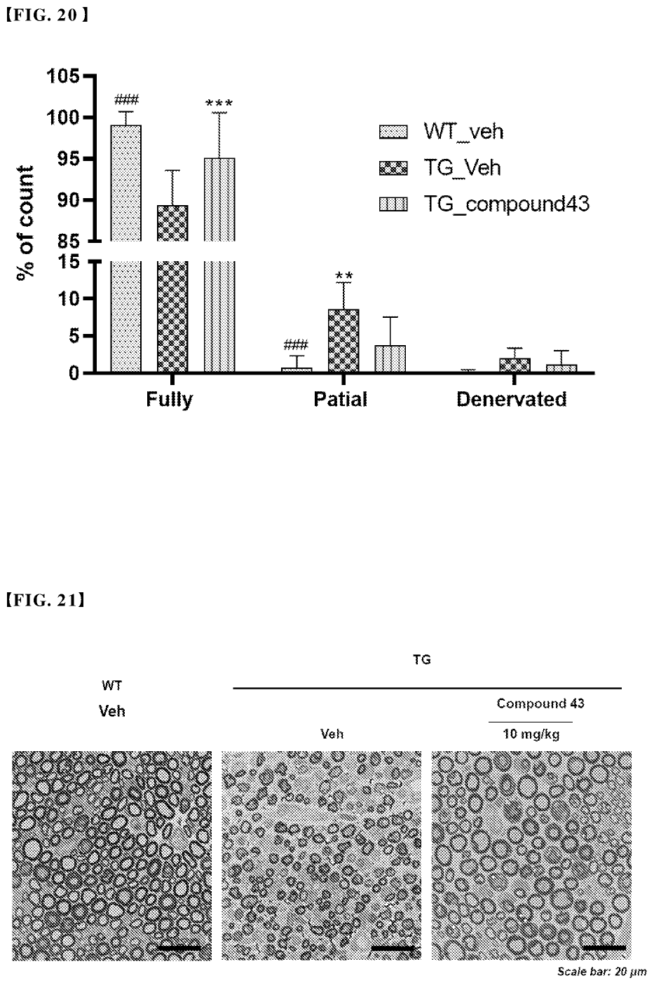

At eight weeks after administering vehicle or drug (compound 43), the sural nerves were collected at 0.5 hours after the final drug administration and then fixed overnight in 2.5% glutaraldehyde solution. The fixed samples were subjected to general tissue processing for image analysis, sectioned with a thickness of 0.5 m, and stained with toluidine blue. Histological evaluation was performed on imaging files taken with an optical microscopy. In each sample, morphological changes of myelin, such as demyelination, remyelination, etc., and pathological changes, including a reduction in axon diameter, were evaluated. The axon and myelin diameters were analyzed with Image J software and a G-ratio thereof was calculated as the ratio of an internal axon diameter to a total external diameter, and the results are shown in FIGS. 21 to 23.

Data were represented as mean±SEM. Statistical significance between groups was tested using one-way ANOVA and post hoc analysis was performed by Dunnett's test. All statistical analyses were performed with GraphPad Prism 9.0.

As understood from FIG. 21, it could be confirmed that the axon diameter and the myelin sheath thickness are decreased in C3 CMT1A mice compared to WT mice, but both the axon diameter and the myelin sheath thickness are increased in the mice dosed with compound 43.

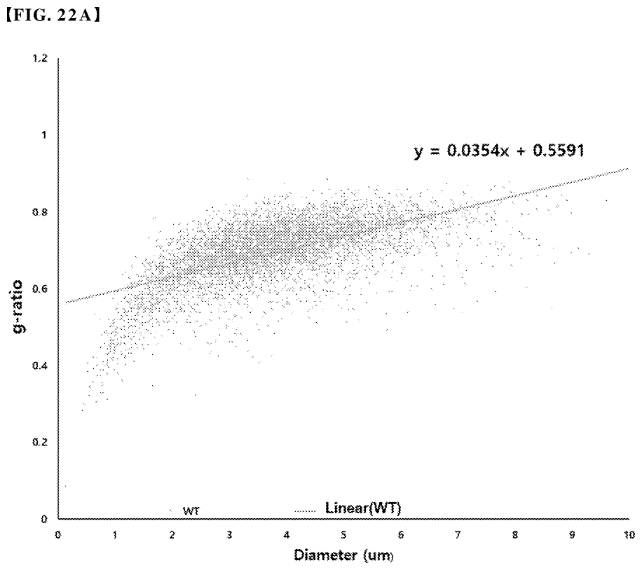

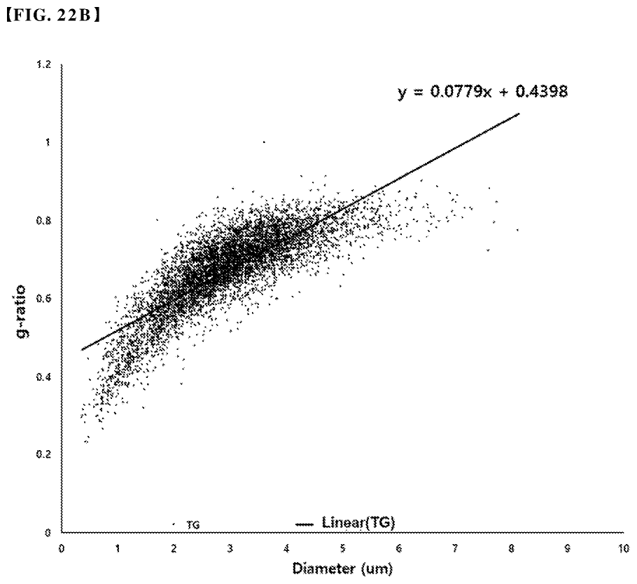

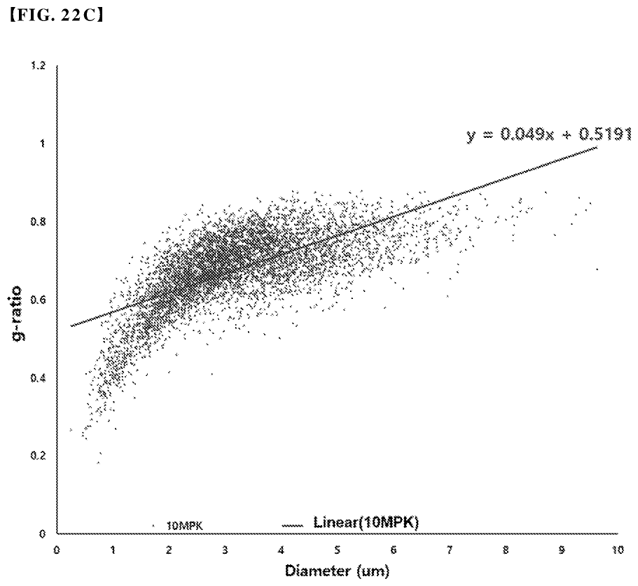

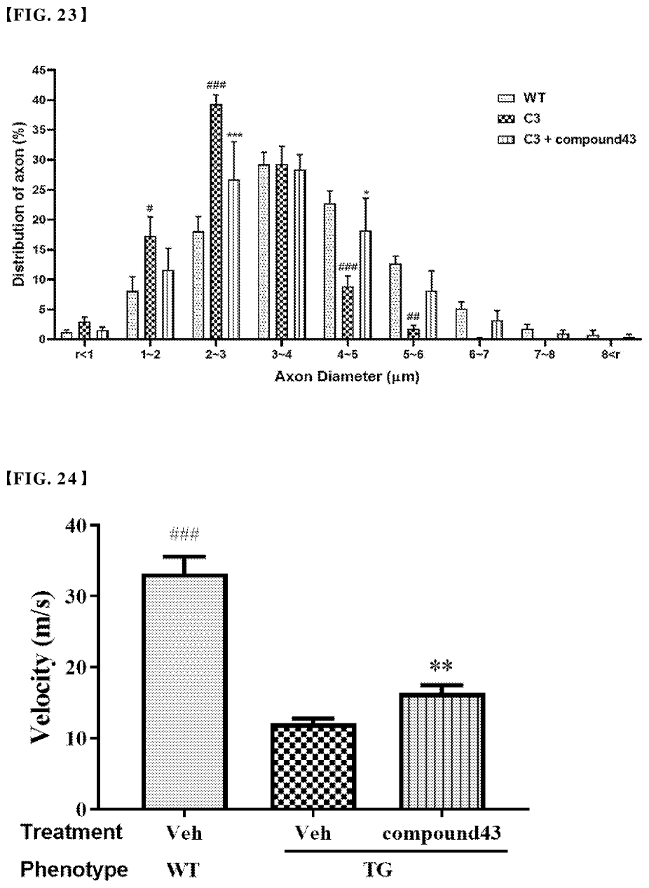

In addition, as understood from FIGS. 22 and 23, it was confirmed that C3 CMT1A mice do not have a significant difference in the proportion of demyelinated fibers compared with WT mice, but show a significant difference in the increase of the slop of the g-ratio and the decrease in the axon diameter in the C3 mice treated with vehicle. In this case, the g-ratio of C3 CMT1A dosed with compound 43 had a lower slope than that of vehicle-treated animals, which is related to the reduction of abnormal myelination by compound 43. In addition, compound 43 showed a tendency to increase the proportion of axons with a large diameter ([#p<0.05, ###p<0.001, Veh vs WT], [*p<0.05, **p<0.01, Veh vs compound 43]).

### <Experimental Example 9> Analysis of Effect on Sensory Nerves 2

This experiment was made to evaluate the efficacy of the compound of the present disclosure by confirming the effect of the compound of the present disclosure on the animal sensory nerves in CMT mice.

### <Experimental Example 9-1> 10-Week-Old CMT1A Mouse Model

Ten-week-old CMT1A mice were prepared in the same way as shown in Experimental Example 8-1. Particularly, the mice were separated and dosed with a vehicle or drug in the same manner as in table 12, and the number of mice in each group was five mice.

### <Experimental Example 9-2> Electrophysiological Evaluation

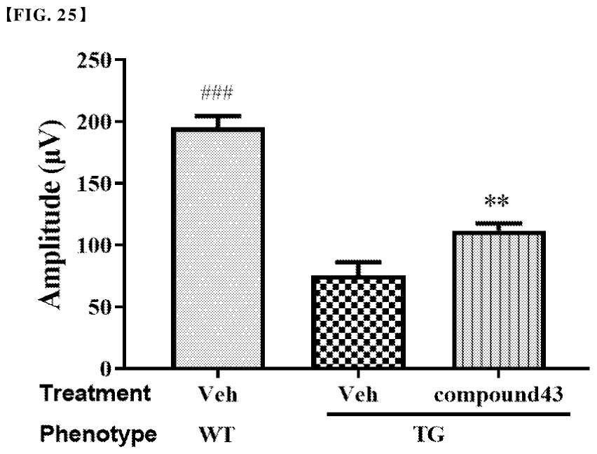

To perform an electrophysiological evaluation of sensory nerves, a stimulation cathode was placed at the tip of the tail of the mouse, a recording electrode was placed on the tail proximal to the body 30 mm from the stimulation cathode, and a ground electrode was also placed on a leg of the animal. The tail was stimulated to obtain sensory nerve conduction velocity (SNCV) and sensory nerve action potential (SNAP) amplitude values. The study results were obtained by Nicolet Viking Quest (Natsu Medical, Inc), and the results are shown in FIGS. 24 and 25 below.

Data were represented as mean±SEM(standard error of measurement). Statistical significance between groups was tested using one-way ANOVA and post hoc analysis was performed by Dunnett's test. All statistical analyses were performed with GraphPad Prism 9.0.

As confirmed from FIGS. 24 and 25, it was found that the C3 CMT1A mice show a significant reduction in the sensory nerve conduction velocity (SNCV) and sensory nerve action potential (SNAP) amplitude values compared with the WT mice in the electrophysiological examination of sensory nerves. In this case, compound 43 significantly increased both sensory nerve conduction velocity (SNCV) and sensory nerve action potential (SNAP) amplitude ([#p<0.05, ###p<0.001, Veh vs WT], [*p<0.05, **p<0.01, Veh vs compound 43]).

The present disclosure provides a pharmaceutical composition, a method, and a use as follow:

Item 1. A pharmaceutical composition for preventing or treating Charcot-Marie-Tooth (CMT) disease associated with peripheral nervous systems (PNS), comprising a compound represented by the above-mentioned formula I the above, optical isomers thereof or pharmaceutically acceptable salts thereof as an active ingredient.

Item 2. The pharmaceutical composition of item 1, wherein the compound represented by formula I is at least one selected from the group consisting of the above-mentioned compound 1 to 450 which is described in the above-mentioned Table A.

Item 3. The pharmaceutical composition of item 1 or 2, wherein the compound represented by formula I is at least one selected from the group consisting of the compound 43, the compound 232, the compound 239, the compound 243 and the compound 286 compounds which is described in the above-mentioned Table B.

Item 4. A method for preventing or treating Charcot-Marie-Tooth disease associated with the peripheral nervous system, including administering a compound represented by above formula I, optical isomers thereof or pharmaceutically acceptable salts thereof described in item 1 to 3 into an individual.

Item 5. A use of the compound represented by the above formula I, optical isomers thereof or pharmaceutically acceptable salts thereof described in items 1 to 3 for preventing or treating Charcot-Marie-Tooth disease associated with the peripheral nervous system.

Item 6. A use of the compound represented by above formula I, optical isomers thereof or pharmaceutically acceptable salts thereof described in items 1 to 3 in preparing a medicament for preventing or treating Charcot-Marie-Tooth disease associated with the peripheral nervous system.

Item 7. The pharmaceutical composition according to any one of items 1 to 3, the method according to item 4, or the use according to item 5 or 6, wherein the Charcot-Marie-Tooth disease associated with the peripheral nervous system is at least one selected from the group consisting of CMT1 type, CMT2 type, CMT4 type, CMTX, degerine-sottas syndrome (DSN), congenital hypomyelination (CH), hereditary neuropathy with liability to pressure palsy (HNPP) and giant axonal neuropathy (GAN).

Item 8. The pharmaceutical composition according to any one of items 1 to 3, the method according to item 4, or the use according to item 5 or 6, wherein the Charcot-Marie-Tooth disease associated with the peripheral nervous system PNS is at least one selected from the group consisting of CMT1A, CMT2D and CMT2F.

Item 9. The pharmaceutical composition according to any one of items 1 to 3, 7 and 8, wherein the pharmaceutical composition is orally administered.

Item 10. The method according to any one of items 4, 7 and 8, or the use according to any one of items 5 to 8, wherein the compound represented by above formula I, optical isomers thereof or pharmaceutically acceptable salts thereof described in item 1 to 3 is orally administered.

Item 11. The pharmaceutical composition according to any one of items 1 to 3 and 7 to 9, the method according to any one of items 4, 7, 8 and 10, or the use according to any one of items 5 to 8 and 10, the composition, the method or the use may prevent or treat the symptom associated with degeneration of the peripheral nervous systems in a subject with Charcot-Marie-Tooth disease.

Item 12. The pharmaceutical composition according to any one of item 1 to 3 and 7 to 9, the method according to any one of items 4, 7, 9 and 10, or the use according to any one of items 5 to 8 and 10, the composition, the method or the use may prevent or treat the symptom associated with dysfunction and/or death of peripheral nerve cells in a subject with Charcot-Marie-Tooth disease.

Item 13. The pharmaceutical composition according to any one of item 1 to 3 and 7 to 9, the method according to any one of items 4, 7, 9 and 10, or the use according to any one of items 5 to 8 and 10, the composition, the method or the use may prevent or treat a degenerative peripheral neuropathy that is caused by dysfunction and/or death of peripheral nerve cells in a subject with Charcot-Marie-Tooth disease.

While specific portions of the present invention have been described in detail above, it is apparent to those skilled in the art that such detailed descriptions are set forth to illustrate exemplary embodiments only, but are not construed to limit the scope of the present invention. Thus, it should be understood that the substantial scope of the present invention is defined by the accompanying claims and equivalents thereto.

1. A pharmaceutical composition for preventing or treating Charcot-Marie-Tooth (CMT) disease associated with peripheral nervous systems (PNS), comprising a compound represented by formula I below, optical isomers thereof or pharmaceutically acceptable salts thereof as an active ingredient: 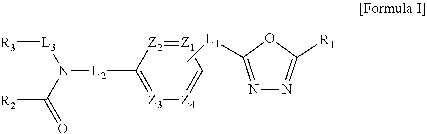
in formula I,
wherein L₁, L₂ or L₃ are each independently a bond or —(C₁-C₂ alkylene)-;
R₁ is —CX₂H or —CX₃;
R₂ is —NR^{A}R^{B}, —OR^{C}, 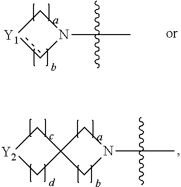
(wherein, at least one H of 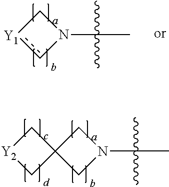
may be substituted with —X, —OH, —O(C₁-C₄ alkyl), —NR^{D}R^{E}, —(C₁-C₄ alkyl), —CF₃, —CF₂H, —CN, -aryl, -heteroaryl, —(C₁-C₄ alkyl)-aryl or —(C₁-C₄ alkyl)-heteroaryl, [wherein at least one H of the -aryl, -heteroaryl, —(C₁-C₄ alkyl)-aryl or —(C₁-C₄ alkyl)-heteroaryl may be substituted with —X, —OH, —CF₃ or —CF₂H]};
R₃ is —H, —(C₁-C₄ alkyl), —(C₁-C₄ alkyl)-O(C₁-C₄ alkyl), —(C₁-C₄ alkyl)-C(═O)—O(C₁-C₄ alkyl), —(C₃-C₇ cycloalkyl), —(C₂-C₆ cycloheteroalkyl), -aryl, -heteroaryl, -adamantyl, 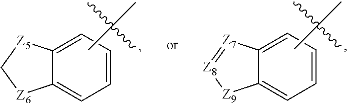
{wherein, at least one H of —(C₁-C₄ alkyl) may be substituted with —X or —OH,
at least one H of -aryl or -heteroaryl each independently may be substituted with —X, —OH, —O(C₁-C₄ alkyl), —OCF₃, —O-aryl, —NR^{D}R^{E}, —(C₁-C₄ alkyl), —CF₃, —CF₂H, —C(═O)—(C₁-C₄ alkyl), —C(═O)—O(C₁-C₄ alkyl), —C(═O)—NR^{D}R^{E}, —S(═O)₂—(C₁-C₄ alkyl), aryl, heteroaryl, 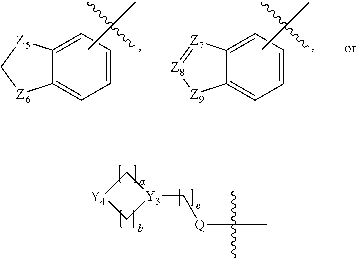
[wherein, at least one H of 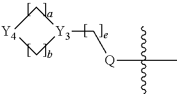
may be substituted with —X, —(C₁-C₄ alkyl), —NR^{D}R^{E}, —CF₃ or —CF₂H], at least one H of —(C₃-C₇ cycloalkyl), —(C₂-C₆ cycloheteroalkyl), adamantyl, 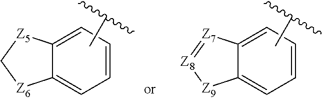
may be each independently substituted with —X, —OH or —(C₁-C₄ alkyl)};
Y₁, Y₂ and Y₄ are each independently —CH₂—, —NR^{F}—, —O—, —C(═O)— or —S(═O)₂—;
Y₃ is —CH— or —N—;
Z₁ to Z₄ are each independently N or CR^{Z}, {wherein at least three of Z₁ to Z₄ may not be simultaneously N, and R^{Z} is —H, —X or —O(C₁-C₄ alkyl)};
Z₅ and Z₆ are each independently —CH₂— or —O—;
Z₇ and Z₈ are each independently ═CH— or ═N—;
Z₉ is —NR^{G}— or —S—;
R^{A} and R^{B} are each independently —H, —(C₁-C₄ alkyl), —(C₁-C₄ alkyl)-OH, —(C₁-C₄ alkyl)-NR^{D}R^{E}, -aryl, —(C₁-C₄ alkyl)-aryl, -heteroaryl, —(C₁-C₄ aryl)-heteroaryl, —(C₃-C₇ cycloalkyl), —(C₂-C₆ heterocycloalkyl) or 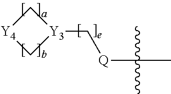
{wherein, at least one H of the —(C₁-C₄ alkyl), —(C₁-C₄ alkyl)-OH or —(C₁-C₄ alkyl)-NR^{D}R^{E} may be substituted with —X,
at least one H of the -aryl, —(C₁-C₄ alkyl)-aryl, -heteroaryl, —(C₁-C₄ alkyl)-heteroaryl, —(C₃-C₇ cycloalkyl) or —(C₂-C₆ heterocycloalkyl) may be substituted with —X, —OH, —O(C₁-C₄ alkyl), —(C₁-C₄ alkyl), —CF₃, —CF₂H or —CN, at least one H of 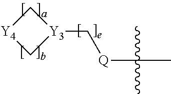
may be substituted with —X, —OH, —O(C₁-C₄ alkyl), —(C₁-C₄ alkyl), —CF₃, —CF₂H, —CN, —(C₂-C₆ heterocycloalkyl), -aryl, —(C₁-C₄ alkyl)-aryl, -heteroaryl or -heteroaryl-(C₁-C₄ alkyl)};
R^{C} is —(C₁-C₄ alkyl), -aryl, —(C₁-C₄ alkyl)-aryl, -heteroaryl or —(C₁-C₄ alkyl)-heteroaryl, {wherein, at least one H of —(C₁-C₄ alkyl) may be substituted with —X or —OH, at least one H of -aryl, —(C₁-C₄ alkyl)-aryl, -heteroaryl or —(C₁-C₄ alkyl)-heteroaryl may be substituted with —X, —OH, —CF₃ or —CF₂H};
R^{D} and R^{E} are each independently —H, —(C₁-C₄ alkyl), -aryl or —(C₁-C₄ alkyl)-aryl, {wherein, at least one H of —(C₁-C₄ alkyl) may be substituted with —X or —OH, at least one H of -aryl or —(C₁-C₄ alkyl)-aryl may be substituted with —X, —OH, —CF₃ or —CF₂H};
R^{F} is —H, —(C₁-C₆ alkyl), —(C₁-C₄ alkyl)-OH, —(C₁-C₄ alkyl)-O—(C₁-C₄ alkyl), —C(═O)—(C₁-C₄ alkyl), —C(═O)—O(C₁-C₄ alkyl), —(C₁-C₄ alkyl)-C(═O)—O(C₁-C₄ alkyl), —(C₁-C₄ alkyl)-NR^{D}R^{E}, —S(═O)₂—(C₁-C₄ alkyl), -aryl, —(C₁-C₄ alkyl)-aryl, —(C₂-C₄ alkenyl)-aryl, -heteroaryl, —(C₁-C₄ alkyl)-heteroaryl, —C(═O)—(C₃-C₇ cycloalkyl), —(C₂-C₆ heterocycloalkyl) or —(C₁-C₄ alkyl)-C(═O)—(C₂-C₆ heterocycloalkyl)
{wherein at least one H of —(C₁-C₄ alkyl), —(C₁-C₄ alkyl)-OH, —(C₁-C₄ alkyl)-O—(C₁-C₄ alkyl), —C(═O)—(C₁-C₄ alkyl), —C(═O)—O(C₁-C₄ alkyl), —(C₁-C₄ alkyl)-C(═O)—O(C₁-C₄ alkyl), —(C₁-C₄ alkyl)-NR^{D}R^{E} or —S(═O)₂—(C₁-C₄ alkyl) may be substituted with —X,
at least one H of -aryl, —(C₁-C₄alkyl)-aryl, —(C₂-C₄ alkenyl)-aryl, -heteroaryl, —(C₁-C₄ alkyl)-heteroaryl, —C(═O)—(C₃-C₇ cycloalkyl), —C₂-C₆ heterocycloalkyl or —(C₁-C₄alkyl)-C(═O)—(C₂-C₆heterocycloalkyl) may be substituted with —X, —OH, —CF₃ or —CF₂H};
R^{G} is —H or —(C₁-C₄ alkyl);
Q is —O— or a bond; [symbol]
is a single bond or double bond, {provided that, [symbol] is a double bond, Y₁ is ═CH—};
a to e are each independently an integer of 0, 1, 2, 3 or 4 f provided that, a and b may not be simultaneously 0, and c and d may not be simultaneously 0};
X is each independently F, Cl, Br or I.

2. The pharmaceutical composition of claim 1, wherein in the compound represented by formula I,
L₁, L₂ or L₃ are each independently a bond or —(C₁-C₂alkylene)-;
R₁ is —CX₂H or —CX₃;
R₂ is —NR^{A}R^{B}, —OR^{C}, 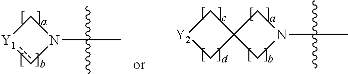
{wherein at least one of H of 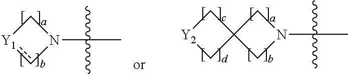
may be substituted with —X, —OH, —NR^{D}R^{E}, —(C₁-C₄ alkyl)};
R₃ is —(C₁-C₄ alkyl), —(C₃-C₇ cycloalkyl), -aryl, -heteroaryl, -adamantyl, 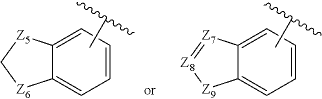
{wherein at least one H of -aryl or -heteroaryl may be each independently substituted with —X, —O(C₁-C₄alkyl), —OCF₃, —O-aryl, —NR^{D}R^{E}, —(C₁-C₄ alkyl), —CF₃, —S(═O)₂—(C₁-C₄alkyl), -aryl, -heteroaryl, 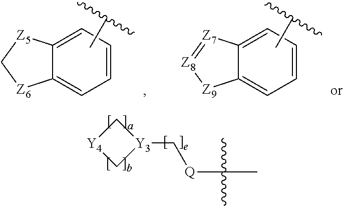
[wherein, at least one H of 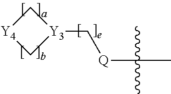
may be substituted with —NR^{D}R^{E} or —(C₁-C₄ alkyl)],
at least one H of 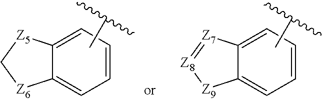
maybe each independently substituted with —(C₁-C₄ alkyl)};
Y₁, Y₂ and Y₄ are each independently —CH₂—, —NR^{F}—, —O—, —C(═O)— or —S(═O)₂—;
Y₃ is —CH— or —N—;
Z₁ to Z₄ is each independently N or CR^{Z} {wherein at least three of Z₁ to Z₄ may not be simultaneously N, and R^{Z} is —H, —X or —O(C₁-C₄ alkyl)};
Z₅ and Z₆ are each independently —CH₂— or —O—;
Z₇ and Z₈ are each independently ═CH— or ═N—;
Z₉ is —NR^{G}— or —S—;
R^{A} and R^{B} are each independently —H, —(C₁-C₄ alkyl), —(C₁-C₄ alkyl)-OH, —(C₁-C₄ alkyl)-NR^{D}R^{E}, -aryl, —(C₁-C₄ alkyl)-aryl, —(C₃-C₇ cycloalkyl) or 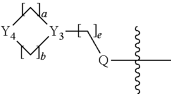
{wherein, at least one H of 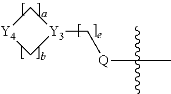
may be substituted with —X, —(C₁-C₄alkyl), —CF₃, —(C₂-C₆ heterocycloalkyl), —(C₁-C₄ alkyl)-aryl, -heteroaryl or heteroaryl-(C₁-C₄ alkyl)};
R^{C} is —(C₁-C₄ alkyl) or -aryl;
R^{D} and R^{E} are each independently —H, —(C₁-C₄alkyl) or —(C₁-C₄ alkyl)-aryl;
R^{F} is —H, —(C₁-C₆alkyl), —(C₁-C₄alkyl)-OH, —(C₁-C₄ alkyl)-O—(C₁-C₄ alkyl), —C(═O)—(C₁-C₄alkyl), —C(═O)—O(C₁-C₄ alkyl), —(C₁-C₄ alkyl)-C(═O)—O(C₁-C₄alkyl), —(C₁-C₄alkyl)-NR^{D}R^{E}, —S(═O)₂—(C₁-C₄ alkyl), -aryl, —(C₁-C₄ alkyl)-aryl, —(C₂-C₄ alkenyl)-aryl, -heteroaryl, —(C₁-C₄ alkyl)-heteroaryl, —C(═O)—(C₃-C₇ cycloalkyl), —(C₂-C₆ heterocycloalkyl) or —(C₁-C₄ alkyl)-C(═O)—(C₂-C₆ heterocycloalkyl)
{wherein at least one H of —(C₁-C₄alkyl) or —C(═O)—O(C₁-C₄alkyl) may be substituted with —X,
at least one H of -aryl may be substituted with —X};
R^{G} is —(C₁-C₄ alkyl);
Q is —O— or a bond; [symbol]
is a single bond or a double bond {provided that [symbol] is a double bond, Y₁ is —CH—};
a to e are each independently an integer of 0, 1, 2, 3 or 4 {provided that a and b may not be simultaneously 0, and c and d may not be simultaneously 0};
X is each independently F, Cl, Br or I.

3. The pharmaceutical composition of claim 1, wherein the compound represented by formula I is the compound represented by formula Ia: 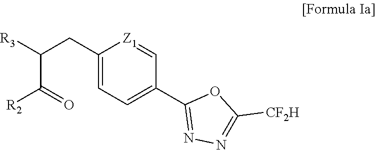
in formula Ia, wherein, 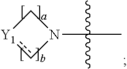
R₂ is
R₃ is -aryl {wherein, at least one H of -aryl may be each independently substituted with —X};
Y₁ is —O— or —S(═O)₂—;
Z₁ is N or CR^{Z} {wherein, R^{Z} is —X};
a and b are each independently an integer of 0, 1, 2, 3 or 4 {wherein, a and b may not be simultaneously 0};
X is each independently F, Cl, Br or I.

4. The pharmaceutical composition of claim 3, wherein in the compound represented by formula Ia,
R₂ is 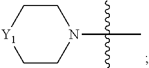
R₃ is -phenyl {wherein, at least one H of -phenyl each independently is substituted with —F or —Cl};
Y₁ is —O— or —S(═O)₂—;
Z₁ is N or CF.

5. A pharmaceutical composition for preventing or treating Charcot-Marie-Tooth (CMT) disease associated with peripheral nervous systems (PNS), comprising a compound, optical isomers thereof or pharmaceutically acceptable salts thereof as an active ingredient, wherein the compound has the following structure:

| Compound | Structure |
|---|---|
| 1 | 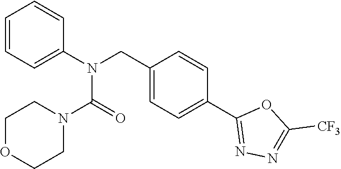 [SMILES: O=C(N1CCOCC1)N(Cc1ccc(-c2nnc(C(F)(F)F)o2)cc1)c1ccccc1] |
| 2 | 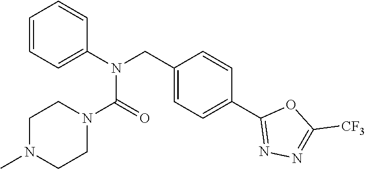 [SMILES: CN1CCN(C(=O)N(Cc2ccc(-c3nnc(C(F)(F)F)o3)cc2)c2ccccc2)CC1] |
| 3 | 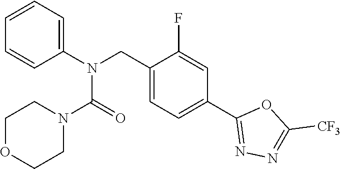 [SMILES: O=C(N1CCOCC1)N(Cc1ccc(-c2nnc(C(F)(F)F)o2)cc1F)c1ccccc1] |
| 4 | 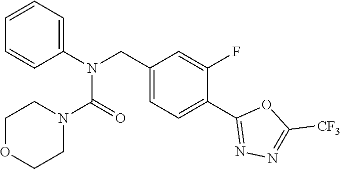 [SMILES: O=C(N1CCOCC1)N(Cc1ccc(-c2nnc(C(F)(F)F)o2)c(F)c1)c1ccccc1] |
| 5 | 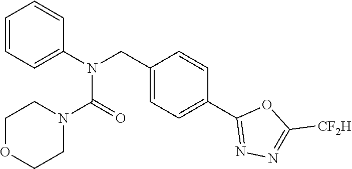 [SMILES: O=C(N1CCOCC1)N(Cc1ccc(-c2nnc(C(F)F)o2)cc1)c1ccccc1] |
| 6 | 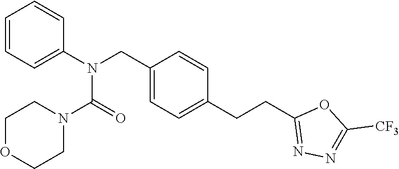 [SMILES: O=C(N1CCOCC1)N(Cc1ccc(CCc2nnc(C(F)(F)F)o2)cc1)c1ccccc1] |
| 7 | 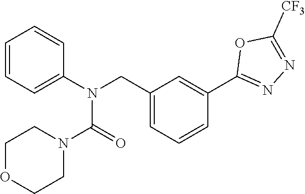 [SMILES: O=C(N1CCOCC1)N(Cc1cccc(-c2nnc(C(F)(F)F)o2)c1)c1ccccc1] |
| 8 | 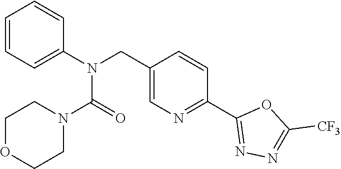 [SMILES: O=C(N1CCOCC1)N(Cc1ccc(-c2nnc(C(F)(F)F)o2)nc1)c1ccccc1] |
| 9 | 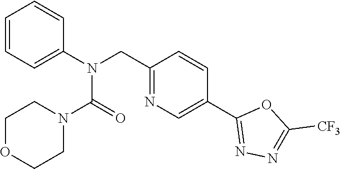 [SMILES: O=C(N1CCOCC1)N(Cc1ccc(-c2nnc(C(F)(F)F)o2)cn1)c1ccccc1] |
| 10 | 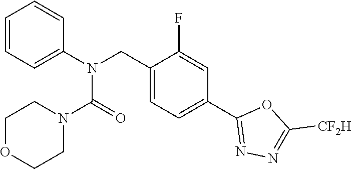 [SMILES: O=C(N1CCOCC1)N(Cc1ccc(-c2nnc(C(F)F)o2)cc1F)c1ccccc1] |
| 11 | 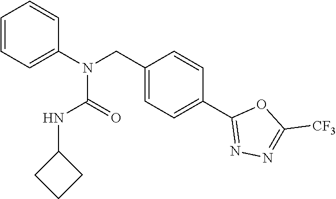 [SMILES: O=C(NC1CCC1)N(Cc1ccc(-c2nnc(C(F)(F)F)o2)cc1)c1ccccc1] |
| 12 | 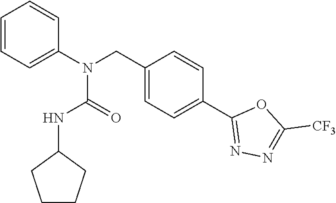 [SMILES: O=C(NC1CCCC1)N(Cc1ccc(-c2nnc(C(F)(F)F)o2)cc1)c1ccccc1] |
| 13 | 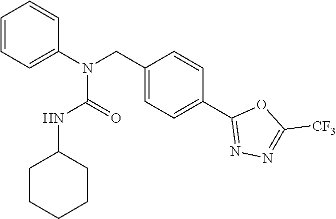 [SMILES: O=C(NC1CCCCC1)N(Cc1ccc(-c2nnc(C(F)(F)F)o2)cc1)c1ccccc1] |
| 14 | 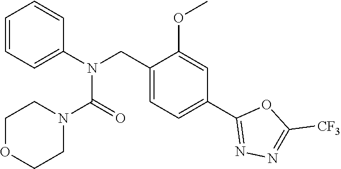 [SMILES: COc1cc(-c2nnc(C(F)(F)F)o2)ccc1CN(C(=O)N1CCOCC1)c1ccccc1] |
| 15 | 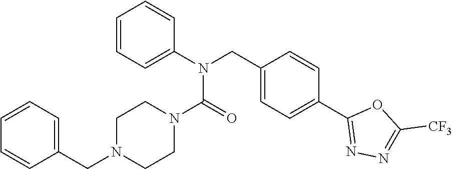 [SMILES: O=C(N1CCN(Cc2ccccc2)CC1)N(Cc1ccc(-c2nnc(C(F)(F)F)o2)cc1)c1ccccc1] |
| 16 | 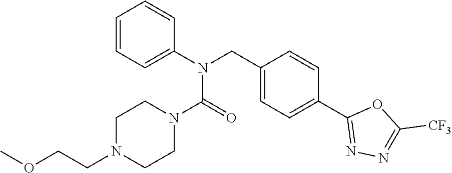 [SMILES: COCCN1CCN(C(=O)N(Cc2ccc(-c3nnc(C(F)(F)F)o3)cc2)c2ccccc2)CC1] |
| 17 | 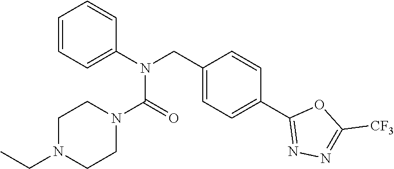 [SMILES: CCN1CCN(C(=O)N(Cc2ccc(-c3nnc(C(F)(F)F)o3)cc2)c2ccccc2)CC1] |
| 18 | 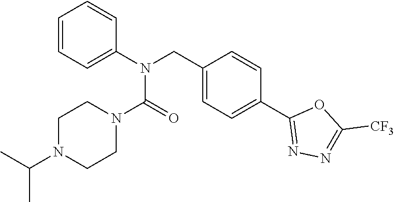 [SMILES: CC(C)N1CCN(C(=O)N(Cc2ccc(-c3nnc(C(F)(F)F)o3)cc2)c2ccccc2)CC1] |
| 19 | 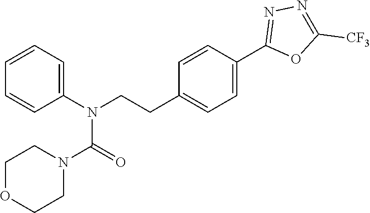 [SMILES: O=C(N1CCOCC1)N(CCc1ccc(-c2nnc(C(F)(F)F)o2)cc1)c1ccccc1] |
| 20 | 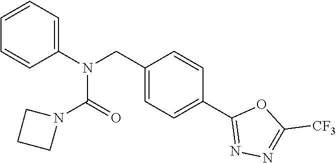 [SMILES: O=C(N1CCC1)N(Cc1ccc(-c2nnc(C(F)(F)F)o2)cc1)c1ccccc1] |
| 21 | 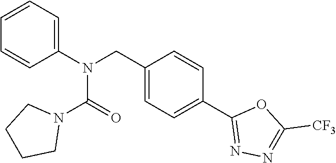 [SMILES: O=C(N1CCCC1)N(Cc1ccc(-c2nnc(C(F)(F)F)o2)cc1)c1ccccc1] |
| 22 | 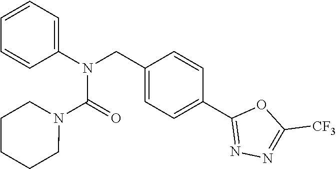 [SMILES: O=C(N1CCCCC1)N(Cc1ccc(-c2nnc(C(F)(F)F)o2)cc1)c1ccccc1] |
| 23 | 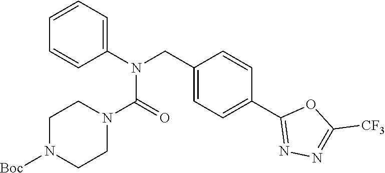 [SMILES: CC(C)(C)OC(=O)N1CCN(C(=O)N(Cc2ccc(-c3nnc(C(F)(F)F)o3)cc2)c2ccccc2)CC1] |
| 24 | 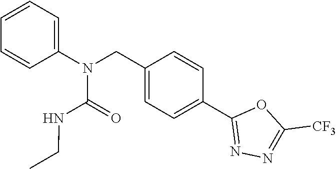 [SMILES: CCNC(=O)N(Cc1ccc(-c2nnc(C(F)(F)F)o2)cc1)c1ccccc1] |
| 25 | 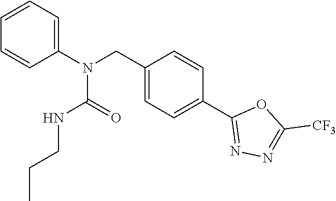 [SMILES: CCCNC(=O)N(Cc1ccc(-c2nnc(C(F)(F)F)o2)cc1)c1ccccc1] |
| 26 | 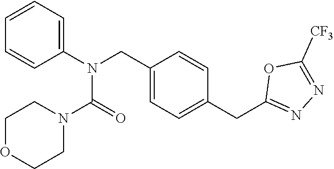 [SMILES: O=C(N1CCOCC1)N(Cc1ccc(Cc2nnc(C(F)(F)F)o2)cc1)c1ccccc1] |
| 27 | 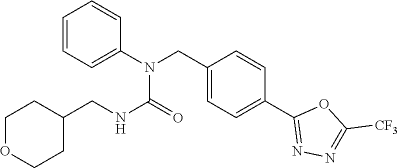 [SMILES: O=C(NCC1CCOCC1)N(Cc1ccc(-c2nnc(C(F)(F)F)o2)cc1)c1ccccc1] |
| 28 | 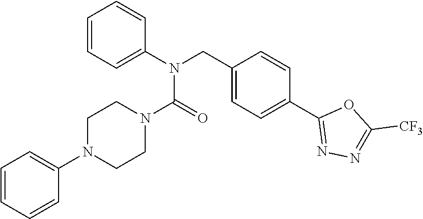 [SMILES: O=C(N1CCN(c2ccccc2)CC1)N(Cc1ccc(-c2nnc(C(F)(F)F)o2)cc1)c1ccccc1] |
| 29 | 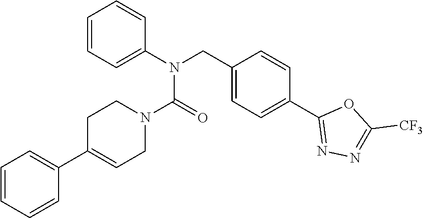 [SMILES: O=C(N1CC=C(c2ccccc2)CC1)N(Cc1ccc(-c2nnc(C(F)(F)F)o2)cc1)c1ccccc1] |
| 30 | 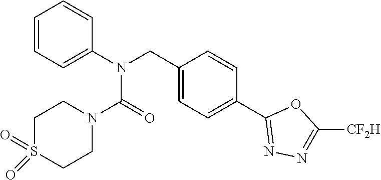 [SMILES: O=C(N1CCS(=O)(=O)CC1)N(Cc1ccc(-c2nnc(C(F)F)o2)cc1)c1ccccc1] |
| 31 | 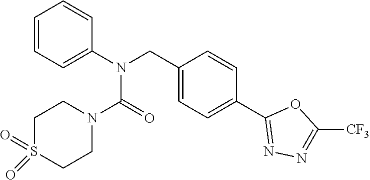 [SMILES: O=C(N1CCS(=O)(=O)CC1)N(Cc1ccc(-c2nnc(C(F)(F)F)o2)cc1)c1ccccc1] |
| 32 | 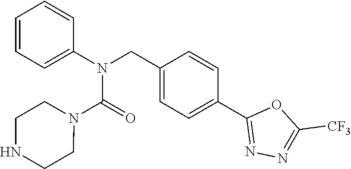 [SMILES: O=C(N1CCNCC1)N(Cc1ccc(-c2nnc(C(F)(F)F)o2)cc1)c1ccccc1] |
| 33 | 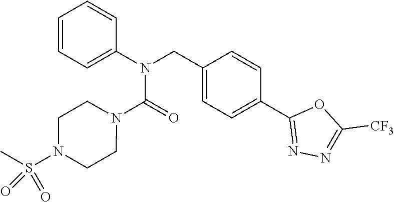 [SMILES: CS(=O)(=O)N1CCN(C(=O)N(Cc2ccc(-c3nnc(C(F)(F)F)o3)cc2)c2ccccc2)CC1] |
| 34 | 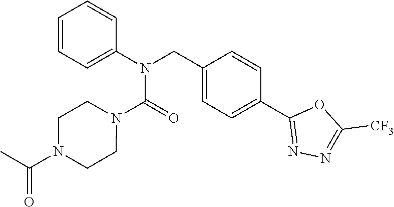 [SMILES: CC(=O)N1CCN(C(=O)N(Cc2ccc(-c3nnc(C(F)(F)F)o3)cc2)c2ccccc2)CC1] |
| 35 | 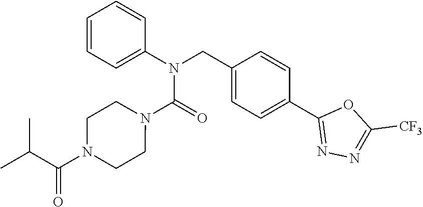 [SMILES: CC(C)C(=O)N1CCN(C(=O)N(Cc2ccc(-c3nnc(C(F)(F)F)o3)cc2)c2ccccc2)CC1] |
| 36 | 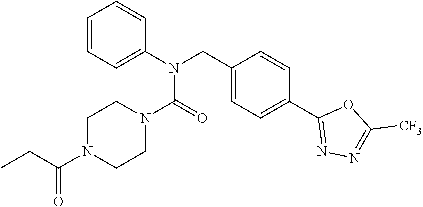 [SMILES: CCC(=O)N1CCN(C(=O)N(Cc2ccc(-c3nnc(C(F)(F)F)o3)cc2)c2ccccc2)CC1] |
| 37 | 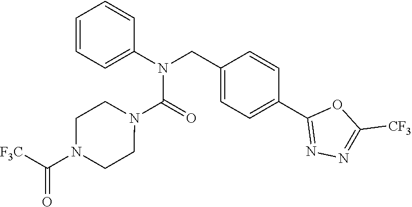 [SMILES: O=C(N1CCN(C(=O)C(F)(F)F)CC1)N(Cc1ccc(-c2nnc(C(F)(F)F)o2)cc1)c1ccccc1] |
| 38 | 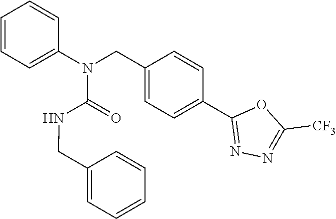 [SMILES: O=C(NCc1ccccc1)N(Cc1ccc(-c2nnc(C(F)(F)F)o2)cc1)c1ccccc1] |
| 39 | 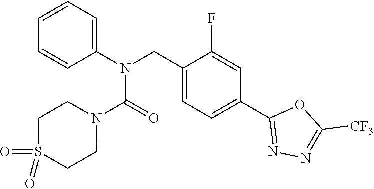 [SMILES: O=C(N1CCS(=O)(=O)CC1)N(Cc1ccc(-c2nnc(C(F)(F)F)o2)cc1F)c1ccccc1] |
| 40 | 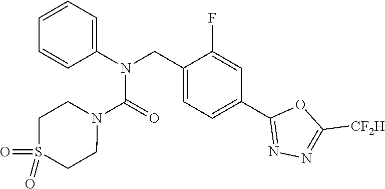 [SMILES: O=C(N1CCS(=O)(=O)CC1)N(Cc1ccc(-c2nnc(C(F)F)o2)cc1F)c1ccccc1] |
| 41 | 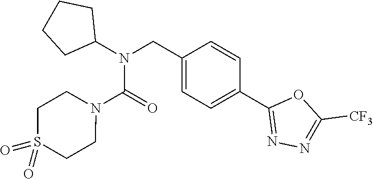 [SMILES: O=C(N1CCS(=O)(=O)CC1)N(Cc1ccc(-c2nnc(C(F)(F)F)o2)cc1)C1CCCC1] |
| 42 | 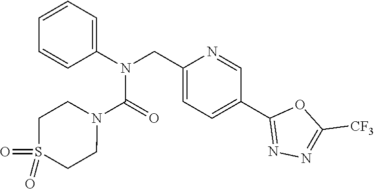 [SMILES: O=C(N1CCS(=O)(=O)CC1)N(Cc1ccc(-c2nnc(C(F)(F)F)o2)cn1)c1ccccc1] |
| 43 | 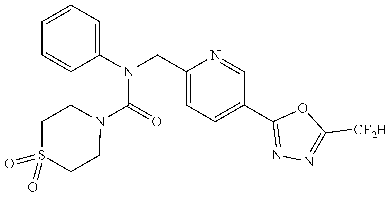 [SMILES: O=C(N1CCS(=O)(=O)CC1)N(Cc1ccc(-c2nnc(C(F)F)o2)cn1)c1ccccc1] |
| 44 | 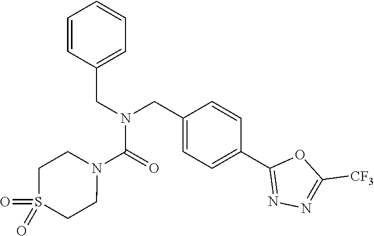 [SMILES: O=C(N1CCS(=O)(=O)CC1)N(Cc1ccccc1)Cc1ccc(-c2nnc(C(F)(F)F)o2)cc1] |
| 45 | 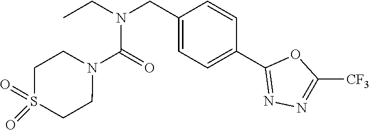 [SMILES: CCN(Cc1ccc(-c2nnc(C(F)(F)F)o2)cc1)C(=O)N1CCS(=O)(=O)CC1] |
| 46 | 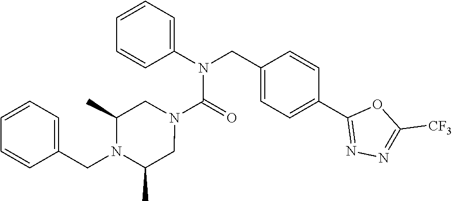 [SMILES: C[C@@H]1CN(C(=O)N(Cc2ccc(-c3nnc(C(F)(F)F)o3)cc2)c2ccccc2)C[C@H](C)N1Cc1ccccc1] |
| 47 | 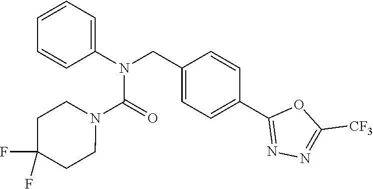 [SMILES: O=C(N1CCC(F)(F)CC1)N(Cc1ccc(-c2nnc(C(F)(F)F)o2)cc1)c1ccccc1] |
| 48 | 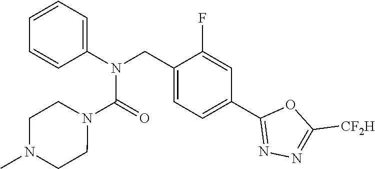 [SMILES: CN1CCN(C(=O)N(Cc2ccc(-c3nnc(C(F)F)o3)cc2F)c2ccccc2)CC1] |
| 49 | 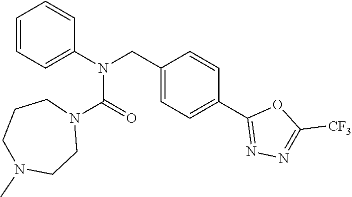 [SMILES: CN1CCCN(C(=O)N(Cc2ccc(-c3nnc(C(F)(F)F)o3)cc2)c2ccccc2)CC1] |
| 50 | 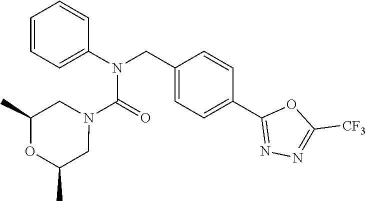 [SMILES: C[C@@H]1CN(C(=O)N(Cc2ccc(-c3nnc(C(F)(F)F)o3)cc2)c2ccccc2)C[C@H](C)O1] |
| 51 | 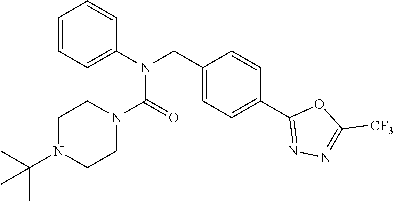 [SMILES: CC(C)(C)N1CCN(C(=O)N(Cc2ccc(-c3nnc(C(F)(F)F)o3)cc2)c2ccccc2)CC1] |
| 52 | 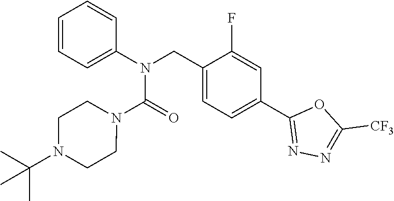 [SMILES: CC(C)(C)N1CCN(C(=O)N(Cc2ccc(-c3nnc(C(F)(F)F)o3)cc2F)c2ccccc2)CC1] |
| 53 | 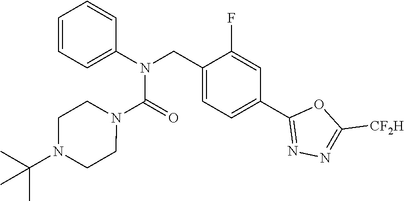 [SMILES: CC(C)(C)N1CCN(C(=O)N(Cc2ccc(-c3nnc(C(F)F)o3)cc2F)c2ccccc2)CC1] |
| 54 | 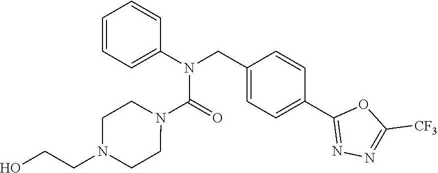 [SMILES: O=C(N1CCN(CCO)CC1)N(Cc1ccc(-c2nnc(C(F)(F)F)o2)cc1)c1ccccc1] |
| 55 | 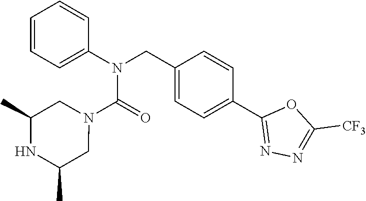 [SMILES: C[C@@H]1CN(C(=O)N(Cc2ccc(-c3nnc(C(F)(F)F)o3)cc2)c2ccccc2)C[C@H](C)N1] |
| 56 | 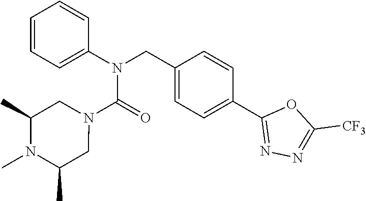 [SMILES: C[C@@H]1CN(C(=O)N(Cc2ccc(-c3nnc(C(F)(F)F)o3)cc2)c2ccccc2)C[C@H](C)N1C] |
| 57 | 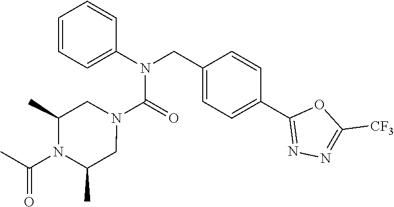 [SMILES: CC(=O)N1[C@H](C)CN(C(=O)N(Cc2ccc(-c3nnc(C(F)(F)F)o3)cc2)c2ccccc2)C[C@@H]1C] |
| 58 | 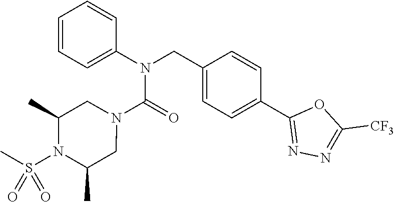 [SMILES: C[C@@H]1CN(C(=O)N(Cc2ccc(-c3nnc(C(F)(F)F)o3)cc2)c2ccccc2)C[C@H](C)N1S(C)(=O)=O] |
| 59 | 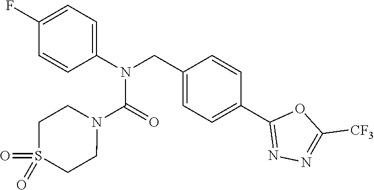 [SMILES: O=C(N1CCS(=O)(=O)CC1)N(Cc1ccc(-c2nnc(C(F)(F)F)o2)cc1)c1ccc(F)cc1] |
| 60 | 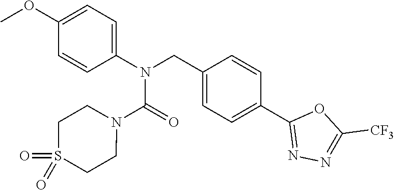 [SMILES: COc1ccc(N(Cc2ccc(-c3nnc(C(F)(F)F)o3)cc2)C(=O)N2CCS(=O)(=O)CC2)cc1] |
| 61 | 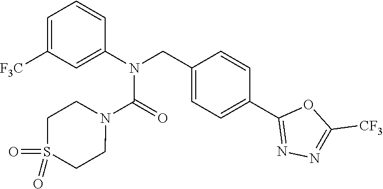 [SMILES: O=C(N1CCS(=O)(=O)CC1)N(Cc1ccc(-c2nnc(C(F)(F)F)o2)cc1)c1cccc(C(F)(F)F)c1] |
| 62 | 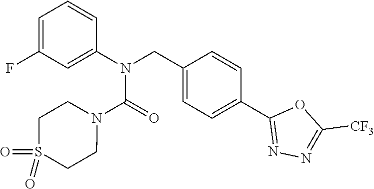 [SMILES: O=C(N1CCS(=O)(=O)CC1)N(Cc1ccc(-c2nnc(C(F)(F)F)o2)cc1)c1cccc(F)c1] |
| 63 | 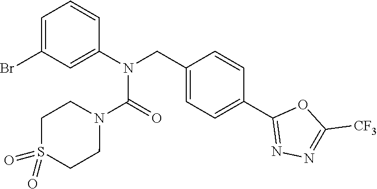 [SMILES: O=C(N1CCS(=O)(=O)CC1)N(Cc1ccc(-c2nnc(C(F)(F)F)o2)cc1)c1cccc(Br)c1] |
| 64 | 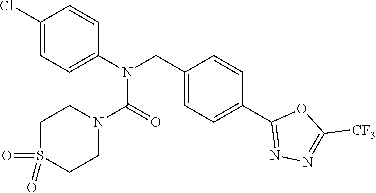 [SMILES: O=C(N1CCS(=O)(=O)CC1)N(Cc1ccc(-c2nnc(C(F)(F)F)o2)cc1)c1ccc(Cl)cc1] |
| 65 | 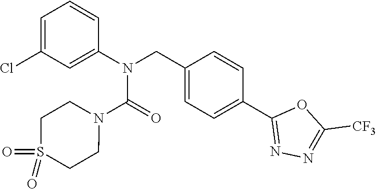 [SMILES: O=C(N1CCS(=O)(=O)CC1)N(Cc1ccc(-c2nnc(C(F)(F)F)o2)cc1)c1cccc(Cl)c1] |
| 66 | 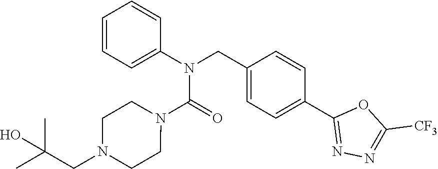 [SMILES: CC(C)(O)CN1CCN(C(=O)N(Cc2ccc(-c3nnc(C(F)(F)F)o3)cc2)c2ccccc2)CC1] |
| 67 | 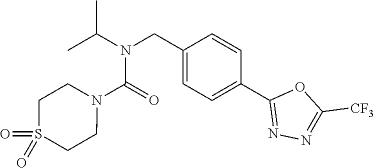 [SMILES: CC(C)N(Cc1ccc(-c2nnc(C(F)(F)F)o2)cc1)C(=O)N1CCS(=O)(=O)CC1] |
| 68 | 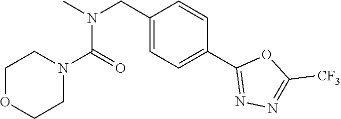 [SMILES: CN(Cc1ccc(-c2nnc(C(F)(F)F)o2)cc1)C(=O)N1CCOCC1] |
| 69 | 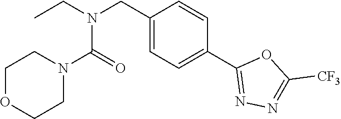 [SMILES: CCN(Cc1ccc(-c2nnc(C(F)(F)F)o2)cc1)C(=O)N1CCOCC1] |
| 70 | 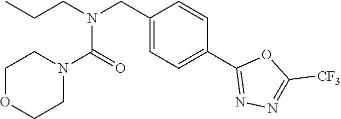 [SMILES: CCCN(Cc1ccc(-c2nnc(C(F)(F)F)o2)cc1)C(=O)N1CCOCC1] |
| 71 | 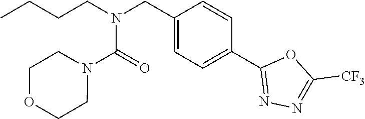 [SMILES: CCCCN(Cc1ccc(-c2nnc(C(F)(F)F)o2)cc1)C(=O)N1CCOCC1] |
| 72 | 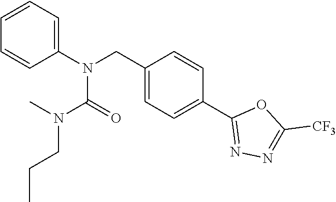 [SMILES: CCCN(C)C(=O)N(Cc1ccc(-c2nnc(C(F)(F)F)o2)cc1)c1ccccc1] |
| 73 | 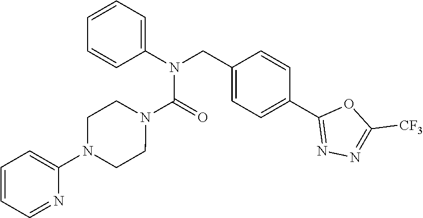 [SMILES: O=C(N1CCN(c2ccccn2)CC1)N(Cc1ccc(-c2nnc(C(F)(F)F)o2)cc1)c1ccccc1] |
| 74 | 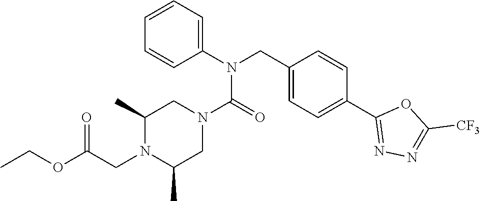 [SMILES: CCOC(=O)CN1[C@H](C)CN(C(=O)N(Cc2ccc(-c3nnc(C(F)(F)F)o3)cc2)c2ccccc2)C[C@@H]1C] |
| 75 | 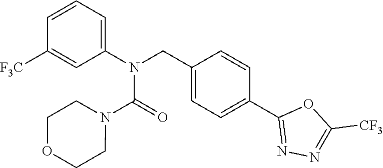 [SMILES: O=C(N1CCOCC1)N(Cc1ccc(-c2nnc(C(F)(F)F)o2)cc1)c1cccc(C(F)(F)F)c1] |
| 76 | 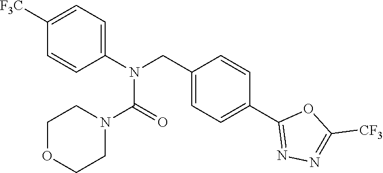 [SMILES: O=C(N1CCOCC1)N(Cc1ccc(-c2nnc(C(F)(F)F)o2)cc1)c1ccc(C(F)(F)F)cc1] |
| 77 | 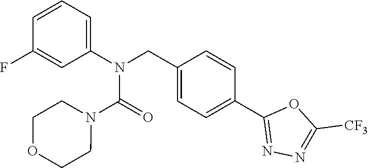 [SMILES: O=C(N1CCOCC1)N(Cc1ccc(-c2nnc(C(F)(F)F)o2)cc1)c1cccc(F)c1] |
| 78 | 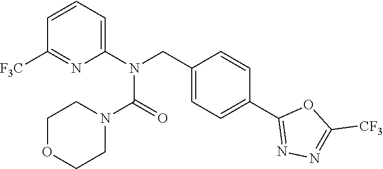 [SMILES: O=C(N1CCOCC1)N(Cc1ccc(-c2nnc(C(F)(F)F)o2)cc1)c1cccc(C(F)(F)F)n1] |
| 79 | 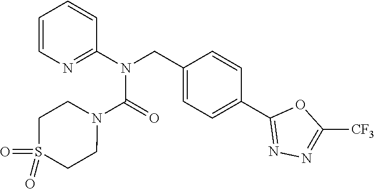 [SMILES: O=C(N1CCS(=O)(=O)CC1)N(Cc1ccc(-c2nnc(C(F)(F)F)o2)cc1)c1ccccn1] |
| 80 | 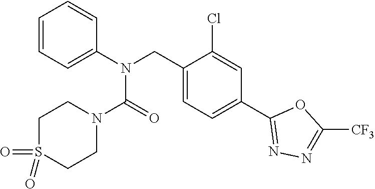 [SMILES: O=C(N1CCS(=O)(=O)CC1)N(Cc1ccc(-c2nnc(C(F)(F)F)o2)cc1Cl)c1ccccc1] |
| 81 | 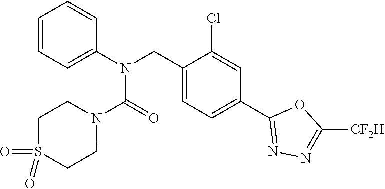 [SMILES: O=C(N1CCS(=O)(=O)CC1)N(Cc1ccc(-c2nnc(C(F)F)o2)cc1Cl)c1ccccc1] |
| 82 | 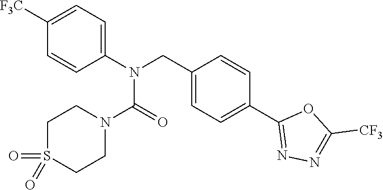 [SMILES: O=C(N1CCS(=O)(=O)CC1)N(Cc1ccc(-c2nnc(C(F)(F)F)o2)cc1)c1ccc(C(F)(F)F)cc1] |
| 83 | 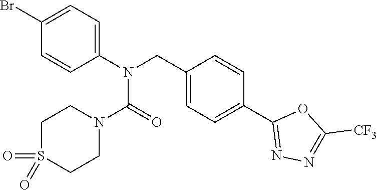 [SMILES: O=C(N1CCS(=O)(=O)CC1)N(Cc1ccc(-c2nnc(C(F)(F)F)o2)cc1)c1ccc(Br)cc1] |
| 84 | 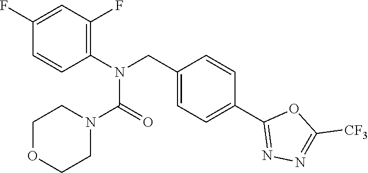 [SMILES: O=C(N1CCOCC1)N(Cc1ccc(-c2nnc(C(F)(F)F)o2)cc1)c1ccc(F)cc1F] |
| 85 | 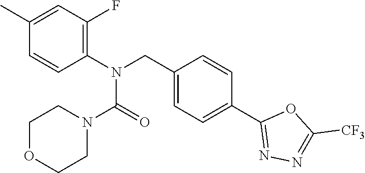 [SMILES: Cc1ccc(N(Cc2ccc(-c3nnc(C(F)(F)F)o3)cc2)C(=O)N2CCOCC2)c(F)c1] |
| 86 | 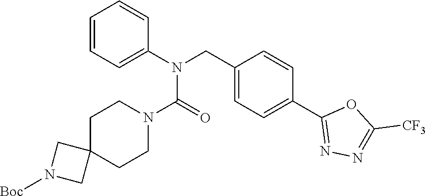 [SMILES: CC(C)(C)OC(=O)N1CC2(CCN(C(=O)N(Cc3ccc(-c4nnc(C(F)(F)F)o4)cc3)c3ccccc3)CC2)C1] |
| 87 | 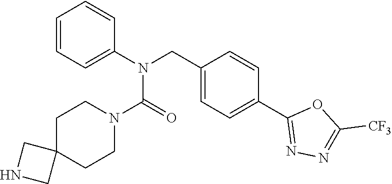 [SMILES: O=C(N1CCC2(CC1)CNC2)N(Cc1ccc(-c2nnc(C(F)(F)F)o2)cc1)c1ccccc1] |
| 88 | 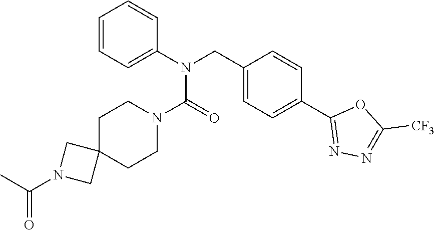 [SMILES: CC(=O)N1CC2(CCN(C(=O)N(Cc3ccc(-c4nnc(C(F)(F)F)o4)cc3)c3ccccc3)CC2)C1] |
| 89 | 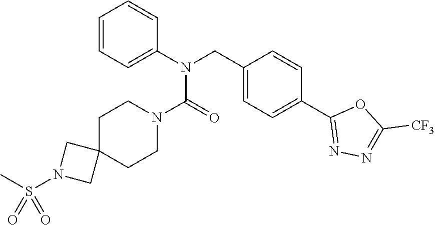 [SMILES: CS(=O)(=O)N1CC2(CCN(C(=O)N(Cc3ccc(-c4nnc(C(F)(F)F)o4)cc3)c3ccccc3)CC2)C1] |
| 90 | 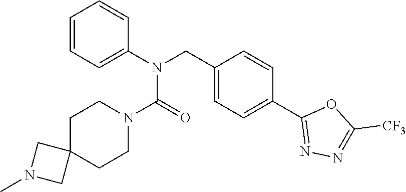 [SMILES: CN1CC2(CCN(C(=O)N(Cc3ccc(-c4nnc(C(F)(F)F)o4)cc3)c3ccccc3)CC2)C1] |
| 91 | 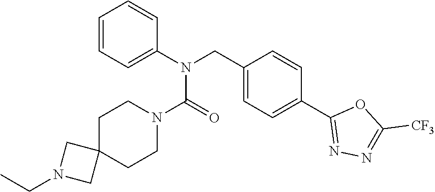 [SMILES: CCN1CC2(CCN(C(=O)N(Cc3ccc(-c4nnc(C(F)(F)F)o4)cc3)c3ccccc3)CC2)C1] |
| 92 | 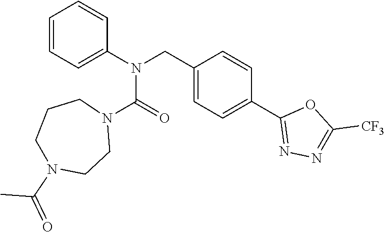 [SMILES: CC(=O)N1CCCN(C(=O)N(Cc2ccc(-c3nnc(C(F)(F)F)o3)cc2)c2ccccc2)CC1] |
| 93 | 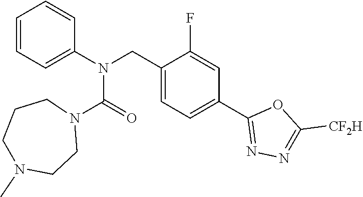 [SMILES: CN1CCCN(C(=O)N(Cc2ccc(-c3nnc(C(F)F)o3)cc2F)c2ccccc2)CC1] |
| 94 | 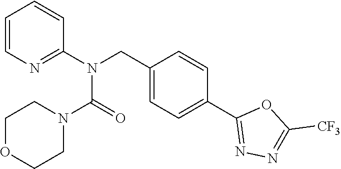 [SMILES: O=C(N1CCOCC1)N(Cc1ccc(-c2nnc(C(F)(F)F)o2)cc1)c1ccccn1] |
| 95 | 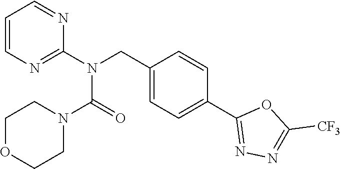 [SMILES: O=C(N1CCOCC1)N(Cc1ccc(-c2nnc(C(F)(F)F)o2)cc1)c1ncccn1] |
| 96 | 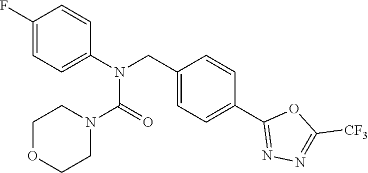 [SMILES: O=C(N1CCOCC1)N(Cc1ccc(-c2nnc(C(F)(F)F)o2)cc1)c1ccc(F)cc1] |
| 97 | 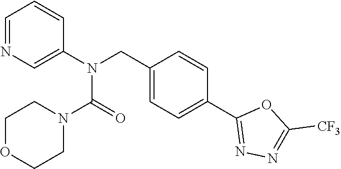 [SMILES: O=C(N1CCOCC1)N(Cc1ccc(-c2nnc(C(F)(F)F)o2)cc1)c1cccnc1] |
| 98 | 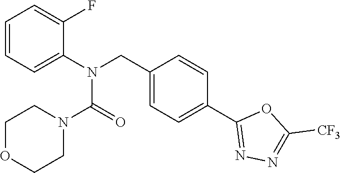 [SMILES: O=C(N1CCOCC1)N(Cc1ccc(-c2nnc(C(F)(F)F)o2)cc1)c1ccccc1F] |
| 99 | 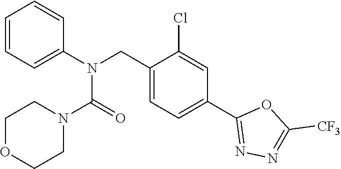 [SMILES: O=C(N1CCOCC1)N(Cc1ccc(-c2nnc(C(F)(F)F)o2)cc1Cl)c1ccccc1] |
| 100 | 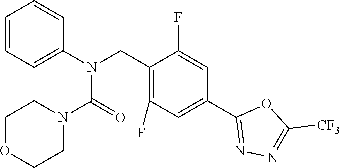 [SMILES: O=C(N1CCOCC1)N(Cc1c(F)cc(-c2nnc(C(F)(F)F)o2)cc1F)c1ccccc1] |
| 101 | 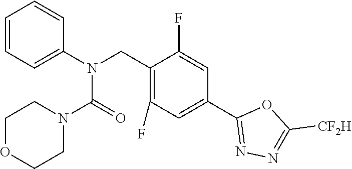 [SMILES: O=C(N1CCOCC1)N(Cc1c(F)cc(-c2nnc(C(F)F)o2)cc1F)c1ccccc1] |
| 102 | 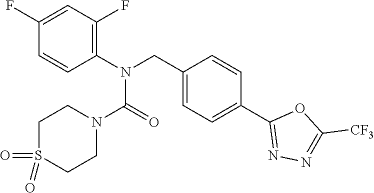 [SMILES: O=C(N1CCS(=O)(=O)CC1)N(Cc1ccc(-c2nnc(C(F)(F)F)o2)cc1)c1ccc(F)cc1F] |
| 103 | 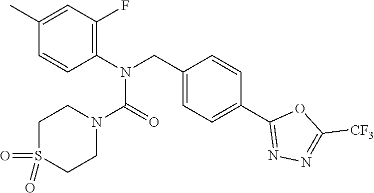 [SMILES: Cc1ccc(N(Cc2ccc(-c3nnc(C(F)(F)F)o3)cc2)C(=O)N2CCS(=O)(=O)CC2)c(F)c1] |
| 104 | 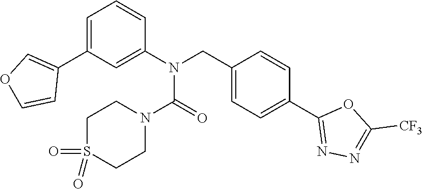 [SMILES: O=C(N1CCS(=O)(=O)CC1)N(Cc1ccc(-c2nnc(C(F)(F)F)o2)cc1)c1cccc(-c2ccoc2)c1] |
| 105 | 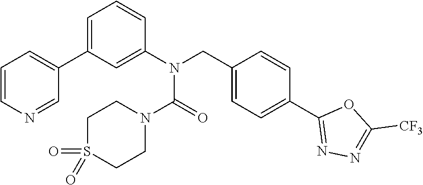 [SMILES: O=C(N1CCS(=O)(=O)CC1)N(Cc1ccc(-c2nnc(C(F)(F)F)o2)cc1)c1cccc(-c2cccnc2)c1] |
| 106 | 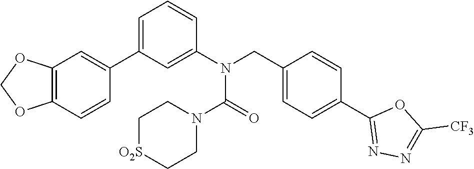 [SMILES: O=C(N1CCS(=O)(=O)CC1)N(Cc1ccc(-c2nnc(C(F)(F)F)o2)cc1)c1cccc(-c2ccc3c(c2)OCO3)c1] |
| 107 | 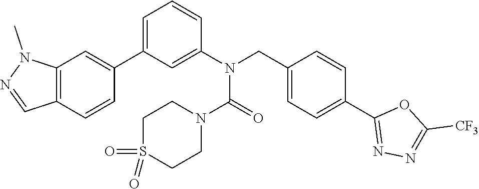 [SMILES: Cn1ncc2ccc(-c3cccc(N(Cc4ccc(-c5nnc(C(F)(F)F)o5)cc4)C(=O)N4CCS(=O)(=O)CC4)c3)cc21] |
| 108 | 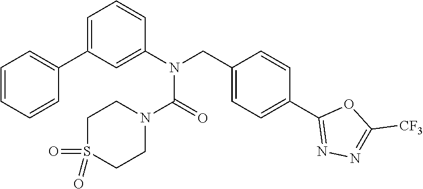 [SMILES: O=C(N1CCS(=O)(=O)CC1)N(Cc1ccc(-c2nnc(C(F)(F)F)o2)cc1)c1cccc(-c2ccccc2)c1] |
| 109 | 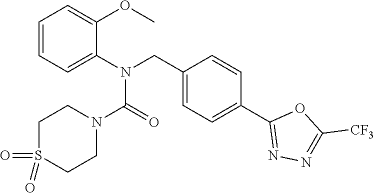 [SMILES: COc1ccccc1N(Cc1ccc(-c2nnc(C(F)(F)F)o2)cc1)C(=O)N1CCS(=O)(=O)CC1] |
| 110 | 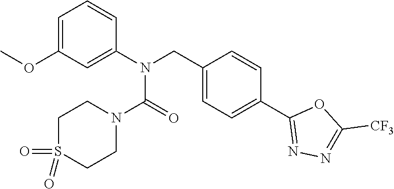 [SMILES: COc1cccc(N(Cc2ccc(-c3nnc(C(F)(F)F)o3)cc2)C(=O)N2CCS(=O)(=O)CC2)c1] |
| 111 | 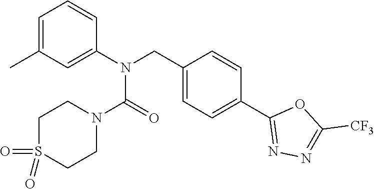 [SMILES: Cc1cccc(N(Cc2ccc(-c3nnc(C(F)(F)F)o3)cc2)C(=O)N2CCS(=O)(=O)CC2)c1] |
| 112 | 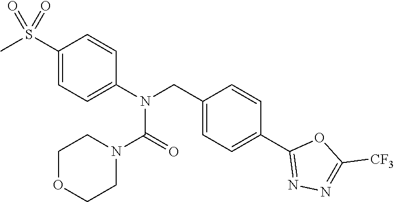 [SMILES: CS(=O)(=O)c1ccc(N(Cc2ccc(-c3nnc(C(F)(F)F)o3)cc2)C(=O)N2CCOCC2)cc1] |
| 113 | 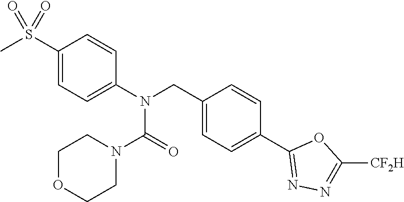 [SMILES: CS(=O)(=O)c1ccc(N(Cc2ccc(-c3nnc(C(F)F)o3)cc2)C(=O)N2CCOCC2)cc1] |
| 114 | 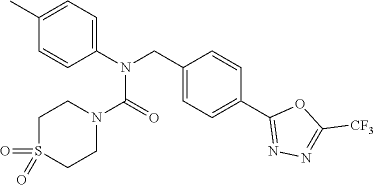 [SMILES: Cc1ccc(N(Cc2ccc(-c3nnc(C(F)(F)F)o3)cc2)C(=O)N2CCS(=O)(=O)CC2)cc1] |
| 115 | 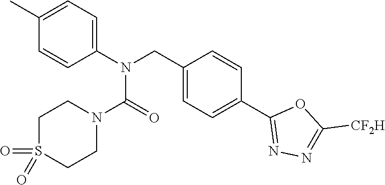 [SMILES: Cc1ccc(N(Cc2ccc(-c3nnc(C(F)F)o3)cc2)C(=O)N2CCS(=O)(=O)CC2)cc1] |
| 116 | 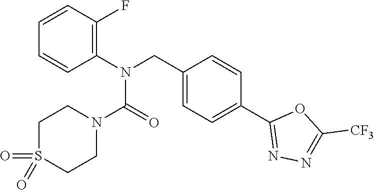 [SMILES: O=C(N1CCS(=O)(=O)CC1)N(Cc1ccc(-c2nnc(C(F)(F)F)o2)cc1)c1ccccc1F] |
| 117 | 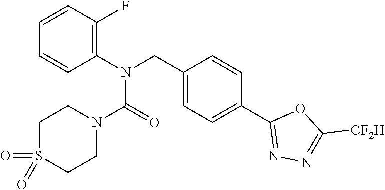 [SMILES: O=C(N1CCS(=O)(=O)CC1)N(Cc1ccc(-c2nnc(C(F)F)o2)cc1)c1ccccc1F] |
| 118 | 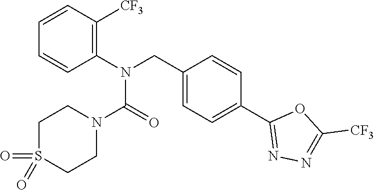 [SMILES: O=C(N1CCS(=O)(=O)CC1)N(Cc1ccc(-c2nnc(C(F)(F)F)o2)cc1)c1ccccc1C(F)(F)F] |
| 119 | 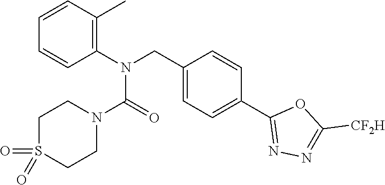 [SMILES: Cc1ccccc1N(Cc1ccc(-c2nnc(C(F)F)o2)cc1)C(=O)N1CCS(=O)(=O)CC1] |
| 120 | 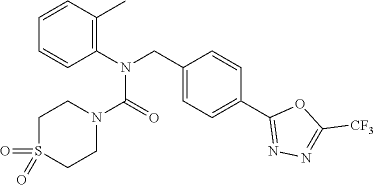 [SMILES: Cc1ccccc1N(Cc1ccc(-c2nnc(C(F)(F)F)o2)cc1)C(=O)N1CCS(=O)(=O)CC1] |
| 121 | 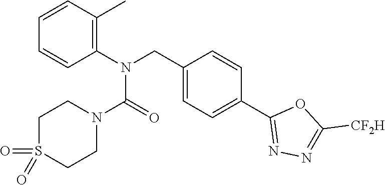 [SMILES: Cc1ccccc1N(Cc1ccc(-c2nnc(C(F)F)o2)cc1)C(=O)N1CCS(=O)(=O)CC1] |
| 122 | 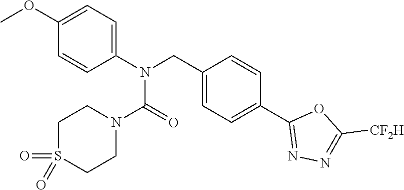 [SMILES: COc1ccc(N(Cc2ccc(-c3nnc(C(F)F)o3)cc2)C(=O)N2CCS(=O)(=O)CC2)cc1] |
| 123 | 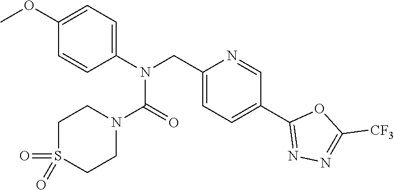 [SMILES: COc1ccc(N(Cc2ccc(-c3nnc(C(F)(F)F)o3)cn2)C(=O)N2CCS(=O)(=O)CC2)cc1] |
| 124 | 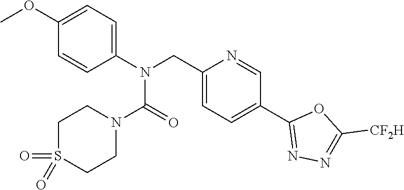 [SMILES: COc1ccc(N(Cc2ccc(-c3nnc(C(F)F)o3)cn2)C(=O)N2CCS(=O)(=O)CC2)cc1] |
| 125 | 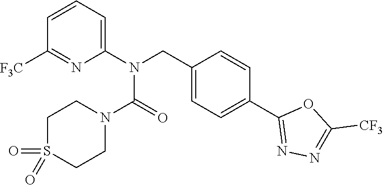 [SMILES: O=C(N1CCS(=O)(=O)CC1)N(Cc1ccc(-c2nnc(C(F)(F)F)o2)cc1)c1cccc(C(F)(F)F)n1] |
| 126 | 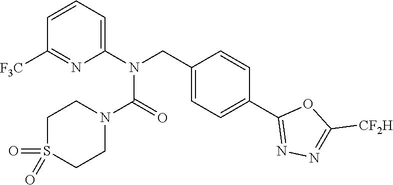 [SMILES: O=C(N1CCS(=O)(=O)CC1)N(Cc1ccc(-c2nnc(C(F)F)o2)cc1)c1cccc(C(F)(F)F)n1] |
| 127 | 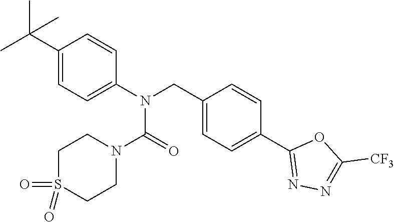 [SMILES: CC(C)(C)c1ccc(N(Cc2ccc(-c3nnc(C(F)(F)F)o3)cc2)C(=O)N2CCS(=O)(=O)CC2)cc1] |
| 128 | 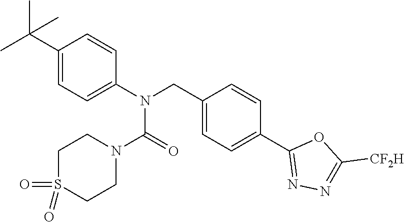 [SMILES: CC(C)(C)c1ccc(N(Cc2ccc(-c3nnc(C(F)F)o3)cc2)C(=O)N2CCS(=O)(=O)CC2)cc1] |
| 129 | 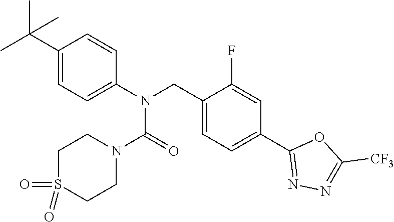 [SMILES: CC(C)(C)c1ccc(N(Cc2ccc(-c3nnc(C(F)(F)F)o3)cc2F)C(=O)N2CCS(=O)(=O)CC2)cc1] |
| 130 | 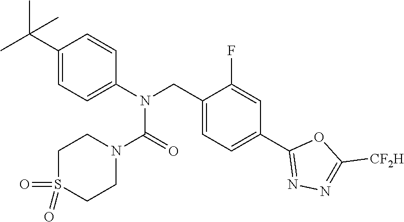 [SMILES: CC(C)(C)c1ccc(N(Cc2ccc(-c3nnc(C(F)F)o3)cc2F)C(=O)N2CCS(=O)(=O)CC2)cc1] |
| 131 | 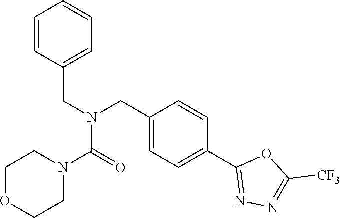 [SMILES: O=C(N1CCOCC1)N(Cc1ccccc1)Cc1ccc(-c2nnc(C(F)(F)F)o2)cc1] |
| 132 | 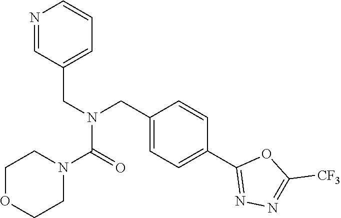 [SMILES: O=C(N1CCOCC1)N(Cc1ccc(-c2nnc(C(F)(F)F)o2)cc1)Cc1cccnc1] |
| 133 | 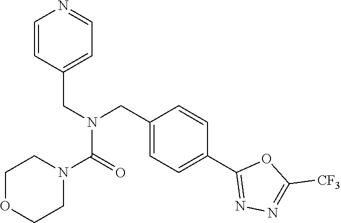 [SMILES: O=C(N1CCOCC1)N(Cc1ccncc1)Cc1ccc(-c2nnc(C(F)(F)F)o2)cc1] |
| 134 | 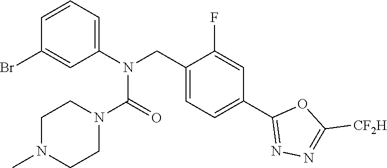 [SMILES: CN1CCN(C(=O)N(Cc2ccc(-c3nnc(C(F)F)o3)cc2F)c2cccc(Br)c2)CC1] |
| 135 | 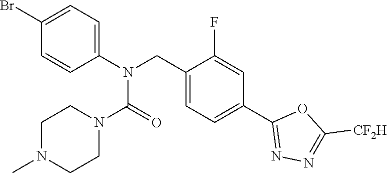 [SMILES: CN1CCN(C(=O)N(Cc2ccc(-c3nnc(C(F)F)o3)cc2F)c2ccc(Br)cc2)CC1] |
| 136 | 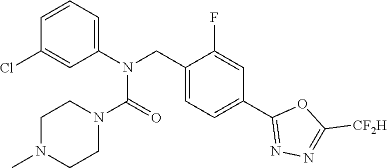 [SMILES: CN1CCN(C(=O)N(Cc2ccc(-c3nnc(C(F)F)o3)cc2F)c2cccc(Cl)c2)CC1] |
| 137 | 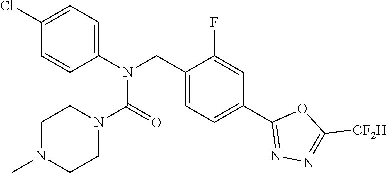 [SMILES: CN1CCN(C(=O)N(Cc2ccc(-c3nnc(C(F)F)o3)cc2F)c2ccc(Cl)cc2)CC1] |
| 138 | 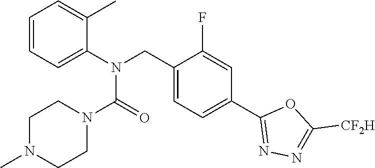 [SMILES: Cc1ccccc1N(Cc1ccc(-c2nnc(C(F)F)o2)cc1F)C(=O)N1CCN(C)CC1] |
| 139 | 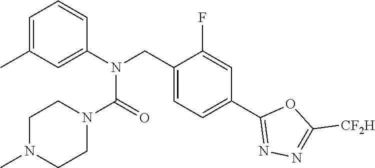 [SMILES: Cc1cccc(N(Cc2ccc(-c3nnc(C(F)F)o3)cc2F)C(=O)N2CCN(C)CC2)c1] |
| 140 | 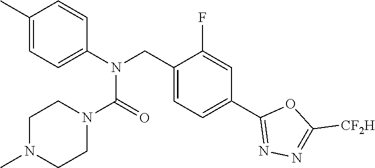 [SMILES: Cc1ccc(N(Cc2ccc(-c3nnc(C(F)F)o3)cc2F)C(=O)N2CCN(C)CC2)cc1] |
| 141 | 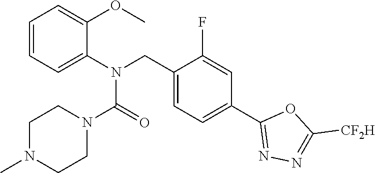 [SMILES: COc1ccccc1N(Cc1ccc(-c2nnc(C(F)F)o2)cc1F)C(=O)N1CCN(C)CC1] |
| 142 | 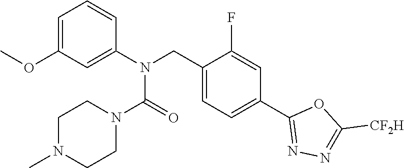 [SMILES: COc1cccc(N(Cc2ccc(-c3nnc(C(F)F)o3)cc2F)C(=O)N2CCN(C)CC2)c1] |
| 143 | 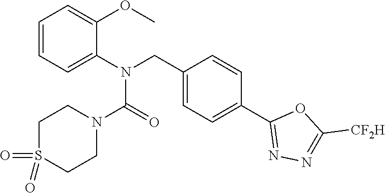 [SMILES: COc1ccccc1N(Cc1ccc(-c2nnc(C(F)F)o2)cc1)C(=O)N1CCS(=O)(=O)CC1] |
| 144 | 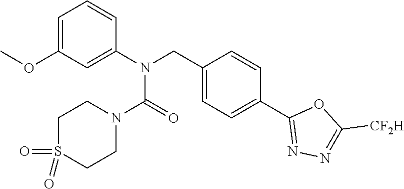 [SMILES: COc1cccc(N(Cc2ccc(-c3nnc(C(F)F)o3)cc2)C(=O)N2CCS(=O)(=O)CC2)c1] |
| 145 | 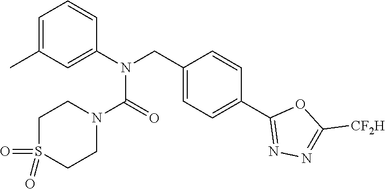 [SMILES: Cc1cccc(N(Cc2ccc(-c3nnc(C(F)F)o3)cc2)C(=O)N2CCS(=O)(=O)CC2)c1] |
| 146 | 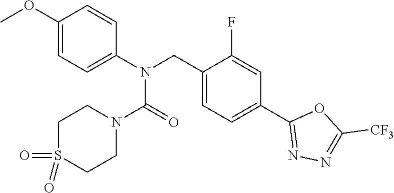 [SMILES: COc1ccc(N(Cc2ccc(-c3nnc(C(F)(F)F)o3)cc2F)C(=O)N2CCS(=O)(=O)CC2)cc1] |
| 147 | 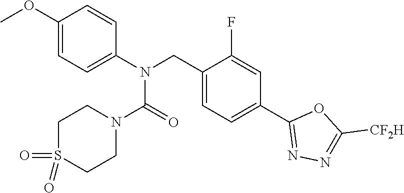 [SMILES: COc1ccc(N(Cc2ccc(-c3nnc(C(F)F)o3)cc2F)C(=O)N2CCS(=O)(=O)CC2)cc1] |
| 148 | 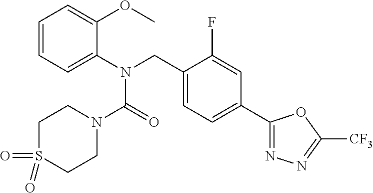 [SMILES: COc1ccccc1N(Cc1ccc(-c2nnc(C(F)(F)F)o2)cc1F)C(=O)N1CCS(=O)(=O)CC1] |
| 149 | 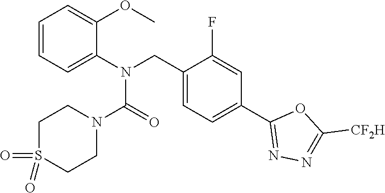 [SMILES: COc1ccccc1N(Cc1ccc(-c2nnc(C(F)F)o2)cc1F)C(=O)N1CCS(=O)(=O)CC1] |
| 150 | 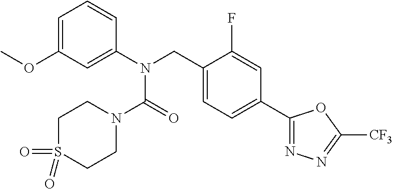 [SMILES: COc1cccc(N(Cc2ccc(-c3nnc(C(F)(F)F)o3)cc2F)C(=O)N2CCS(=O)(=O)CC2)c1] |
| 151 | 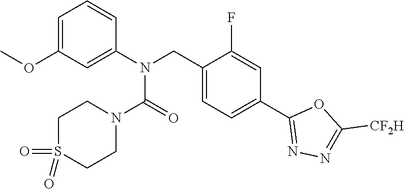 [SMILES: COc1cccc(N(Cc2ccc(-c3nnc(C(F)F)o3)cc2F)C(=O)N2CCS(=O)(=O)CC2)c1] |
| 152 | 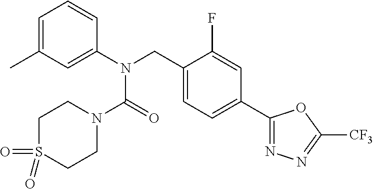 [SMILES: Cc1cccc(N(Cc2ccc(-c3nnc(C(F)(F)F)o3)cc2F)C(=O)N2CCS(=O)(=O)CC2)c1] |
| 153 | 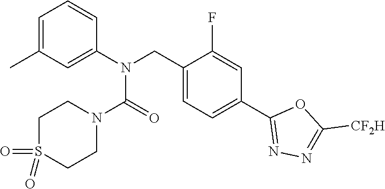 [SMILES: Cc1cccc(N(Cc2ccc(-c3nnc(C(F)F)o3)cc2F)C(=O)N2CCS(=O)(=O)CC2)c1] |
| 154 | 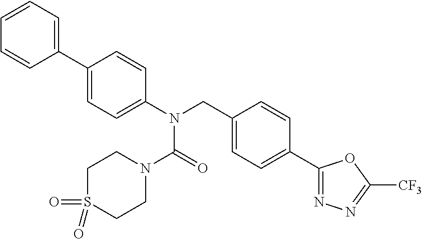 [SMILES: O=C(N1CCS(=O)(=O)CC1)N(Cc1ccc(-c2nnc(C(F)(F)F)o2)cc1)c1ccc(-c2ccccc2)cc1] |
| 155 | 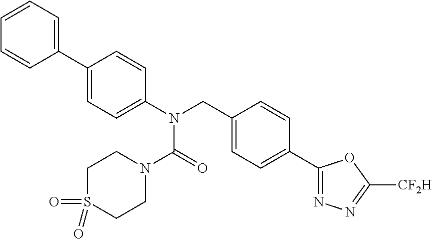 [SMILES: O=C(N1CCS(=O)(=O)CC1)N(Cc1ccc(-c2nnc(C(F)F)o2)cc1)c1ccc(-c2ccccc2)cc1] |
| 156 | 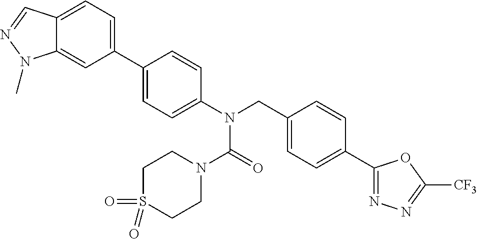 [SMILES: Cn1ncc2ccc(-c3ccc(N(Cc4ccc(-c5nnc(C(F)(F)F)o5)cc4)C(=O)N4CCS(=O)(=O)CC4)cc3)cc21] |
| 157 | 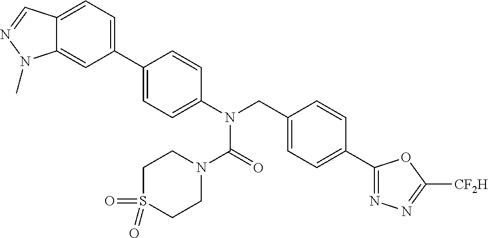 [SMILES: Cn1ncc2ccc(-c3ccc(N(Cc4ccc(-c5nnc(C(F)F)o5)cc4)C(=O)N4CCS(=O)(=O)CC4)cc3)cc21] |
| 158 | 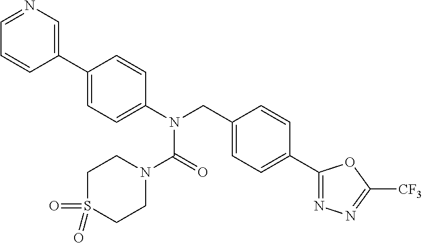 [SMILES: O=C(N1CCS(=O)(=O)CC1)N(Cc1ccc(-c2nnc(C(F)(F)F)o2)cc1)c1ccc(-c2cccnc2)cc1] |
| 159 | 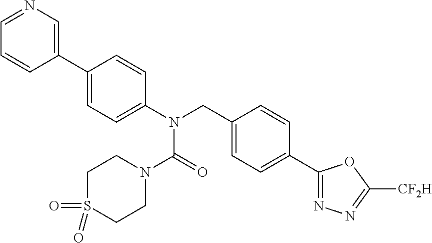 [SMILES: O=C(N1CCS(=O)(=O)CC1)N(Cc1ccc(-c2nnc(C(F)F)o2)cc1)c1ccc(-c2cccnc2)cc1] |
| 160 | 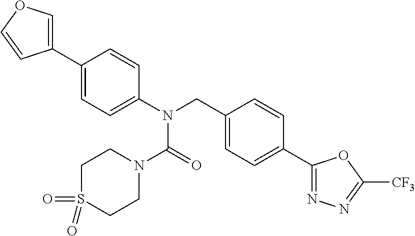 [SMILES: O=C(N1CCS(=O)(=O)CC1)N(Cc1ccc(-c2nnc(C(F)(F)F)o2)cc1)c1ccc(-c2ccoc2)cc1] |
| 161 | 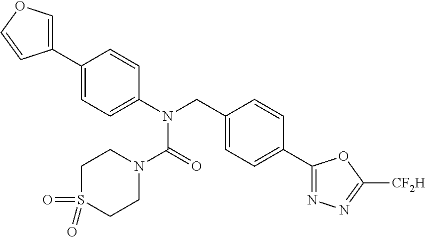 [SMILES: O=C(N1CCS(=O)(=O)CC1)N(Cc1ccc(-c2nnc(C(F)F)o2)cc1)c1ccc(-c2ccoc2)cc1] |
| 162 | 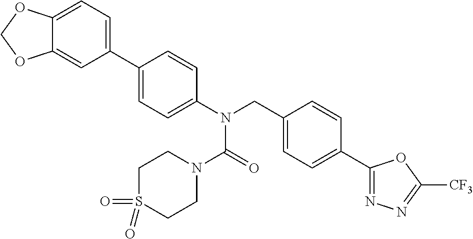 [SMILES: O=C(N1CCS(=O)(=O)CC1)N(Cc1ccc(-c2nnc(C(F)(F)F)o2)cc1)c1ccc(-c2ccc3c(c2)OCO3)cc1] |
| 163 | 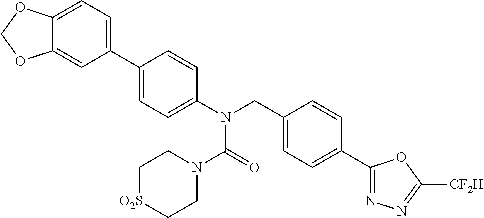 [SMILES: O=C(N1CCS(=O)(=O)CC1)N(Cc1ccc(-c2nnc(C(F)F)o2)cc1)c1ccc(-c2ccc3c(c2)OCO3)cc1] |
| 164 | 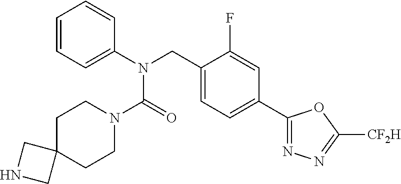 [SMILES: O=C(N1CCC2(CC1)CNC2)N(Cc1ccc(-c2nnc(C(F)F)o2)cc1F)c1ccccc1] |
| 165 | 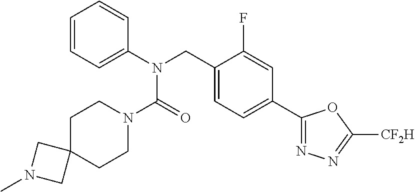 [SMILES: CN1CC2(CCN(C(=O)N(Cc3ccc(-c4nnc(C(F)F)o4)cc3F)c3ccccc3)CC2)C1] |
| 166 | 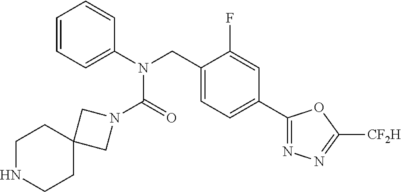 [SMILES: O=C(N1CC2(CCNCC2)C1)N(Cc1ccc(-c2nnc(C(F)F)o2)cc1F)c1ccccc1] |
| 167 | 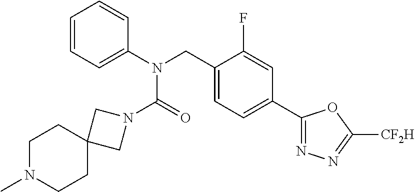 [SMILES: CN1CCC2(CC1)CN(C(=O)N(Cc1ccc(-c3nnc(C(F)F)o3)cc1F)c1ccccc1)C2] |
| 168 | 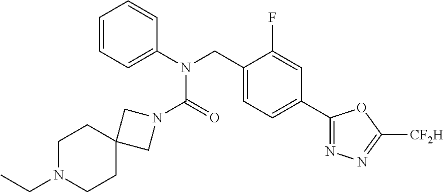 [SMILES: CCN1CCC2(CC1)CN(C(=O)N(Cc1ccc(-c3nnc(C(F)F)o3)cc1F)c1ccccc1)C2] |
| 169 | 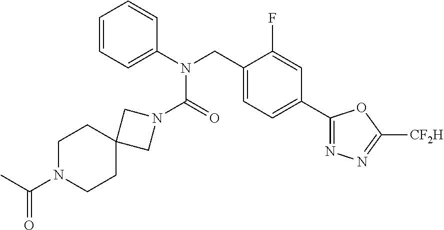 [SMILES: CC(=O)N1CCC2(CC1)CN(C(=O)N(Cc1ccc(-c3nnc(C(F)F)o3)cc1F)c1ccccc1)C2] |
| 170 | 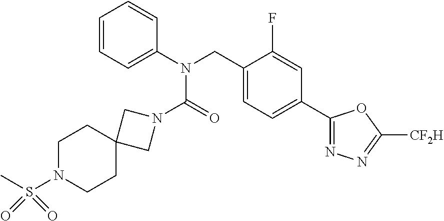 [SMILES: CS(=O)(=O)N1CCC2(CC1)CN(C(=O)N(Cc1ccc(-c3nnc(C(F)F)o3)cc1F)c1ccccc1)C2] |
| 171 | 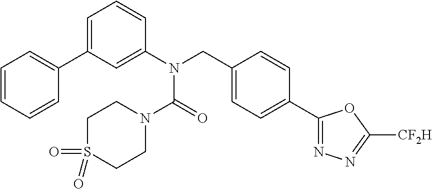 [SMILES: O=C(N1CCS(=O)(=O)CC1)N(Cc1ccc(-c2nnc(C(F)F)o2)cc1)c1cccc(-c2ccccc2)c1] |
| 172 | 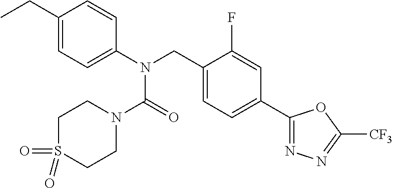 [SMILES: CCc1ccc(N(Cc2ccc(-c3nnc(C(F)(F)F)o3)cc2F)C(=O)N2CCS(=O)(=O)CC2)cc1] |
| 173 | 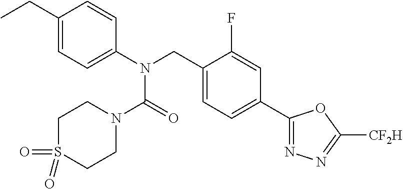 [SMILES: CCc1ccc(N(Cc2ccc(-c3nnc(C(F)F)o3)cc2F)C(=O)N2CCS(=O)(=O)CC2)cc1] |
| 174 | 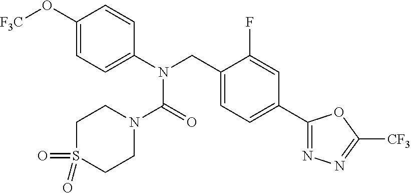 [SMILES: O=C(N1CCS(=O)(=O)CC1)N(Cc1ccc(-c2nnc(C(F)(F)F)o2)cc1F)c1ccc(OC(F)(F)F)cc1] |
| 175 | 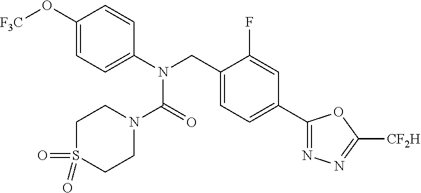 [SMILES: O=C(N1CCS(=O)(=O)CC1)N(Cc1ccc(-c2nnc(C(F)F)o2)cc1F)c1ccc(OC(F)(F)F)cc1] |
| 176 | 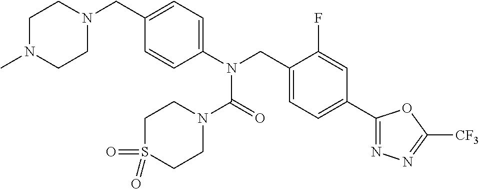 [SMILES: CN1CCN(Cc2ccc(N(Cc3ccc(-c4nnc(C(F)(F)F)o4)cc3F)C(=O)N3CCS(=O)(=O)CC3)cc2)CC1] |
| 177 | 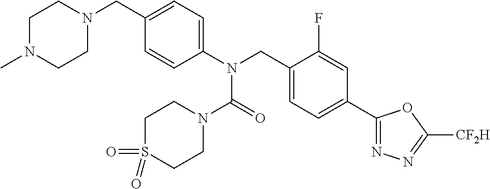 [SMILES: CN1CCN(Cc2ccc(N(Cc3ccc(-c4nnc(C(F)F)o4)cc3F)C(=O)N3CCS(=O)(=O)CC3)cc2)CC1] |
| 178 | 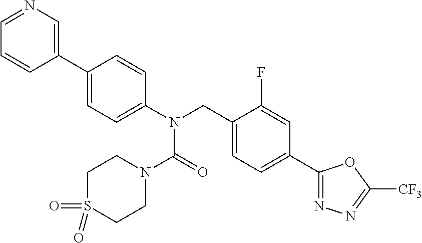 [SMILES: O=C(N1CCS(=O)(=O)CC1)N(Cc1ccc(-c2nnc(C(F)(F)F)o2)cc1F)c1ccc(-c2cccnc2)cc1] |
| 179 | 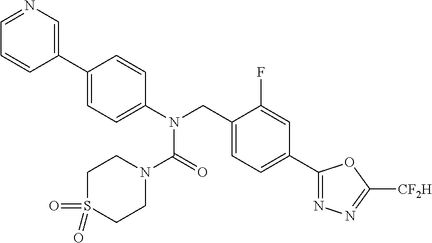 [SMILES: O=C(N1CCS(=O)(=O)CC1)N(Cc1ccc(-c2nnc(C(F)F)o2)cc1F)c1ccc(-c2cccnc2)cc1] |
| 180 | 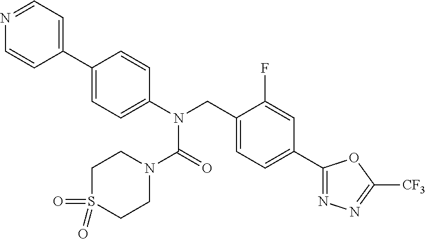 [SMILES: O=C(N1CCS(=O)(=O)CC1)N(Cc1ccc(-c2nnc(C(F)(F)F)o2)cc1F)c1ccc(-c2ccncc2)cc1] |
| 181 | 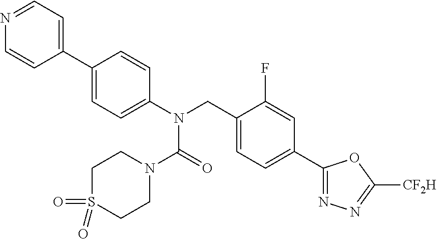 [SMILES: O=C(N1CCS(=O)(=O)CC1)N(Cc1ccc(-c2nnc(C(F)F)o2)cc1F)c1ccc(-c2ccncc2)cc1] |
| 182 | 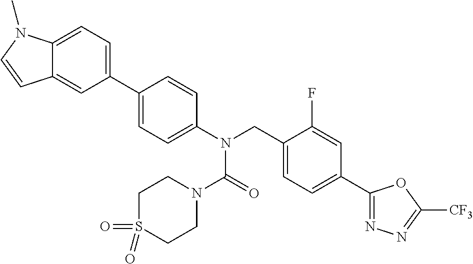 [SMILES: Cn1ccc2cc(-c3ccc(N(Cc4ccc(-c5nnc(C(F)(F)F)o5)cc4F)C(=O)N4CCS(=O)(=O)CC4)cc3)ccc21] |
| 183 | 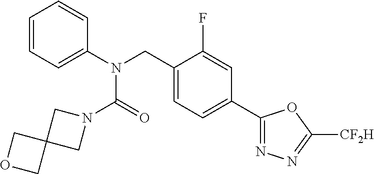 [SMILES: O=C(N1CC2(COC2)C1)N(Cc1ccc(-c2nnc(C(F)F)o2)cc1F)c1ccccc1] |
| 184 | 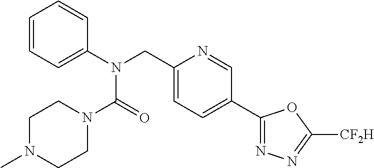 [SMILES: CN1CCN(C(=O)N(Cc2ccc(-c3nnc(C(F)F)o3)cn2)c2ccccc2)CC1] |
| 185 | 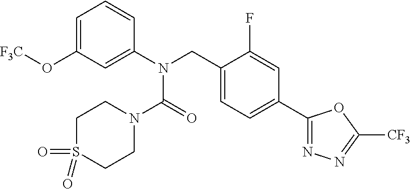 [SMILES: O=C(N1CCS(=O)(=O)CC1)N(Cc1ccc(-c2nnc(C(F)(F)F)o2)cc1F)c1cccc(OC(F)(F)F)c1] |
| 186 | 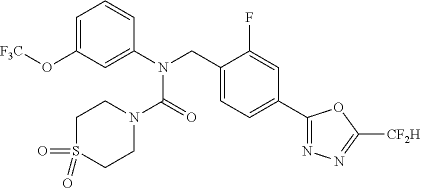 [SMILES: O=C(N1CCS(=O)(=O)CC1)N(Cc1ccc(-c2nnc(C(F)F)o2)cc1F)c1cccc(OC(F)(F)F)c1] |
| 187 | 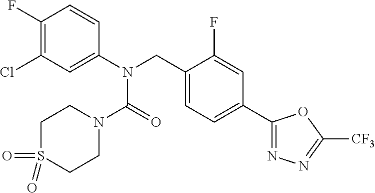 [SMILES: O=C(N1CCS(=O)(=O)CC1)N(Cc1ccc(-c2nnc(C(F)(F)F)o2)cc1F)c1ccc(F)c(Cl)c1] |
| 188 | 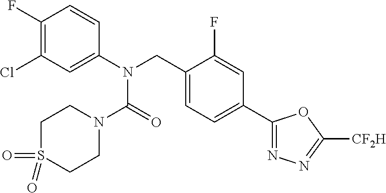 [SMILES: O=C(N1CCS(=O)(=O)CC1)N(Cc1ccc(-c2nnc(C(F)F)o2)cc1F)c1ccc(F)c(Cl)c1] |
| 189 | 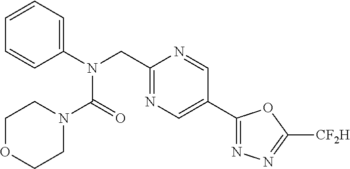 [SMILES: O=C(N1CCOCC1)N(Cc1ncc(-c2nnc(C(F)F)o2)cn1)c1ccccc1] |
| 190 | 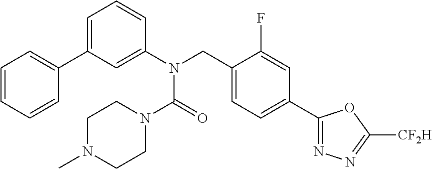 [SMILES: CN1CCN(C(=O)N(Cc2ccc(-c3nnc(C(F)F)o3)cc2F)c2cccc(-c3ccccc3)c2)CC1] |
| 191 | 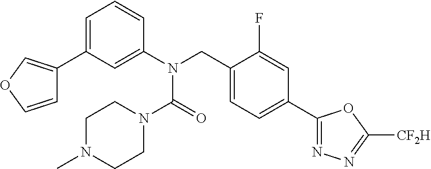 [SMILES: CN1CCN(C(=O)N(Cc2ccc(-c3nnc(C(F)F)o3)cc2F)c2cccc(-c3ccoc3)c2)CC1] |
| 192 | 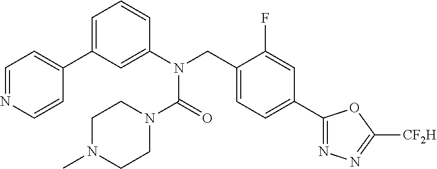 [SMILES: CN1CCN(C(=O)N(Cc2ccc(-c3nnc(C(F)F)o3)cc2F)c2cccc(-c3ccncc3)c2)CC1] |
| 193 | 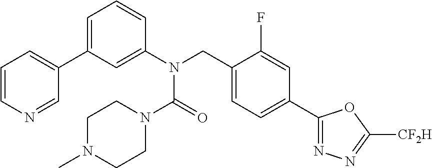 [SMILES: CN1CCN(C(=O)N(Cc2ccc(-c3nnc(C(F)F)o3)cc2F)c2cccc(-c3cccnc3)c2)CC1] |
| 194 | 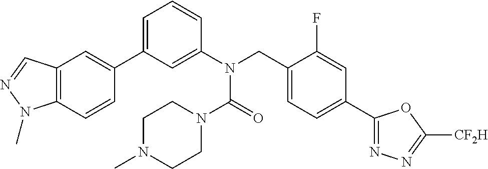 [SMILES: CN1CCN(C(=O)N(Cc2ccc(-c3nnc(C(F)F)o3)cc2F)c2cccc(-c3ccc4c(cnn4C)c3)c2)CC1] |
| 195 | 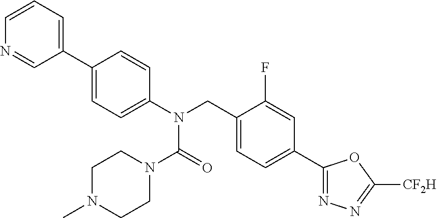 [SMILES: CN1CCN(C(=O)N(Cc2ccc(-c3nnc(C(F)F)o3)cc2F)c2ccc(-c3cccnc3)cc2)CC1] |
| 196 | 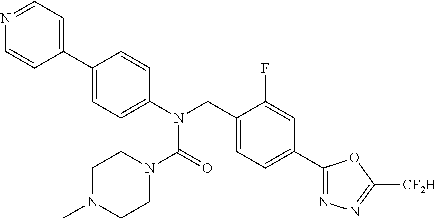 [SMILES: CN1CCN(C(=O)N(Cc2ccc(-c3nnc(C(F)F)o3)cc2F)c2ccc(-c3ccncc3)cc2)CC1] |
| 197 | 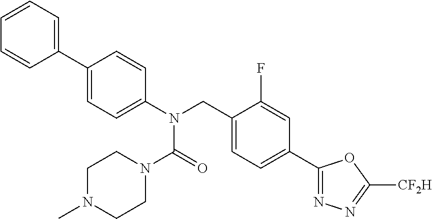 [SMILES: CN1CCN(C(=O)N(Cc2ccc(-c3nnc(C(F)F)o3)cc2F)c2ccc(-c3ccccc3)cc2)CC1] |
| 198 | 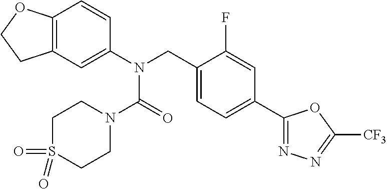 [SMILES: O=C(N1CCS(=O)(=O)CC1)N(Cc1ccc(-c2nnc(C(F)(F)F)o2)cc1F)c1ccc2c(c1)CCO2] |
| 199 | 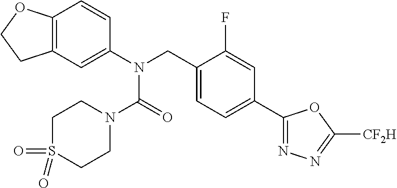 [SMILES: O=C(N1CCS(=O)(=O)CC1)N(Cc1ccc(-c2nnc(C(F)F)o2)cc1F)c1ccc2c(c1)CCO2] |
| 200 | 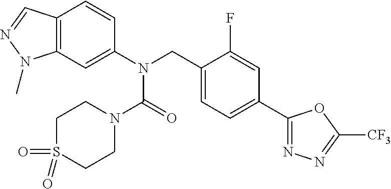 [SMILES: Cn1ncc2ccc(N(Cc3ccc(-c4nnc(C(F)(F)F)o4)cc3F)C(=O)N3CCS(=O)(=O)CC3)cc21] |
| 201 | 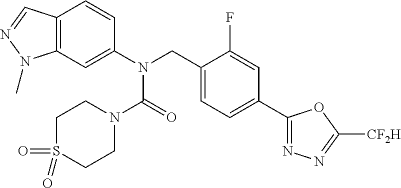 [SMILES: Cn1ncc2ccc(N(Cc3ccc(-c4nnc(C(F)F)o4)cc3F)C(=O)N3CCS(=O)(=O)CC3)cc21] |
| 202 | 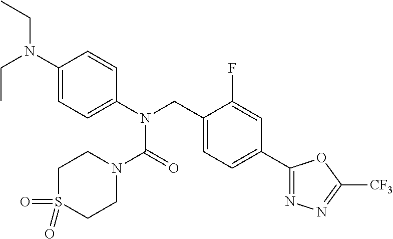 [SMILES: CCN(CC)c1ccc(N(Cc2ccc(-c3nnc(C(F)(F)F)o3)cc2F)C(=O)N2CCS(=O)(=O)CC2)cc1] |
| 203 | 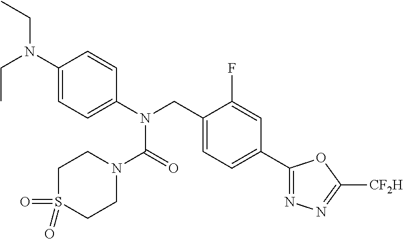 [SMILES: CCN(CC)c1ccc(N(Cc2ccc(-c3nnc(C(F)F)o3)cc2F)C(=O)N2CCS(=O)(=O)CC2)cc1] |
| 204 | 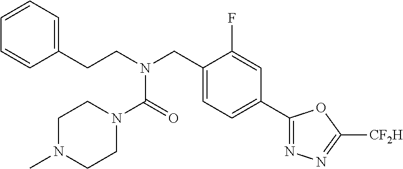 [SMILES: CN1CCN(C(=O)N(CCc2ccccc2)Cc2ccc(-c3nnc(C(F)F)o3)cc2F)CC1] |
| 205 | 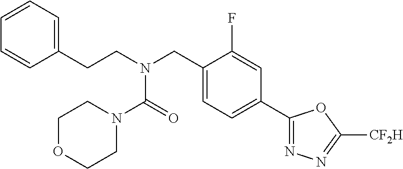 [SMILES: O=C(N1CCOCC1)N(CCc1ccccc1)Cc1ccc(-c2nnc(C(F)F)o2)cc1F] |
| 206 | 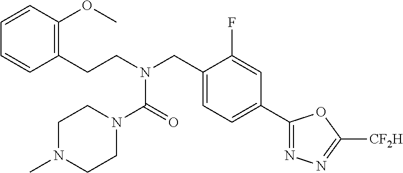 [SMILES: COc1ccccc1CCN(Cc1ccc(-c2nnc(C(F)F)o2)cc1F)C(=O)N1CCN(C)CC1] |
| 207 | 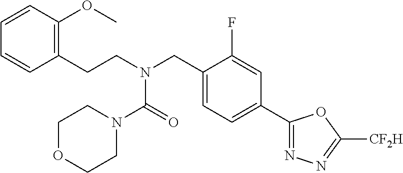 [SMILES: COc1ccccc1CCN(Cc1ccc(-c2nnc(C(F)F)o2)cc1F)C(=O)N1CCOCC1] |
| 208 | 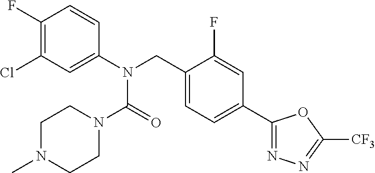 [SMILES: CN1CCN(C(=O)N(Cc2ccc(-c3nnc(C(F)(F)F)o3)cc2F)c2ccc(F)c(Cl)c2)CC1] |
| 209 | 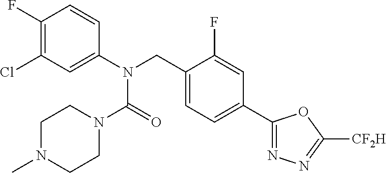 [SMILES: CN1CCN(C(=O)N(Cc2ccc(-c3nnc(C(F)F)o3)cc2F)c2ccc(F)c(Cl)c2)CC1] |
| 210 | 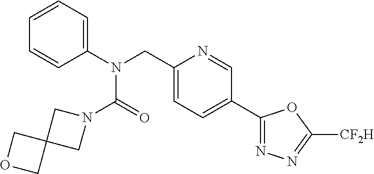 [SMILES: O=C(N1CC2(COC2)C1)N(Cc1ccc(-c2nnc(C(F)F)o2)cn1)c1ccccc1] |
| 211 | 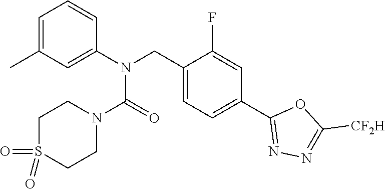 [SMILES: Cc1cccc(N(Cc2ccc(-c3nnc(C(F)F)o3)cc2F)C(=O)N2CCS(=O)(=O)CC2)c1] |
| 212 | 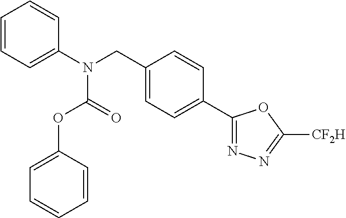 [SMILES: O=C(Oc1ccccc1)N(Cc1ccc(-c2nnc(C(F)F)o2)cc1)c1ccccc1] |
| 213 | 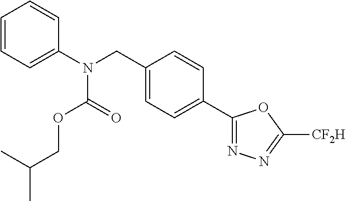 [SMILES: CC(C)COC(=O)N(Cc1ccc(-c2nnc(C(F)F)o2)cc1)c1ccccc1] |
| 214 | 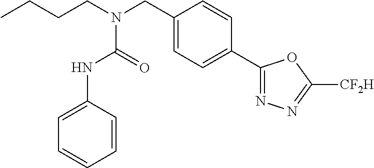 [SMILES: CCCCN(Cc1ccc(-c2nnc(C(F)F)o2)cc1)C(=O)Nc1ccccc1] |
| 215 | 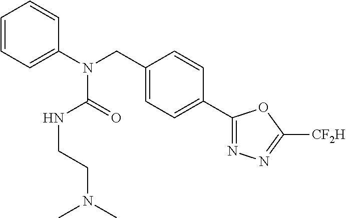 [SMILES: CN(C)CCNC(=O)N(Cc1ccc(-c2nnc(C(F)F)o2)cc1)c1ccccc1] |
| 216 | 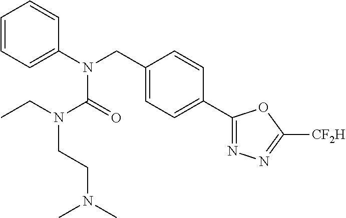 [SMILES: CCN(CCN(C)C)C(=O)N(Cc1ccc(-c2nnc(C(F)F)o2)cc1)c1ccccc1] |
| 217 | 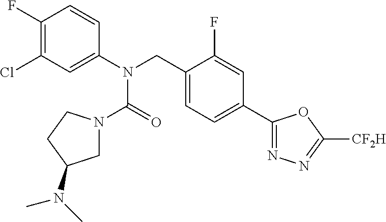 [SMILES: CN(C)[C@H]1CCN(C(=O)N(Cc2ccc(-c3nnc(C(F)F)o3)cc2F)c2ccc(F)c(Cl)c2)C1] |
| 218 | 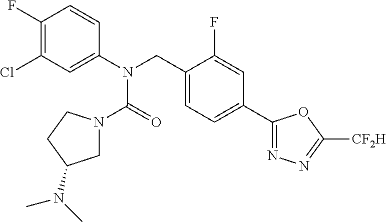 [SMILES: CN(C)[C@@H]1CCN(C(=O)N(Cc2ccc(-c3nnc(C(F)F)o3)cc2F)c2ccc(F)c(Cl)c2)C1] |
| 219 | 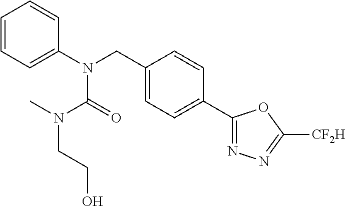 [SMILES: CN(CCO)C(=O)N(Cc1ccc(-c2nnc(C(F)F)o2)cc1)c1ccccc1] |
| 220 | 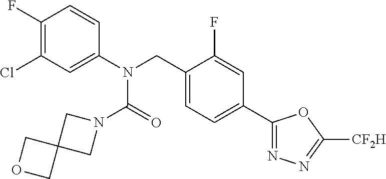 [SMILES: O=C(N1CC2(COC2)C1)N(Cc1ccc(-c2nnc(C(F)F)o2)cc1F)c1ccc(F)c(Cl)c1] |
| 221 | 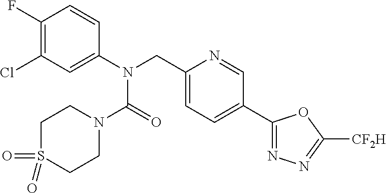 [SMILES: O=C(N1CCS(=O)(=O)CC1)N(Cc1ccc(-c2nnc(C(F)F)o2)cn1)c1ccc(F)c(Cl)c1] |
| 222 | 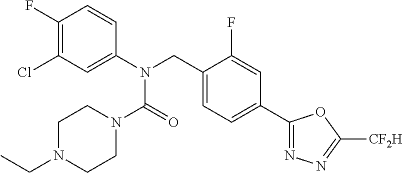 [SMILES: CCN1CCN(C(=O)N(Cc2ccc(-c3nnc(C(F)F)o3)cc2F)c2ccc(F)c(Cl)c2)CC1] |
| 223 | 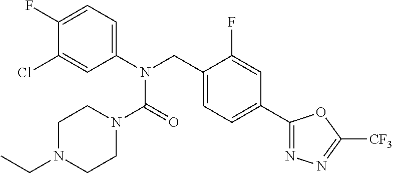 [SMILES: CCN1CCN(C(=O)N(Cc2ccc(-c3nnc(C(F)(F)F)o3)cc2F)c2ccc(F)c(Cl)c2)CC1] |
| 224 | 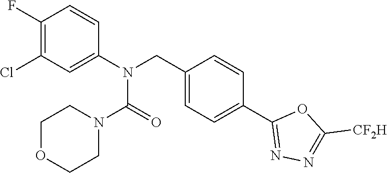 [SMILES: O=C(N1CCOCC1)N(Cc1ccc(-c2nnc(C(F)F)o2)cc1)c1ccc(F)c(Cl)c1] |
| 225 | 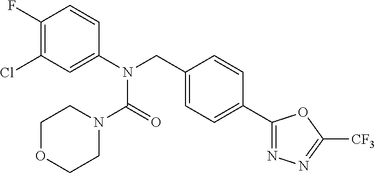 [SMILES: O=C(N1CCOCC1)N(Cc1ccc(-c2nnc(C(F)(F)F)o2)cc1)c1ccc(F)c(Cl)c1] |
| 226 | 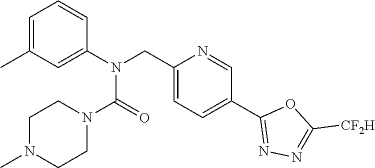 [SMILES: Cc1cccc(N(Cc2ccc(-c3nnc(C(F)F)o3)cn2)C(=O)N2CCN(C)CC2)c1] |
| 227 | 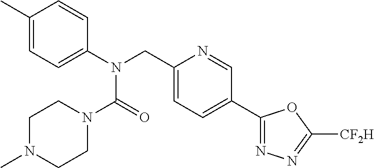 [SMILES: Cc1ccc(N(Cc2ccc(-c3nnc(C(F)F)o3)cn2)C(=O)N2CCN(C)CC2)cc1] |
| 228 | 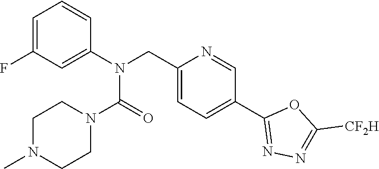 [SMILES: CN1CCN(C(=O)N(Cc2ccc(-c3nnc(C(F)F)o3)cn2)c2cccc(F)c2)CC1] |
| 229 | 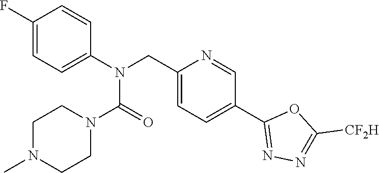 [SMILES: CN1CCN(C(=O)N(Cc2ccc(-c3nnc(C(F)F)o3)cn2)c2ccc(F)cc2)CC1] |
| 230 | 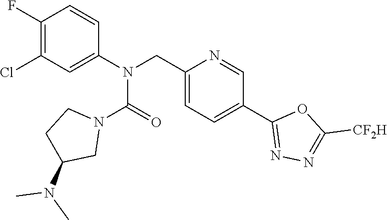 [SMILES: CN(C)[C@H]1CCN(C(=O)N(Cc2ccc(-c3nnc(C(F)F)o3)cn2)c2ccc(F)c(Cl)c2)C1] |
| 231 | 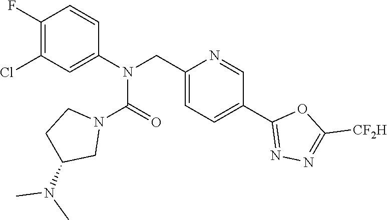 [SMILES: CN(C)[C@@H]1CCN(C(=O)N(Cc2ccc(-c3nnc(C(F)F)o3)cn2)c2ccc(F)c(Cl)c2)C1] |
| 232 | 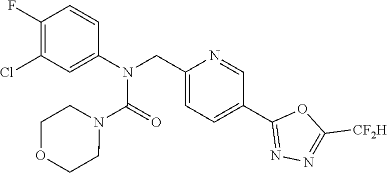 [SMILES: O=C(N1CCOCC1)N(Cc1ccc(-c2nnc(C(F)F)o2)cn1)c1ccc(F)c(Cl)c1] |
| 233 | 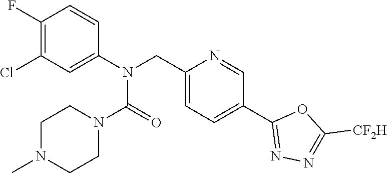 [SMILES: CN1CCN(C(=O)N(Cc2ccc(-c3nnc(C(F)F)o3)cn2)c2ccc(F)c(Cl)c2)CC1] |
| 234 | 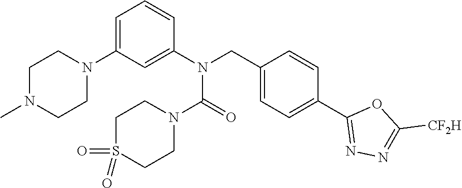 [SMILES: CN1CCN(c2cccc(N(Cc3ccc(-c4nnc(C(F)F)o4)cc3)C(=O)N3CCS(=O)(=O)CC3)c2)CC1] |
| 235 | 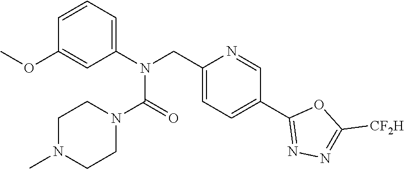 [SMILES: COc1cccc(N(Cc2ccc(-c3nnc(C(F)F)o3)cn2)C(=O)N2CCN(C)CC2)c1] |
| 236 | 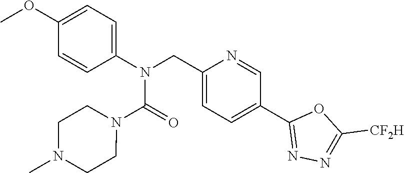 [SMILES: COc1ccc(N(Cc2ccc(-c3nnc(C(F)F)o3)cn2)C(=O)N2CCN(C)CC2)cc1] |
| 237 | 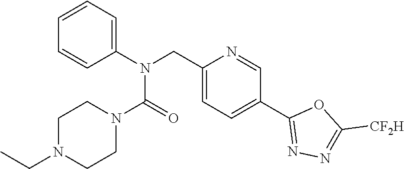 [SMILES: CCN1CCN(C(=O)N(Cc2ccc(-c3nnc(C(F)F)o3)cn2)c2ccccc2)CC1] |
| 238 | 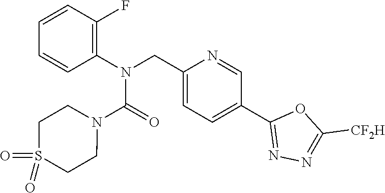 [SMILES: O=C(N1CCS(=O)(=O)CC1)N(Cc1ccc(-c2nnc(C(F)F)o2)cn1)c1ccccc1F] |
| 239 | 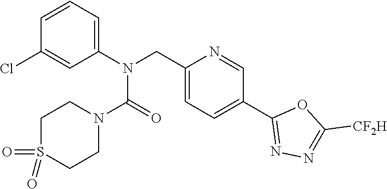 [SMILES: O=C(N1CCS(=O)(=O)CC1)N(Cc1ccc(-c2nnc(C(F)F)o2)cn1)c1cccc(Cl)c1] |
| 240 | 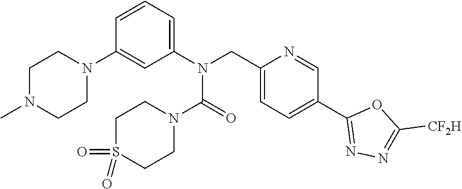 [SMILES: CN1CCN(c2cccc(N(Cc3ccc(-c4nnc(C(F)F)o4)cn3)C(=O)N3CCS(=O)(=O)CC3)c2)CC1] |
| 241 | 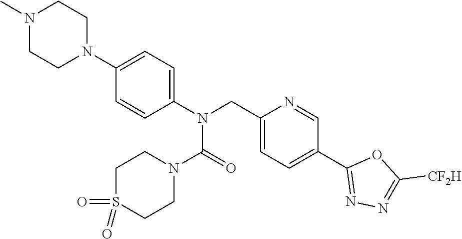 [SMILES: CN1CCN(c2ccc(N(Cc3ccc(-c4nnc(C(F)F)o4)cn3)C(=O)N3CCS(=O)(=O)CC3)cc2)CC1] |
| 242 | 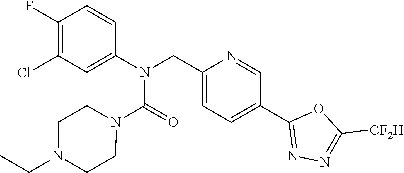 [SMILES: CCN1CCN(C(=O)N(Cc2ccc(-c3nnc(C(F)F)o3)cn2)c2ccc(F)c(Cl)c2)CC1] |
| 243 | 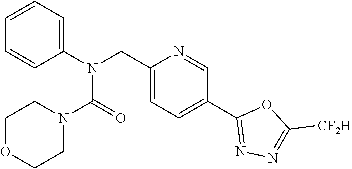 [SMILES: O=C(N1CCOCC1)N(Cc1ccc(-c2nnc(C(F)F)o2)cn1)c1ccccc1] |
| 2444 | 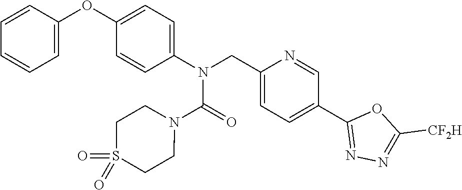 [SMILES: O=C(N1CCS(=O)(=O)CC1)N(Cc1ccc(-c2nnc(C(F)F)o2)cn1)c1ccc(Oc2ccccc2)cc1] |
| 245 | 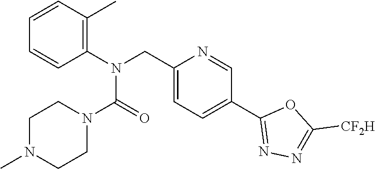 [SMILES: Cc1ccccc1N(Cc1ccc(-c2nnc(C(F)F)o2)cn1)C(=O)N1CCN(C)CC1] |
| 246 | 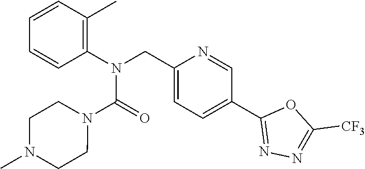 [SMILES: Cc1ccccc1N(Cc1ccc(-c2nnc(C(F)(F)F)o2)cn1)C(=O)N1CCN(C)CC1] |
| 247 | 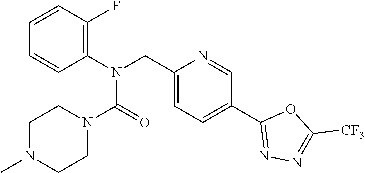 [SMILES: CN1CCN(C(=O)N(Cc2ccc(-c3nnc(C(F)(F)F)o3)cn2)c2ccccc2F)CC1] |
| 248 | 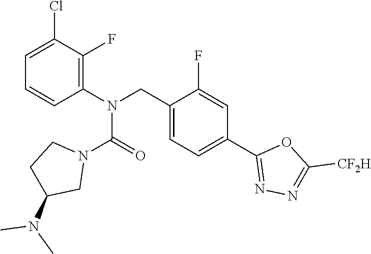 [SMILES: CN(C)[C@H]1CCN(C(=O)N(Cc2ccc(-c3nnc(C(F)F)o3)cc2F)c2cccc(Cl)c2F)C1] |
| 249 | 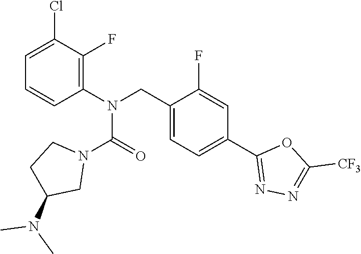 [SMILES: CN(C)[C@H]1CCN(C(=O)N(Cc2ccc(-c3nnc(C(F)(F)F)o3)cc2F)c2cccc(Cl)c2F)C1] |
| 250 | 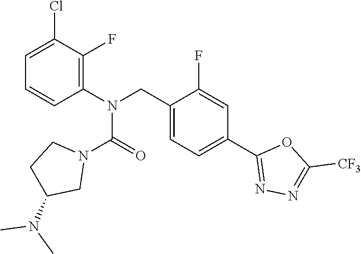 [SMILES: CN(C)[C@@H]1CCN(C(=O)N(Cc2ccc(-c3nnc(C(F)(F)F)o3)cc2F)c2cccc(Cl)c2F)C1] |
| 251 | 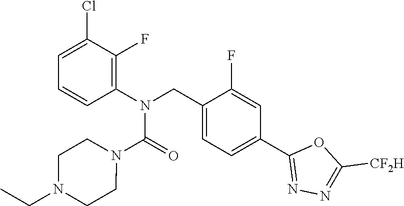 [SMILES: CCN1CCN(C(=O)N(Cc2ccc(-c3nnc(C(F)F)o3)cc2F)c2cccc(Cl)c2F)CC1] |
| 252 | 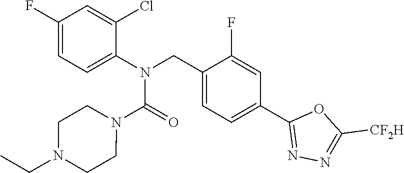 [SMILES: CCN1CCN(C(=O)N(Cc2ccc(-c3nnc(C(F)F)o3)cc2F)c2ccc(F)cc2Cl)CC1] |
| 253 | 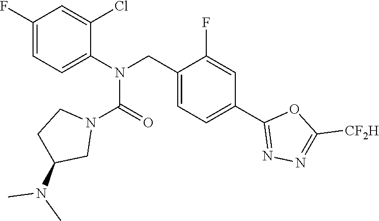 [SMILES: CN(C)[C@H]1CCN(C(=O)N(Cc2ccc(-c3nnc(C(F)F)o3)cc2F)c2ccc(F)cc2Cl)C1] |
| 254 | 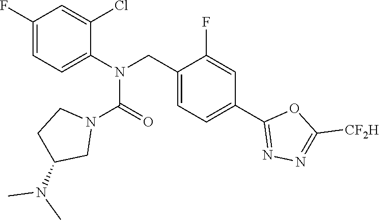 [SMILES: CN(C)[C@@H]1CCN(C(=O)N(Cc2ccc(-c3nnc(C(F)F)o3)cc2F)c2ccc(F)cc2Cl)C1] |
| 255 | 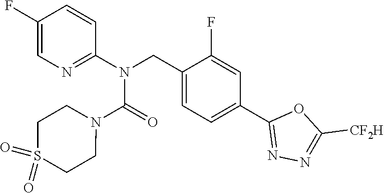 [SMILES: O=C(N1CCS(=O)(=O)CC1)N(Cc1ccc(-c2nnc(C(F)F)o2)cc1F)c1ccc(F)cn1] |
| 256 | 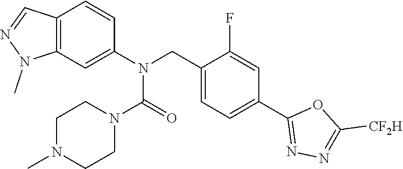 [SMILES: CN1CCN(C(=O)N(Cc2ccc(-c3nnc(C(F)F)o3)cc2F)c2ccc3cnn(C)c3c2)CC1] |
| 257 | 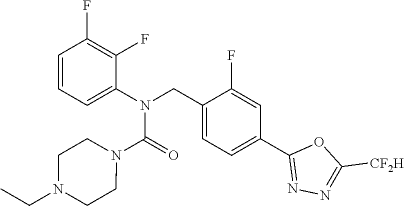 [SMILES: CCN1CCN(C(=O)N(Cc2ccc(-c3nnc(C(F)F)o3)cc2F)c2cccc(F)c2F)CC1] |
| 258 | 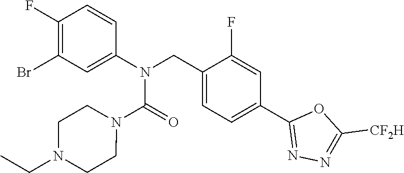 [SMILES: CCN1CCN(C(=O)N(Cc2ccc(-c3nnc(C(F)F)o3)cc2F)c2ccc(F)c(Br)c2)CC1] |
| 259 | 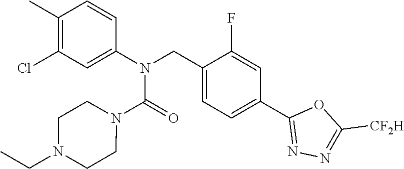 [SMILES: CCN1CCN(C(=O)N(Cc2ccc(-c3nnc(C(F)F)o3)cc2F)c2ccc(C)c(Cl)c2)CC1] |
| 260 | 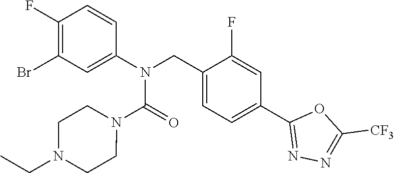 [SMILES: CCN1CCN(C(=O)N(Cc2ccc(-c3nnc(C(F)(F)F)o3)cc2F)c2ccc(F)c(Br)c2)CC1] |
| 261 | 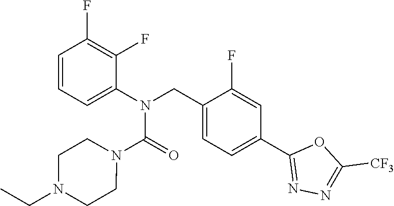 [SMILES: CCN1CCN(C(=O)N(Cc2ccc(-c3nnc(C(F)(F)F)o3)cc2F)c2cccc(F)c2F)CC1] |
| 262 | 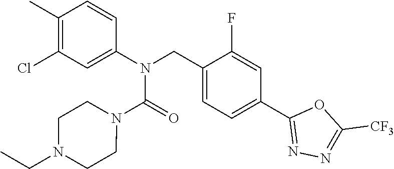 [SMILES: CCN1CCN(C(=O)N(Cc2ccc(-c3nnc(C(F)(F)F)o3)cc2F)c2ccc(C)c(Cl)c2)CC1] |
| 263 | 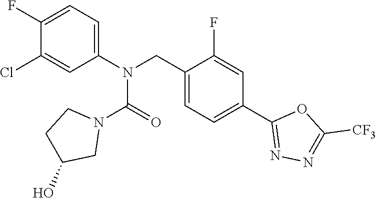 [SMILES: O=C(N1CC[C@@H](O)C1)N(Cc1ccc(-c2nnc(C(F)(F)F)o2)cc1F)c1ccc(F)c(Cl)c1] |
| 264 | 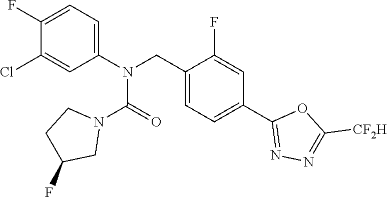 [SMILES: O=C(N1CC[C@H](F)C1)N(Cc1ccc(-c2nnc(C(F)F)o2)cc1F)c1ccc(F)c(Cl)c1] |
| 265 | 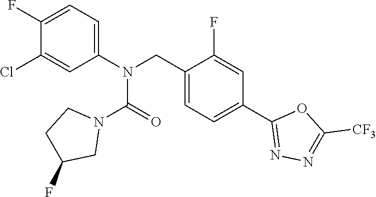 [SMILES: O=C(N1CC[C@H](F)C1)N(Cc1ccc(-c2nnc(C(F)(F)F)o2)cc1F)c1ccc(F)c(Cl)c1] |
| 266 | 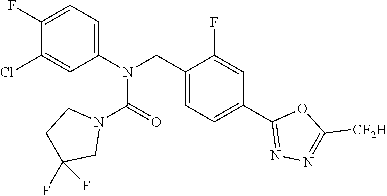 [SMILES: O=C(N1CCC(F)(F)C1)N(Cc1ccc(-c2nnc(C(F)F)o2)cc1F)c1ccc(F)c(Cl)c1] |
| 267 | 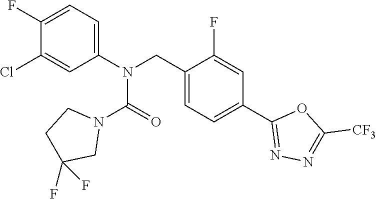 [SMILES: O=C(N1CCC(F)(F)C1)N(Cc1ccc(-c2nnc(C(F)(F)F)o2)cc1F)c1ccc(F)c(Cl)c1] |
| 268 | 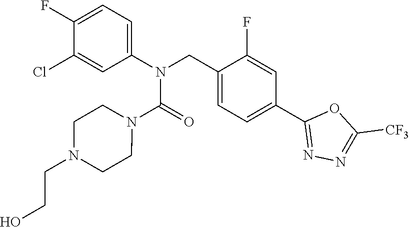 [SMILES: O=C(N1CCN(CCO)CC1)N(Cc1ccc(-c2nnc(C(F)(F)F)o2)cc1F)c1ccc(F)c(Cl)c1] |
| 269 | 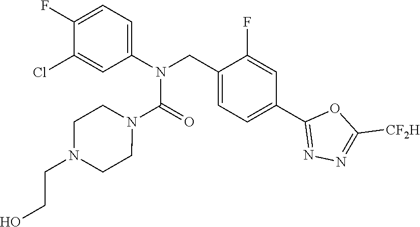 [SMILES: O=C(N1CCN(CCO)CC1)N(Cc1ccc(-c2nnc(C(F)F)o2)cc1F)c1ccc(F)c(Cl)c1] |
| 270 | 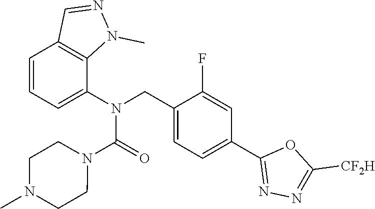 [SMILES: CN1CCN(C(=O)N(Cc2ccc(-c3nnc(C(F)F)o3)cc2F)c2cccc3cnn(C)c23)CC1] |
| 271 | 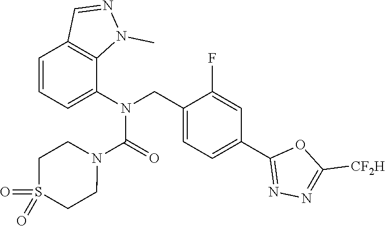 [SMILES: Cn1ncc2cccc(N(Cc3ccc(-c4nnc(C(F)F)o4)cc3F)C(=O)N3CCS(=O)(=O)CC3)c21] |
| 272 | 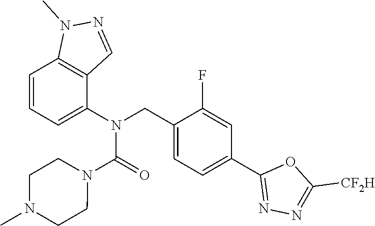 [SMILES: CN1CCN(C(=O)N(Cc2ccc(-c3nnc(C(F)F)o3)cc2F)c2cccc3c2cnn3C)CC1] |
| 273 | 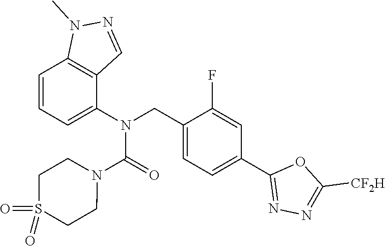 [SMILES: Cn1ncc2c(N(Cc3ccc(-c4nnc(C(F)F)o4)cc3F)C(=O)N3CCS(=O)(=O)CC3)cccc21] |
| 274 | 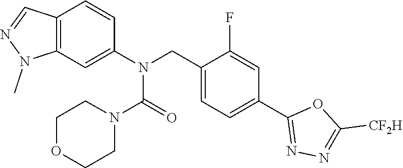 [SMILES: Cn1ncc2ccc(N(Cc3ccc(-c4nnc(C(F)F)o4)cc3F)C(=O)N3CCOCC3)cc21] |
| 275 | 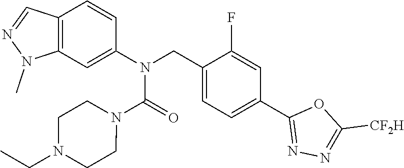 [SMILES: CCN1CCN(C(=O)N(Cc2ccc(-c3nnc(C(F)F)o3)cc2F)c2ccc3cnn(C)c3c2)CC1] |
| 276 | 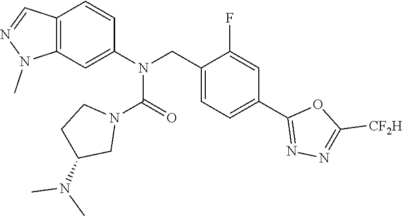 [SMILES: CN(C)[C@@H]1CCN(C(=O)N(Cc2ccc(-c3nnc(C(F)F)o3)cc2F)c2ccc3cnn(C)c3c2)C1] |
| 277 | 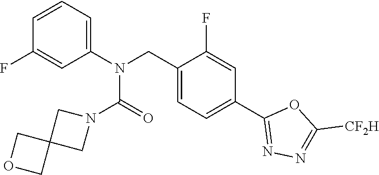 [SMILES: O=C(N1CC2(COC2)C1)N(Cc1ccc(-c2nnc(C(F)F)o2)cc1F)c1cccc(F)c1] |
| 278 | 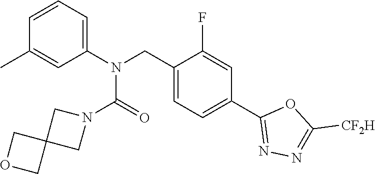 [SMILES: Cc1cccc(N(Cc2ccc(-c3nnc(C(F)F)o3)cc2F)C(=O)N2CC3(COC3)C2)c1] |
| 279 | 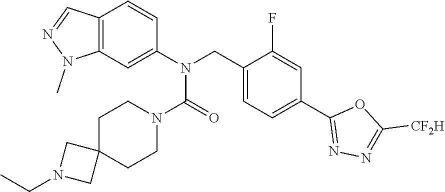 [SMILES: CCN1CC2(CCN(C(=O)N(Cc3ccc(-c4nnc(C(F)F)o4)cc3F)c3ccc4cnn(C)c4c3)CC2)C1] |
| 280 | 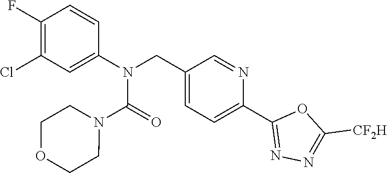 [SMILES: O=C(N1CCOCC1)N(Cc1ccc(-c2nnc(C(F)F)o2)nc1)c1ccc(F)c(Cl)c1] |
| 281 | 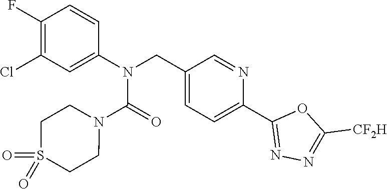 [SMILES: O=C(N1CCS(=O)(=O)CC1)N(Cc1ccc(-c2nnc(C(F)F)o2)nc1)c1ccc(F)c(Cl)c1] |
| 282 | 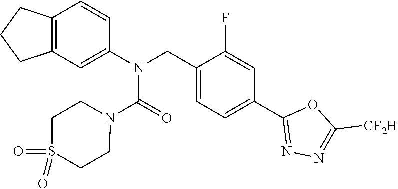 [SMILES: O=C(N1CCS(=O)(=O)CC1)N(Cc1ccc(-c2nnc(C(F)F)o2)cc1F)c1ccc2c(c1)CCC2] |
| 283 | 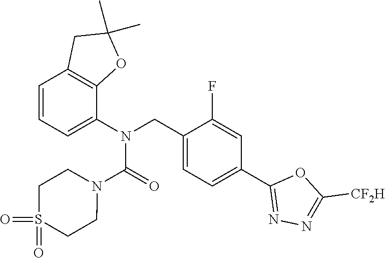 [SMILES: CC1(C)Cc2cccc(N(Cc3ccc(-c4nnc(C(F)F)o4)cc3F)C(=O)N3CCS(=O)(=O)CC3)c2O1] |
| 284 | 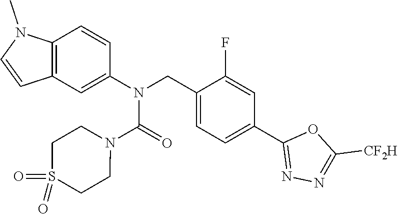 [SMILES: Cn1ccc2cc(N(Cc3ccc(-c4nnc(C(F)F)o4)cc3F)C(=O)N3CCS(=O)(=O)CC3)ccc21] |
| 285 | 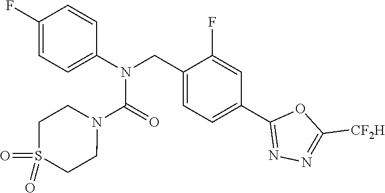 [SMILES: O=C(N1CCS(=O)(=O)CC1)N(Cc1ccc(-c2nnc(C(F)F)o2)cc1F)c1ccc(F)cc1] |
| 286 | 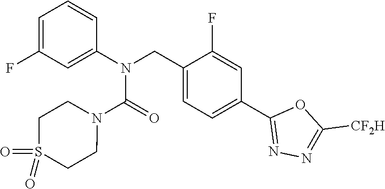 [SMILES: O=C(N1CCS(=O)(=O)CC1)N(Cc1ccc(-c2nnc(C(F)F)o2)cc1F)c1cccc(F)c1] |
| 287 | 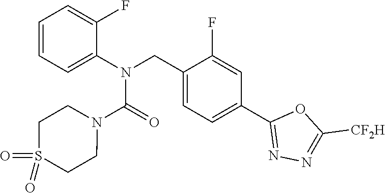 [SMILES: O=C(N1CCS(=O)(=O)CC1)N(Cc1ccc(-c2nnc(C(F)F)o2)cc1F)c1ccccc1F] |
| 288 | 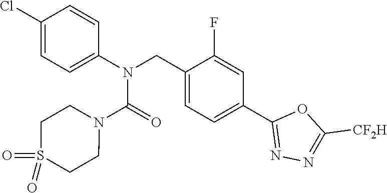 [SMILES: O=C(N1CCS(=O)(=O)CC1)N(Cc1ccc(-c2nnc(C(F)F)o2)cc1F)c1ccc(Cl)cc1] |
| 289 | 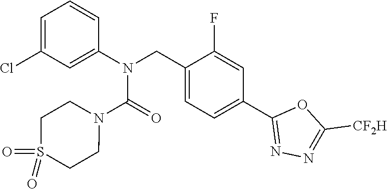 [SMILES: O=C(N1CCS(=O)(=O)CC1)N(Cc1ccc(-c2nnc(C(F)F)o2)cc1F)c1cccc(Cl)c1] |
| 290 | 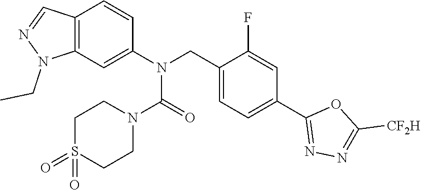 [SMILES: CCn1ncc2ccc(N(Cc3ccc(-c4nnc(C(F)F)o4)cc3F)C(=O)N3CCS(=O)(=O)CC3)cc21] |
| 291 | 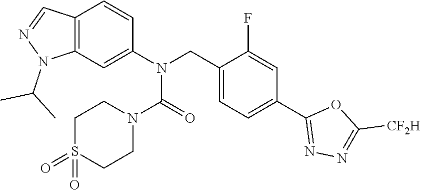 [SMILES: CC(C)n1ncc2ccc(N(Cc3ccc(-c4nnc(C(F)F)o4)cc3F)C(=O)N3CCS(=O)(=O)CC3)cc21] |
| 292 | 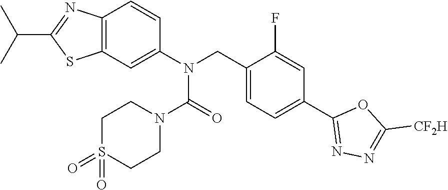 [SMILES: CC(C)c1nc2ccc(N(Cc3ccc(-c4nnc(C(F)F)o4)cc3F)C(=O)N3CCS(=O)(=O)CC3)cc2s1] |
| 293 | 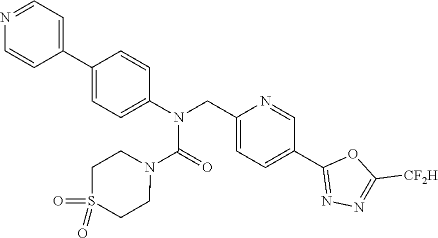 [SMILES: O=C(N1CCS(=O)(=O)CC1)N(Cc1ccc(-c2nnc(C(F)F)o2)cn1)c1ccc(-c2ccncc2)cc1] |
| 294 | 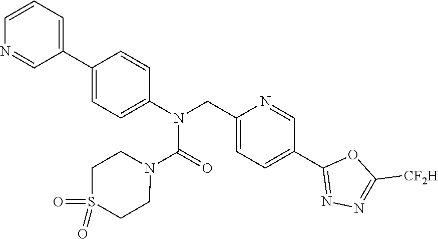 [SMILES: O=C(N1CCS(=O)(=O)CC1)N(Cc1ccc(-c2nnc(C(F)F)o2)cn1)c1ccc(-c2cccnc2)cc1] |
| 295 | 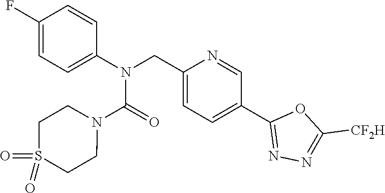 [SMILES: O=C(N1CCS(=O)(=O)CC1)N(Cc1ccc(-c2nnc(C(F)F)o2)cn1)c1ccc(F)cc1] |
| 296 | 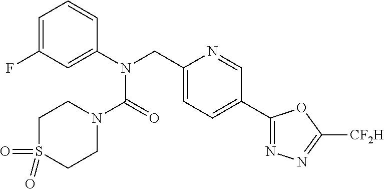 [SMILES: O=C(N1CCS(=O)(=O)CC1)N(Cc1ccc(-c2nnc(C(F)F)o2)cn1)c1cccc(F)c1] |
| 297 | 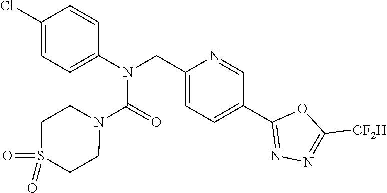 [SMILES: O=C(N1CCS(=O)(=O)CC1)N(Cc1ccc(-c2nnc(C(F)F)o2)cn1)c1ccc(Cl)cc1] |
| 298 | 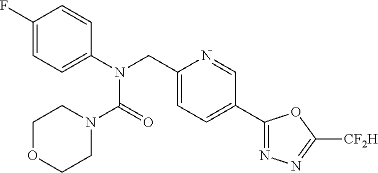 [SMILES: O=C(N1CCOCC1)N(Cc1ccc(-c2nnc(C(F)F)o2)cn1)c1ccc(F)cc1] |
| 299 | 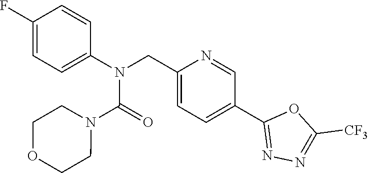 [SMILES: O=C(N1CCOCC1)N(Cc1ccc(-c2nnc(C(F)(F)F)o2)cn1)c1ccc(F)cc1] |
| 300 | 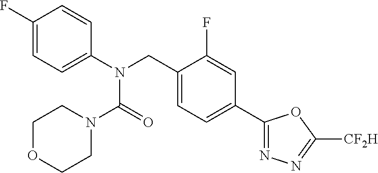 [SMILES: O=C(N1CCOCC1)N(Cc1ccc(-c2nnc(C(F)F)o2)cc1F)c1ccc(F)cc1] |
| 301 | 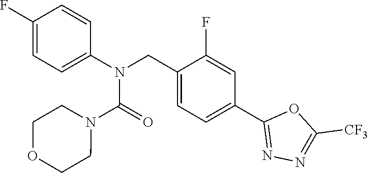 [SMILES: O=C(N1CCOCC1)N(Cc1ccc(-c2nnc(C(F)(F)F)o2)cc1F)c1ccc(F)cc1] |
| 302 | 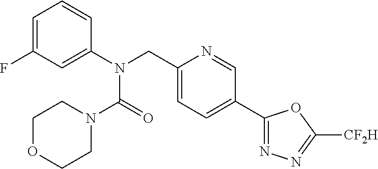 [SMILES: O=C(N1CCOCC1)N(Cc1ccc(-c2nnc(C(F)F)o2)cn1)c1cccc(F)c1] |
| 303 | 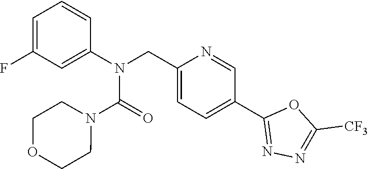 [SMILES: O=C(N1CCOCC1)N(Cc1ccc(-c2nnc(C(F)(F)F)o2)cn1)c1cccc(F)c1] |
| 304 | 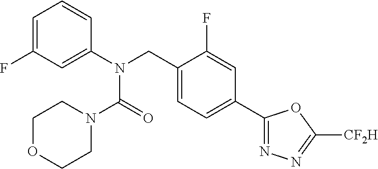 [SMILES: O=C(N1CCOCC1)N(Cc1ccc(-c2nnc(C(F)F)o2)cc1F)c1cccc(F)c1] |
| 305 | 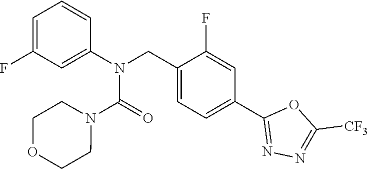 [SMILES: O=C(N1CCOCC1)N(Cc1ccc(-c2nnc(C(F)(F)F)o2)cc1F)c1cccc(F)c1] |
| 306 | 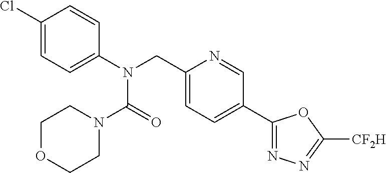 [SMILES: O=C(N1CCOCC1)N(Cc1ccc(-c2nnc(C(F)F)o2)cn1)c1ccc(Cl)cc1] |
| 307 | 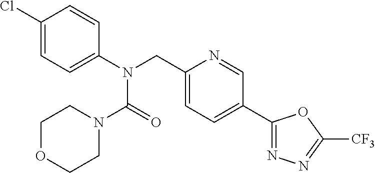 [SMILES: O=C(N1CCOCC1)N(Cc1ccc(-c2nnc(C(F)(F)F)o2)cn1)c1ccc(Cl)cc1] |
| 308 | 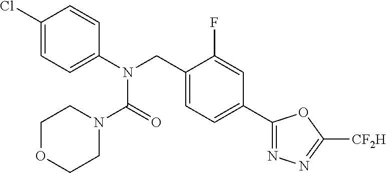 [SMILES: O=C(N1CCOCC1)N(Cc1ccc(-c2nnc(C(F)F)o2)cc1F)c1ccc(Cl)cc1] |
| 309 | 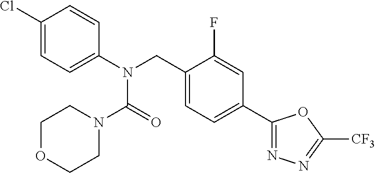 [SMILES: O=C(N1CCOCC1)N(Cc1ccc(-c2nnc(C(F)(F)F)o2)cc1F)c1ccc(Cl)cc1] |
| 310 | 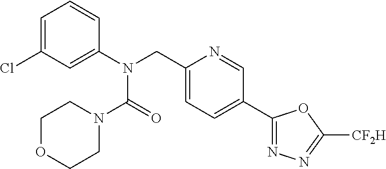 [SMILES: O=C(N1CCOCC1)N(Cc1ccc(-c2nnc(C(F)F)o2)cn1)c1cccc(Cl)c1] |
| 311 | 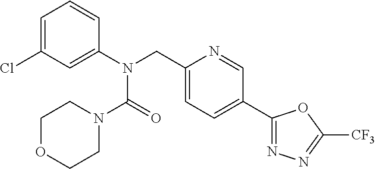 [SMILES: O=C(N1CCOCC1)N(Cc1ccc(-c2nnc(C(F)(F)F)o2)cn1)c1cccc(Cl)c1] |
| 312 | 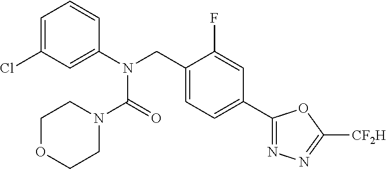 [SMILES: O=C(N1CCOCC1)N(Cc1ccc(-c2nnc(C(F)F)o2)cc1F)c1cccc(Cl)c1] |
| 313 | 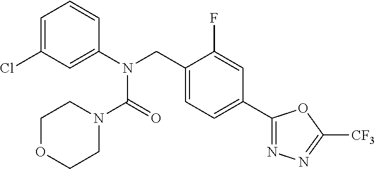 [SMILES: O=C(N1CCOCC1)N(Cc1ccc(-c2nnc(C(F)(F)F)o2)cc1F)c1cccc(Cl)c1] |
| 314 | 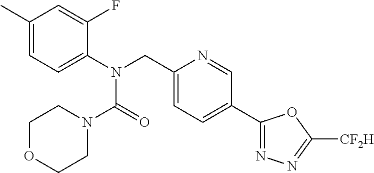 [SMILES: Cc1ccc(N(Cc2ccc(-c3nnc(C(F)F)o3)cn2)C(=O)N2CCOCC2)c(F)c1] |
| 315 | 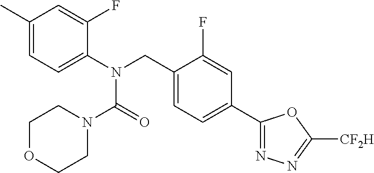 [SMILES: Cc1ccc(N(Cc2ccc(-c3nnc(C(F)F)o3)cc2F)C(=O)N2CCOCC2)c(F)c1] |
| 316 | 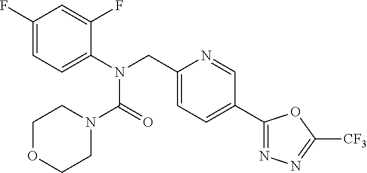 [SMILES: O=C(N1CCOCC1)N(Cc1ccc(-c2nnc(C(F)(F)F)o2)cn1)c1ccc(F)cc1F] |
| 317 | 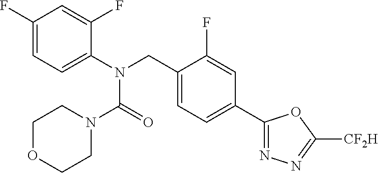 [SMILES: O=C(N1CCOCC1)N(Cc1ccc(-c2nnc(C(F)F)o2)cc1F)c1ccc(F)cc1F] |
| 318 | 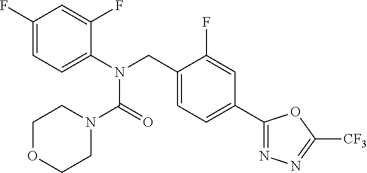 [SMILES: O=C(N1CCOCC1)N(Cc1ccc(-c2nnc(C(F)(F)F)o2)cc1F)c1ccc(F)cc1F] |
| 319 | 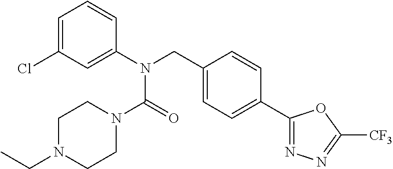 [SMILES: CCN1CCN(C(=O)N(Cc2ccc(-c3nnc(C(F)(F)F)o3)cc2)c2cccc(Cl)c2)CC1] |
| 320 | 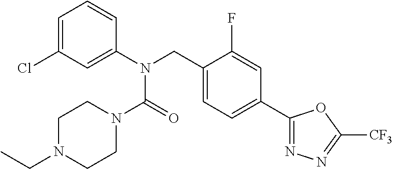 [SMILES: CCN1CCN(C(=O)N(Cc2ccc(-c3nnc(C(F)(F)F)o3)cc2F)c2cccc(Cl)c2)CC1] |
| 321 | 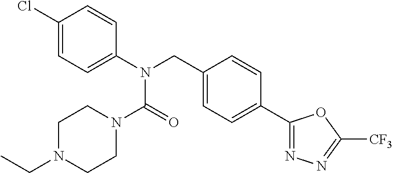 [SMILES: CCN1CCN(C(=O)N(Cc2ccc(-c3nnc(C(F)(F)F)o3)cc2)c2ccc(Cl)cc2)CC1] |
| 322 | 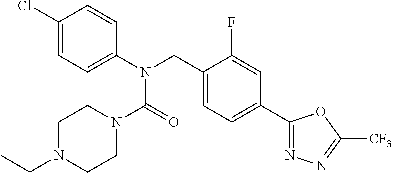 [SMILES: CCN1CCN(C(=O)N(Cc2ccc(-c3nnc(C(F)(F)F)o3)cc2F)c2ccc(Cl)cc2)CC1] |
| 323 | 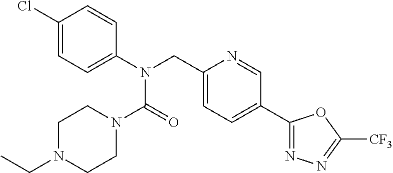 [SMILES: CCN1CCN(C(=O)N(Cc2ccc(-c3nnc(C(F)(F)F)o3)cn2)c2ccc(Cl)cc2)CC1] |
| 324 | 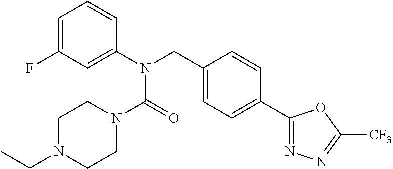 [SMILES: CCN1CCN(C(=O)N(Cc2ccc(-c3nnc(C(F)(F)F)o3)cc2)c2cccc(F)c2)CC1] |
| 325 | 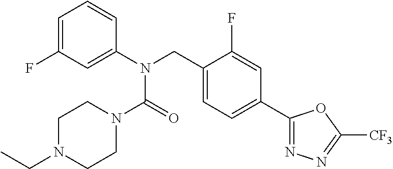 [SMILES: CCN1CCN(C(=O)N(Cc2ccc(-c3nnc(C(F)(F)F)o3)cc2F)c2cccc(F)c2)CC1] |
| 326 | 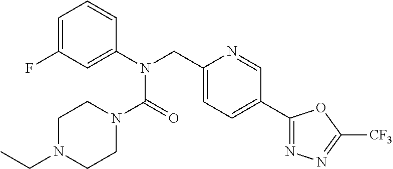 [SMILES: CCN1CCN(C(=O)N(Cc2ccc(-c3nnc(C(F)(F)F)o3)cn2)c2cccc(F)c2)CC1] |
| 327 | 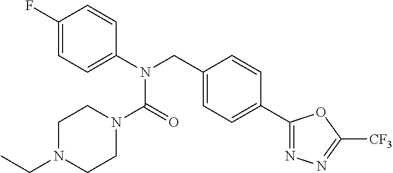 [SMILES: CCN1CCN(C(=O)N(Cc2ccc(-c3nnc(C(F)(F)F)o3)cc2)c2ccc(F)cc2)CC1] |
| 328 | 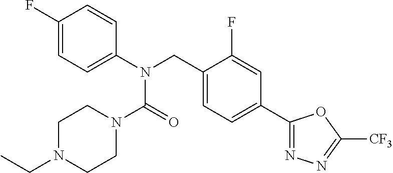 [SMILES: CCN1CCN(C(=O)N(Cc2ccc(-c3nnc(C(F)(F)F)o3)cc2F)c2ccc(F)cc2)CC1] |
| 329 | 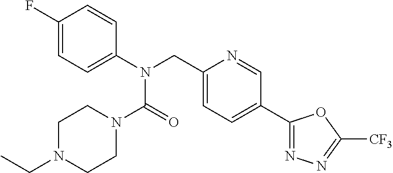 [SMILES: CCN1CCN(C(=O)N(Cc2ccc(-c3nnc(C(F)(F)F)o3)cn2)c2ccc(F)cc2)CC1] |
| 330 | 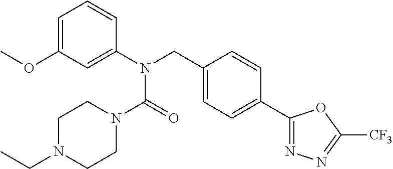 [SMILES: CCN1CCN(C(=O)N(Cc2ccc(-c3nnc(C(F)(F)F)o3)cc2)c2cccc(OC)c2)CC1] |
| 331 | 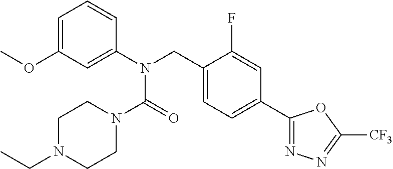 [SMILES: CCN1CCN(C(=O)N(Cc2ccc(-c3nnc(C(F)(F)F)o3)cc2F)c2cccc(OC)c2)CC1] |
| 332 | 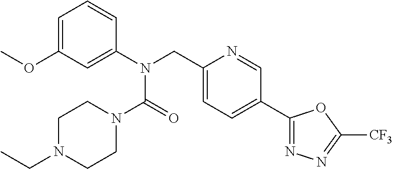 [SMILES: CCN1CCN(C(=O)N(Cc2ccc(-c3nnc(C(F)(F)F)o3)cn2)c2cccc(OC)c2)CC1] |
| 333 | 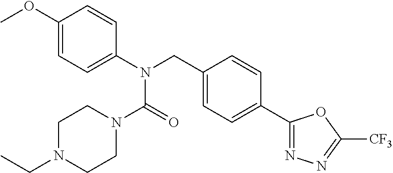 [SMILES: CCN1CCN(C(=O)N(Cc2ccc(-c3nnc(C(F)(F)F)o3)cc2)c2ccc(OC)cc2)CC1] |
| 334 | 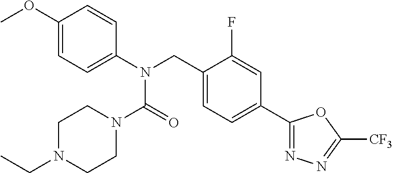 [SMILES: CCN1CCN(C(=O)N(Cc2ccc(-c3nnc(C(F)(F)F)o3)cc2F)c2ccc(OC)cc2)CC1] |
| 335 | 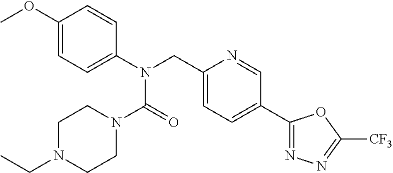 [SMILES: CCN1CCN(C(=O)N(Cc2ccc(-c3nnc(C(F)(F)F)o3)cn2)c2ccc(OC)cc2)CC1] |
| 336 | 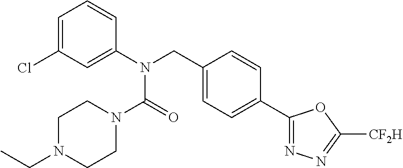 [SMILES: CCN1CCN(C(=O)N(Cc2ccc(-c3nnc(C(F)F)o3)cc2)c2cccc(Cl)c2)CC1] |
| 337 | 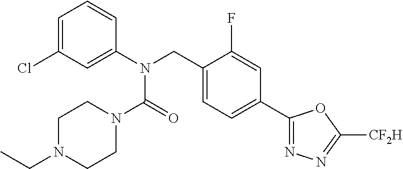 [SMILES: CCN1CCN(C(=O)N(Cc2ccc(-c3nnc(C(F)F)o3)cc2F)c2cccc(Cl)c2)CC1] |
| 338 | 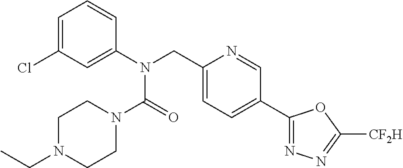 [SMILES: CCN1CCN(C(=O)N(Cc2ccc(-c3nnc(C(F)F)o3)cn2)c2cccc(Cl)c2)CC1] |
| 339 | 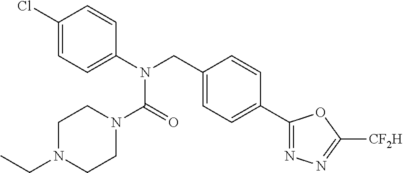 [SMILES: CCN1CCN(C(=O)N(Cc2ccc(-c3nnc(C(F)F)o3)cc2)c2ccc(Cl)cc2)CC1] |
| 340 | 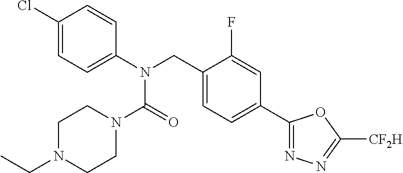 [SMILES: CCN1CCN(C(=O)N(Cc2ccc(-c3nnc(C(F)F)o3)cc2F)c2ccc(Cl)cc2)CC1] |
| 341 | 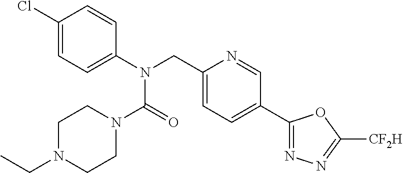 [SMILES: CCN1CCN(C(=O)N(Cc2ccc(-c3nnc(C(F)F)o3)cn2)c2ccc(Cl)cc2)CC1] |
| 342 | 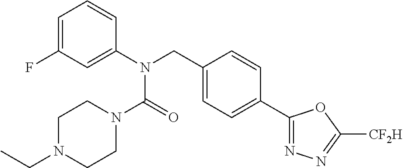 [SMILES: CCN1CCN(C(=O)N(Cc2ccc(-c3nnc(C(F)F)o3)cc2)c2cccc(F)c2)CC1] |
| 343 | 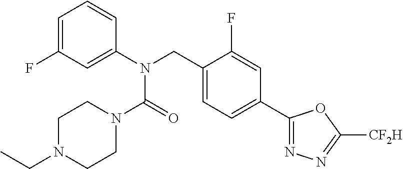 [SMILES: CCN1CCN(C(=O)N(Cc2ccc(-c3nnc(C(F)F)o3)cc2F)c2cccc(F)c2)CC1] |
| 344 | 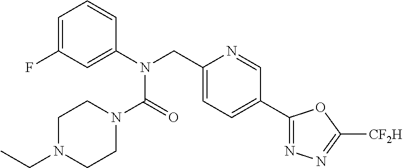 [SMILES: CCN1CCN(C(=O)N(Cc2ccc(-c3nnc(C(F)F)o3)cn2)c2cccc(F)c2)CC1] |
| 345 | 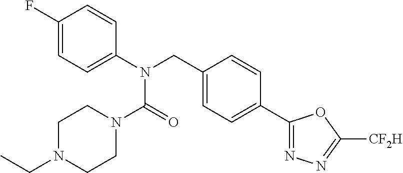 [SMILES: CCN1CCN(C(=O)N(Cc2ccc(-c3nnc(C(F)F)o3)cc2)c2ccc(F)cc2)CC1] |
| 346 | 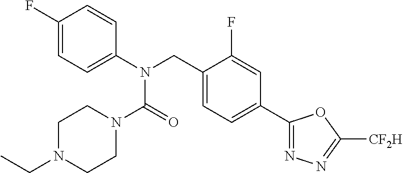 [SMILES: CCN1CCN(C(=O)N(Cc2ccc(-c3nnc(C(F)F)o3)cc2F)c2ccc(F)cc2)CC1] |
| 347 | 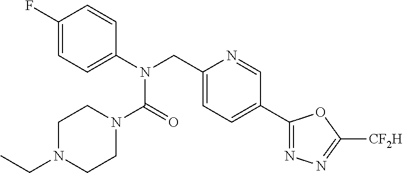 [SMILES: CCN1CCN(C(=O)N(Cc2ccc(-c3nnc(C(F)F)o3)cn2)c2ccc(F)cc2)CC1] |
| 348 | 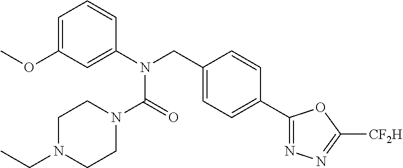 [SMILES: CCN1CCN(C(=O)N(Cc2ccc(-c3nnc(C(F)F)o3)cc2)c2cccc(OC)c2)CC1] |
| 349 | 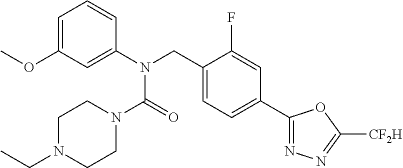 [SMILES: CCN1CCN(C(=O)N(Cc2ccc(-c3nnc(C(F)F)o3)cc2F)c2cccc(OC)c2)CC1] |
| 350 | 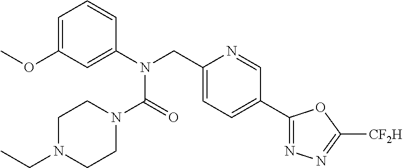 [SMILES: CCN1CCN(C(=O)N(Cc2ccc(-c3nnc(C(F)F)o3)cn2)c2cccc(OC)c2)CC1] |
| 351 | 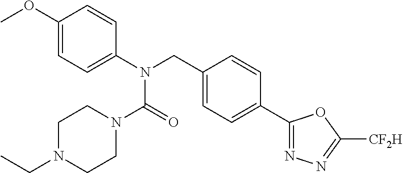 [SMILES: CCN1CCN(C(=O)N(Cc2ccc(-c3nnc(C(F)F)o3)cc2)c2ccc(OC)cc2)CC1] |
| 352 | 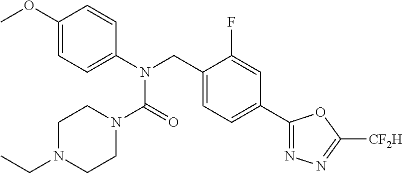 [SMILES: CCN1CCN(C(=O)N(Cc2ccc(-c3nnc(C(F)F)o3)cc2F)c2ccc(OC)cc2)CC1] |
| 353 | 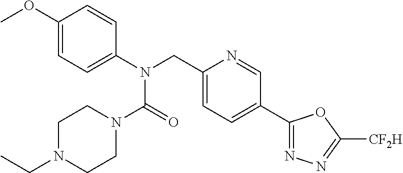 [SMILES: CCN1CCN(C(=O)N(Cc2ccc(-c3nnc(C(F)F)o3)cn2)c2ccc(OC)cc2)CC1] |
| 354 | 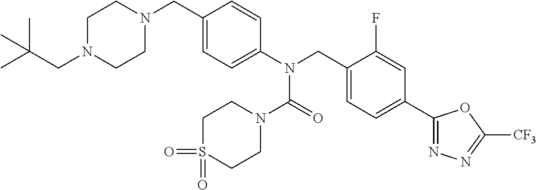 [SMILES: CC(C)(C)CN1CCN(Cc2ccc(N(Cc3ccc(-c4nnc(C(F)(F)F)o4)cc3F)C(=O)N3CCS(=O)(=O)CC3)cc2)CC1] |
| 355 | 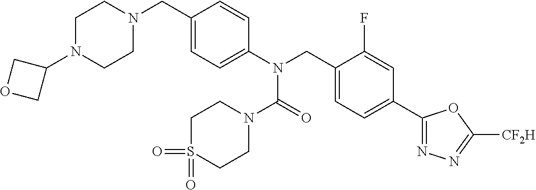 [SMILES: O=C(N1CCS(=O)(=O)CC1)N(Cc1ccc(-c2nnc(C(F)F)o2)cc1F)c1ccc(CN2CCN(C3COC3)CC2)cc1] |
| 356 | 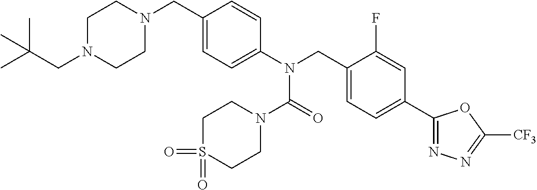 [SMILES: CC(C)(C)CN1CCN(Cc2ccc(N(Cc3ccc(-c4nnc(C(F)(F)F)o4)cc3F)C(=O)N3CCS(=O)(=O)CC3)cc2)CC1] |
| 357 | 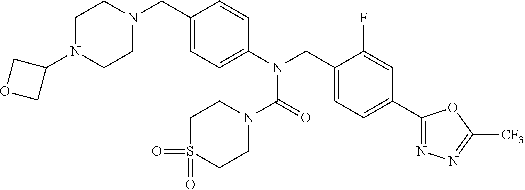 [SMILES: O=C(N1CCS(=O)(=O)CC1)N(Cc1ccc(-c2nnc(C(F)(F)F)o2)cc1F)c1ccc(CN2CCN(C3COC3)CC2)cc1] |
| 358 | 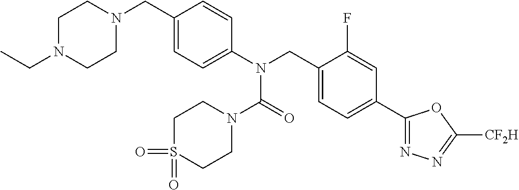 [SMILES: CCN1CCN(Cc2ccc(N(Cc3ccc(-c4nnc(C(F)F)o4)cc3F)C(=O)N3CCS(=O)(=O)CC3)cc2)CC1] |
| 359 | 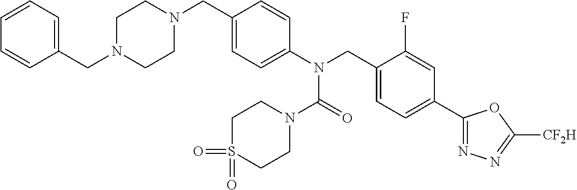 [SMILES: O=C(N1CCS(=O)(=O)CC1)N(Cc1ccc(-c2nnc(C(F)F)o2)cc1F)c1ccc(CN2CCN(Cc3ccccc3)CC2)cc1] |
| 360 | 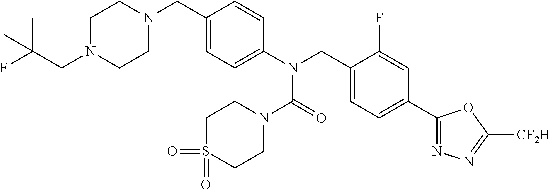 [SMILES: CC(C)(F)CN1CCN(Cc2ccc(N(Cc3ccc(-c4nnc(C(F)F)o4)cc3F)C(=O)N3CCS(=O)(=O)CC3)cc2)CC1] |
| 361 | 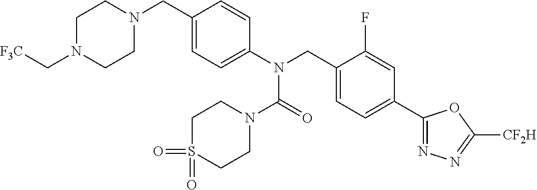 [SMILES: O=C(N1CCS(=O)(=O)CC1)N(Cc1ccc(-c2nnc(C(F)F)o2)cc1F)c1ccc(CN2CCN(CC(F)(F)F)CC2)cc1] |
| 362 | 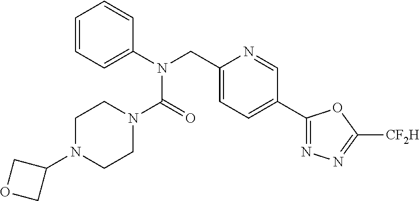 [SMILES: O=C(N1CCN(C2COC2)CC1)N(Cc1ccc(-c2nnc(C(F)F)o2)cn1)c1ccccc1] |
| 363 | 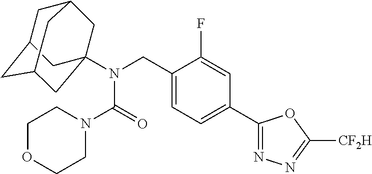 [SMILES: O=C(N1CCOCC1)N(Cc1ccc(-c2nnc(C(F)F)o2)cc1F)C12CC3CC(CC(C3)C1)C2] |
| 364 | 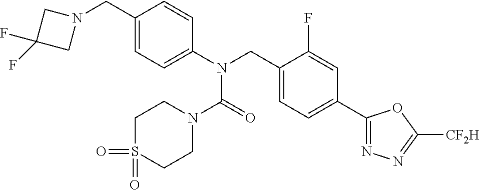 [SMILES: O=C(N1CCS(=O)(=O)CC1)N(Cc1ccc(-c2nnc(C(F)F)o2)cc1F)c1ccc(CN2CC(F)(F)C2)cc1] |
| 365 | 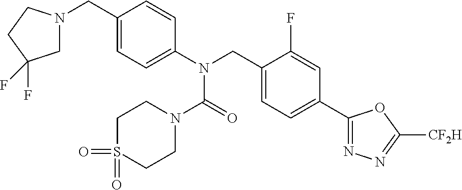 [SMILES: O=C(N1CCS(=O)(=O)CC1)N(Cc1ccc(-c2nnc(C(F)F)o2)cc1F)c1ccc(CN2CCC(F)(F)C2)cc1] |
| 366 | 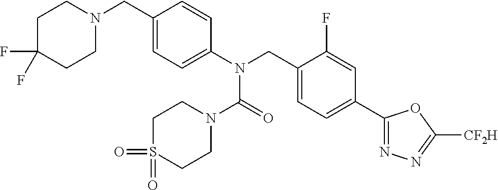 [SMILES: O=C(N1CCS(=O)(=O)CC1)N(Cc1ccc(-c2nnc(C(F)F)o2)cc1F)c1ccc(CN2CCC(F)(F)CC2)cc1] |
| 367 | 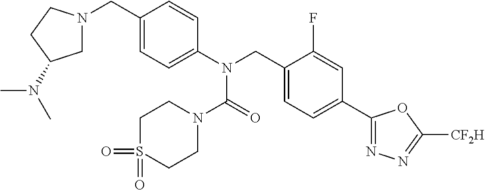 [SMILES: CN(C)[C@@H]1CCN(Cc2ccc(N(Cc3ccc(-c4nnc(C(F)F)o4)cc3F)C(=O)N3CCS(=O)(=O)CC3)cc2)C1] |
| 368 | 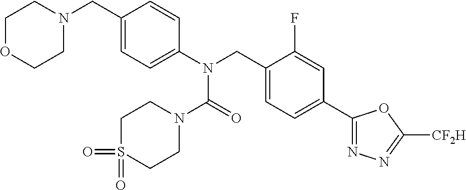 [SMILES: O=C(N1CCS(=O)(=O)CC1)N(Cc1ccc(-c2nnc(C(F)F)o2)cc1F)c1ccc(CN2CCOCC2)cc1] |
| 369 | 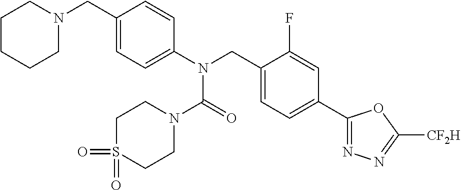 [SMILES: O=C(N1CCS(=O)(=O)CC1)N(Cc1ccc(-c2nnc(C(F)F)o2)cc1F)c1ccc(CN2CCCCC2)cc1] |
| 370 | 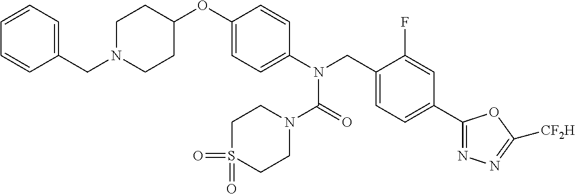 [SMILES: O=C(N1CCS(=O)(=O)CC1)N(Cc1ccc(-c2nnc(C(F)F)o2)cc1F)c1ccc(OC2CCN(Cc3ccccc3)CC2)cc1] |
| 371 | 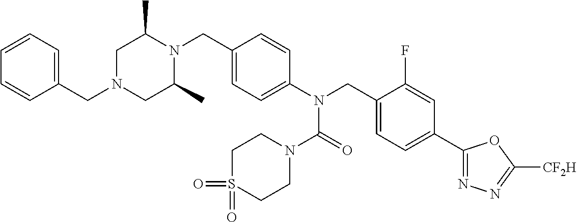 [SMILES: C[C@@H]1CN(Cc2ccccc2)C[C@H](C)N1Cc1ccc(N(Cc2ccc(-c3nnc(C(F)F)o3)cc2F)C(=O)N2CCS(=O)(=O)CC2)cc1] |
| 372 | 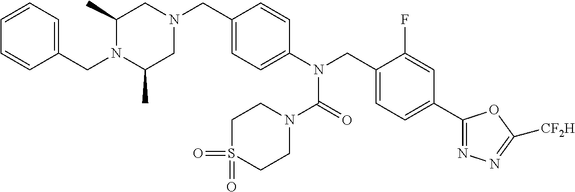 [SMILES: C[C@@H]1CN(Cc2ccc(N(Cc3ccc(-c4nnc(C(F)F)o4)cc3F)C(=O)N3CCS(=O)(=O)CC3)cc2)C[C@H](C)N1Cc1ccccc1] |
| 373 | 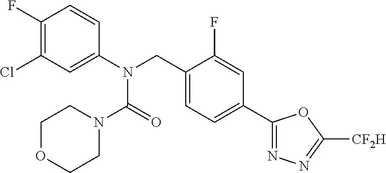 [SMILES: O=C(N1CCOCC1)N(Cc1ccc(-c2nnc(C(F)F)o2)cc1F)c1ccc(F)c(Cl)c1] |
| 374 | 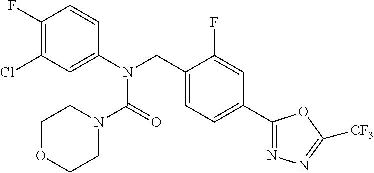 [SMILES: O=C(N1CCOCC1)N(Cc1ccc(-c2nnc(C(F)(F)F)o2)cc1F)c1ccc(F)c(Cl)c1] |
| 375 | 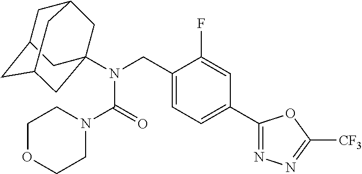 [SMILES: O=C(N1CCOCC1)N(Cc1ccc(-c2nnc(C(F)(F)F)o2)cc1F)C12CC3CC(CC(C3)C1)C2] |
| 376 | 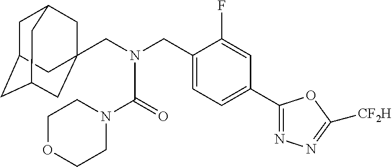 [SMILES: O=C(N1CCOCC1)N(Cc1ccc(-c2nnc(C(F)F)o2)cc1F)CC12CC3CC(CC(C3)C1)C2] |
| 377 | 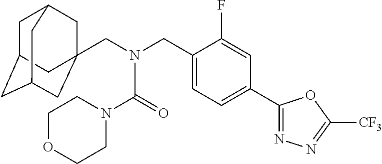 [SMILES: O=C(N1CCOCC1)N(Cc1ccc(-c2nnc(C(F)(F)F)o2)cc1F)CC12CC3CC(CC(C3)C1)C2] |
| 378 | 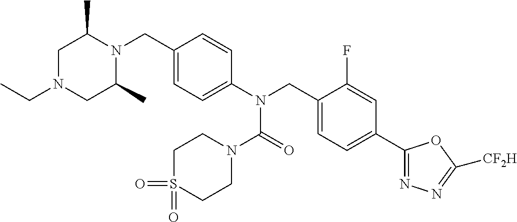 [SMILES: CCN1C[C@@H](C)N(Cc2ccc(N(Cc3ccc(-c4nnc(C(F)F)o4)cc3F)C(=O)N3CCS(=O)(=O)CC3)cc2)[C@@H](C)C1] |
| 379 | 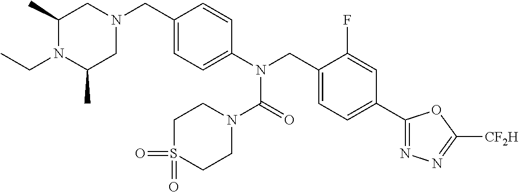 [SMILES: CCN1[C@H](C)CN(Cc2ccc(N(Cc3ccc(-c4nnc(C(F)F)o4)cc3F)C(=O)N3CCS(=O)(=O)CC3)cc2)C[C@@H]1C] |
| 380 | 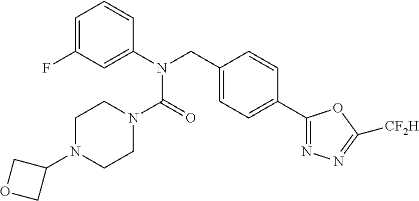 [SMILES: O=C(N1CCN(C2COC2)CC1)N(Cc1ccc(-c2nnc(C(F)F)o2)cc1)c1cccc(F)c1] |
| 381 | 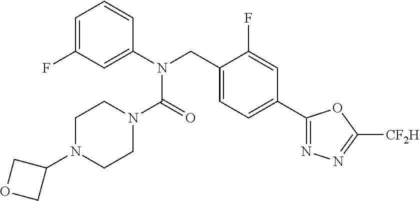 [SMILES: O=C(N1CCN(C2COC2)CC1)N(Cc1ccc(-c2nnc(C(F)F)o2)cc1F)c1cccc(F)c1] |
| 382 | 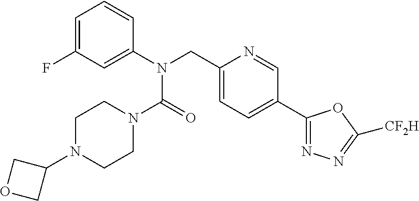 [SMILES: O=C(N1CCN(C2COC2)CC1)N(Cc1ccc(-c2nnc(C(F)F)o2)cn1)c1cccc(F)c1] |
| 383 | 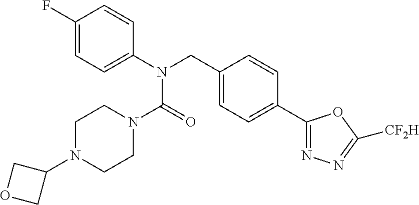 [SMILES: O=C(N1CCN(C2COC2)CC1)N(Cc1ccc(-c2nnc(C(F)F)o2)cc1)c1ccc(F)cc1] |
| 384 | 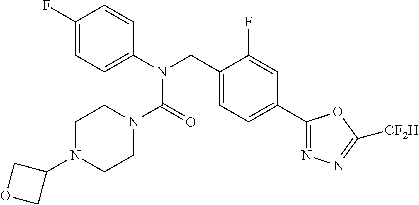 [SMILES: O=C(N1CCN(C2COC2)CC1)N(Cc1ccc(-c2nnc(C(F)F)o2)cc1F)c1ccc(F)cc1] |
| 385 | 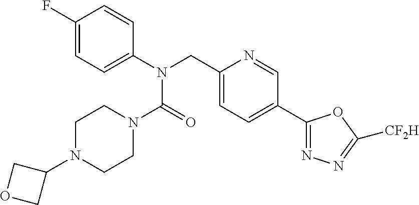 [SMILES: O=C(N1CCN(C2COC2)CC1)N(Cc1ccc(-c2nnc(C(F)F)o2)cn1)c1ccc(F)cc1] |
| 386 | 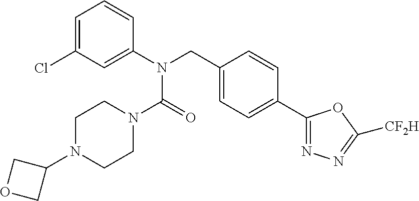 [SMILES: O=C(N1CCN(C2COC2)CC1)N(Cc1ccc(-c2nnc(C(F)F)o2)cc1)c1cccc(Cl)c1] |
| 387 | 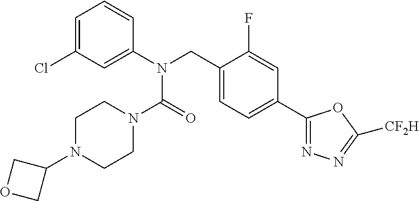 [SMILES: O=C(N1CCN(C2COC2)CC1)N(Cc1ccc(-c2nnc(C(F)F)o2)cc1F)c1cccc(Cl)c1] |
| 388 | 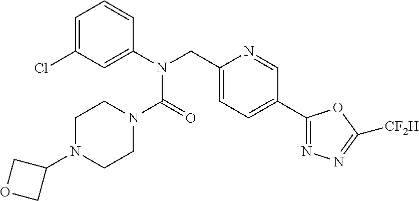 [SMILES: O=C(N1CCN(C2COC2)CC1)N(Cc1ccc(-c2nnc(C(F)F)o2)cn1)c1cccc(Cl)c1] |
| 389 | 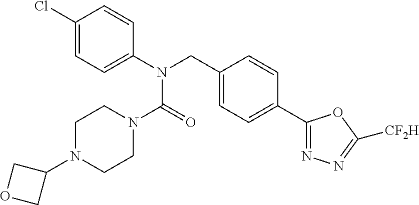 [SMILES: O=C(N1CCN(C2COC2)CC1)N(Cc1ccc(-c2nnc(C(F)F)o2)cc1)c1ccc(Cl)cc1] |
| 390 | 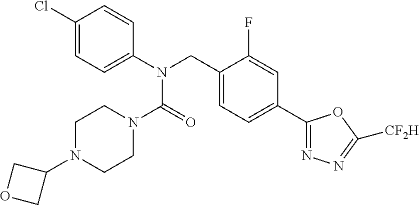 [SMILES: O=C(N1CCN(C2COC2)CC1)N(Cc1ccc(-c2nnc(C(F)F)o2)cc1F)c1ccc(Cl)cc1] |
| 391 | 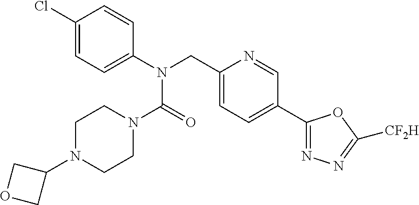 [SMILES: O=C(N1CCN(C2COC2)CC1)N(Cc1ccc(-c2nnc(C(F)F)o2)cn1)c1ccc(Cl)cc1] |
| 392 | 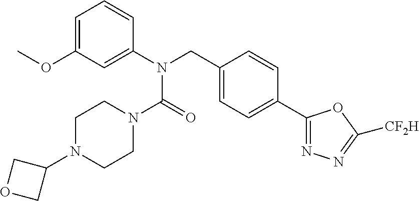 [SMILES: COc1cccc(N(Cc2ccc(-c3nnc(C(F)F)o3)cc2)C(=O)N2CCN(C3COC3)CC2)c1] |
| 393 | 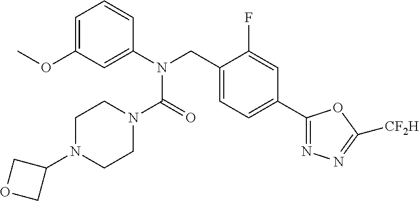 [SMILES: COc1cccc(N(Cc2ccc(-c3nnc(C(F)F)o3)cc2F)C(=O)N2CCN(C3COC3)CC2)c1] |
| 394 | 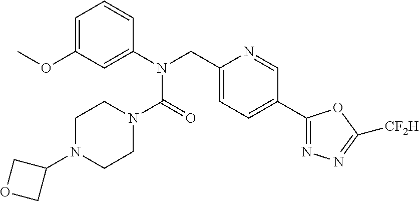 [SMILES: COc1cccc(N(Cc2ccc(-c3nnc(C(F)F)o3)cn2)C(=O)N2CCN(C3COC3)CC2)c1] |
| 395 | 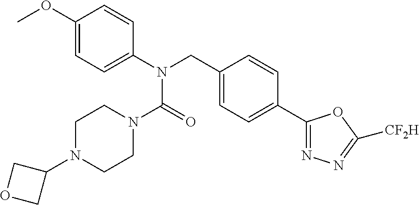 [SMILES: COc1ccc(N(Cc2ccc(-c3nnc(C(F)F)o3)cc2)C(=O)N2CCN(C3COC3)CC2)cc1] |
| 396 | 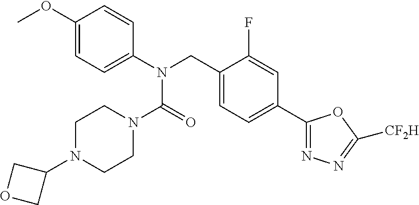 [SMILES: COc1ccc(N(Cc2ccc(-c3nnc(C(F)F)o3)cc2F)C(=O)N2CCN(C3COC3)CC2)cc1] |
| 397 | 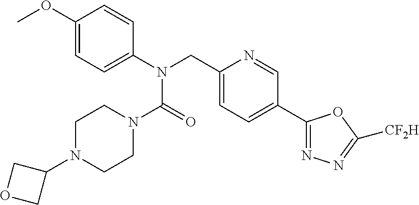 [SMILES: COc1ccc(N(Cc2ccc(-c3nnc(C(F)F)o3)cn2)C(=O)N2CCN(C3COC3)CC2)cc1] |
| 398 | 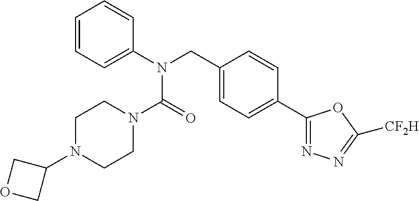 [SMILES: O=C(N1CCN(C2COC2)CC1)N(Cc1ccc(-c2nnc(C(F)F)o2)cc1)c1ccccc1] |
| 399 | 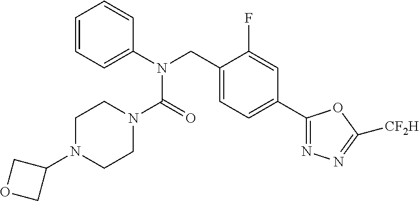 [SMILES: O=C(N1CCN(C2COC2)CC1)N(Cc1ccc(-c2nnc(C(F)F)o2)cc1F)c1ccccc1] |
| 400 | 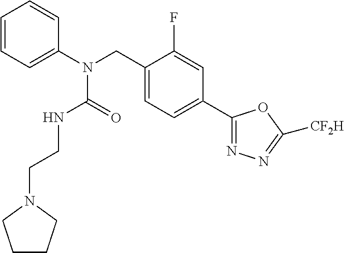 [SMILES: O=C(NCCN1CCCC1)N(Cc1ccc(-c2nnc(C(F)F)o2)cc1F)c1ccccc1] |
| 401 | 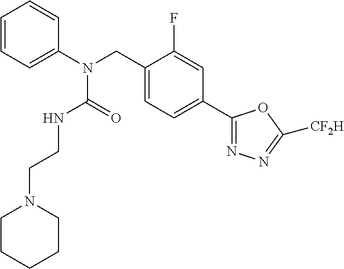 [SMILES: O=C(NCCN1CCCCC1)N(Cc1ccc(-c2nnc(C(F)F)o2)cc1F)c1ccccc1] |
| 402 | 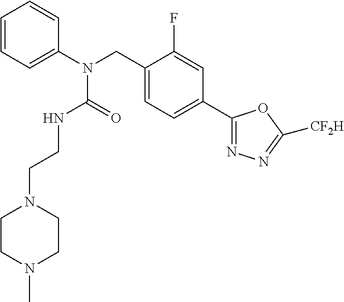 [SMILES: CN1CCN(CCNC(=O)N(Cc2ccc(-c3nnc(C(F)F)o3)cc2F)c2ccccc2)CC1] |
| 403 | 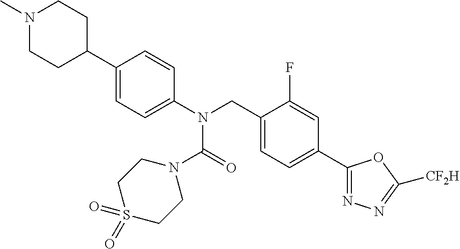 [SMILES: CN1CCC(c2ccc(N(Cc3ccc(-c4nnc(C(F)F)o4)cc3F)C(=O)N3CCS(=O)(=O)CC3)cc2)CC1] |
| 404 | 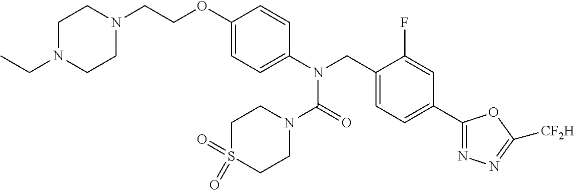 [SMILES: CCN1CCN(CCOc2ccc(N(Cc3ccc(-c4nnc(C(F)F)o4)cc3F)C(=O)N3CCS(=O)(=O)CC3)cc2)CC1] |
| 405 | 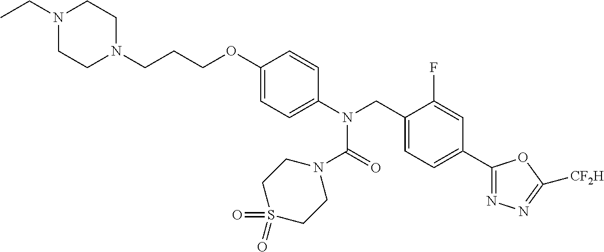 [SMILES: CCN1CCN(CCCOc2ccc(N(Cc3ccc(-c4nnc(C(F)F)o4)cc3F)C(=O)N3CCS(=O)(=O)CC3)cc2)CC1] |
| 406 | 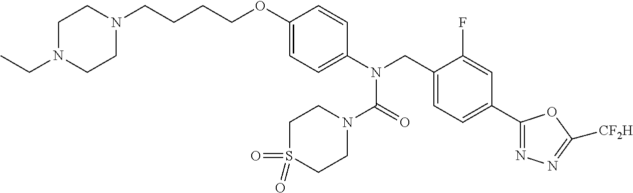 [SMILES: CCN1CCN(CCCCOc2ccc(N(Cc3ccc(-c4nnc(C(F)F)o4)cc3F)C(=O)N3CCS(=O)(=O)CC3)cc2)CC1] |
| 407 | 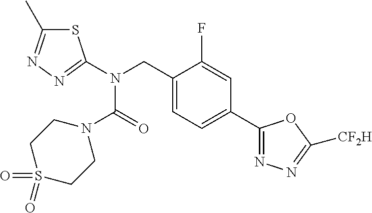 [SMILES: Cc1nnc(N(Cc2ccc(-c3nnc(C(F)F)o3)cc2F)C(=O)N2CCS(=O)(=O)CC2)s1] |
| 408 | 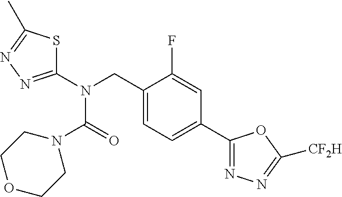 [SMILES: Cc1nnc(N(Cc2ccc(-c3nnc(C(F)F)o3)cc2F)C(=O)N2CCOCC2)s1] |
| 409 | 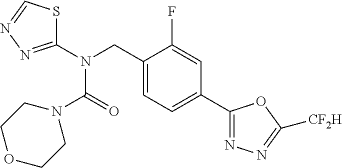 [SMILES: O=C(N1CCOCC1)N(Cc1ccc(-c2nnc(C(F)F)o2)cc1F)c1nncs1] |
| 410 | 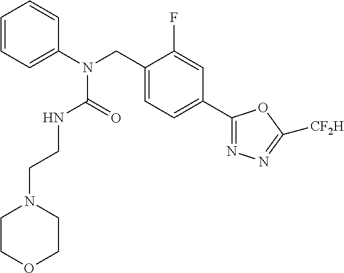 [SMILES: O=C(NCCN1CCOCC1)N(Cc1ccc(-c2nnc(C(F)F)o2)cc1F)c1ccccc1] |
| 411 | 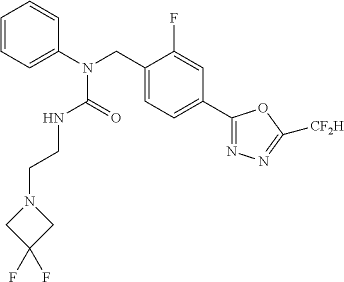 [SMILES: O=C(NCCN1CC(F)(F)C1)N(Cc1ccc(-c2nnc(C(F)F)o2)cc1F)c1ccccc1] |
| 412 | 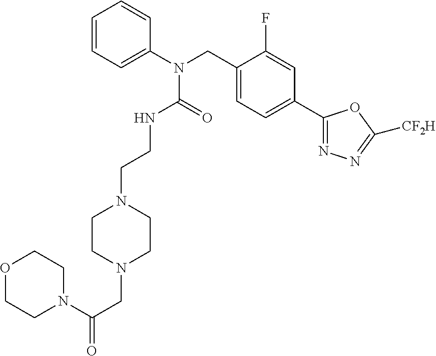 [SMILES: O=C(CN1CCN(CCNC(=O)N(Cc2ccc(-c3nnc(C(F)F)o3)cc2F)c2ccccc2)CC1)N1CCOCC1] |
| 413 | 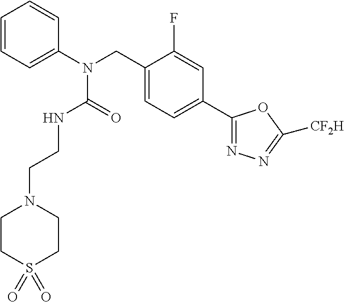 [SMILES: O=C(NCCN1CCS(=O)(=O)CC1)N(Cc1ccc(-c2nnc(C(F)F)o2)cc1F)c1ccccc1] |
| 414 | 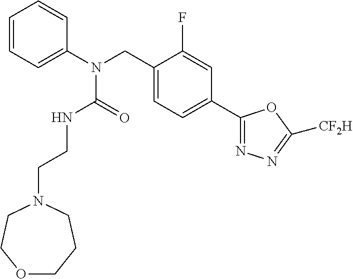 [SMILES: O=C(NCCN1CCCOCC1)N(Cc1ccc(-c2nnc(C(F)F)o2)cc1F)c1ccccc1] |
| 415 | 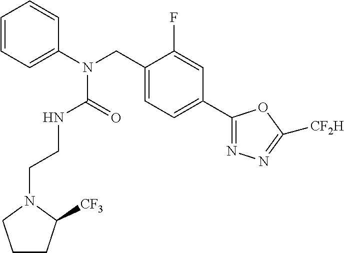 [SMILES: O=C(NCCN1CCC[C@@H]1C(F)(F)F)N(Cc1ccc(-c2nnc(C(F)F)o2)cc1F)c1ccccc1] |
| 416 | 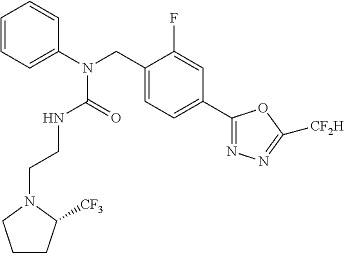 [SMILES: O=C(NCCN1CCC[C@H]1C(F)(F)F)N(Cc1ccc(-c2nnc(C(F)F)o2)cc1F)c1ccccc1] |
| 417 | 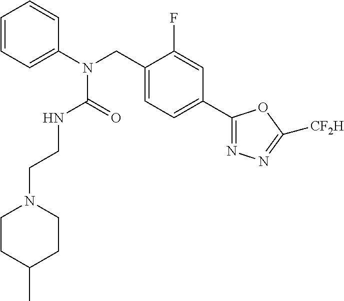 [SMILES: CC1CCN(CCNC(=O)N(Cc2ccc(-c3nnc(C(F)F)o3)cc2F)c2ccccc2)CC1] |
| 418 | 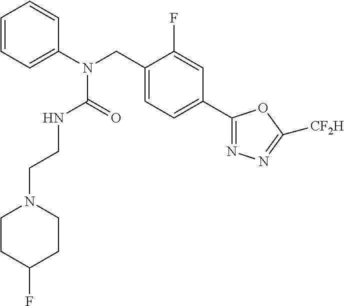 [SMILES: O=C(NCCN1CCC(F)CC1)N(Cc1ccc(-c2nnc(C(F)F)o2)cc1F)c1ccccc1] |
| 419 | 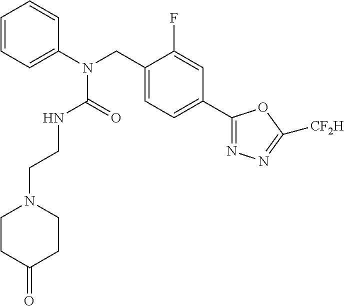 [SMILES: O=C1CCN(CCNC(=O)N(Cc2ccc(-c3nnc(C(F)F)o3)cc2F)c2ccccc2)CC1] |
| 420 | 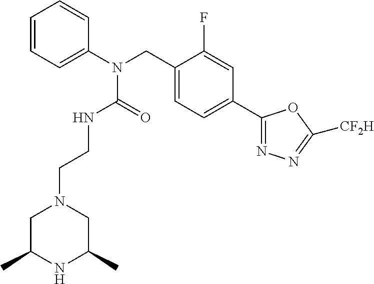 [SMILES: C[C@@H]1CN(CCNC(=O)N(Cc2ccc(-c3nnc(C(F)F)o3)cc2F)c2ccccc2)C[C@H](C)N1] |
| 421 | 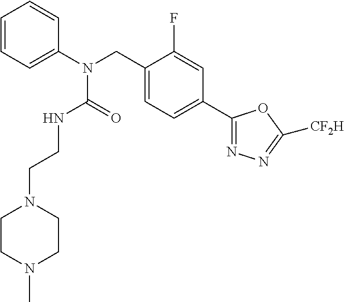 [SMILES: CN1CCN(CCNC(=O)N(Cc2ccc(-c3nnc(C(F)F)o3)cc2F)c2ccccc2)CC1] |
| 422 | 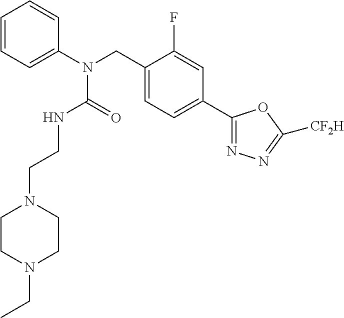 [SMILES: CCN1CCN(CCNC(=O)N(Cc2ccc(-c3nnc(C(F)F)o3)cc2F)c2ccccc2)CC1] |
| 423 | 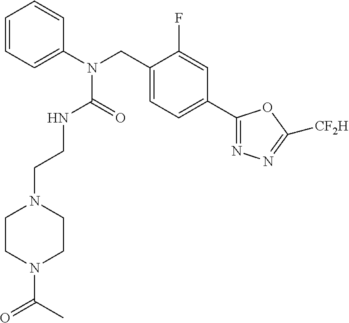 [SMILES: CC(=O)N1CCN(CCNC(=O)N(Cc2ccc(-c3nnc(C(F)F)o3)cc2F)c2ccccc2)CC1] |
| 424 | 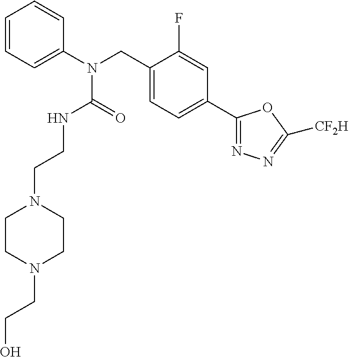 [SMILES: O=C(NCCN1CCN(CCO)CC1)N(Cc1ccc(-c2nnc(C(F)F)o2)cc1F)c1ccccc1] |
| 425 | 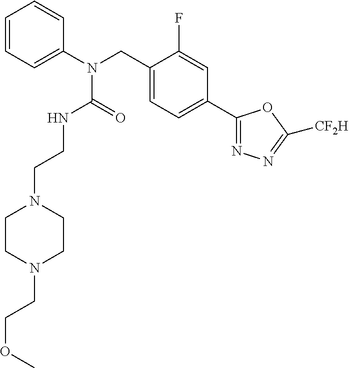 [SMILES: COCCN1CCN(CCNC(=O)N(Cc2ccc(-c3nnc(C(F)F)o3)cc2F)c2ccccc2)CC1] |
| 426 | 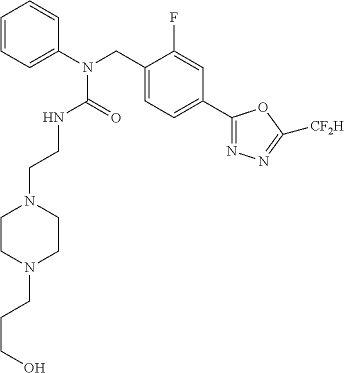 [SMILES: O=C(NCCN1CCN(CCCO)CC1)N(Cc1ccc(-c2nnc(C(F)F)o2)cc1F)c1ccccc1] |
| 427 | 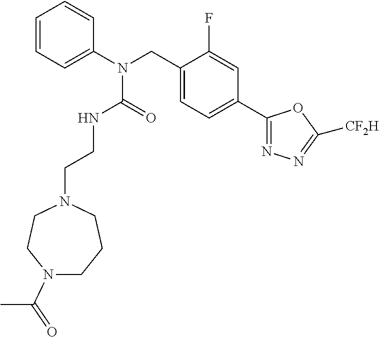 [SMILES: CC(=O)N1CCCN(CCNC(=O)N(Cc2ccc(-c3nnc(C(F)F)o3)cc2F)c2ccccc2)CC1] |
| 428 | 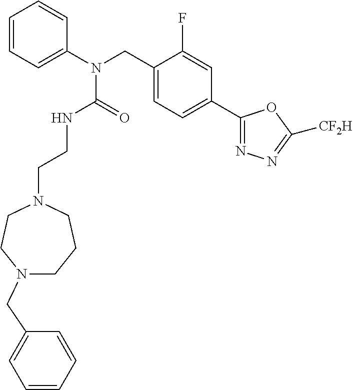 [SMILES: O=C(NCCN1CCCN(Cc2ccccc2)CC1)N(Cc1ccc(-c2nnc(C(F)F)o2)cc1F)c1ccccc1] |
| 429 | 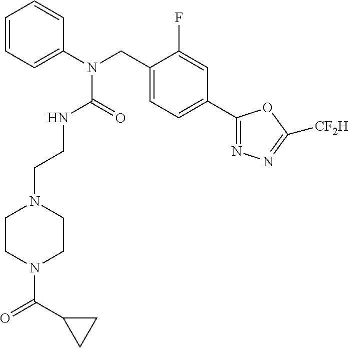 [SMILES: O=C(C1CC1)N1CCN(CCNC(=O)N(Cc2ccc(-c3nnc(C(F)F)o3)cc2F)c2ccccc2)CC1] |
| 430 | 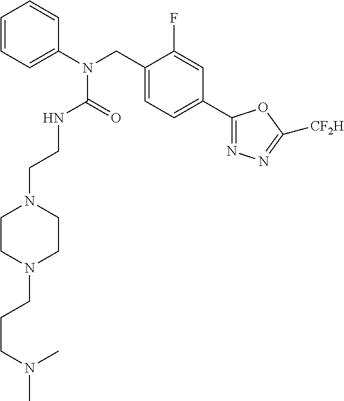 [SMILES: CN(C)CCCN1CCN(CCNC(=O)N(Cc2ccc(-c3nnc(C(F)F)o3)cc2F)c2ccccc2)CC1] |
| 431 | 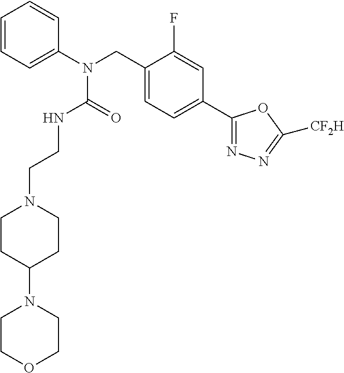 [SMILES: O=C(NCCN1CCC(N2CCOCC2)CC1)N(Cc1ccc(-c2nnc(C(F)F)o2)cc1F)c1ccccc1] |
| 432 | 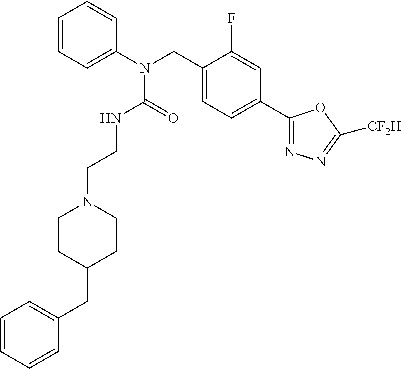 [SMILES: O=C(NCCN1CCC(Cc2ccccc2)CC1)N(Cc1ccc(-c2nnc(C(F)F)o2)cc1F)c1ccccc1] |
| 433 | 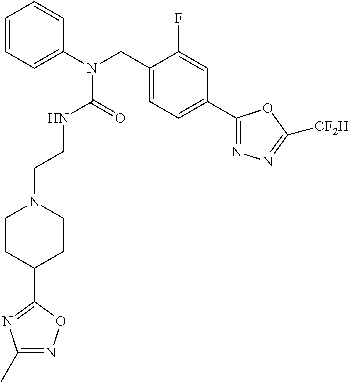 [SMILES: Cc1noc(C2CCN(CCNC(=O)N(Cc3ccc(-c4nnc(C(F)F)o4)cc3F)c3ccccc3)CC2)n1] |
| 434 | 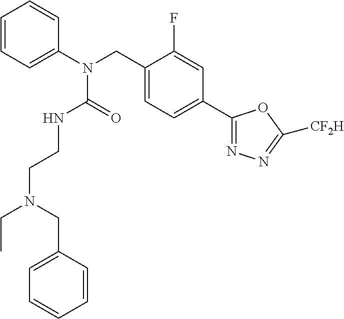 [SMILES: CCN(CCNC(=O)N(Cc1ccc(-c2nnc(C(F)F)o2)cc1F)c1ccccc1)Cc1ccccc1] |
| 435 | 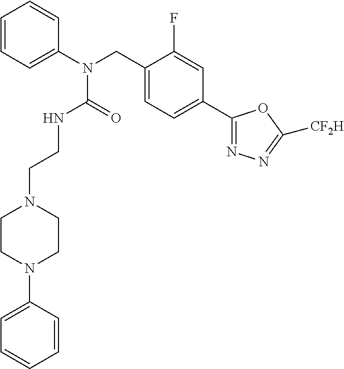 [SMILES: O=C(NCCN1CCN(c2ccccc2)CC1)N(Cc1ccc(-c2nnc(C(F)F)o2)cc1F)c1ccccc1] |
| 436 | 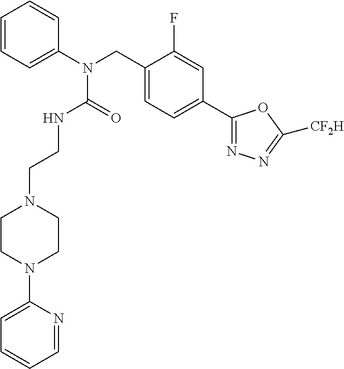 [SMILES: O=C(NCCN1CCN(c2ccccn2)CC1)N(Cc1ccc(-c2nnc(C(F)F)o2)cc1F)c1ccccc1] |
| 437 | 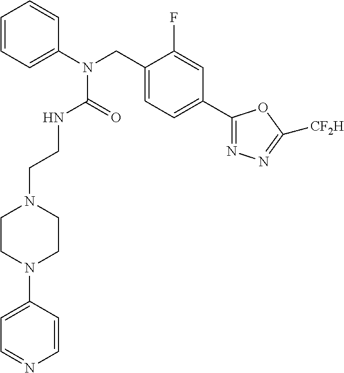 [SMILES: O=C(NCCN1CCN(c2ccncc2)CC1)N(Cc1ccc(-c2nnc(C(F)F)o2)cc1F)c1ccccc1] |
| 438 | 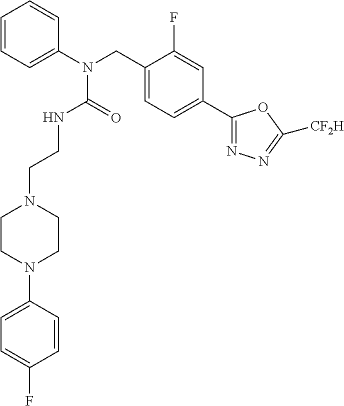 [SMILES: O=C(NCCN1CCN(c2ccc(F)cc2)CC1)N(Cc1ccc(-c2nnc(C(F)F)o2)cc1F)c1ccccc1] |
| 439 | 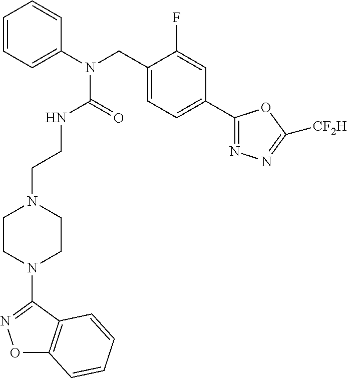 [SMILES: O=C(NCCN1CCN(c2noc3ccccc23)CC1)N(Cc1ccc(-c2nnc(C(F)F)o2)cc1F)c1ccccc1] |
| 440 | 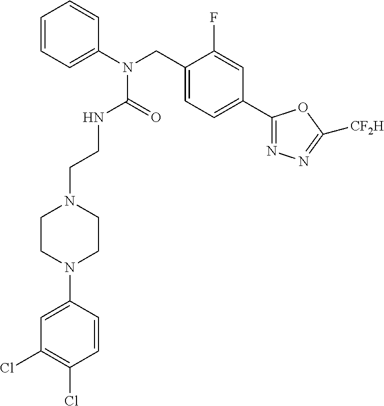 [SMILES: O=C(NCCN1CCN(c2ccc(Cl)c(Cl)c2)CC1)N(Cc1ccc(-c2nnc(C(F)F)o2)cc1F)c1ccccc1] |
| 441 | 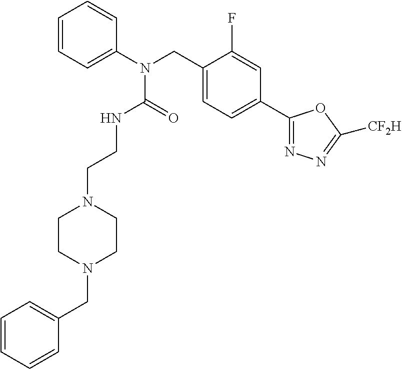 [SMILES: O=C(NCCN1CCN(Cc2ccccc2)CC1)N(Cc1ccc(-c2nnc(C(F)F)o2)cc1F)c1ccccc1] |
| 442 | 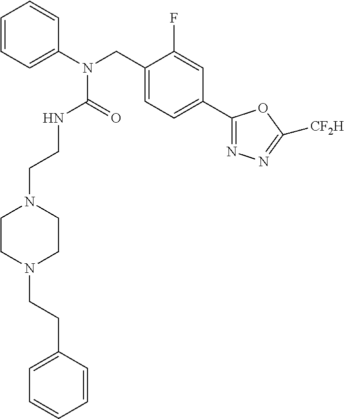 [SMILES: O=C(NCCN1CCN(CCc2ccccc2)CC1)N(Cc1ccc(-c2nnc(C(F)F)o2)cc1F)c1ccccc1] |
| 443 | 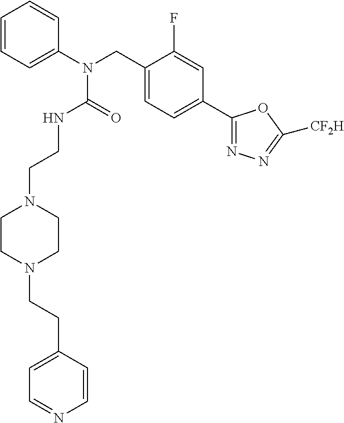 [SMILES: O=C(NCCN1CCN(CCc2ccncc2)CC1)N(Cc1ccc(-c2nnc(C(F)F)o2)cc1F)c1ccccc1] |
| 444 | 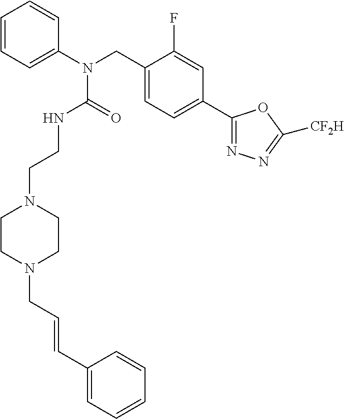 [SMILES: O=C(NCCN1CCN(C/C=C/c2ccccc2)CC1)N(Cc1ccc(-c2nnc(C(F)F)o2)cc1F)c1ccccc1] |
| 445 | 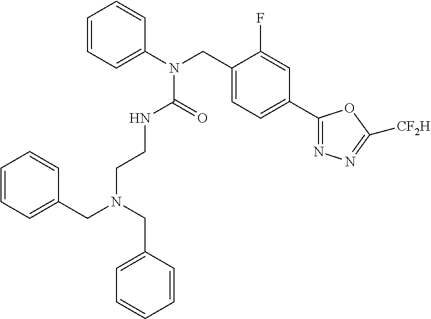 [SMILES: O=C(NCCN(Cc1ccccc1)Cc1ccccc1)N(Cc1ccc(-c2nnc(C(F)F)o2)cc1F)c1ccccc1] |
| 446 | 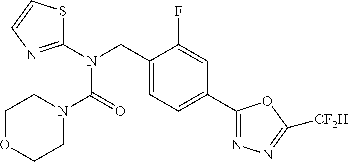 [SMILES: O=C(N1CCOCC1)N(Cc1ccc(-c2nnc(C(F)F)o2)cc1F)c1nccs1] |
| 447 | 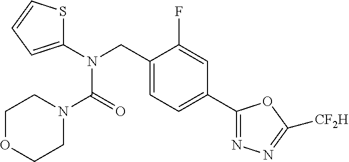 [SMILES: O=C(N1CCOCC1)N(Cc1ccc(-c2nnc(C(F)F)o2)cc1F)c1cccs1] |
| 448 | 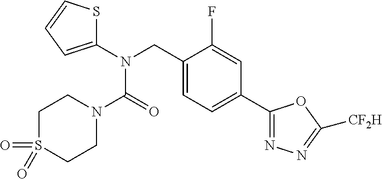 [SMILES: O=C(N1CCS(=O)(=O)CC1)N(Cc1ccc(-c2nnc(C(F)F)o2)cc1F)c1cccs1] |
| 449 | 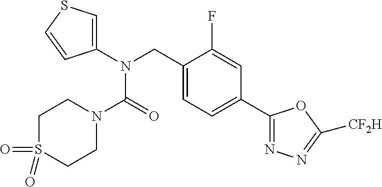 [SMILES: O=C(N1CCS(=O)(=O)CC1)N(Cc1ccc(-c2nnc(C(F)F)o2)cc1F)c1ccsc1] |
| 450 | 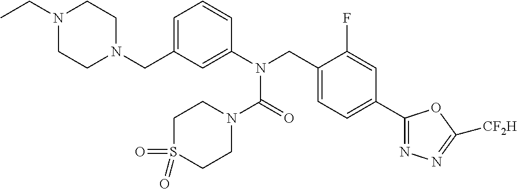 [SMILES: CCN1CCN(Cc2cccc(N(Cc3ccc(-c4nnc(C(F)F)o4)cc3F)C(=O)N3CCS(=O)(=O)CC3)c2)CC1] |

6. A pharmaceutical composition for preventing or treating Charcot-Marie-Tooth (CMT) disease associated with peripheral nervous systems (PNS), comprising a compound, optical isomers thereof or pharmaceutically acceptable salts thereof as an active ingredient, wherein the compound has the following structure:

| Com- | |
|---|---|
| pound | Structure |
| 43 | 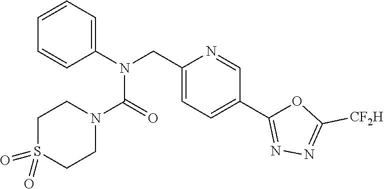 [SMILES: O=C(N1CCS(=O)(=O)CC1)N(Cc1ccc(-c2nnc(C(F)F)o2)cn1)c1ccccc1] |
| 232 | 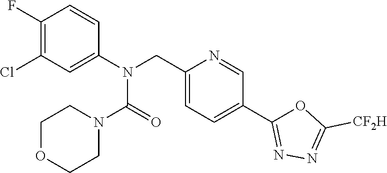 [SMILES: O=C(N1CCOCC1)N(Cc1ccc(-c2nnc(C(F)F)o2)cn1)c1ccc(F)c(Cl)c1] |
| 239 | 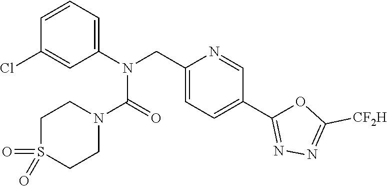 [SMILES: O=C(N1CCS(=O)(=O)CC1)N(Cc1ccc(-c2nnc(C(F)F)o2)cn1)c1cccc(Cl)c1] |
| 243 | 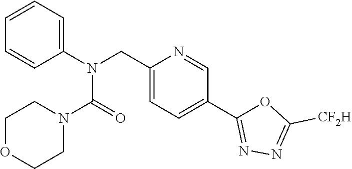 [SMILES: O=C(N1CCOCC1)N(Cc1ccc(-c2nnc(C(F)F)o2)cn1)c1ccccc1] |
| 286 | 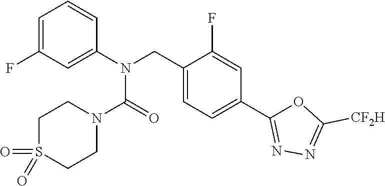 [SMILES: O=C(N1CCS(=O)(=O)CC1)N(Cc1ccc(-c2nnc(C(F)F)o2)cc1F)c1cccc(F)c1] |

7. The pharmaceutical composition of claim 1, wherein the Charcot-Marie-Tooth disease associated with the peripheral nervous system is at least one selected from the group consisting of CMT1 type, CMT2 type, CMT4 type, CMTX, degerine-sottas syndrome (DSN), congenital hypomyelination (CH), hereditary neuropathy with liability to pressure palsy (HNPP) and giant axonal neuropathy (GAN).

8. The pharmaceutical composition of claim 1, wherein the Charcot-Marie-Tooth disease associated with the peripheral nervous system PNS is at least one selected from the group consisting of CMT1A, CMT2D and CMT2F.

9. The pharmaceutical composition of claim 1, wherein the pharmaceutical composition is orally administered.

10. A method for preventing or treating Charcot-Marie-Tooth disease associated with the peripheral nervous system, including administering a compound represented by formula I, optical isomers thereof or pharmaceutically acceptable salts thereof into an individual, wherein the formula I is the same as in claim 1.

11. A method for preventing or treating Charcot-Marie-Tooth disease associated with the peripheral nervous system, including administering a compound, optical isomers thereof or pharmaceutically acceptable salts thereof into an individual, wherein the compound has the following structure:

| Com- | |
|---|---|
| pound | Structure |
| 43 | 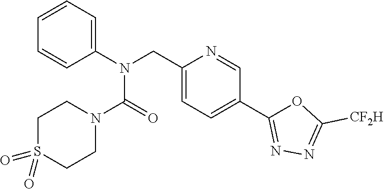 [SMILES: O=C(N1CCS(=O)(=O)CC1)N(Cc1ccc(-c2nnc(C(F)F)o2)cn1)c1ccccc1] |
| 232 | 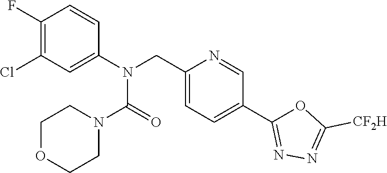 [SMILES: O=C(N1CCOCC1)N(Cc1ccc(-c2nnc(C(F)F)o2)cn1)c1ccc(F)c(Cl)c1] |
| 239 | 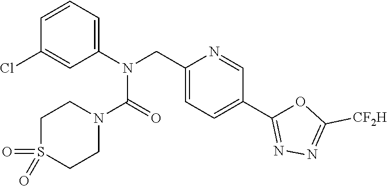 [SMILES: O=C(N1CCS(=O)(=O)CC1)N(Cc1ccc(-c2nnc(C(F)F)o2)cn1)c1cccc(Cl)c1] |
| 243 | 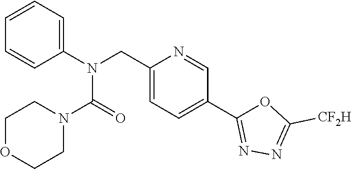 [SMILES: O=C(N1CCOCC1)N(Cc1ccc(-c2nnc(C(F)F)o2)cn1)c1ccccc1] |
| 286 | 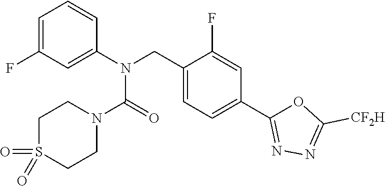 [SMILES: O=C(N1CCS(=O)(=O)CC1)N(Cc1ccc(-c2nnc(C(F)F)o2)cc1F)c1cccc(F)c1] |

12. A use of a compound represented by the above formula I, optical isomers thereof or pharmaceutically acceptable salts thereof for preventing or treating Charcot-Marie-Tooth disease associated with the peripheral nervous system, wherein the formula I is the same as in claim 1.

13. A use of a compound, optical isomers thereof or pharmaceutically acceptable salts thereof for preventing or treating Charcot-Marie-Tooth disease associated with the peripheral nervous system, wherein the compound has the following structure:

| Com- | |
|---|---|
| pound | Structure |
| 43 | 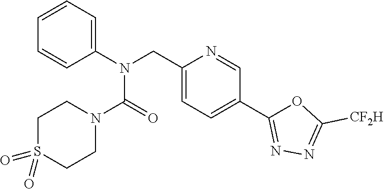 [SMILES: O=C(N1CCS(=O)(=O)CC1)N(Cc1ccc(-c2nnc(C(F)F)o2)cn1)c1ccccc1] |
| 232 | 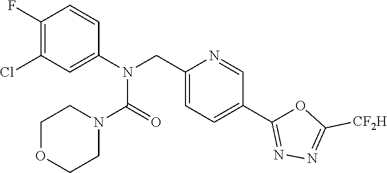 [SMILES: O=C(N1CCOCC1)N(Cc1ccc(-c2nnc(C(F)F)o2)cn1)c1ccc(F)c(Cl)c1] |
| 239 | 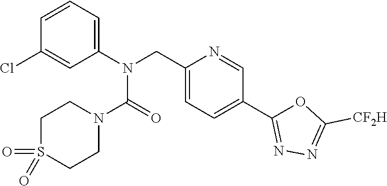 [SMILES: O=C(N1CCS(=O)(=O)CC1)N(Cc1ccc(-c2nnc(C(F)F)o2)cn1)c1cccc(Cl)c1] |
| 243 | 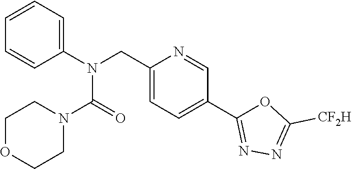 [SMILES: O=C(N1CCOCC1)N(Cc1ccc(-c2nnc(C(F)F)o2)cn1)c1ccccc1] |
| 286 | 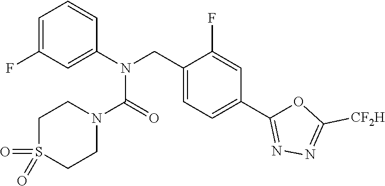 [SMILES: O=C(N1CCS(=O)(=O)CC1)N(Cc1ccc(-c2nnc(C(F)F)o2)cc1F)c1cccc(F)c1] |

14. A use of the compound represented by above formula I, optical isomers thereof or pharmaceutically acceptable salts thereof in preparing a medicament for preventing or treating Charcot-Marie-Tooth disease associated with the peripheral nervous system, wherein the formula I is the same as in claim 1.

15. A use of a compound, optical isomers thereof or pharmaceutically acceptable salts thereof in preparing a medicament for preventing or treating Charcot-Marie-Tooth disease associated with the peripheral nervous system, wherein the compound has the following structure:

| Com- | |
|---|---|
| pound | Structure |
| 43 | 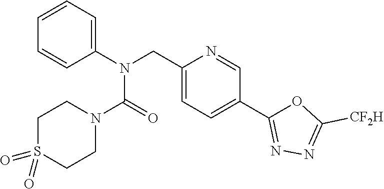 [SMILES: O=C(N1CCS(=O)(=O)CC1)N(Cc1ccc(-c2nnc(C(F)F)o2)cn1)c1ccccc1] |
| 232 | 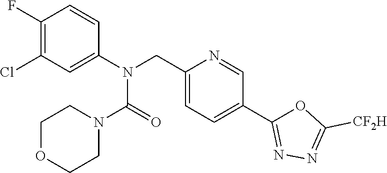 [SMILES: O=C(N1CCOCC1)N(Cc1ccc(-c2nnc(C(F)F)o2)cn1)c1ccc(F)c(Cl)c1] |
| 239 | 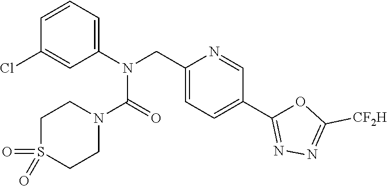 [SMILES: O=C(N1CCS(=O)(=O)CC1)N(Cc1ccc(-c2nnc(C(F)F)o2)cn1)c1cccc(Cl)c1] |
| 243 | 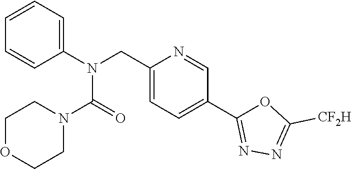 [SMILES: O=C(N1CCOCC1)N(Cc1ccc(-c2nnc(C(F)F)o2)cn1)c1ccccc1] |
| 286 | 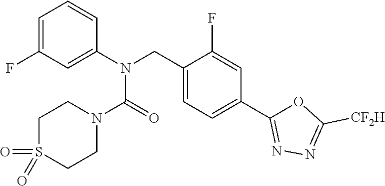 [SMILES: O=C(N1CCS(=O)(=O)CC1)N(Cc1ccc(-c2nnc(C(F)F)o2)cc1F)c1cccc(F)c1] |
